SEQUENCE LISTING (136 sequences)

SEQ ID NO: 1 - DNA, 1040 bp, Trichoderma reesei
  source 1..1040: mol_type = other DNA
atgatggccgacacactcgtccctgaacatcccgatctgtctggctttcctgactccccgggccagaaacgaaagcgcgaactcgaaagccccaaccttgggcgcgcaaagcgtgcagcccctcctgcgaccatgtcagcgtcaccgggggacacagccgccttcatggataatgccatggagtcgcaggcggccgcggtgagcggcgtcaatgttgccgacttcaacgcccttcagcaagccgccgctgccgaccacaccgacgccacagacccttccaacgcgaccagcacggcccaggcggccctgggcatgtacccgacccttcatgtgccgccgtcgaccgaggagcagttctctgcgcagggtcccgtcgagcccgaccacggccacgaaaacgcgttcaagcatgacacgcccttcaccacagacgtcacacagcagcccgatccgatgatggacccgtccgtcaaccagaacgcgccgccgcctccgccgaatggtgtcccgactcaccctcctccaccgccgcagcacagatacgcagtgccgccgccgcccaaccccaaaccaagcgttggctcagaggagtggcataagatgcgcaaggacaaccacaaagaaggtaagatggaggactcgcctcgcctcgccaggtaggcatccgtgggattgagtttggcattaacgcgaaagcagtggagcgtcggcgtcgcgagaccatcaacgaaggcatcaacgagctggccaagatcgtgccgggatgcgaaaagaacaagggatcgatcctgcagcgggctgttagcttcatcacccagctcaaggagaacgagcaacagaatatcgaaaagtggacgctggaaaagctcctgaccgagcaagccattacggagctgagcgcgtccaacgacaagctcaagcaggagtgcgagcgactctaccgagaactcgagacgtggaagcgagttgcgcagcaggccggtctggagcccccggccgcgcccaaggaagagcccaatgctggcatgccatcgagttag

SEQ ID NO: 2 - protein, 321 aa, Trichoderma reesei
MMADTLVPEHPDLSGFPDSPGQKRKRELESPNLGRAKRAAPPATMSASPGDTAAFMDNAMESQAAAVSGVNVADFNALQQAAAADHTDATDPSNATSTAQAALGMYPTLHVPPSTEEQFSAQGPVEPDHGHENAFKHDTPFTTDVTQQPDPMMDPSVNQNAPPPPPNGVPTHPPPPPQHRYAVPPPPNPKPSVGSEEWHKMRKDNHKEVERRRRETINEGINELAKIVPGCEKNKGSILQRAVSFITQLKENEQQNIEKWTLEKLLTEQAITELSASNDKLKQECERLYRELETWKRVAQQAGLEPPAAPKEEPNAGMPSS

SEQ ID NO: 3 - protein, 52 aa, Trichoderma reesei
KDNHKEVERRRRETINEGINELAKIVPGCEKNKGSILQRAVSFITQLKENEQ

SEQ ID NO: 4 - DNA, 1397 bp, Trichoderma reesei
  source 1..1397: mol_type = other DNA
atgatctggtcggcaagcggagcagacgacaaccttgtcaacgacattcataccagaattttcaggccatcatcccgctctgcgcgccctcagactaataatccaagcatcgatagccggcgcagctgcaattcgtcgcaggccgaggacctcacaagccgacggccagctgccgctcccttccccagaggccgcacctcgaccgctgccgctgcttccgcctcgcctcgacagccagcttctcccgctctccggcgcatcgcgcagctcaaggccttgtatcggcgtcacccaaaactgcagctggggtacatatccgacctgcaggccaccatgagcgtcgcatacgagccgcggtcctttatccacgagggcgcatacccctccatgggcgaggagcacgaccatgttgccgagcagcgatttaccgattccgatgttgccgagcacctcagccattataccgcagaggcatcgctgttgccggacgacagaggcgtcatgggcgatgagagggacatgctggcagatgacgatgccgtggtgccggaagcgtctcagctcgacctgggcgcgcccgagttctcatcgcctctaccagtggaactacatgcgcctccgctaccagagcccataccagaaagcaaggaacctctaccgacagacatatcctccccgacactacgcttgaagcagatccccaagccagagcgtgaagttcacaagaagtcagatggaaaattccactgcccactggatgactgcaaagaggaagtccgcgcattctctcgcaagtgcgagtggaagtaagtcatcccccccttactcgtcttgccttgcgcccaagccaacacttccacagcaagcacatggacaagcacgaacgcccctatcgctgcccagccgagggatgcgagaatttgcccggtttcacctactcgggcgggcttctccgacacgagcgagaggttcatgggaagcacggcgggcccaagaagaccgtcaactgcccgcatcccaactgcaagcggcacactggcaagggcttctcgcgacaggagaatcttaacgagcatcttcggcgagtgcacacaaatcccgaggccacttccgcatctccaccgcccgaggccgtcgcgactcccgaggacaacgacagcgacaagtctgggacgaagcgcaagcgccgcatgagcagccagggcgtcgacgagctgtctgatctccgagacgaaatcaagcgagtgcgcgaggaaaacgaaaagctcaagtccgagatggaccagcagtcgcagcactccctggccatgatggcgcaaattgccgagctgcaggacgcgttgcgtcacagcctcaatcctcatgcgctcggggcgccaacggctcagatgatctag

SEQ ID NO: 5 - protein, 445 aa, Trichoderma reesei
MIWSASGADDNLVNDIHTRIFRPSSRSARPQTNNPSIDSRRSCNSSQAEDLTSRRPAAAPFPRGRTSTAAAASASPRQPASPALRRIAQLKALYRRHPKLQLGYISDLQATMSVAYEPRSFIHEGAYPSMGEEHDHVAEQRFTDSDVAEHLSHYTAEASLLPDDRGVMGDERDMLADDDAVVPEASQLDLGAPEFSSPLPVELHAPPLPEPIPESKEPLPTDISSPTLRKQIPKPEREVHKKSDGKFHCPLDDCKEEVRAFSRKCEWNKHMDKHERPYRCPAEGCENLPGFTYSGGLLRHEREVHGKHGGPKKTVNCPHPNCKRHTGKGFSRQENLNEHLRRVHTNPEATSASPPPEAVATPEDNDSDKSGTKRKRRMSSQGVDELSDLRDEIKRVREENEKLKSEMDQQSQHSLAMMAQIAELQDALRHSLNPHALGAPTAQMI

SEQ ID NO: 6 - protein, 91 aa, Trichoderma reesei
CKEEVRAFSRKCEWNKHMDKHERPYRCPAEGCENLPGFTYSGGLLRHEREVHGKHGGPKKTVNCPHPNCKRHTGKGFSRQENLNEHLRRVH

SEQ ID NO: 7 - DNA, 2626 bp, Trichoderma reesei
  source 1..2626: mol_type = other DNA
atgtcgacggctaaagcgaccaagaagagcgcgttttcctgcgagccatgtcgccgccgcaaggtaagcctggctgcttggagatcgtgttattggcgtgtctctggctttgctgggagggatggagagagtgtaagagaggcatcttggagctgactctgctggtggcgttaggtcaagtgcggcggcgagcagcccatgtgtcaacgctgcgttgctcgcaacgatgattgcgtctacaaattgtgtgacttgatatctcactccgggcacatctacatctgcctagtagcgtctgatcttggcggctgacacccatctgcaggaacccgacgctgtcctacacgcaacggctggaggatcgcatcaaagagctggaggagcagcttgcacaagtcacggctgcaacaccgccaccggcggccattggcagcaaatcccctccatcaagtcatccaagccctacagcctcggggagcggtcagcaagacacccggcagcagatcgatgacagcatctcccggagcttccgcggcctcaagatagaccaaaagggcggcgtcacctaccatgggaccacgagcttcttccatctccccagtgaccgtagctctgcggcactaactgccgacatgcacgcagccgcgactgacattgaaactcagcggagagagcgactagtttcgaacgcgtggcagcagcgcttcttggaggagactgctggggttcccgtacgtgtccaagtgtggtggtcgtcgcttgacagctcaagtctcatgccttaactctctcgcaggaaccctttcagacgcttctgaatgttcactggtgttggatacagcccctcttcaacttcatctaccgaccagcatttacccgtacgaaacccttgtagcttgtcatgacttttcccgccccctttcttctcttccctgatatttaccaatcatgtaggcgatatgcagtccatggggccgtactactcacatacgcttctcaatgctgttctctcccactccatacgctgggcgaagagcgaccccaagacaaagcagattctcgatgagtcgtacgacggaggcgccgtctttggcaagcacgcacgcagcatgcttttcgacgagctcagcaggggagtatgcacgatcccgacggtccaaacactacttctcctaagcgcacaggaatgcagccttgggaacacaacccaagcctggacgtatagtggtctggcgtttcggctgattgaccacttggggatatgtgtggatggagatcgatatatcaactcggtccacttcaccgacgaagacttggaaatccggcatcgagtcttttggtcgtgctatttctgggacaagatcatcagtctgtatcttggtcgatcgccatcactcaagcacacgacggtctctccccctcagattatgtgtaagtctgattgtcgatactcatctcttgtcgttgtcgctcacattggcggtggttggttggtcgttgatgctgacggctgtttcttataagtcgacgactctgctgaaaacgagctgtgggttcccttcggggcgcctccactccaaaccaccccctggaagtacccacctgctactgctcattcggcttcttgctttctgagcatgtgccggctgtcagtcatcttcaacgagatattgattcatatgtacgacccgctcatgcagaacaccgagtccgaggtgctcgagtgcctgacgacccaggagccggccctgcagcaatggtgggatgagcttgctccatatctcaagctcgagcctatggctctgcccagcttggccccaccgtctcacatcgtcacgaccaagtaagttcttgccgtcccaagtgccgaacgccgttctccgttctccgttaaccggtccaaatagctgcctctatcacacgttcaaggtgctattgtaccgtccaatgctcacccggagaatactggagggcgatgttgcgtcgaaccagcgctacctcgtcgagtgcgtatcgtccgccacggccatcatcgctatttttgatctcttctgccgaacctttaccatgaactactgtgtgctgtcgctggcttacagcgtctacattgcttcgtcgatattcctgctacaggttcaggcggctccgggcgacggacaagctctagggcggctcaactactgtatgcaggccttgaagcaagtcaagactttcagcccgggtaagtcgagtattggcgcgactttgcttcttttgtttttctgcttgtcgttcggctcgatcctgacgttttgtagtcatcggcagcgctatcagcgcgctgaatagggaactctcggctgtcggcatctcgctggacgtgccccagcaccagcagccacccccaatccccgtcactgagtttcctccaatagccccaggatttccggccgtcgatgtgcccgtcgatgcgtcgtcttcgcagcctcagcctgctcatccgctgtttcaggttccctcaacccacagctatggtgcccattcaatggccatggacccaggcatctttgaggccatgtcgtccctggagccactcagcgtccgagttggggccctttcccctacggaaaatcaggactcgttttga

SEQ ID NO: 8 - protein, 686 aa, Trichoderma reesei
MSTAKATKKSAFSCEPCRRRKVKCGGEQPMCQRCVARNDDCVYKLNPTLSYTQRLEDRIKELEEQLAQVTAATPPPAAIGSKSPPSSHPSPTASGSGQQDTRQQIDDSISRSFRGLKIDQKGGVTYHGTTSFFHLPSDRSSAALTADMHAAATDIETQRRERLVSNAWQQRFLEETAGVPEPFQTLLNVHWCWIQPLFNFIYRPAFTRDMQSMGPYYSHTLLNAVLSHSIRWAKSDPKTKQILDESYDGGAVFGKHARSMLFDELSRGVCTIPTVQTLLLLSAQECSLGNTTQAWTYSGLAFRLIDHLGICVDGDRYINSVHFTDEDLEIRHRVFWSCYFWDKIISLYLGRSPSLKHTTVSPPQIMFDDSAENELWVPFGAPPLQTTPWKYPPATAHSASCFLSMCRLSVIFNEILIHMYDPLMQNTESEVLECLTTQEPALQQWWDELAPYLKLEPMALPSLAPPSHIVTTNCLYHTFKVLLYRPMLTRRILEGDVASNQRYLVECVSSATAIIAIFDLFCRTFTMNYCVLSLAYSVYIASSIFLLQVQAAPGDGQALGRLNYCMQALKQVKTFSPVIGSAISALNRELSAVGISLDVPQHQQPPPIPVTEFPPIAPGFPAVDVPVDASSSQPQPAHPLFQVPSTHSYGAHSMAMDPGIFEAMSSLEPLSVRVGALSPTENQDSF

SEQ ID NO: 9 - protein, 28 aa, Trichoderma reesei
CEPCRRRKVKCGGEQPMCQRCVARNDDC

SEQ ID NO: 10 - DNA, 2851 bp, Trichoderma reesei
  source 1..2851: mol_type = other DNA
atgccgggcatcctgcctatgaaagtcatcaaggtgggcaacagcgcccagagccgcatcgcccaggcctgcgaccgctgccgcagcaagaagattcgctgcgacggcgtccggccgacatgttcacaatgtgcctcagtcggcttcgagtgccgcaccagcgacaagctgagccggcgtgctttcccgcgcggatacacggagtcgctggaggagcgggtgcgcgcgctcgagagcgaggtccgcgagctcaaggacctgctagacgagaaggatgagaaaatcgaaatgttgtcgacgatgcactcaaacaatcacaacggcccacgacggaggccgtcagtcacatccatggccacgcccgtgtctccggatggcccaagggactcgcccaccatgaaggaggacacgttccgagtgcaggctacacaattgttgctgggcgtcgagaactcagaatcgtactttatgggttcttcaagcgggcgcgcctttatcggtatgtccatgcccttgatcctttgccacacatgacgttcccttcgcattctccagtgcacatcgtatagatgcacacgcatcgctctttgctccccaacgcccgtggcctcaaattctgacgtatctctctttatagcctccctcaagcgcaagatgcaagagagcggcaagccatgcagtgactttaccccagatgcctttctacacatccaaggctgcactccgctcgtagccaagctgccagaccacatctccagaatccctcctcgcatcttctcggacagatgcgtcaacgtcttcttccaggaatgggcacccctcttccctgtcctccacaagcccacctttctccgcatctatgaagagtttgtcaatgaccccgagagggtcaagtgcaactacaagctcgcacagctctacctcgtcttttcccttgccggcctctcgtccgagtctccagacattccgcaggtggctgcctgcgagtaccaatggacgagagcgctcgagtctctgatgcttgagaacaccatgagcacgttacagtgccttgtgttggctctgctgtactgcaccgttcgagccgactatcgtcgcctccagcactacaagtgcgtagccgtgggcctttctcaccggctgggtctgcaccagagccaaaagcgcttctcctttggcgccttgacgcttgagactaggaaaaaggtcttttggacgctgtacgccctggacaccatgacggctgccatcactggcctccccaagatgctcaaggaggatgacgtccaagcggaatatccatccgacactgatgacgagtacgtgacggagaagggattcctgcccacgctgccaggagagtttacccgcatctcgagcgccctggccctgttcaaggcaacccgcattctcgctcgtgtcttggacaagaactatcctgcgagtgccagctatgagctctcattgcagcagatgggcgcctttgaggccgagctggatggctggtaccaagacttgccagcccacttgaggctcaactttgcccagggcaagccgtcgcgagatgtcactggcagccggtcacctcttttggtaagtgaaagcatggtatggtttccaatatactgagcaaggggctaacacagacgggcgacagtcactcacttactactatatccgcaccctcatctaccgcccggccgttggatccaacctcggccccaaggctgcgccggcactcatttccatcagcgaatccagcaagcacattatccagattgcgcagctcctggaagagcggagcctgtccttttccttttgcatgaacaagtgcgatctgctggccgtgtgcgctcttactctcctctaccaggccattgacctgaagcgcgacagcaagctattgcgcgaggacgagcgcctggttaatgacgtccttcagacgctccgcaagaaccagtcgccggggtttgtggacctggaacgagccgccagcgtgctcatcaccgtcaccgacccgtcgtcgccatcgctgatgaacaagcacgtccgcaagtcgtcgatgacatcgggtccccgtcgctcatcgcctccgacgagcttccccaagcaaaactcgccgcaaatggccgggcagttcccggcatcggcgagcgagacggacttgacccgtcagcaggacaagcgctggatgggcaacgtggccgtccttcaaggccccaatgtaaatggccagcaggcgccgtcgcgccagtcgtttgatgggcaccggcaagagcgccagccgtcggtatcgcagatggctagagtttctcccagcgcccagccaaggcagaatctcgactatctgtcgctcaatgacgcttccttcacgaacaagcacgccacgtctccctccggcagcatgcagggacagcacatgggggtgcagaacggcggccagggctacatgaaccacggacagcagggtgccaaaacgcccaacatgtccagctccgactgggaagcgctggttggatccatggacagtgggatgaacaacgtgtacgacgtgatttacggaggcgccagcatcgaggctgccatggcgagtcactcgactggcagcgactggtcgccagacgcatttgacctaagcaacttcaacattggcgagttcggaagcaacccggcgccgccgcagagcgtgctgagcatgagcgacgagagcctcagctcgggggaggaagtttcgccttcagagctggggctgagcctgggaaacgttgactacggcaagcagctgcagggagtcaactaccacaacggcgacggattttcaacaaacgggcttcacgggctgtccttgtga

SEQ ID NO: 11 - protein, 882 aa, Trichoderma reesei
MPGILPMKVIKVGNSAQSRIAQACDRCRSKKIRCDGVRPTCSQCASVGFECRTSDKLSRRAFPRGYTESLEERVRALESEVRELKDLLDEKDEKIEMLSTMHSNNHNGPRRRPSVTSMATPVSPDGPRDSPTMKEDTFRVQATQLLLGVENSESYFMGSSSGRAFIASLKRKMQESGKPCSDFTPDAFLHIQGCTPLVAKLPDHISRIPPRIFSDRCVNVFFQEWAPLFPVLHKPTFLRIYEEFVNDPERVKCNYKLAQLYLVFSLAGLSSESPDIPQVAACEYQWTRALESLMLENTMSTLQCLVLALLYCTVRADYRRLQHYKCVAVGLSHRLGLHQSQKRFSFGALTLETRKKVFWTLYALDTMTAAITGLPKMLKEDDVQAEYPSDTDDEYVTEKGFLPTLPGEFTRISSALALFKATRILARVLDKNYPASASYELSLQQMGAFEAELDGWYQDLPAHLRLNFAQGKPSRDVTGSRSPLLSLTYYYIRTLIYRPAVGSNLGPKAAPALISISESSKHIIQIAQLLEERSLSFSFCMNKCDLLAVCALTLLYQAIDLKRDSKLLREDERLVNDVLQTLRKNQSPGFVDLERAASVLITVTDPSSPSLMNKHVRKSSMTSGPRRSSPPTSFPKQNSPQMAGQFPASASETDLTRQQDKRWMGNVAVLQGPNVNGQQAPSRQSFDGHRQERQPSVSQMARVSPSAQPRQNLDYLSLNDASFTNKHATSPSGSMQGQHMGVQNGGQGYMNHGQQGAKTPNMSSSDWEALVGSMDSGMNNVYDVIYGGASIEAAMASHSTGSDWSPDAFDLSNFNIGEFGSNPAPPQSVLSMSDESLSSGEEVSPSELGLSLGNVDYGKQLQGVNYHNGDGFSTNGLHGLSL

SEQ ID NO: 12 - protein, 28 aa, Trichoderma reesei
CDRCRSKKIRCDGVRPTCSQCASVGFEC

SEQ ID NO: 13 - DNA, 2093 bp, Trichoderma reesei
  source 1..2093: mol_type = other DNA
atgtcaacaacaacaacaaacaacaccacaatgccaccccccaccccgccaaagccgccgcagcaagtctgcgacaactgccgcttccgcaagatgaagtgcgaccgcggcctcccctgcagcaactgcgtcgtgtcgtccatcccgtgccgatacctccacacgctgcggcgcaaggggccccggggcggcaagggccggcggctgtcgcagatcaggcagggcctgacggagccggacaggaatcactttgaggtcagcacgccggggagcgcgcagcagatgcagtttggggtgctgggggagcaacagcaggcggcggcggcggcggcggcggcggcgagcaggaggagtaggaatggtggtggtgctgctgctggtggaggagctgctggaggaggaggacaaggaggaggacatggaggagcatcacagcagcagcagaatccgttgctggccctggcgacggcgacggagctcgacaggcagcggctgtccgtggcgctggcggcgcacgtccaggtcttcatgaagcacctcttccccatcatgcccgtcgtcaacgaccgggagctgctggccgactcgctgcggctggactcgctgtcgccctcgcggtacgccttcctgctgtcgctctgcgcggcgacgcgcatccagctcaagctcgacaacgccgaggagtacgacgagacggcggtgggcatcaacacgtcgctggactcgccgctcacgggcgaggccctgctgggcgccgcggagcaggcgaggctgcagttcaacgtggtggacgacttgagcctcgacacgctgctcacgtcctttttcctctttgcgggctatggcaacctggagaagcatacgcacgcttggttctacctgaaccaggccatttccctggccctgtcgattgggctgcatagcgaggctgcgtatagcaacctggaggagagcgagcgggagattcgacggcggatcttctggctgctctttgtgacggagaggtgagagaggttctcttttcacagcagatattttcacacaagagagagagggagagaggggacactgattctggttccgtaggacatacgccctgcagcaccgccggccggtgatgctgcgcaacacaatctccaagccgcaaatcgtcgactcggactgccccattgtcatgcacgactttgtcaaccacgtccgcctcttcgagctgctgccctgctcgctgtacgagtggcagccgcacacggacgggtgccagccgcagggcgtcgcgctgagccacgccatcagcaacaagctgtcggccgtgcagcccgccgagagcgtcatggagagccagcgcttcgacacgctcgtcacgcagcagtggctgcgcgtgtccatgtggcggctggcgtttggcagcaagcccagcctcgtctacgggcaggggctgatgcccgccttggggctgcccttcgaggcgggcaagtcgattatgaaggccctggggtctgtgagccagagctccaaggactgtcacgggattgccattgtacgtattatcgaacattgccagactctcgacaaaaacgtcgacaggggtgtttgctaactgaaatgtgtttgatgaatgtatataggagcagaagctgtttgacattggaatgagcctcgccgacgcttccatgtccgcctcgcccgtcccctcgttcgagttcggcccccacgacctgctctgctccgtcgtaaagttcctcggccgcatccgcggctgccagtccaacctgctgcccaagctgctcaagcactcggagctcatcctcggcttctccagccccctggtggccggcatcgacatccactggcccgcgctgctggaggagcatcccgccgccgccgccgacgtcggcgacctggctgctgctcatcatcatcatcatcatcacgctcagggggacgagtcctcggcgagcagcagcgcggggagctggcaggatacgtgcgacttggcgatgctggagcctggggcgggagcgtttatggggttggggtgcgaggaggcgtttgatgagcaggtgcacatgtctgggcaggtggagatggcatga

SEQ ID NO: 14 - protein, 640 aa, Trichoderma reesei
MSTTTTNNTTMPPPTPPKPPQQVCDNCRFRKMKCDRGLPCSNCVVSSIPCRYLHTLRRKGPRGGKGRRLSQIRQGLTEPDRNHFEVSTPGSAQQMQFGVLGEQQQAAAAAAAAASRRSRNGGGAAAGGGAAGGGGQGGGHGGASQQQQNPLLALATATELDRQRLSVALAAHVQVFMKHLFPIMPVVNDRELLADSLRLDSLSPSRYAFLLSLCAATRIQLKLDNAEEYDETAVGINTSLDSPLTGEALLGAAEQARLQFNVVDDLSLDTLLTSFFLFAGYGNLEKHTHAWFYLNQAISLALSIGLHSEAAYSNLEESEREIRRRIFWLLFVTERTYALQHRRPVMLRNTISKPQIVDSDCPIVMHDFVNHVRLFELLPCSLYEWQPHTDGCQPQGVALSHAISNKLSAVQPAESVMESQRFDTLVTQQWLRVSMWRLAFGSKPSLVYGQGLMPALGLPFEAGKSIMKALGSVSQSSKDCHGIAIEQKLFDIGMSLADASMSASPVPSFEFGPHDLLCSVVKFLGRIRGCQSNLLPKLLKHSELILGFSSPLVAGIDIHWPALLEEHPAAAADVGDLAAAHHHHHHHAQGDESSASSSAGSWQDTCDLAMLEPGAGAFMGLGCEEAFDEQVHMSGQVEMA

SEQ ID NO: 15 - protein, 27 aa, Trichoderma reesei
CDNCRFRKMKCDRGLPCSNCVVSSIPC

SEQ ID NO: 16 - DNA, 2257 bp, Trichoderma reesei
  source 1..2257: mol_type = other DNA
atggacatgaagacatcatcgagtaccccacgcatccgcaagaagaggattccggtaagcatatgccgtctgagcagcccaggggccgctgtcatgtcacgttgacctcggtagctcggcatgctccgttgctgatcgtgttggtggacagaaatcctgcaccgcatgtcgccgctccaaggtgagaggctgaactcgaagcctatagttcgtgagctcaaagttctgactgacagctaatcccaggctaaatgcgatggtaagagaccatgctcacggtaggtcttctatgtgggctgaatcggctgtgcctcgatcccagattaacggttacatccagatgcagcgacctgaagaagatctgcacttttattgatcctcccaaacaggctcatgagatgtatgcttcttgcgttaccgattccaatttgtcgatcgcacacgcaactgaccacgctcgcctcactcatgcagccggatagaggagcttgagcaacaggttgcggcgctcaaagataggcttcgagcggatgcattggcacaaagcactcaagcagagaccggcttccagcacaccagccagccaggcattggcgacaccccaatctcatactcacctcgggccacttttgtcagccccgatagagtcgcctcagcattctgcatacgttcatcacacacccaatcaccgaccaatacgacacttgccagaaaacacaccagatctcactttgagattgggtctgtgacattaccagattgcctggatgctggcctgctaagtctcgagcaagctcgtcactacttctccatcttcttccaaggatgtgaccactttgttccggtgctcgatcctcgctatgactcattcgaaagcattcgcgcaagaagcagtctcctgtttggggcaatatgtgcagttggctgccgcgttgtgacggggtctgaaacccagcaatggcatatgctcgatttccatgtcaagaggatgctgacttgcgccctggccagtccgtccatggcctcactcgagactattcaggcactgttggtccgctcgtgctatgcatcagagaggtctctccttgtcgcagctgcaacgcgcatggctatcgatttggactttgcaagttcctacgatgagatggtcaatcggtctgtggccccggcagcacggggagtatcctcggcaagtctggatcaagatacaatcaccttgatgcgaagagtgaggacatggctccatctgtttgtgttggggcaaatattgcatgtcgatgctgctgacctggcgaccttcaagtttgttggggatttacggagatgccgcattatactgaagagcccggccgcaactgagctcgatctgtccttgttctcccaagtcgagctcaatgctgtccgtggcaggatctatgactcgttgtctggcctcgtgggattcagcgatgaagacgcaatgatggacgtcgttcgcgaggcaaagatcgacattgctctatggtatgatgattgggagcacatcttcgagaagcaaagcgctcaagcgccatggcttggtgtcaacttgcgcgcacaaaaatgttggtcagagaacatggccttttgccgagtcctgcgggcgtctggtgtcgagaatgtggacttcatgtcgcccgctcaaaagtcagttgtcgccatggcaaaagatgccttggaggagcacctggatatcatgatcagagagccgaggctctatttacgcaatctgcgcttcgcaatggactttgtctgggcaaagtgcgcattttgctacttactgctgctcaaactttccattctgttgccggaaagtaaggggcgcagcagtcgagagcttgtagaacacggcaatatcctcctggccgagctaagtgaagccagcggagggaatcatagcggaagccggagcagcactggaaaacagtatcttcagcttctacaggtggggatcgggaagttcagctgtgccacccaggaagcccacgaagtacttgcgagcacggcaaatgacgacagttttgcaactgctcgacggacgcctacattggcaacacagaatcgaatggagctggactcgtttgttccggaacaattcgtctttgagtgggatttccctgggctgacgttgttttcctcatcggcgaccagggtttcgtggcttgatgatatccttgcagaggcactcaacgggagtgaggatgcatttgtttggttaccaacagacaatgacaactga

SEQ ID NO: 17 - protein, 652 aa, Trichoderma reesei
MDMKTSSSTPRIRKKRIPKSCTACRRSKAKCDGKRPCSRCSDLKKICTFIDPPKQAHEIRIEELEQQVAALKDRLRADALAQSTQAETGFQHTSQPGIGDTPISYSPRATFVSPDRVASAFCIRSSHTQSPTNTTLARKHTRSHFEIGSVTLPDCLDAGLLSLEQARHYFSIFFQGCDHFVPVLDPRYDSFESIRARSSLLFGAICAVGCRVVTGSETQQWHMLDFHVKRMLTCALASPSMASLETIQALLVRSCYASERSLLVAAATRMAIDLDFASSYDEMVNRSVAPAARGVSSASLDQDTITLMRRVRTWLHLFVLGQILHVDAADLATFKFVGDLRRCRIILKSPAATELDLSLFSQVELNAVRGRIYDSLSGLVGFSDEDAMMDVVREAKIDIALWYDDWEHIFEKQSAQAPWLGVNLRAQKCWSENMAFCRVLRASGVENVDFMSPAQKSVVAMAKDALEEHLDIMIREPRLYLRNLRFAMDFVWAKCAFCYLLLLKLSILLPESKGRSSRELVEHGNILLAELSEASGGNHSGSRSSTGKQYLQLLQVGIGKFSCATQEAHEVLASTANDDSFATARRTPTLATQNRMELDSFVPEQFVFEWDFPGLTLFSSSATRVSWLDDILAEALNGSEDAFVWLPTDNDN

SEQ ID NO: 18 - protein, 27 aa, Trichoderma reesei
CTACRRSKAKCDGKRPCSRCSDLKKIC

SEQ ID NO: 19 - DNA, 2248 bp, Trichoderma reesei
  source 1..2248: mol_type = other DNA
atgcttcagcataaggtatggtctatatggttgtgcatattgcctaccactgctgaagattgattcgatgcctcagatgccaaaagcgggagcgaacatgcacgagagtcagcagcggcagagcggcggcaggccgtacagatctcacctgcggccggcgtgtgtgccgtgccgccgcagaaagtcgcgatgccagaccgcaggcgatgctgccgtctgcctgacctgccgtgcccatcagaccgaatgctactttcccggcgactcgcgggaatctgcatcttcggaacctgcccgccgacgccgccgtgtggaagcccggccctcgtcttctgagccttccggagcgagccccctgagggttgcacaggccgcggcatcattccgcgagggctccaaggccaatacatctgcgcctgtggttgggccagtgccgaatattcctccgagacctatcgccggcatgtcgagaaccggtcaaacctatcacgaccccgaggaagagtcaacgctggcactgggctccgacgatgaccagcacctcaatctacacatagtaggacctgctgctacgaacgacagccaggtgttgtccacatatctctcgggtatccccggtgcaacccggagtacgcggatgattatcccggagcccgcaagctgttcgcggccagtgctgtttaccgaggttcagaagaggccgttcggcgtcaagttgaatcgtagcccttcagctgagaagctggagatcattgagaaactcctggagcctcataacgaggctgttattgacgagttggtcgtcctctctttgacatctttgccttctcacgtagctgactcttcttcttctgctgtagatacttcagaaaagtgaatgtgtgccttccgctgttggatgaagcctcattccgccgtcaataccaggaagataagacgcggatctcgccggcgctcatggcctgcctgtacgcgcatacgattgtgtattggcagagctcgcctattctgagccgctacaggtgcccagagagccgcttcatctggaaccttgccaacgaggcagtatattccgagctccatctatcacctggcatgtctatcatcaaagcgatcgtgctgaatgtaggcggacgccctactacctctctgataggcaacggggtgttactagggtctgcagtttcaatggcacattctctagggctgaatcacaatcctctcccgtggaagattccgcagcaggaaaagtatcttcgcatgaagatttggtgggcgctgttggtccatgacagatggtacgtgaatttcgcctgtgaacggccaagacggattagaagggactgaagaaaatgggaatggagagagaagccacaatgctgacttccagtcatgaatctgcaggacaagcctggcgcacggcacgccgcctcacatccagagatcacagtacgatgtgcctccgccgactctggagtatcttggcgagactctgacagagaccaacaacccacggtctgagctgagagccaacgtctttatctcgctggtaacgctgagcgaagtattgggccggtttttgcaatatgtgtactgcgttgggcgagataagccgacgaccaccgatctcgagcatgcattgcatcaatggattgagacgctggagggcccctgtcgtcggattgtcctccgcgggtctcatctggacgtcaccggggcagcaaaccttcgcctggcattcctgactgctaaactgcttctgcagcggatacagctcgaggccgagaagcaaggagactgcgtcaacgaagagggtgtaatgaaccgctattcggaagcacgcatgacttccgaggagattctcatgctcatccaggacctccagccggagaatctcggtgacttctggatgtccgtgagcgccttttccttcccggccgccgtcaactttttgctacgatgcgcgctggagacggagaattctcccgaaggcctcgcccagagcccctcgttcaaaatcgcccgcgatctcatcaccacgctccgctcgcaccaggagcagcacaggtgggatctgggcgacgtctgccttgcccagcacgccgagattgtagacaagatgctcacgggcgcggtgccggacgagcagggcggcaacaatagcagcttggacctgcaggactttgacgtatcgatcctggaccacatcttcccaagtgtatgggaccctttgcagaatgcttggtag

SEQ ID NO: 20 - protein, 671 aa, Trichoderma reesei
MLQHKMPKAGANMHESQQRQSGGRPYRSHLRPACVPCRRRKSRCQTAGDAAVCLTCRAHQTECYFPGDSRESASSEPARRRRRVEARPSSSEPSGASPLRVAQAAASFREGSKANTSAPVVGPVPNIPPRPIAGMSRTGQTYHDPEEESTLALGSDDDQHLNLHIVGPAATNDSQVLSTYLSGIPGATRSTRMIIPEPASCSRPVLFTEVQKRPFGVKLNRSPSAEKLEIIEKLLEPHNEAVIDEYFRKVNVCLPLLDEASFRRQYQEDKTRISPALMACLYAHTIVYWQSSPILSRYRCPESRFIWNLANEAVYSELHLSPGMSIIKAIVLNVGGRPTTSLIGNGVLLGSAVSMAHSLGLNHNPLPWKIPQQEKYLRMKIWWALLVHDRWTSLAHGTPPHIQRSQYDVPPPTLEYLGETLTETNNPRSELRANVFISLVTLSEVLGRFLQYVYCVGRDKPTTTDLEHALHQWIETLEGPCRRIVLRGSHLDVTGAANLRLAFLTAKLLLQRIQLEAEKQGDCVNEEGVMNRYSEARMTSEEILMLIQDLQPENLGDFWMSVSAFSFPAAVNFLLRCALETENSPEGLAQSPSFKIARDLITTLRSHQEQHRWDLGDVCLAQHAEIVDKMLTGAVPDEQGGNNSSLDLQDFDVSILDHIFPSVWDPLQNAW

SEQ ID NO: 21 - protein, 54 aa, Trichoderma reesei
VCLAQHAEIVDKMLTGAVPDEQGGNNSSLDLQDFDVSILDHIFPSVWDPLQNAW

SEQ ID NO: 22 - DNA, 3891 bp, Trichoderma reesei
  source 1..3891: mol_type = other DNA
atgacttcggccggcgcccacgaccagctcgacggccgtctcgacgatgccgcacccgcacccgcagcctcactctcactctcgcccatgcccaggcccaggcccgacccccagcccgagcccgagcctgcacctgcagcttccgcttccacgacgtcggcggcgcctctgtttgctgctgctgctgctgctgcttcggacgcagatgcggatgctgctgcctctgccgctgcgcaacgactgcccgaccaccccaaagccgcagctgcgtcgatgctgcccgcggccgcgcaccagccggcgcaagagcacccacggcagccggagctcgagctccagcaatcacagcccggaaacagaacagaagaagcatccgtgtcggttagcgacaaggcgacatcatccggtccgcagcctggctctcacgacggcacggcgcctcctgcagccgcaacagccgtcgcatcaacgtccacaccagcgccgccctcgtcgctgccagcagattctgctgcccccccacagcaggcgcgtgctttagaagagaacccaaaagcttcaatcgctgccgcaccctcgacgacgacgacaacaacaacaacaacgacgacgacgacgacagcgccgccgccgccgccgcctccaacctttaacggtgtagccaaggtcgagagtggcttagagcagtccgttgtaccaaacgcaaacacatctggagtcggagagtctgctgggctgaaagctacaattgagtcggacggacacgggggccaagacgttgtaatggcagacgctcctggagctaccacgaccgctgctgcgacggcgccgcacctatcgccttctgttgtgcaacctgcccaggtgacatcggcagaccaattcccagcctcaacaccgttcgcatcccagcctgctggcatcgaccaccgccacgcatacatgatgatggccctggcctccatgtctgctgcgccgcccgccatgtcgccgccgcccaccgtcaccccctctcagatgaccctgcccaaccctgccgaagaagggtactttggcctgggctctaacaacaacctgcgccagtctgaccccgacatgggcctcgagtcgtttgctcgtgtagagttcgccgacagcgtctttcagatgaccacctacgctgtcatcattggtcgtgaccagcgagctctggaacaggccaggcgggatggaaagcgtgccgaggagtatcaaaggcgtgtcgaagaaaatgcgagcaaaggccttccccccccatcccccattcctctgcctcaggaccgtcacaagtttagtaaatcgtacgtcagtgaggaaggtggcatgcttggcccagaatcagatagcgaagataatgggcgtccagcgaagcggcgcaagaccagcactacgggatcgtcgcagcagaatgaaaacgagaaccctcaagataacatcatatccaaccgccaatacgtttctcacactcctggcgctgctgcagtcgatctggcctcgctaaggccgtcaccctaccatgttcctttcatcggcattcactctcccggcccgaatatcgcgagcaaaaccaaggccatctcccgggagcatctcaagattgcctacaacccggacgagggtgtattcgaggcaatacccttacacaagaatggtttcttctgcgaggatgttcattacaagagcgaaaaagttgtactcaggtgcggcgatcgccttcagatcaaggacgttgggttcaggttcatcatcaacggcgtggaacgaggacgaaccggcgccgaagaatatcaggaagaggatgccaccaagaaacgccacgctcagggcggaaagagcatgagcttcgactttacacaatcacatggcaacggccagatccaggacacgagtgatgagctgtcggatgtggatgttagccccgcggaactgtccgactttggcggggacgatgagggtgagggagaagaagaggagaaggtcgcagaagagggcggtgaggaggaagaggaggaagaggaagaggaagaaggagacgatgaagacgaggaggaggccgatgccgacgtaccggaagcatccatcgagggcgactctgagatcaaggacgaaggggaagtgaatcacgacctctcgatgcccgagataccaaggaagaggggccctggcagaccgcccaagaatggcatcatgtcgaagagagagcagcgcttgcttaagaagcagcagcaagaaatggccaagaagacactgcctcaagcgccgccggttgagccgcctatcaagcgcaaagttggccggccgaggaaacacccgctgccggaagacggagtggaccgtcccgaaaagcgcaaatacaagccgcggaagcccaagaacgaagacggcgccgaaggctccgatgccgagagacgagcgagggagaagaaggagaagaaggcacggcccaagtctcctcctctggagctgaagattgaggactacaccgaggagcagctccagaagccgaacaagaactacggtgtgcttattgacgaaacattgacggcggctggccccgatggtctcacattgaagcagatctacaagagaatctgccaaagatacccgtggttttatttccacaccgagacgaaggggtgggagagtagtgtacgccataatctgattgggaacgaggcgtttaggaaggatgaaacgacgaacctgtggtctcgagtgcccggtgtcgagctggatgccggcaagaagcgaaaggcatcctccccagaccgaggactggcggccgcgcaggcctatgggcattactcgtatccctatgccgcccagcacatggctcctggagcgcatcctggctacccccccagccaggcacaggccccggcatatcaggctcccacgtacgcggcgcagccgccgagccatgcagcgcgtccagcgccgtacggcccatcacaatcaccccctgttcaccaaccggttcagccggccgtgggcgctccgcccccggctccagccccgatccagctaccgggatatggtcctccggcggccccggctcgccctcagcttggtgtgccccagcctggagcttacagctcgccctatgcctcgcggcctcctcccttggcaagcccggcagtcaaggccgaggacggcggcgccggcgtaccagctgcggctgccgttgggtctgtccagcagccaccccggccaagcatttcgcccgctgccgccgcggagccaaaggcggccacctcgggggcggcaccagcagcgcagaagacgactgtccaggctgccgctcacccgccgcaacctcgaccacaggcggcgcctgcgcggccgattatcgagccgcgtcttcttggcgcagtcgtcggcctcaaaaacggcctcgtggacaatctcaagaaggccggcaatcccaaggccgaggccattgtcatgtcggccctgaacaggtgcattgggctgaagaaagaagcgaccgaaaacgacaagatggaggccatctgcatcaaaggaatccggcaagtcattgacggcttcaccaagggcaagagtcccactcctggaagctccacctcattgcccactgatacgccgcctgttttcgagcccaaggtattggctgcgctgaatgggctcaaggatgcctctgtcaatgccatcaagtccggtctgggagaggccaaggctgaggctgtgacgttgtccgccattgatcgagtgattggccttgctgatgcgagcattatgcccaaggcggccgagggcgagccggcgagcaactttgagggggtcgagcagcatctcatgaagtcgatccggcagctcctccagggcatgaatcagaagttacagggcgattga

SEQ ID NO: 23 - protein, 1296 aa, Trichoderma reesei
MTSAGAHDQLDGRLDDAAPAPAASLSLSPMPRPRPDPQPEPEPAPAASASTTSAAPLFAAAAAAASDADADAAASAAAQRLPDHPKAAAASMLPAAAHQPAQEHPRQPELELQQSQPGNRTEEASVSVSDKATSSGPQPGSHDGTAPPAAATAVASTSTPAPPSSLPADSAAPPQQARALEENPKASIAAAPSTTTTTTTTTTTTTTAPPPPPPPTFNGVAKVESGLEQSVVPNANTSGVGESAGLKATIESDGHGGQDVVMADAPGATTTAAATAPHLSPSVVQPAQVTSADQFPASTPFASQPAGIDHRHAYMMMALASMSAAPPAMSPPPTVTPSQMTLPNPAEEGYFGLGSNNNLRQSDPDMGLESFARVEFADSVFQMTTYAVIIGRDQRALEQARRDGKRAEEYQRRVEENASKGLPPPSPIPLPQDRHKFSKSYVSEEGGMLGPESDSEDNGRPAKRRKTSTTGSSQQNENENPQDNIISNRQYVSHTPGAAAVDLASLRPSPYHVPFIGIHSPGPNIASKTKAISREHLKIAYNPDEGVFEAIPLHKNGFFCEDVHYKSEKVVLRCGDRLQIKDVGFRFIINGVERGRTGAEEYQEEDATKKRHAQGGKSMSFDFTQSHGNGQIQDTSDELSDVDVSPAELSDFGGDDEGEGEEEEKVAEEGGEEEEEEEEEEEGDDEDEEEADADVPEASIEGDSEIKDEGEVNHDLSMPEIPRKRGPGRPPKNGIMSKREQRLLKKQQQEMAKKTLPQAPPVEPPIKRKVGRPRKHPLPEDGVDRPEKRKYKPRKPKNEDGAEGSDAERRAREKKEKKARPKSPPLELKIEDYTEEQLQKPNKNYGVLIDETLTAAGPDGLTLKQIYKRICQRYPWFYFHTETKGWESSVRHNLIGNEAFRKDETTNLWSRVPGVELDAGKKRKASSPDRGLAAAQAYGHYSYPYAAQHMAPGAHPGYPPSQAQAPAYQAPTYAAQPPSHAARPAPYGPSQSPPVHQPVQPAVGAPPPAPAPIQLPGYGPPAAPARPQLGVPQPGAYSSPYASRPPPLASPAVKAEDGGAGVPAAAAVGSVQQPPRPSISPAAAAEPKAATSGAAPAAQKTTVQAAAHPPQPRPQAAPARPIIEPRLLGAVVGLKNGLVDNLKKAGNPKAEAIVMSALNRCIGLKKEATENDKMEAICIKGIRQVIDGFTKGKSPTPGSSTSLPTDTPPVFEPKVLAALNGLKDASVNAIKSGLGEAKAEAVTLSAIDRVIGLADASIMPKAAEGEPASNFEGVEQHLMKSIRQLLQGMNQKLQGD

SEQ ID NO: 24 - protein, 64 aa, Trichoderma reesei
QKPNKNYGVLIDETLTAAGPDGLTLKQIYKRICQRYPWFYFHTETKGWESSVRHNLIGNEAFRK

SEQ ID NO: 25 - DNA, 2021 bp, Trichoderma reesei
  source 1..2021: mol_type = other DNA
atgggtagaagaaagattgagatcaaggccatcgccgatgaccgcaaccgctccgtgtatgttgctgtttccacgccgagcccaataccgcgacggccccgagactaataccagtattccttaggaccttcctcaagagaaagggtggtctcttcaaaaaggcacacgagctctccgtcctatgttccgttgatgtcgccgtcttcatctttggcaacaacaagaagctgtacgagtactcttcaaaggacatgggacagctcattaccagataccaatacgtacgtttcaccgagggctgtgtcgtcgcacgacattgcttcctaccaaggggcatcgcttatatcaacctcggacgcgattttgctgacaagatggcatagcatggtggccccaacgaacacaagggcccttccgactacaacagcggccagaacaacgatgaagacgacgaagatggtgatgggacgcctcctcgaggcgcggatggaatggaggcgcacatgatgcctgcccattaccaaggccagcctcctcaacccttcccccagcttcgccatcagacagcctcggcgtcgccccccgtacccaacggcgtgccattcccaggtcatcctcacctccagcgaagcctcacgccgcaacccgccggcccgtcgcggccaaactcgaggggtgacatgcgccgcatgggccatggcatgatgccgcagcccgtcgggcctcatggacctcatgggccgcaccccggcatcacctacatgcccacgccacccatctataacccggcggctagcaaccagtccagcctcatgccgccccagcctggtccttacccgtaccagccgccacctcagccatacatggaagaacggaggccatctgcacctcctgccttcagtgctcatcctcctccccaaccactaacagtcggcccgcgcacagagaatccctcccctcagccacctcaccagttgctgcctcctcagtccatgccccctcatacctcgccgcagccagcacgtcgtcacctggacccccagcctccgccgccggtcgaaccaagggcagacgccccggagaagccgaaggcgcccctcatcaacacggacacggctatcaagaagatgtcccagcgcaagtcacacagcatctttacgccaatcgaggaaaaccggtccatcttatcccagcacttggcctcctttgcctcggaaccccagtctatcaagaacgagtccgcggccatggccgcagcaaaccccaaccgatcccagtcggctgacaacggacccgccgccgccgctcggaacggagaacctacgccgcatctgcagcaacgctccaatctgcgcaacacgtcggtgtcctcccttcctgatacgccgtcgcaggccagcagcctgaagatcaacacggccgtggggggtacaaggccgaagccgcctcgcctgactgtgcagattcccgacggtggctctgaggccgggagcgcaacgggcgactcaaactcacctcggattccgacagacgctgcgtccaacgcgccgcatcggcacaacagcgtggtcctcccgccgccgtctccgtctgccacgacacttttgtccgcaggggccaccggaccccccaatccgtttgcccggccggtgcctcagcagaacgtcaacggcgacacgcccgtttccgcacttccctctcggtttttgaacaatgagctgctgcccagccccagcagtttctatccggaatggaacttcagaggcagcgacaacaatacgctccccagccccctcaactttgccacgcccgtggtgggcacagggccaagcttcctccgagacgacagccatttgttgagcagcagcgggaacgcaggctccagcgcagcaagtggcaatcagggctcaacacaaccatcaggaattgccaagcggaagagtccagatctcgaggggcctggccaagacgagaaccaacagacggctagcgatcctaagcgcttgaaggtggaacgatag

SEQ ID NO: 26 - protein, 616 aa, Trichoderma reesei
MGRRKIEIKAIADDRNRSVTFLKRKGGLFKKAHELSVLCSVDVAVFIFGNNKKLYEYSSKDMGQLITRYQYHGGPNEHKGPSDYNSGQNNDEDDEDGDGTPPRGADGMEAHMMPAHYQGQPPQPFPQLRHQTASASPPVPNGVPFPGHPHLQRSLTPQPAGPSRPNSRGDMRRMGHGMMPQPVGPHGPHGPHPGITYMPTPPIYNPAASNQSSLMPPQPGPYPYQPPPQPYMEERRPSAPPAFSAHPPPQPLTVGPRTENPSPQPPHQLLPPQSMPPHTSPQPARRHLDPQPPPPVEPRADAPEKPKAPLINTDTAIKKMSQRKSHSIFTPIEENRSILSQHLASFASEPQSIKNESAAMAAANPNRSQSADNGPAAAARNGEPTPHLQQRSNLRNTSVSSLPDTPSQASSLKINTAVGGTRPKPPRLTVQIPDGGSEAGSATGDSNSPRIPTDAASNAPHRHNSVVLPPPSPSATTLLSAGATGPPNPFARPVPQQNVNGDTPVSALPSRFLNNELLPSPSSFYPEWNFRGSDNNTLPSPLNFATPVVGTGPSFLRDDSHLLSSSGNAGSSAASGNQGSTQPSGIAKRKSPDLEGPGQDENQQTASDPKRLKVER

SEQ ID NO: 27 - protein, 69 aa, Trichoderma reesei
GRRKIEIKAIADDRNRSVTFLKRKGGLFKKAHELSVLCSVDVAVFIFGNNKKLYEYSSKDMGQLITRYQ

SEQ ID NO: 28 - DNA, 2863 bp, Trichoderma reesei
  source 1..2863: mol_type = other DNA
atgctcagcggcgctgactcggcccagggccgcccccgcaagttcaccatccagagccagtttggccggcctcgacagccccggagccgcaagaatcggccctgcgacgcctgccggaagcggaagacggcctgtgtcatcacctcggagcctccttgtaagtcccatcgccctccgactgctcgggcttcccctcttctccttgctgtgtgtgcccctttgtctatcgacctgttgccagtggacgtcgagtcaagccgagccgctgagcctcacgcgtagaaccaccggcatgtaccttatgctgcgcgtgtctgctgcgcgtcagggtgggttgatgtcgggaaccagcctcggcacttcccgtactcctcagcagctgggtcgcacgtgtccagccgtcgctgacctcggatgattggcaccgttgttgctggctggcaccatcaaggactccatcgtctggctttgggcagctatggagctcgagcagcgtcactggcaccacggaggcgtgcgctgcttttccaagtgggagctcgcggagcgttccatcccgcctgatggtcgataatccgcccacatgctgtcgtctctggccccctacggctcctgctccgtcccaccgcgctgcacatactatagtcagctcccgtcttgcagcgccgccgagtgctaaaactgtggttgcaggtctcttctgtaagaacaggggtctggtctgcaaatctacatcgactcctgatgaggtaccccctcagcacgagagtgccggcgaaagtgtcgctggcaatgccgcctccgacgccgagcttgtcaattccccggccatgggctcttcgccgagcgttacacgccgcagtgtctcgcattccagccctgcagacacgattcagtcgtctgcgggcctcagtgctgctggagatgcaagtgtaagccagcagccatctgtgccctcggccatggtacaccaggggttggttcgccagtcagggccgcctgcggatggcctggtccaatccccatatgacgcctctcccccggtccctgtggcagcaaacctccctctacatacgctcgaggacaacttcaacaggactgcgcactctatgggcccgtcggccgagcaggacacgtatctcttagatgcctttaggtgtctcataatcagcgaaaaggacgagatcgatgccaacatcatccaggtgtatcatggtggccagtcggcggacgatcgtccagttcacttcttgctcctggaaaatgagaatcccgagtttaccaatgaggcaaagcaagctgcctcagacggaacagagaggcttgtatggccttatggtgaacgcttgatacggttatacttcaagcacgtccatcccatataccccgtcgtcaccaaggcgcgattccttcgccaatacaaggccaacaggatgaggatccccgcctcgttacgcggcgccatctacggcctggcatgcgttttctgggaccgtgactcaaccgttacagagccatgccccttccagcagcatcaatgcatcgaccacgcccacgaggctttgaggcgcgagcatgagaatcccaatctcttcagccttcaggcgggtcttctgctccatcacatcacgcctcctgaaatcgacagcgttgagacaccaagcatgtggaccatgacggcgcaaaccactgcctgtgcccagacgatgggcctccatcaagatcccacccgctggagtattgagccatgggaaaagagggttaggaagaagctgtggtgggcagtatacgcttcggattgctggtctgcaatctgccacgggaaccctacccatatcagcccagattcgttcagcaccatcccaccagacatggacgacattcgggatgatgagactgtgcctgaagacttgcgatacctggtcgagcccgaggatgccatctttcaggcttcggtgggagctcgattcctgcactacatcaatctgaccattgtcttgagagaggtgttagattgcaccttgtaagtccgagcaatccctgcataagatgttgttgctggcagttgctcacacgactttcttgatacagtcaggttcgacctaacccgtcaaccctcatcaatagggcaacccagctagtgattctgagagagaagttcatagactggcatggccttcttccgcaatgccttaccgtcgattccgaaagaggccaagacgggatcagcaactgtgagattatcggcaacctgataccgaagttcggacacaagcactaacttgtcgccaaggccccctgcatctcacctattacgcgagccaggcactcctgtacagaggattgatgtatcctgctaccagagaggccaaagccgatcccgagtcaagccttcgcaagtggtttgttccagctcttggcgagtttcaaagctttgtgtgcttctttgattccattactgaggaagatctgcgtggcttttggtgtcgtcgtaggtcttgctcctcttcttcttccttttgtctcgatgtggtcttctggccctgggatttgactgtacatgacaactttgactaacggggaaccagatgctcgatcccaactgatcttgtgtggcaactttctcatctacctcttccttctggccgctcaccagcagcacattgaggctgcatatcaactcttgcaagagttccaccactctctgaagcgacttggggaaaccacttatacccctggacggcttcttctccggcctgtcaggctcaggttggattccttctttcgacaggcggccacgattttgcgagggagcgaggcgcccgttctatcgcatataaacggaacaggccagacgctgtga

SEQ ID NO: 29 - protein, 700 aa, Trichoderma reesei
MLSGADSAQGRPRKFTIQSQFGRPRQPRSRKNRPCDACRKRKTACVITSEPPCLFCKNRGLVCKSTSTPDEVPPQHESAGESVAGNAASDAELVNSPAMGSSPSVTRRSVSHSSPADTIQSSAGLSAAGDASVSQQPSVPSAMVHQGLVRQSGPPADGLVQSPYDASPPVPVAANLPLHTLEDNFNRTAHSMGPSAEQDTYLLDAFRCLIISEKDEIDANIIQVYHGGQSADDRPVHFLLLENENPEFTNEAKQAASDGTERLVWPYGERLIRLYFKHVHPIYPVVTKARFLRQYKANRMRIPASLRGAIYGLACVFWDRDSTVTEPCPFQQHQCIDHAHEALRREHENPNLFSLQAGLLLHHITPPEIDSVETPSMWTMTAQTTACAQTMGLHQDPTRWSIEPWEKRVRKKLWWAVYASDCWSAICHGNPTHISPDSFSTIPPDMDDIRDDETVPEDLRYLVEPEDAIFQASVGARFLHYINLTIVLREVLDCTFQVRPNPSTLINRATQLVILREKFIDWHGLLPQCLTVDSERGQDGISNCPLHLTYYASQALLYRGLMYPATREAKADPESSLRKWFVPALGEFQSFVCFFDSITEEDLRGFWCRHARSQLILCGNFLIYLFLLAAHQQHIEAAYQLLQEFHHSLKRLGETTYTPGRLLLRPVRLRLDSFFRQAATILRGSEAPVLSHINGTGQTL

SEQ ID NO: 30 - protein, 29 aa, Trichoderma reesei
CDACRKRKTACVITSEPPCLFCKNRGLVC

SEQ ID NO: 31 - DNA, 2178 bp, Trichoderma reesei
  source 1..2178: mol_type = other DNA
atgcctacgaaaaaggtgaaggacagtgagcggcggcgatgcgccagggcgtgcacaaactgcaaacgacgcaaggagcgctgcgacggaagacaaccttgtcggcgctgcatagagaggggggccgtctcagaatgcagtctgaggccccctcggcgctctttctctgtggcttccgctctgccaccaagtccgttgtccgagtgttcagaccctcgatctacaaccatcgagtctacgaatggttcgctgtcaaacgatgccgatggaccagctcatggccttcagcatgagatggcactgcggttccctcatttctcgaggctgataaaggatgacagaggcaccttcatgttcattggcgactctgccaaccacagcttccttcagaacgtccgccggattgttcgcaggcacgtgggtgattgcatcttcgtcaatgatccgctacgccatcacatggttgaggcatgtccaagcaaaggccacaatatgatactcgacaatggtacggcaccgccaccaaaaccaaccgaggatgaagcaaggactttggttctgtggtatcgacaagctacgcattccatcctcgagctgtttgacgatgccactatatgcgacaacatcctcaactggttgcgtcttcctcagcagcctgttgacgcgctgagtgccacatattccttgatactcgccattggtgctcaggcgtgtcctgaagactacgacgaaacagcggagaagcacttcaactacggccgatatgtgacgctggctactctcatggacgaccccagcattgccaccactcaatgccatgttctcatagccatgtatctcctggcccagtcacgcagaaacgctgccttcatgtacttagggatagccgtgcgtggcgcttatgcactgggtctacacagagaagatgtttctgcgctgtatgatacggccgaatacacgacccgagaacgtttgtggaaaggtatcaggactctggatctctttatgagtgcttcactcggtaggcctccggcgaccctggagactcgggacacttcatcgaaagagcactattcagctatgaacgacttgtccttcatatttgagaacatcttgaccgaggtgtatgccaaacggaagatcacaacagagtgtttgacgaagatcagcgagcttcatcgcagctgggccgccagattcagtagtggccagagcgtggagggcattgacatacatgctcataaagggacaccgagaaatcaagagatggcaccaaatattggacaaatccacctcaaagaggcatactattggacgatcatgctgctatctcggccatttctcatcgactacgtttcttcagtatcgccaccagagatggacgcacctcccacttcatcccgtcatgcgtccacaccgtcaccatcacgaaatatactcgctcaagcatgtgttgactctgcggtacgaacaattgagcttatagagccgcttctaacgagcaacctgacaccaaaaaggctgcccttcatcgtgaattcagcattcgcggcttcgcttatacttggactttgtctctttagtgatttggaagggtctatgcccgtcaggaggaagcgacttggagaagcgtgcaagatattggaaaagttcagtgtgcacgacctggtcacgaagcgttacttaggtatcgtggagaagatgatcggcgcatgcgacacgttcctggagctgcaacagcggaagaggatggagtacaataacgacttgatctcgaggctctttggtcgggttgatgcagagccgccagcgagagatgccgatccaacccacgactcctctcgaaatcacatggcacagtggcagggattctgggcttctgagtctgcagctgccaaccaaccaacaccattgccgacagcaagcggcggtatggtccataacaagacgaactttggggggattgatgaagcacattatcacgaagagcagaacagaggcacgatgatgcagcaagatgatggtggcaacccgtcgcttcaggatctcggcgatatcggaagcttcttgtccccgaggacactgtggttcgatgcgtacgatagtggcgcactgtttcctatggtcggaagcagtatgttggaatttgcaagcctggagggagatcaaagcgcgtag

SEQ ID NO: 32 - protein, 725 aa, Trichoderma reesei
MPTKKVKDSERRRCARACTNCKRRKERCDGRQPCRRCIERGAVSECSLRPPRRSFSVASALPPSPLSECSDPRSTTIESTNGSLSNDADGPAHGLQHEMALRFPHFSRLIKDDRGTFMFIGDSANHSFLQNVRRIVRRHVGDCIFVNDPLRHHMVEACPSKGHNMILDNGTAPPPKPTEDEARTLVLWYRQATHSILELFDDATICDNILNWLRLPQQPVDALSATYSLILAIGAQACPEDYDETAEKHFNYGRYVTLATLMDDPSIATTQCHVLIAMYLLAQSRRNAAFMYLGIAVRGAYALGLHREDVSALYDTAEYTTRERLWKGIRTLDLFMSASLGRPPATLETRDTSSKEHYSAMNDLSFIFENILTEVYAKRKITTECLTKISELHRSWAARFSSGQSVEGIDIHAHKGTPRNQEMAPNIGQIHLKEAYYWTIMLLSRPFLIDYVSSVSPPEMDAPPTSSRHASTPSPSRNILAQACVDSAVRTIELIEPLLTSNLTPKRLPFIVNSAFAASLILGLCLFSDLEGSMPVRRKRLGEACKILEKFSVHDLVTKRYLGIVEKMIGACDTFLELQQRKRMEYNNDLISRLFGRVDAEPPARDADPTHDSSRNHMAQWQGFWASESAAANQPTPLPTASGGMVHNKTNFGGIDEAHYHEEQNRGTMMQQDDGGNPSLQDLGDIGSFLSPRTLWFDAYDSGALFPMVGSSMLEFASLEGDQSA

SEQ ID NO: 33 - protein, 29 aa, Trichoderma reesei
CTNCKRRKERCDGRQPCRRCIERGAVSEC

SEQ ID NO: 34 - DNA, 2287 bp, Trichoderma reesei
  source 1..2287: mol_type = other DNA
atgaatgcttgcctcccatgccgaaaggtaaagatgaaatgccggcaaggcagccaaccggcttctaaatgcgagcggtgcgcgcggaagtctctcgactgcgtgtttcaacggcatcgtagaggccggaagcctgggacgaagtgagtgagacattccgggagccttagagtcccagacgatgagaattgacgagacttccatactttgtaggatcgccaagcggaacacgcctacagcgccgcgtgccgcaaattcagctgtgaacaagacgccgggctacagcgctccatcgatggtagaaacagtaccggctgaggagcctggcaacttacagccatccggagtgctcaatcaggaggcaatgcgtggcaaattctcgctgcgccgaatactgagcaccgcagatagtgatgctatggaaccaccggaagcatctcccccaggccgaccagaagatcccatcgagatgggcatcctcagcctctccattgctaaatcacttttcgagaagtttgtgaatcactagctacggcgttatcttcccgttaaagactaatgcgaggctttcttgtagtttcattctggttctcaacccgtatataagtcagctggatccccatctgcacactttcttctacgtccgccaaaagtcgcccttcttattcacagctgttcttgcaatgtctgcaaaggcgttcaatcccatcgtatataatgcgctgtatgaccatgcgcaacacctctacaccgaagtctttcggaatggcctcaagtcaacagagattgtccaggccatcctcatcctcacctattggaagcaaccacaagatacgcgagcatggacatcgataggatatgccattcgcatgtgtatggacatggggtggcacaagctggcaccctactccacatccagtcgtctcaccatgggcgaagccaggcgacgcgaaattcgtaacattgagcggacgtggtatgtgctatttgtctatgatcgaaggtatgttagcccgtagcccccctatataaagtgctacaactaagaatgtcccagtatcagtctccaaaccggacggccttggatgattgagcgcaacgccttcatagagtcgatcgacacttggtgcactgatgccatcgcagaccgcaacgacgacttgcttggtgcctttgtcaatctgagactgatgagcacctcaatcttttctcttcatgctcccagccgttcgcgcggcgaaagagtacctctggaggatatggaaaccctactttcgctgaagaaagccagcattgagagatgggagcgccactgggtccaagaggtggacaaaaagctgttgcccgaagaagagacgtgccacaaattcctgatccggttctacgggatgcattttcgattgcagctcttctcgttgccgctccaagacgtgctttcctcagcatactcggaatcatctctccatttggacatcatctgggccgcattcacaggtgccatggacatgctgaagctggtgtcacaacattcgacccagctgtattttgctcaggactccattcatgtcatgactgcctatagcgcggcgtttctcgtcaaggtaagtcctactctacatcttttatacccgtggtctcaggaggctttgcgatcctaatgttggaggtcgatttctttagcttctcatatctgcacccaatacagtagtaaaggaaatagagtcctcaacaatatctgtcattcgttctgccgctcaagccttctctcaccaggcgtcagctccaggaacgagctgcgaattgcaagcacgatttcttcacaatattgcgacaaaggttgctcagagaaatagaaaggagaatgtcgcaacgcctacaatcacctctggcctagatcgacatgcatctgaagaccatgatcactcagagtcatccaagctaccattcttaccaccagaagtcaaccaggccatgccccagccgcttgagcaacctctccccgacgtcttcatctttccgcatcctgacttggatccgttattcatggacgacggtgcatgggctgatattctggcaagtgccgcgttcaacactcaaaacggtatgttattggcgggctgatgtaatgactgtatattgaggcttcactactttcgtgacacaccgtcctccccgcatcatatcgaaaaatgactccataacctttgtacatcatggatgcaacggaacaatagtcgtttccgagtggctgaatgtgcgaatgccttctctgataattttcagttga

SEQ ID NO: 35 - protein, 617 aa, Trichoderma reesei
MNACLPCRKVKMKCRQGSQPASKCERCARKSLDCVFQRHRRGRKPGTKIAKRNTPTAPRAANSAVNKTPGYSAPSMVETVPAEEPGNLQPSGVLNQEAMRGKFSLRRILSTADSDAMEPPEASPPGRPEDPIEMGILSLSIAKSLFENFILVLNPYISQLDPHLHTFFYVRQKSPFLFTAVLAMSAKAFNPIVYNALYDHAQHLYTEVFRNGLKSTEIVQAILILTYWKQPQDTRAWTSIGYAIRMCMDMGWHKLAPYSTSSRLTMGEARRREIRNIERTWYVLFVYDRSISLQTGRPWMIERNAFIESIDTWCTDAIADRNDDLLGAFVNLRLMSTSIFSLHAPSRSRGERVPLEDMETLLSLKKASIERWERHWVQEVDKKLLPEEETCHKFLIRFYGMHFRLQLFSLPLQDVLSSAYSESSLHLDIIWAAFTGAMDMLKLVSQHSTQLYFAQDSIHVMTAYSAAFLVKEIESSTISVIRSAAQAFSHQASAPGTSCELQARFLHNIATKVAQRNRKENVATPTITSGLDRHASEDHDHSESSKLPFLPPEVNQAMPQPLEQPLPDVFIFPHPDLDPLFMDDGAWADILASAAFNTQNVVSEWLNVRMPSLIIFS

SEQ ID NO: 36 - protein, 31 aa, Trichoderma reesei
CLPCRKVKMKCRQGSQPASKCERCARKSLDC

SEQ ID NO: 37 - DNA, 2364 bp, Trichoderma reesei
  source 1..2364: mol_type = other DNA
atggagtcgagcaccacgacctacaagcgacggcgaccggctacagcgtgtgtcgagtgtcgacgcagaaaaatcagatgcgatagtgcaacaccctgtggcccatgcagaaagcttccaaacaccaggtgcatttacaacaacgctcgcttccaaccatttgcatacaacatcaaccgccgcaccaacaataatataaaccctcagctttcagaactcggatatgcccagccaagaaaacaacattcgattttcggagatagcgattctgggcatctcgacctcttctctcttgagacgacgacagctacgacaactacgagtgacggggggacaagcctcgaaacctttctggggctcaatgacgcgttctgcaattcagctgctgcacattcagccggcgatgcattccaagatgcaaacgctgggccattttcggatgtatctactgctgtctccgggcacttgtcaacacggagcgacgaccaaacgtcttggacctcgacgcactcgctgttagatcaaggacaatctgattcccccatcgaaactgctcacgaccagttcgccgattgctgggaagaagccatcaccaagtgcgaaaggttacaatttctactccatgggctaagaaattcgaatgggccgatagcactacaaccgagagacgagcacttgcggccgctgatagagagatttgttgagctggaacgtcacgcaaaagcaaaaaggccctcggcttcctcagtggcgctgaactgggccgtgaacctcgatccatctgtcaacgcggtaagaaagctacttcctccgcaagagatctgtgacaagctgctcgatgcatatctcaatacctttgaatcggtattgcggattctacacgtcccagattttcggcatgattataaacgattctgggccggatctccgtcacactgcccgccggaagttgaagaggtgtttgtgtgcaagctcatgatgtgcatcgccctgggcgctttcgtctgccccagtctcaatgctgcggctgacggtcttcctgaacagcgacaagagggcagcagtttgcgagaacaagcaatacaatgggtcgcctacgggaagcagtggctggcacgaaagatgatggcagggtcccgagcagacaccaatactgcacaagttgtgtgtctcctggcgttgtcccggcacacaggcccgatccagacggcctcgatcggctcgctgtggtctcctggggaccatgacctgacccgagttgcgatgcagatgggattgcaccgggaccctaccatagtcgatgcagatatgccggcccgagaggtcgagctccgtcggcggttatgggctacgatgctggagttgtccctgcagctgtcccttgacaacggcctccctgccccaatctctccagaaagctacgattgcgcgccgccgtccgaattcacagacgacgaggctaccgatgccgacgctgatccggtatcatgcgcctccctcacgagctttctcttggactacggtttgccgaggacgaacccattggtcattctggcacgaactcagcgcctacggctccagatcttacacgtggtgcattctcctggtgctgccaaggcctttgaagataaacaccggctttcggccgagttgagggctgtctactctgcagagattcagcggttcttgtctttgcaaaagtcggcctcagtctttcaactcaagctgttggacgccttcacgctgccatttgtgcttgcaccacacgccaaattcgctgcctcctccacaaccaacccggcgtactacttttctcgcaaagtgcggatggaggtctcgctgctgcttttggccagccaactgcccggaccaaaggcaccggacaatgggcttggcgaactcatccctgacgctgtcagcatgaccgccttggccaccggaaccaccatatcaacaggcgcctcggccgcatccgccggtccagatgcgttaacggctcttcggatacactgccaaggacatctagcaacgttgcagtggcatgctattttagccctctgtctcgacatgatcagcgagctcgaggatagctttttccctgccatcaacggtgcaagctggcggcagctccaagagacgatccagacgtacgtcacagtcctcgagcaacgggtgcgaacctcggcaggggccgtatcggcacgcgagttccttttctgctcttgtgcagaagcatacattggcgcgatgatgaggggggacagggaggtggaccaggttattgcatcggctggctattccgcgctgacgctgtgttgtgagatcatggagcaggggttacaccagcggtgcacggttgtttag

SEQ ID NO: 38 - protein, 787 aa, Trichoderma reesei
MESSTTTYKRRRPATACVECRRRKIRCDSATPCGPCRKLPNTRCIYNNARFQPFAYNINRRTNNNINPQLSELGYAQPRKQHSIFGDSDSGHLDLFSLETTTATTTTSDGGTSLETFLGLNDAFCNSAAAHSAGDAFQDANAGPFSDVSTAVSGHLSTRSDDQTSWTSTHSLLDQGQSDSPIETAHDQFADCWEEAITKCERLQFLLHGLRNSNGPIALQPRDEHLRPLIERFVELERHAKAKRPSASSVALNWAVNLDPSVNAVRKLLPPQEICDKLLDAYLNTFESVLRILHVPDFRHDYKRFWAGSPSHCPPEVEEVFVCKLMMCIALGAFVCPSLNAAADGLPEQRQEGSSLREQAIQWVAYGKQWLARKMMAGSRADTNTAQVVCLLALSRHTGPIQTASIGSLWSPGDHDLTRVAMQMGLHRDPTIVDADMPAREVELRRRLWATMLELSLQLSLDNGLPAPISPESYDCAPPSEFTDDEATDADADPVSCASLTSFLLDYGLPRTNPLVILARTQRLRLQILHVVHSPGAAKAFEDKHRLSAELRAVYSAEIQRFLSLQKSASVFQLKLLDAFTLPFVLAPHAKFAASSTTNPAYYFSRKVRMEVSLLLLASQLPGPKAPDNGLGELIPDAVSMTALATGTTISTGASAASAGPDALTALRIHCQGHLATLQWHAILALCLDMISELEDSFFPAINGASWRQLQETIQTYVTVLEQRVRTSAGAVSAREFLFCSCAEAYIGAMMRGDREVDQVIASAGYSALTLCCEIMEQGLHQRCTVV

SEQ ID NO: 39 - protein, 28 aa, Trichoderma reesei
CVECRRRKIRCDSATPCGPCRKLPNTRC

SEQ ID NO: 40 - DNA, 3164 bp, Trichoderma reesei
  source 1..3164: mol_type = other DNA
atggaggaggacacgggcagccagacgccgggccctggaagcggccctggaagtggcccaggcagcggcccaggaagcggcccaggaagcggccaagggagcaccagcaccggcactggcaccggcagcggcgcgacgccgcagtcccaacaggcgcagtcgcaggccggcccgtcaacgtccaactcgctgctgcagctgtccaagcgcagacgcggcctcggcgtcgtcacgcccaatgcctgcacggaatgccgcaagaagcgagccaaggtgagccctgctgtgaacgggaacttgaggcgggagagacgctttggcctcgtgtgtgtgtgtgcctctactacatgtagtagaggtatgggcatggacgaccttatcgacctaccttacctaggtagacacatgcaaaggcgatgtcatagcttctttcctggccgctcactgttacctgaccatgactctcgcttgcccatgctatcagacctgtctatcaaaatgcgtgggcacgcacccaacgaacaattgctgacagctccgtgtgtgtgcgtttcttgtagtgcgacggccagaaaccctgcgggcgctgcaagtcgcaaaaggacgccgagtgcatctacgaaatccccatccgccagtctaaagaacacctccgcaacgaaatcgagaacctgcgcctgcggcagcgctcctccgaccaggtctttgccgccctgatacagccgcagctctgggaggaggtgctggcgcgcctgcgcggcggccagtccgtcgagagcatctccgagtggctggccagcgtccctcatccctcggcgccttcaggagccgcggcgtacgacacgaccatgggccacgacctggcctccatggccgcctttggcgacacctcggtggcgccgctccccggcatggccctcggcatcgggccggggtcgctgtctatacccggcctgtccggcgagttggggggcaacgcggccagcccctggcagttctcgtcgcagagccaggccggctcgaccaggagcggctcccacccggacttgatgagctgggcagccaccgacatgaatgcgccgccgtcgcgcgtcggctcctgggccgagagcatgcagacggaacatctcggcggcagtggtggtagtggtggtggtcctggtagtggtgctggtggcggcggcggcggcggcggcgagggcataccgcgatatcgtgggctcgagcaggtgctgtcgccgctcaacgagcaccaggccatgaagccgcccagcggcacctggaccaccatcaccgacgacgtcaacctggttcagcacctgttggccctctacttttgctgggagtatcccacctttgcctccctcagcaaggagcacttcctccgcgacttccaggccgggagacaccgctactgctcctccatactcgtcaacgccctgctcgccctcggctgccgcttctctacccagcccatgacgagggccaaccccaacgacccctactcctccggcgaccacttcttcaaggagtgccagcggctcttctacgaggagacggaccaccactccttgaccaccatccaggccctgggcatcatgtccatccgcgaggccagctgcggccgagactcggagagctggtactacgcggggcagagcatacggctggccgtcgagatgggcctgcaccgcatccatgacgatgacgacgaggacgagctcgccgtccagtccgccaccttttggggagcctttgccctcgaccagtgagtacaaagcagtccgatatgtcattgatttcgagcgcaaagcgtccaaaagagacgatcttctgaatgcgccccgtatacagggagcaacggctaaccccccctcttcctcaatctcagcgcatggtccctggccaccggctcgctgccgcagtgctcgtgcttccctcacctgccgcccaagccggccattattggcgacattgaagcctcgctatgggtaccgtataccgacgacggtgaggaccgcacgaccacctttggcgtagattcgaaggggctgcctactaacgtgtcagaaggcgcgccgttgcagcggtcgtgcgagcagccatctaacgtgcggtcagtatacaagtgcttctgcgagctgagcgagcttgtccaccagtccctctacgtgctccattctcccggaaggccccttaccgcacgggatctgctgaatatatacactcagtacctcaactggtacgaccgcatacccgaggtgttgaggctggggcacaactttacgccggcggtgttgttcgcgcagtgagtctaacgattcttgtttcttctctttgctcattggccatctcaacttcccttttttccttcactgtactttattctccattcttttcttcgccccttttctttcttctatcatattttcctggtggacgctgacgtctgttactactcgatcacaaataacagcatgtactatcacttcgccatacttctccttttcaggcccctgatcaagctgcgcatcatcggatccaaggtgtcgccgcgagacgtctgctcgcaagccgccgatgcgatccagggcctgctgacctcgtacgcgcagctctacacgctgcgccggacgccgtcgtttgtcccctatttcgtcctgacgtccgccatcatgcacctggccatcggggccgcgtcggtcaagaccgagggccacgagacgagcgacgagaaaatgaagaaggccgccaagatggacccgcgcgtgtccgaggccctcagcaaaggcatcgccgacctgacggagatggcgccgtgccaccactttgccgagcaagcgctcaacatcctgcgctatctggccaagaagtggaacatcgaggtcaacgtcaacggcgacaagacgccgccggaagacgccgacaagctggtgcggccgtactcgagcagcctcaacttcttcgcgcccaacgtccaggagaacgactacatgtgcggctgggggcccggcgccgagatcgtagagacgctgcagccgcccagcctcaagaagacggccgagctggtggagaacccgctcttctggccgttccccatgcagggcagaccgctgctgcccagcggcgaggagctggagcaggcgggcttcagcaggctttga

SEQ ID NO: 41 - protein, 798 aa, Trichoderma reesei
MEEDTGSQTPGPGSGPGSGPGSGPGSGPGSGQGSTSTGTGTGSGATPQSQQAQSQAGPSTSNSLLQLSKRRRGLGVVTPNACTECRKKRAKCDGQKPCGRCKSQKDAECIYEIPIRQSKEHLRNEIENLRLRQRSSDQVFAALIQPQLWEEVLARLRGGQSVESISEWLASVPHPSAPSGAAAYDTTMGHDLASMAAFGDTSVAPLPGMALGIGPGSLSIPGLSGELGGNAASPWQFSSQSQAGSTRSGSHPDLMSWAATDMNAPPGTWTTITDDVNLVQHLLALYFCWEYPTFASLSKEHFLRDFQAGRHRYCSSILVNALLALGCRFSTQPMTRANPNDPYSSGDHFFKECQRLFYEETDHHSLTTIQALGIMSIREASCGRDSESWYYAGQSIRLAVEMGLHRIHDDDDEDELAVQSATFWGAFALDHAWSLATGSLPQCSCFPHLPPKPAIIGDIEASLWVPYTDDGEDRTTTFGVDSKGLPTNVSEGAPLQRSCEQPSNVRSVYKCFCELSELVHQSLYVLHSPGRPLTARDLLNIYTQYLNWYDRIPEVLRLGHNFTPAVLFAHMYYHFAILLLFRPLIKLRIIGSKVSPRDVCSQAADAIQGLLTSYAQLYTLRRTPSFVPYFVLTSAIMHLAIGAASVKTEGHETSDEKMKKAAKMDPRVSEALSKGIADLTEMAPCHHFAEQALNILRYLAKKWNIEVNVNGDKTPPEDADKLVRPYSSSLNFFAPNVQENDYMCGWGPGAEIVETLQPPSLKKTAELVENPLFWPFPMQGRPLLPSGEELEQAGFSRL

SEQ ID NO: 42 - protein, 28 aa, Trichoderma reesei
CTECRKKRAKCDGQKPCGRCKSQKDAEC

SEQ ID NO: 43 - DNA, 1604 bp, Trichoderma reesei
  source 1..1604: mol_type = other DNA
atggcagaggatcgacatcgccaggagtatcctcagcaacaacggcccgaggatatcaagcagcaccaacctctgcccccgtactaccaccacgtccctcctcgggacgcaccccctgcggataccgaaagcagtgcccattctcgaagcaccatggcaacctcagtcacattgccgtccatccacgactcccggcccagcggctacgggcctcctcttccagctggccgaggctacgtgagcgacccgcgatatgcgtctccgaatgccgtcaatggctatccaccacccggaagccagcagcagccggccgcatacctccctcctctgcagcctcagtcagaccctcggcctgcataccctccacccgagccgcgaggtgcatattacgacgaccgccgtcctcctccgcatccggcctatcatgaccagtactccgcagactattactaccgtggactcccacaagcccaccacaacggctatgctcacgattacgcgcgccctcctcccggctacgcacaagactatggtcagccgggtatggaccgccctcgagaagctcctaggcagcggacctccattgcctgcaggtattgcagaaaacgaaaggtttgtcacgcagtgtgcattaatcagcccttgtgtgtgtctgtggttgtcaccccgcctgttgaaatccatcagtagctaacccgtgctcccttggcgctctccagattcgctgcagcggccaccagaatgcccctggcggcaagtgtcagaactgcgcccgtatgaaccaggaatgcatcttccagcccgtgagctcgtcaagctccacggcctttatccctgtctcggccgtccccggaggcgtgccgcccgggacgcagttattcggagcctacggccagcctctcgccccgagcaccgtgcctcaccctcatcctcaccctcaccctcacccgcatcacccgcaacaccacccgcaacatccgtctcacgcctatcctccgccggcaggtccgccccaccacccgcccgggccttattaccacccgccggcccagtcacccaccgattcatattcctcttacggggacctgaggggagacccgagaggagatatgaggggggacctgcggggggaacccagaggggagctcagggcagatgaccagaaggtagggcgcagacgtcgcaggtcgtcgcaagatctggatgacgcatataggctgcctcctcctcgagctggcgggatggacgacgatcaccggcgaagatcgcccgccgagctttccaacaatagcagccccagcagcttaagctactcgcgttactcgggagctgccagacacagcccccgaaacccagctctaccgcacccctccagtgtcggcactgctgatgggcgctcccccatcacgcagcaaaatggatcaagcggatcatcgactccggcgcgatcgggccctcagaccagccagtcgcagcagtcaagctccatcatgagtctcagcaacctcgtggacaagaacgacatcgacaagacgatgattgagcgcctcaaccgacctcgagacactaattctggccagagccagagagaggcctctcgctaa

SEQ ID NO: 44 - protein, 498 aa, Trichoderma reesei
MAEDRHRQEYPQQQRPEDIKQHQPLPPYYHHVPPRDAPPADTESSAHSRSTMATSVTLPSIHDSRPSGYGPPLPAGRGYVSDPRYASPNAVNGYPPPGSQQQPAAYLPPLQPQSDPRPAYPPPEPRGAYYDDRRPPPHPAYHDQYSADYYYRGLPQAHHNGYAHDYARPPPGYAQDYGQPGMDRPREAPRQRTSIACRYCRKRKIRCSGHQNAPGGKCQNCARMNQECIFQPVSSSSSTAFIPVSAVPGGVPPGTQLFGAYGQPLAPSTVPHPHPHPHPHPHHPQHHPQHPSHAYPPPAGPPHHPPGPYYHPPAQSPTDSYSSYGDLRGDPRGDMRGDLRGEPRGELRADDQKVGRRRRRSSQDLDDAYRLPPPRAGGMDDDHRRRSPAELSNNSSPSSLSYSRYSGAARHSPRNPALPHPSSVGTADGRSPITQQNGSSGSSTPARSGPQTSQSQQSSSIMSLSNLVDKNDIDKTMIERLNRPRDTNSGQSQREASR

SEQ ID NO: 45 - protein, 32 aa, Trichoderma reesei
CRYCRKRKIRCSGHQNAPGGKCQNCARMNQEC

SEQ ID NO: 46 - DNA, 2823 bp, Trichoderma reesei
  source 1..2823: mol_type = other DNA
atgtcagcagctgacgataaagcacagctgaatgctccacaacccagttcaaagcccaagaaaccactctcttgcctctcttgtaaagtccggaagctgaaatgtgaccatgagagaccggcttgcacgcgatgcgccaaagccaacagcgagtgcatttatccggaagctcggcgcaagccgaagttgctgcaaagcaatgtcaaggagctggaggctcgcataggtatgttttgagagcttcccttgctcagatgcacgcgtcttctggagcagttggaccgctgtaactcacaaataccaatagcccaggttgaaggatatctcaaagaggttggccaaagccgtcctgacactactagcaaagacatccttgtcggcggactggacagcgaagattgtgtcagtagcaaagaccaaccgagcaacgggatagcgtgggaagacgacgccgcttttcaaaacctcaacggtctgcccagcacctttgctggcgaggatcagctatttagccatgaagtgattggggacaatgaggctccggaggggacgcaaccgctattccgacctgcaggacttgataattatgctggcgattcgttcgagagtggccagctgattgatctcggcatgtcagaggcactgccgccttctgaaatgatagaagatctgtgagtcttcatggcgctatatcttacctacctctagcgacaggcttctcaccattagtacctaggcacacctccttcttctctacgcaatatcattatatgccggcagtccatcctggccgctattacaaagcgttctacggcaattcgctgaacaagccaccaatgtgcttgcagtatgctatctgggcaatgggcgccttgtggcatcctaaatacgaccgaatcgctgatgtcttttataaaagagccagacattacgccgagactgacgaattgaaggtgggaatctctatcaaaggagtggcctagctatcgctaacgcgcagaaagggtgacggggagcattttatcacaatagcacatgcccaaacctgggtcctcatcactgcctatgaagccagagctatgctttacacaagagcatccatgagcgccgctcgaggctccagacttgtgcaaatgctggctctcgacaggatagatgcgccagatgaccttctccctccgagcctgggacccccaatcgactggaccgaattggaggagcgcagacgagtcttttggatcgctttttccaacgatgcccagggaagtatagcgactggttggaatagcatcattaggacggaagaggtgagttcctcactcccatagcctggaaccatgacaggtctaaacaaacacagataatgactcggcttccggctagcgaagcggcctttgcctcaggccaagaagaaactgcacctttcttgtatgaggttatgaaaggcgcaccatatagtggctttactgggttcctcgttatcaacgagatgctcaagtccatcatgtcacacgttcatctcatcaagtcgtccgaccgacctgaagatgccatcaatggtgctttctggcagaggcaccggcgtttagataaccaattgtccaccctgttcctgttcatgccggaaagcttccgtcttcctgacaacatacgagaccctctcgctacatatatcaacctgaactttcacgcatgcgtgatatgcttgcaccacgttgctctcgagaagatcgaaacccaccaactcgatgattcgttgagaaaggagtgtcacagtctgctcaaaaatgctgctgaggaaattgccagcattgctaagctgacatcacaccgctcctctttgtttgtacgggaccccgtatcgacattgaagtacgaggctgacatacaatatagagcaaccccctgtgcgccttctccctctactgtgcgacgaccgtctacgtgtacctcgccaaacaggacccttcctcaggaatcaactccaaagacatgtccaacctagaactcatcatcaacgtcatggaagccataagccgcatccacttgattacaagcgccttcctacagcaagcatgtctcgacattgataagaatggcctctcgagcatcatcaagctgcccaatctttatcaataccgggacctattcggcggccctgcctctagcatccctctcatggcacggagtccaatatcgaggcacacgcagatgagccctccattgcctgggcggcttcccttagacaaacctgtgggacatttgcggccgatgaatttaaggatgacaaagtcagtcccgttgctaacgggagtgacagcggccatgagccggctggacattaccgattgcttcaagccgtctctgggtgccatatcccgaaatctggagcctcgttctacggacaacactcacaaaaggaaacgacggccttcggcttctcagttctctgcacacagaagtggtgagctcgtcggggaaacggctctcagtgactacacaggtttcgaacattcaatgcatctctcaatggacaatgagggagaacaattaggagaaggttctcgactctgtggcacaacaaacacaccacattttgcacatttgcctcttggagaagcagcaagcaggggtgcggagtctgacaggagccacggagtttccaaggaagctgaggcgtctcagtgtccccttgatatgccatttacagcgtggcaaaccattgacaacgagactctagccttttccgaggttactgcaagaattcctgatatgcatggcgacgaattggatttcatgggaagccgggggttcggaccatga

SEQ ID NO: 47 - protein, 840 aa, Trichoderma reesei
MSAADDKAQLNAPQPSSKPKKPLSCLSCKVRKLKCDHERPACTRCAKANSECIYPEARRKPKLLQSNVKELEARIAQVEGYLKEVGQSRPDTTSKDILVGGLDSEDCVSSKDQPSNGIAWEDDAAFQNLNGLPSTFAGEDQLFSHEVIGDNEAPEGTQPLFRPAGLDNYAGDSFESGQLIDLGMSEALPPSEMIEDLHTSFFSTQYHYMPAVHPGRYYKAFYGNSLNKPPMCLQYAIWAMGALWHPKYDRIADVFYKRARHYAETDELKGDGEHFITIAHAQTWVLITAYEARAMLYTRASMSAARGSRLVQMLALDRIDAPDDLLPPSLGPPIDWTELEERRRVFWIAFSNDAQGSIATGWNSIIRTEEIMTRLPASEAAFASGQEETAPFLYEVMKGAPYSGFTGFLVINEMLKSIMSHVHLIKSSDRPEDAINGAFWQRHRRLDNQLSTLFLFMPESFRLPDNIRDPLATYINLNFHACVICLHHVALEKIETHQLDDSLRKECHSLLKNAAEEIASIAKLTSHRSSLFSNPLCAFSLYCATTVYVYLAKQDPSSGINSKDMSNLELIINVMEAISRIHLITSAFLQQACLDIDKNGLSSIIKLPNLYQYRDLFGGPASSIPLMARSPISRHTQMSPPLPGRLPLDKPVGHLRPMNLRMTKSVPLLTGVTAAMSRLDITDCFKPSLGAISRNLEPRSTDNTHKRKRRPSASQFSAHRSGELVGETALSDYTGFEHSMHLSMDNEGEQLGEGSRLCGTTNTPHFAHLPLGEAASRGAESDRSHGVSKEAEASQCPLDMPFTAWQTIDNETLAFSEVTARIPDMHGDELDFMGSRGFGP

SEQ ID NO: 48 - protein, 28 aa, Trichoderma reesei
CLSCKVRKLKCDHERPACTRCAKANSEC

SEQ ID NO: 49 - DNA, 469 bp, Trichoderma reesei
  source 1..469: mol_type = other DNA
atgggtgtcggaaacaagcgcacgctcacaaaaacgagacggaagacgcggtagggcatttattatatggatggggccattaccctcaagctgacctttggtagggacgtggaccaagtcatctctgatctccgatctcacaaacacctgaagcaatacaaagacaccaaagcgccggaggacctccccgggctggggaagaactactgcgttgcatgcgccaggtggttcgacactccctttaccctggctacccacgagcgcggaaagccgcacaagagaaggtaagtctgcgtttgcactatgcctctggttttcttcgcctcggctgacggactctgctagattgaagcagctgcgagacgaatccaaggccacctcaaaatccgaacagtcgaacgtcctcgaatccgacaagccagagacggcgcaagactctactgatggcggcgtcgaaatggtcctgtag

SEQ ID NO: 50 - protein, 117 aa, Trichoderma reesei
MGVGNKRTLTKTRRKTRDVDQVISDLRSHKHLKQYKDTKAPEDLPGLGKNYCVACARWFDTPFTLATHERGKPHKRRLKQLRDESKATSKSEQSNVLESDKPETAQDSTDGGVEMVL

SEQ ID NO: 51 - protein, 34 aa, Trichoderma reesei
GKNYCVACARWFDTPFTLATHERGKPHKRRLKQL

SEQ ID NO: 52 - DNA, 1494 bp, Trichoderma reesei
  source 1..1494: mol_type = other DNA
atgaccagccacgaaggaggtcgacccaaaggaaagattcccaaatacaggacctcctgcgacaactgccaaacggccaaagtcaagtgcggccatgagaagccatcatgccgtcgctgctcggtgcaccgcgtggattgcgtgtacagcttgtcgcgtcggatgggccgtccaagggccaaaaaggtcgccgcgaatgactcaaatcaagggcagccagccacgtctgagcctaacgaggatggaactgcaaaggctgcctctccgaaggatgcggctgaagacgctgggccgccaccgatgcccgtagccgagactcgcatcgtggaggctgagcctggcgagccattggcgtctgagactgagcagataccggagagttgggtgcctttatttggtgaactgggacagctcaacgcagcggaagccatggatggcctggagaactcggaacagcaggaagatgatgcgatggagtttctgtcaggtgagactccaatggagcttggtgatatggctgctgcattcgccgggatgccctcattcttggacagtctgtcggactccctcgatccacaagccacaacatcagcggtgcctgcggacttgttcggacctcaggagatggtgtcatttggcctgccagttgcaacttccggggacgagacagttggtctgtcctcaacgcagcaacaccaagcaagcttgccatcagagccatgcacttgcaagcactcctccgagtcatctcgccgcgaatcgcggtccgctccaagcgacagagaccagtcaagtcagctatcaagtcttgcatcagccttgttttcagaagcgagctactctcctcatgaagcgaccccatgctctccgtccagtatttcgcaccccagcgatgcgtcaccgagccttcaacccagctgtgacggcgactccgtattgtcctggcggaaaacgcaggtcgtgctgagacaaggacgctgcaactgcatggatacaataacagagcgcatcgcgagtctcaagacagagcagcagagctcgtgtctcatgcccatggactgcgtattgatgctggagaaggaagtgcaggattcgctgtgtcttttgcacaaatgcaaaaattgtcggctcgatagcttcgtgcatctgctggcgcttgtcagtgtccgcatgatgctcgatatcctgcaaaagactgctcgcagcgagtttgtctccagggctaaatcgtcatctgacgactcttacgagggcgtttccctctgcatcgggacgtatacagttccatctcgagtacgatgcaggtttctccgcaaggcgttacaggcacggtttcataagctggcaggtttggtggaagagcgagaaaggctgttgactgggaagaagcaggatagtttttcaaagtcggcgtctttgcttgtgggtgatatttcacggggcttgagaacgattatggggtggattgagctgtggaatgcgagacatgcgtag

SEQ ID NO: 53 - protein, 497 aa, Trichoderma reesei
MTSHEGGRPKGKIPKYRTSCDNCQTAKVKCGHEKPSCRRCSVHRVDCVYSLSRRMGRPRAKKVAANDSNQGQPATSEPNEDGTAKAASPKDAAEDAGPPPMPVAETRIVEAEPGEPLASETEQIPESWVPLFGELGQLNAAEAMDGLENSEQQEDDAMEFLSGETPMELGDMAAAFAGMPSFLDSLSDSLDPQATTSAVPADLFGPQEMVSFGLPVATSGDETVGLSSTQQHQASLPSEPCTCKHSSESSRRESRSAPSDRDQSSQLSSLASALFSEASYSPHEATPCSPSSISHPSDASPSLQPSCDGDSVLSWRKTQVVLRQGRCNCMDTITERIASLKTEQQSSCLMPMDCVLMLEKEVQDSLCLLHKCKNCRLDSFVHLLALVSVRMMLDILQKTARSEFVSRAKSSSDDSYEGVSLCIGTYTVPSRVRCRFLRKALQARFHKLAGLVEERERLLTGKKQDSFSKSASLLVGDISRGLRTIMGWIELWNARHA

SEQ ID NO: 54 - protein, 28 aa, Trichoderma reesei
CDNCQTAKVKCGHEKPSCRRCSVHRVDC

SEQ ID NO: 55 - DNA, 2399 bp, Trichoderma reesei
  source 1..2399: mol_type = other DNA
atgacagcaagtacggggaagagatcttaccgcgcatgtgtcaggtgccgccgtcggaagacaaaatgcgatctgtaagcaattgcagcaaactctcatggtgtccgactactgaaacaaagggattatatcctcgcagaggcggcattggggagccgagaaaggcgtcgtgcgtctcctgcttacagtcgaatagccagtgtgtactggctgagtctcgcagaggggggaatttccggcgctacgaaccgaacaagaagcatcctgcaaagaatcgcaaggcccccaagcgcgtcccagatgcaccacctatggcacttgaatccatggagagggaaccaaagcgcaacggcgaaagattgaatggcgatgctattgtcgggcagctggtctccgagcttcgaaatccgtcagacgcattgcatatactcgctcaatcagaaaagggcgagtcggctgcgtcgtccacgttgccggagggggagcccaacaatctctcaaatggaccgtcaaacgctacatttcttgatgagtataagttggtcgagagcggactcatccaccgtaccgccatcttcgaattgttgcatatgtcagtcaaccgtcacaaagtgaacgttgcccgacctttacttctaactcgggcattagttattcccgcaactatcatccttattgtcccattgtgccggcatacatgcttggcccgtctgctattgacagaattctcaaatccgactttttcctgcttacagtcgtcctcacaatcgcgtctagagactcgccatcccactcgctcatccatcgatactgctgggattacgcccaaaggctcttgttagaagtgctcctcggacatccctggaccctcaagtctcgaacagttgagagcctgctgcttttgtcggaatggcttccgcatattgagctgacgcatggagcgctgaaggaggataaaagtatcttccttgaagacagaacatcatggtccctcgtgggtttggcggttcgacaagcatacttgatgcgcttggatcaaggtgctttcccgaataccgtgatgcagcaggagaccaaagaacaggcagagcataaacgactcatctggacatgtgagtctcgtttaacggagagatcaatcctcccttggctctcactacgcattgcctttctttcccgctaacctgagccttctcttggcccgtgcagtcgtctacattgcagatcgacagatatccgttcgcttggggcagtccttttggtctcgcggtccgtctctttccacacgcttcacatcgaaagactttcctagcctgcagccttgcttccacgacagccaggatgattatgcgtctgtccaccaggctacgatggagctaatccagctcatgcacaacgcccagggcatcctatatgcctcaccgaagcgcacgttagcgatcgtccatgatggcgattacagtcgttacctcgacgacttccagcgagcagcaatgaactggcaagcgacctggaacgacctcgccgtgtcgcccagagtcaaacgttctttgtccctcatctacgaatacctatgtctttacgtcaatgcattctcctttcaagcggttttgacaagattgtcgaccccaccgcagccttctggtcacagtcattgtactggacagaatgggaagcctttcagcaaaggcataatgaactctgccgacggaagatacgtttgggacgctattacggcggctaacaatacgctgaagctcatgaacgagttcgaacctgagcaagagctatgctacttgccgtatcgttactatttgtaagcatatccagtatgaggtatgcactgtgtcgtgttaacctttattggaagatacggcgtatatgccgcagtattcctgcacagagccgattgcgctggagcctttcaacctgtcaatagacgtcaagaaactgcaacgttggttcacaagtttatctcggcgttggacaagactacacccaacagatctcacatcggccacagatatagccagctactcaggaatctttggcaaaaaggacaacgcggatcggaaaaagctaggaacacgtgcgaggcatctatagtcctgcttccgaccgcaaccaaacagcagctaccaaggccagaattgctttcggctccatcaccttcttcagcaaccagctgtgaccatagcaacgactcagcagaggcaggtttgcttcatccccagatttctgccttgcatccggaacttccagattcctcttcaatgcaagagtatccatttgggcccttcatcaatatgccagactttgagctagcgagttatggaaaccccatgaacgatatagcacactcctctctcaattggggcacaaacttcattgcattatag

SEQ ID NO: 56 - protein, 681 aa, Trichoderma reesei
MTASTGKRSYRACVRCRRRKTKCDLGGIGEPRKASCVSCLQSNSQCVLAESRRGGNFRRYEPNKKHPAKNRKAPKRVPDAPPMALESMEREPKRNGERLNGDAIVGQLVSELRNPSDALHILAQSEKGESAASSTLPEGEPNNLSNGPSNATFLDEYKLVESGLIHRTAIFELLHIYSRNYHPYCPIVPAYMLGPSAIDRILKSDFFLLTVVLTIASRDSPSHSLIHRYCWDYAQRLLLEVLLGHPWTLKSRTVESLLLLSEWLPHIELTHGALKEDKSIFLEDRTSWSLVGLAVRQAYLMRLDQGAFPNTVMQQETKEQAEHKRLIWTFVYIADRQISVRLGQSFWSRGPSLSTRFTSKDFPSLQPCFHDSQDDYASVHQATMELIQLMHNAQGILYASPKRTLAIVHDGDYSRYLDDFQRAAMNWQATWNDLAVSPRVKRSLSLIYEYLCLYVNAFSFQAVLTRLSTPPQPSGHSHCTGQNGKPFSKGIMNSADGRYVWDAITAANNTLKLMNEFEPEQELCYLPMRRQETATLVHKFISALDKTTPNRSHIGHRYSQLLRNLWQKGQRGSEKARNTCEASIVLLPTATKQQLPRPELLSAPSPSSATSCDHSNDSAEAGLLHPQISALHPELPDSSSMQEYPFGPFINMPDFELASYGNPMNDIAHSSLNWGTNFIAL

SEQ ID NO: 57 - protein, 34 aa, Trichoderma reesei
CVRCRRRKTKCDLGGIGEPRKASCVSCLQSNSQC

SEQ ID NO: 58 - DNA, 2322 bp, Trichoderma reesei
  source 1..2322: mol_type = other DNA
atgagttcatcggcttcgtctagctctgccagagccgagcgacgcaaattccgacgaggcacgacaagctgctgggaatgcaaacggcgcaaaacacgatgccacttcgaaaagagtggctcaagcgcttgcgaggcttgcgagcgccgtggatgcaggtgtgtcctgcagcatgtcgaggagccgtcttcagtcacagaggcagcggaccgttcgattcgcgctcaagatgctggacatcaattgggtcatcttgaggacttggttggtcgactggtacgtcagtcgactcttcccgctgcttcaatccgtgattctgggcatgtcctgcaaacccaaaatccacccagagaattgcagagcagtgcatcaaccaaccatgcatcccgacgaaccccacaactacaacactcttccgactctcgacgcagattgcttcccaagccgggccacaggccaacgcagagctcctttagtcactcaacggacgataatctcgagaccttgaaccaaccaactcagacctcaacaccccgtccgtcatcactggtcaatacggccgatgccttgctgctgccgcctcacccaaccaatggtgctctgcggtgcagctccgtgactatgctcttgcagtccatactcccgtccgccaacgtcaccactcgcatcatgcatcaaaacaagctgctgatgatgtcgatgcacgtcttccggggcttgtccaccgacccttttcgatctgtagcccctcgacaaatctccctcttggcttctccaatgtcgagcaacgccgacccgattgatctagctcgacgactaattcagctcgcaatctgccttcagcagtcagatcgcacatcggagatctctgcgctctgcctcgatcggtcgaatagtgacgttgccggccaattttttgaggctgcttctcgttatgtcacctctcaggactctcttgtcgtctcctttggtggcatagagacattgatgcttgaaggactttaccacatctcgacgggcgagatgcggcttgcttggctcgtgtttcgccgagcgataggcattgctcacctgctgggcctccacgtcccgcaccggcggcgtcacagcaagcagatgccgatctgcttggcgactctctggtttcgcctcatattcacggagcgctacctggcgttggtgctctgccttccaatatgtgtccgagacgatagcttcatggacgacttaaatggcctagagcctcaagaacagcttgagcgtgtccaggccaagcttacgagtcgtatcatatccaggaacgaactgatgcaacgggatcgttgcgcaaacgaccttacctacgaccattacaatgtgacgaaggacattgaccacgcgttgaagcaggcggcgcgatctctgtcagcagactggtgggcttttccgagactgaaggatgctcagaccaatgcggagctgaaaaatatgacgtccaaggttgttcttcagttgcatcattacaaccttgtactgttgcttcatctgccgtacatcgtcccgcggctcggaattggctctactagccagggcacccaagactacacaaactccaagctctccgccatgtttgccagtcgtgagctccttacccggtgcctcattccggtcagccctaatcgtgtgtccgcttcaactcgggggaataccctcaaggttatccttgctgccctggcccttctactagctcacctggatagccaccggctaggtcgcgacaatgcttttgatcatcagcgcttaatggatctattgatggtcgaacgagccattcaaggcctggaatccatggctcaaaagcacgatgatgtgctgtgcttctccgccgttctagctctgaagaggcttggcaacatggaggcaacagccgcagacggccttgatctcagcgtttggtgcgagccgagcgagctgatgcttttggagtgcttggtccgagacgaaaaggacacactcatacttgtgtttccatatgttgggaacattcacattgtgcgccagtgccatcaagatgatgcgccaataagcgaggctgcatctagcggcgtaacggagacgaccatcctcaacaatgatctttcgacttcatcgtggcctccattatccttttggcccgacttggcccccatcactcccagccttggtgatacgcagagtaccatacctgatgaccacgaaattcacaactcatacaacctaccaagcggcgatgccgagcgtgtgcatatgccggcgatgccgcggctacttttacaggacctccatgtttcagacgggatttga

SEQ ID NO: 59 - protein, 773 aa, Trichoderma reesei
MSSSASSSSARAERRKFRRGTTSCWECKRRKTRCHFEKSGSSACEACERRGCRCVLQHVEEPSSVTEAADRSIRAQDAGHQLGHLEDLVGRLVRQSTLPAASIRDSGHVLQTQNPPRELQSSASTNHASRRTPQLQHSSDSRRRLLPKPGHRPTQSSFSHSTDDNLETLNQPTQTSTPRPSSLVNTADALLLPPHPTNGALRCSSVTMLLQSILPSANVTTRIMHQNKLLMMSMHVFRGLSTDPFRSVAPRQISLLASPMSSNADPIDLARRLIQLAICLQQSDRTSEISALCLDRSNSDVAGQFFEAASRYVTSQDSLVVSFGGIETLMLEGLYHISTGEMRLAWLVFRRAIGIAHLLGLHVPHRRRHSKQMPICLATLWFRLIFTERYLALVLCLPICVRDDSFMDDLNGLEPQEQLERVQAKLTSRIISRNELMQRDRCANDLTYDHYNVTKDIDHALKQAARSLSADWWAFPRLKDAQTNAELKNMTSKVVLQLHHYNLVLLLHLPYIVPRLGIGSTSQGTQDYTNSKLSAMFASRELLTRCLIPVSPNRVSASTRGNTLKVILAALALLLAHLDSHRLGRDNAFDHQRLMDLLMVERAIQGLESMAQKHDDVLCFSAVLALKRLGNMEATAADGLDLSVWCEPSELMLLECLVRDEKDTLILVFPYVGNIHIVRQCHQDDAPISEAASSGVTETTILNNDLSTSSWPPLSFWPDLAPITPSLGDTQSTIPDDHEIHNSYNLPSGDAERVHMPAMPRLLLQDLHVSDGI

SEQ ID NO: 60 - protein, 31 aa, Trichoderma reesei
CWECKRRKTRCHFEKSGSSACEACERRGCRC

SEQ ID NO: 61 - DNA, 1661 bp, Trichoderma reesei
  source 1..1661: mol_type = other DNA
atgcatcgtggctcgagaagaagctttgggtgtggtgcttgtcggcggatcaagattaaggtgaataggaagcccagagtcttcgaaagtatagctgattcgagttgatgcagtgcgatgaaaaccgcccagtctgtgatcgctgtctgcgagcaggccgagaatgcgtgtatccgcctcaattccactttatcgacacaaacactcgattcgcggtgccagacattcaagaacaacctgctcttccgagaacaggcacagaagatttgaagaccagattggccctgcttcagggagaaggcggcaacgatggaatgatctataagttccagctgctccgttaccgatcgacaactcctccgtcttcgccaaggagttccagccctcccgtactcaaggctcccagtttgtctcgaggagagaagcttgcctcagcgttggctgcttcattcaaaagccctccatatggttaccagctgcatcatctaggccgatacataaccgaaatcccgcgtcgccttggtcacaacgccgccctggatattgcagtagagtgtctgctgcaatctcacacacaattgcttcaagcaattcaatcatctcagcctagctcccagcccagccctgagtacatgagagccctccaaaccttgcagcatgtaattgcagatcctgtcttgggcacttctacagaaacagtttgtgccactatgttgatggcccactacgaggtgagaaggccggatccttgcgctctccaaagagtcatcttctgaccatgcacagatgatggccccagaaagacgctctcaccaatacatagcgcatgcggggggtgcatcgaaactgatacttgtccgcggtccaagacgcgtacgcaccaatttcgagtacaatcttttccgcactcaacgaggccgtattgtgcgtatatggtatctcataagcgtattggcactggagctgataaatagtgttaggtgctcgaggcgttacttcgaggggagcattgcttcttgcttacgggaggatggcaagacatctgcccggagaaatgcagaagcgtgaggttctcaagtgttgactacctctttgagagcatctgcaaaattcccactctcgtgaaatacgttttagattggagagctggcgtatctgaactaggtctgggcgaacttacagattatgggaggaagatacaacaggacctgagagcagcctcgcctcggggctccatgtctgtcagtctcgaggaggatcttggattcgaagagcgcaagtcgtcggcattggatgacgagtgcccgctgagtgttcactttcgctctcacgagggcgccatattccattcgtggcggtggatggccttgctcattgcagacctctgtctggtcaatcttgtggctgatccaagactgcgactgagaattgctgatactgcgcagcgaatctgcatgtgctatgaatatgcaagtctatctaaaccacttggagcccagttcttgcaggtgccattgatcattgcgtacgccgtcagtgaccaaaagcgcaaggactggatcacagacaagatcaacctattgatggggggactttatgtcacatatactgcacagtacttggatagcttgtcgacgatgatattgaagggtatcaaataa

SEQ ID NO: 62 - protein, 498 aa, Trichoderma reesei
MHRGSRRSFGCGACRRIKIKCDENRPVCDRCLRAGRECVYPPQFHFIDTNTRFAVPDIQEQPALPRTGTEDLKTRLALLQGEGGNDGMIYKFQLLRYRSTTPPSSPRSSSPPVLKAPSLSRGEKLASALAASFKSPPYGYQLHHLGRYITEIPRRLGHNAALDIAVECLLQSHTQLLQAIQSSQPSSQPSPEYMRALQTLQHVIADPVLGTSTETVCATMLMAHYEMMAPERRSHQYIAHAGGASKLILVRGPRRVRTNFEYNLFRTQRGRIVLEALLRGEHCFLLTGGWQDICPEKCRSVRFSSVDYLFESICKIPTLVKYVLDWRAGVSELGLGELTDYGRKIQQDLRAASPRGSMSVSLEEDLGFEERKSSALDDECPLSVHFRSHEGAIFHSWRWMALLIADLCLVNLVADPRLRLRIADTAQRICMCYEYASLSKPLGAQFLQVPLIIAYAVSDQKRKDWITDKINLLMGGLYVTYTAQYLDSLSTMILKGIK

SEQ ID NO: 63 - protein, 28 aa, Trichoderma reesei
CGACRRIKIKCDENRPVCDRCLRAGREC

SEQ ID NO: 64 - DNA, 2740 bp, Trichoderma reesei
  source 1..2740: mol_type = other DNA
atggcttccctctaccagccaaggccggtcctcccctctcaggtcatcccacatgcgccagagtacgtcgacgcaagacggaacgttcacggaggcagcagacagcctctgcgggaggcggctggcaacgcccaagcacacagcttcaacagcaacatcccgtgttaccagacccccgtgtcttcgctgcccccgactatcccaactcatatcgtcccctcacagtccatcgaggtcttctactccaggctgcagactccccgccaccagcctcgacaagttcgcaagagccggcaggatatcaaccctctctactattggccggcctttaagcagtaccgcaaccgacaagctcacaaggacacgcaaaaggacaagggcggcgtctggcgccgacccgagctcgaagatgcctttgtcgactgtgagttctgctcatcatcttttctctctgtgtgtagtgcacccggaagctgaccaaagtctgcatgaaccagccgtgctgcttatgcctcacatgggccggcgcaagttctccatgggagggaagctccatggccgaaacatgctcatcagcgagtacatcttcgtcatctgcgtggcgctgcttggagacaaagaaatcttccgcattgacaacagcaatgacagcatcgagcagatgggacgcaaacaagtgtctagtcacatgcaagttgtcaagaagtttttcgaagacctccgatgctgttagttacccaacccttaaccctcgcaaccccacccttgttgagagggaggtctgtttggttactgactggttggaattcctgctgcagttcactttctttttccttctgaagagaagaaggaacccggatctaccaactctgacgattattacgatgaagaaggccaggagtcgttcaagtcgaaccccatcctcacggcgttggccgagggccgtgtgcccgaagtgcggcccaactatgagtacttctcgcagctgctttcgctgcaagggctgatcacggtgcggcccaagacgtgcgagatctatgtgtcgtcggcagagatcaagatccgggacgacgtggcctacgacgcaaacaacacgcccctggaccaagcttcgtttcctcacctgaacaagtacaccaactgcgacgacttccccgatgccctgggcaaggacgtgctgctgcacgagtacactcggtcgctggaccgagcgacgtcagcatgcctcaagacggtgacccggcgatggcaaaaggacgcgccggccatgtacgagacgctggacctgccaacgcgcgacgaggagtgcctgctgctggaggtgtgctcgacgctggagctgcacgagcacgcaaagttcccctcgggatccgagctcacgggctttgttgaggtggccatcacgcagccggagctgcagcaccatcactggaagtgcgtcacccgattgacgcgaccggatgagctgtacggcgacgactcgaagcgcggcgtctacaccaatgagagcggcatccaccaccggggctgcagcgactccaagatgaacgactgcaattgccacatgcggccccggcaggacatccacgtgccctttcctgcggtcgagtgggccagcatcctgagcacggccgtgcaatatcctgacgtggagcaccagcggaagcgggagaagcgccacgaggccaagaagcagcacgatctggatagggccggcagcaagcgaaagcgatccgaggacgagggcgacgccgcatcgtggagccgccgcgagccaacgggcagcgacctgatctgcaaggtggccatgtaccaggagctgtggtcgcgggcgcccgactcgacgcagtggaagaggcaggccatcatcttctggcgcttcaacaccacgaaccagtggtacaagtacagccccgtgttcaagccgtcgggcacctcgtggcgctggctcaccatcaacgaccccatgtcgcgatatcaccaacagaaggcgctcatctacccctcatcctcgtcctcgtccgccgccggcgccgtgcccagggacgccgtcatgtcaccgacgcccagtgtgagccagcagctggcgggcatcatgaacgagaacctgagctcggcgtgggcaagcggcgcgcaggtgcccccggtccagacaaacatgaacgtgtttgactttccaacggggctggccacaccaccgccgacggcgaccgtccatggcccctacgcgggcgggagctttgaccctgtgaacagctacctgccaacagtgagcggcaccacggtgggcggcgcggcgaatgagcgatcgcactcccactcgctggcgttggctcacgggccctcgtactttgagggccaggcaggcttcgcggacatgaagcccatgacggcgccgcagtatctcgccgcgacgacggctgcccccctggatctggcctcgcagctggcattcgacgacacggccgccatccaggggtgggacatggcgcctctcgatgcttggcccggcccagcctctgcgacatccgccgcgggcggcggcgattgggcgactgccactgcctcgaccctgcccaaggcggagcccagtgaccactcggtgatttggaaccctagccaatgggacatgccggcatctgcggttgggggcgaacacgatgacagcccaagggctatgaagaggaggcgaacagagccctcgatgaacacgcatgtgcctcttccaggggcgtggtaa

SEQ ID NO: 65 - protein, 858 aa, Trichoderma reesei
MASLYQPRPVLPSQVIPHAPEYVDARRNVHGGSRQPLREAAGNAQAHSFNSNIPCYQTPVSSLPPTIPTHIVPSQSIEVFYSRLQTPRHQPRQVRKSRQDINPLYYWPAFKQYRNRQAHKDTQKDKGGVWRRPELEDAFVDSVLLMPHMGRRKFSMGGKLHGRNMLISEYIFVICVALLGDKEIFRIDNSNDSIEQMGRKQVSSHMQVVKKFFEDLRCFHFLFPSEEKKEPGSTNSDDYYDEEGQESFKSNPILTALAEGRVPEVRPNYEYFSQLLSLQGLITVRPKTCEIYVSSAEIKIRDDVAYDANNTPLDQASFPHLNKYTNCDDFPDALGKDVLLHEYTRSLDRATSACLKTVTRRWQKDAPAMYETLDLPTRDEECLLLEVCSTLELHEHAKFPSGSELTGFVEVAITQPELQHHHWKCVTRLTRPDELYGDDSKRGVYTNESGIHHRGCSDSKMNDCNCHMRPRQDIHVPFPAVEWASILSTAVQYPDVEHQRKREKRHEAKKQHDLDRAGSKRKRSEDEGDAASWSRREPTGSDLICKVAMYQELWSRAPDSTQWKRQAIIFWRFNTTNQWYKYSPVFKPSGTSWRWLTINDPMSRYHQQKALIYPSSSSSSAAGAVPRDAVMSPTPSVSQQLAGIMNENLSSAWASGAQVPPVQTNMNVFDFPTGLATPPPTATVHGPYAGGSFDPVNSYLPTVSGTTVGGAANERSHSHSLALAHGPSYFEGQAGFADMKPMTAPQYLAATTAAPLDLASQLAFDDTAAIQGWDMAPLDAWPGPASATSAAGGGDWATATASTLPKAEPSDHSVIWNPSQWDMPASAVGGEHDDSPRAMKRRRTEPSMNTHVPLPGAW

SEQ ID NO: 66 - protein, 86 aa, Trichoderma reesei
KGGVWRRPELEDAFVDSVLLMPHMGRRKFSMGGKLHGRNMLISEYIFVICVALLGDKEIFRIDNSNDSIEQMGRKQVSSHMQVVKK

SEQ ID NO: 67 - DNA, 2407 bp, Trichoderma reesei
  source 1..2407: mol_type = other DNA
atgcctgatcggtcctcccaagcaaagggccggccgctgcgccgtgtgctgccggctcccgcccagcaaggcccggcgacagcggctcccgctcctctggcatcgtcgtcgtcgtcgtcgtcgtcgaggaagaacctgacgccagcagcgtgcaacgcgtgccgcaagcaaaagacaaaggttctctctccagctgccccagcactgctccctctctcccttgcatgccagcctactaaaccagccccctcccccggcagtgctccggcgagcggccaagctgctcccgctgcgtccagcgccgaaccgaatgcctctacacgactcagcccggcgagacaacgtctcaggccctcaaacggggctaccgggacctgcgccagcgcacgagcgtccacgaggagctgtttgagctgctgaggaacctgcccgaccgcgaggccgagcacgtcttcaagagaatcagagacggcgccgacgtcaacacgatcctcaaccttatccgagcgggcaacctgctgctgcagatggccgttgcgcccgagacgcggttccgatacgagttcccatatcggtctgagatgccggactattgcattgagaacaacccttacctggactcgatcatctggggcgcggcgtcgttgtatgcgcaggatagtagtaggcggcctaatgctgtcgctgttgctgcttcttcttcttcttcttcttcttctgctgctgggccggctggtgattatggcacggagggatatgaaagcatctacctgaagccgttccatgcggctgaggtcgttgatccccggctgagtgatgccaagatctcggcttggacgtctgtcaccagcgacgacgtgctgatgcgagagctgctaaaggtgtttctgcggtgcgaatacatgttcacatcggcattccaaaaggactacttcttggacgatctcgtagccagacgagacgagttctgctcttcgttgctcgtcaacattatcctcgcttatgcttgcgtacgatagcctccccctccccatcgacacaaaaggtacaccttacagtttctaataacgaggctctcataggtctgctctccacaactttcaaaccgcgccgagtactggaaccccgaaacgctcgtgtatcgcttcctggccgaagcgaagcgcatctgggagctggaatcgggccaaccacacatcgccacgatacaagccgggatactcttcagcgtcttccacaacctctgcggactagatgagattggccagccttatcggcttcaggccatagagctggcccgagagatgcgtcttttcgatagcaccgtgggtgggcgcagcgacaagatgaagaagtgcatggcgtttctagcatggaccctgtttgactgggaatcgtgagtaaccccaaccttctaaaaaacgcctgatacactgaactcatgattctgttcctgcatgtagactcgtcagtttctcctttgtgttttctccgttaatcaaagagccaccaacttggaagctgccggatccctcgaaagaccccagtttctacggagaggtttggctcaagtatcccctgggcaagaacctttcgccgtcgtattttggccaaatctttcgcgcaaagtctcagttccggctcatcatgcacgactttgccatggcagcgtatacggagggctcgcaggtgacggtcgacaaagcgtacgagctccaccagagactgctgcgctggtacgaagccctgcctgctcaactgcagccgaggacaatcgtgcttcctgcgcacttgcaactccagtaggcacctccaaactgtccatttcagtgtcaagtcgctaacacaaagcaacaacagcacttattatcacttcctaatcatcaccatcttcgaaccccttctcgacacagaaaccgaccgagaaccctcccccaaggagatcgtgggcgagtccaaacgccagacccagaccctcatccgcctctactacctccgccacggctacgacgccatggacctcttcctcgttgtccctctcatgatggccgcctccgattgcctcgaggccattgaccggggcaccaccggccgggacctcgaagtcctgcgctccacgctcatcctagtcgcaaagggactctacaaccagcgccgcaaccactacctggctgaggcgctgttccgggtcattcggggccgcatgcggccgtcggaagtcgcactgttgaaggacacgatgagccttgacgagcgggagtgggatgagaggcgggacatggtgcaggctgtgaggagtcagtggccggtgagtgtggtgaagaagaaggaggacttggattcgcatatcttgacgaacctcgtggagaattatggacatctgaacgtggaggatactgttgcgtga

SEQ ID NO: 68 - protein, 709 aa, Trichoderma reesei
MPDRSSQAKGRPLRRVLPAPAQQGPATAAPAPLASSSSSSSSRKNLTPAACNACRKQKTKCSGERPSCSRCVQRRTECLYTTQPGETTSQALKRGYRDLRQRTSVHEELFELLRNLPDREAEHVFKRIRDGADVNTILNLIRAGNLLLQMAVAPETRFRYEFPYRSEMPDYCIENNPYLDSIIWGAASLYAQDSSRRPNAVAVAASSSSSSSSAAGPAGDYGTEGYESIYLKPFHAAEVVDPRLSDAKISAWTSVTSDDVLMRELLKVFLRCEYMFTSAFQKDYFLDDLVARRDEFCSSLLVNIILAYACVCSPQLSNRAEYWNPETLVYRFLAEAKRIWELESGQPHIATIQAGILFSVFHNLCGLDEIGQPYRLQAIELAREMRLFDSTVGGRSDKMKKCMAFLAWTLFDWESLVSFSFVFSPLIKEPPTWKLPDPSKDPSFYGEVWLKYPLGKNLSPSYFGQIFRAKSQFRLIMHDFAMAAYTEGSQVTVDKAYELHQRLLRWYEALPAQLQPRTIVLPAHLQLHTYYHFLIITIFEPLLDTETDREPSPKEIVGESKRQTQTLIRLYYLRHGYDAMDLFLVVPLMMAASDCLEAIDRGTTGRDLEVLRSTLILVAKGLYNQRRNHYLAEALFRVIRGRMRPSEVALLKDTMSLDEREWDERRDMVQAVRSQWPVSVVKKKEDLDSHILTNLVENYGHLNVEDTVA

SEQ ID NO: 69 - protein, 28 aa, Trichoderma reesei
CNACRKQKTKCSGERPSCSRCVQRRTEC

SEQ ID NO: 70 - DNA, 3627 bp, Trichoderma reesei
  source 1..3627: mol_type = other DNA
atgtcttccgcatccgccaccgcccaaggcttcaccctgcctctcgacaccgacaaccaccacgacttcgccttcgaccttgaaccccgcaattcaccggcagcccaacagcgccaagtcaagcagcagcagcagcatcagcatcagcagcagcagcgctcgtctatcgactccaacctgctgtcgcccttcctgtcgcgcgtcaatagcgactccctctcacccgccaatccctcggaaacgcctgccggatccacctcgacgctgagcatataccagtctagcgacttcagtgacttcgacgacgaccccttcttcggtgccaacttcaccaatcccgaggtcggcacaccatcattcctggacgaccagagcgtatgggacctctcactaaacaataccccaccagttgagccccagcaagagcacaatgccaacgacgactcctatcctctgacgccggaccaaacagcctctgtccacacgttgtcgccgggaagtgaagagaggacaaccctcacgccgcccgaccagcatcagcttcccagctccgtctcgccgcagcagctccaaaaggccttcaagccgaatccccttgtcatcccgccacctcagttgacccccagccagaccgacagtggacgatccagcgaggacggcttggcgcctgctcctgtcgatatgcggtgtcaaagtccccgagtcaccatctccgtatgggacaaggacaataacggacccatacacaccgttgagcggacttttgaagacagtccctcgacggtacggggcggcctcgagtccgcgggagacttgatcacattgtccgcgaaccagggacacggccaggcggctactgcagccactgcttcaagggattccataggccaatggcgacgtgaccctgtcaccggccattccggcctagatcccaagaggcggcctggcgaggaaattgcaagcatcaatgagattatcagccgccgagagattgaggagaagaaccaggaagtcggcaagtggctcaccggcaatgcgcatgacaccgttactcccgagaggccgtcccaggaggacattaatgctgcagaagagcaaaatcgcaatagcaacgatggcatcccccttggcgaccagaccgagaacaagtacatccccggccagacgtactatgccccaaatggtgccggccagttgtcggacgaggattatgacattatctatgccaaccgcggctgggctgacgctcccatgcttcactctatcagaaatggcggccaaagccgccatcagccccagagctcgcaggccgccatcgagcggttcgagagaatgtgtcgtgacaacgactctatcatctcccgagctgcaacatggggcactcgccgccgcagcttgcccagcgttgtcgaccttgatattgagggtgtcaccagcggcaacttcttgaagaagctgtccattagtcgaggcggcgataagagcagcaggtccagcagtctctttaaagacattcgcggccttgtccgccgccccagcggctctcacatgcgcaagcgctctcgcagcggacagcgagaaggcgaggctgcgggtgaccaggcggagcccacttcaactcacaataagcgtgatagcatgccgcacctttctcccctatcgcgcacgtccagctggggcaagaaaaaccaggccccgagcatcaacaccgccttggtgtccatgggccagaatattgcctccatcggcacgacgcactcgcgcgctggatccatcagcaccattcctgcgccctcgacctccaagggtccccgtctcaatatcaggaactctctcagaaggccgcgaagcaagtcggacctacccaagccaacagcttcctcctcttccaccaccaacagtaacaccgacggtactcgctccagtctcgcccacttgtggcagagtgttggtggtccgccagtagctgctctgcccgtcgaaccagacgaggatgaggacgaagatgacgatttttacgaagataacgacatgaagatgaaccccaatattattgacgacattacgccgaactttgctggctttcagcaacacatccagaccctgaacccaggactctcaccggccaatagctacctcgtagatcgcattgcgcaccagcaaattgttcgatacaagcagctactaaacaacaaggtcaagcacctcggtctcggcgccaattgcccctgcggtcccctctgcttggcgcttgggggctcagcgaatgtgctgagcgcaagggcaggagatggtaatggcggcagcaaggggctggacgccttgtcggctccctatgacgaggacgacgatgtcacgccgaccgagggcgccatcaatcaggaaagcttcccgaccgacatccccatgccgccgactcagcacctgccggctgagttcgaatgccagctatgctaccagaagaagaagttcctcaagccgtccgactggacgaagcacgtgcacgaggacgtgcagccctttacgtgcacctgggacaagtgccgggatcccaagatcttcaagcgcaaggccgactgggtgcgccacgaaaacgagggccaccgacacttggagtggtggatgtgcgatgtcgaagattgccggcatacgtgctaccgccgcgacaatttcctgcagcacctcgtccgcgagcacaagttccaggagcccaaggtgaaaaccaaggcggccatgaagaaggccggcacggtcgatcccacctggcacaaggttgagcagtgccatgtggaaacgagcaagcagccgcacgaggagccgtgcaggttctgcggcaaagtcttcccgacgtggaagaagttgactgttcatctggccaagcatatggagcaaatctcgcttcccgtgctgcgcttggtagcggccaaggcaaaagaactggcggcggataccatcatcagcccggtccaggatccgcccccacggcacgagataccgctcatgccagagcagaggtcgcctgcccccatgcggtatgtggacaatggccagcagcagcagcagcagcagcagccgctccagcaacaccagcagctttcctccgtaccgtccatggctccccaacagcagccgcaccagcaaccagtgtcgtatgcgcaggcccagggcaccttcatgtatcccgtgatgcccccaggaccgtccttcacgccgttctattctccccaattcgagagcatcggtcacagcctgcatcaacccccgtcgatggccgctagcccgctaggctacgaccccggccggcgggttcaggacatgccagtcacttcggctccgtatgtgcaggggtcgaatccgtatgccaacatgacaatggcaaatgaggtggagccaccattcccacccctgaatgcgctgggcctgcagaacgtgcccgtacaggttgccggcgtcccccctatgggcgcgcagatggcgtacgacaacatgatggacccgtcaagcgtcaacggcagcccattcagcggacacgaaacgctgtcgacctattctcactctcctcaccagggcgcgattcatctagataccaagagctgggatgatagacaggtgtctggatttttctga

SEQ ID NO: 71 - protein, 1208 aa, Trichoderma reesei
MSSASATAQGFTLPLDTDNHHDFAFDLEPRNSPAAQQRQVKQQQQHQHQQQQRSSIDSNLLSPFLSRVNSDSLSPANPSETPAGSTSTLSIYQSSDFSDFDDDPFFGANFTNPEVGTPSFLDDQSVWDLSLNNTPPVEPQQEHNANDDSYPLTPDQTASVHTLSPGSEERTTLTPPDQHQLPSSVSPQQLQKAFKPNPLVIPPPQLTPSQTDSGRSSEDGLAPAPVDMRCQSPRVTISVWDKDNNGPIHTVERTFEDSPSTVRGGLESAGDLITLSANQGHGQAATAATASRDSIGQWRRDPVTGHSGLDPKRRPGEEIASINEIISRREIEEKNQEVGKWLTGNAHDTVTPERPSQEDINAAEEQNRNSNDGIPLGDQTENKYIPGQTYYAPNGAGQLSDEDYDIIYANRGWADAPMLHSIRNGGQSRHQPQSSQAAIERFERMCRDNDSIISRAATWGTRRRSLPSVVDLDIEGVTSGNFLKKLSISRGGDKSSRSSSLFKDIRGLVRRPSGSHMRKRSRSGQREGEAAGDQAEPTSTHNKRDSMPHLSPLSRTSSWGKKNQAPSINTALVSMGQNIASIGTTHSRAGSISTIPAPSTSKGPRLNIRNSLRRPRSKSDLPKPTASSSSTTNSNTDGTRSSLAHLWQSVGGPPVAALPVEPDEDEDEDDDFYEDNDMKMNPNIIDDITPNFAGFQQHIQTLNPGLSPANSYLVDRIAHQQIVRYKQLLNNKVKHLGLGANCPCGPLCLALGGSANVLSARAGDGNGGSKGLDALSAPYDEDDDVTPTEGAINQESFPTDIPMPPTQHLPAEFECQLCYQKKKFLKPSDWTKHVHEDVQPFTCTWDKCRDPKIFKRKADWVRHENEGHRHLEWWMCDVEDCRHTCYRRDNFLQHLVREHKFQEPKVKTKAAMKKAGTVDPTWHKVEQCHVETSKQPHEEPCRFCGKVFPTWKKLTVHLAKHMEQISLPVLRLVAAKAKELAADTIISPVQDPPPRHEIPLMPEQRSPAPMRYVDNGQQQQQQQQPLQQHQQLSSVPSMAPQQQPHQQPVSYAQAQGTFMYPVMPPGPSFTPFYSPQFESIGHSLHQPPSMAASPLGYDPGRRVQDMPVTSAPYVQGSNPYANMTMANEVEPPFPPLNALGLQNVPVQVAGVPPMGAQMAYDNMMDPSSVNGSPFSGHETLSTYSHSPHQGAIHLDTKSWDDRQVSGFF

SEQ ID NO: 72 - protein, 21 aa, Trichoderma reesei
CRFCGKVFPTWKKLTVHLAKH

SEQ ID NO: 73 - DNA, 897 bp, Trichoderma reesei
  source 1..897: mol_type = other DNA
atgcacggcagcgacgccagcggccgcagagtctcgctgctcaacgatgacgaggtcaccagagtgattgctcgccccccagccttcacctggccgtccgcgcaatctcagcactacggcagtgactcttcgctaccaaacacccctgcgctcctccgttcggactctttcgactctcagctcagcgaagccgccatctcgcccttgacgccccccggcgacagctactacggaagcgttcggcctctgacttatgccttcgggcgcgagtacgctgaaggtcctcccactaagcgccctgcctacgggccttgcagccccccggcctctggcgaagaggacagctccgctgcatcttgtgcccccgagcgccccgtcaagcgctacccctgccgctaccgtgaccttcatgggtgcgaaaagaccttcaccacctcggggcacgcttctcgccactccaagatccacactgcggaaaaggcggtccattgccaatatccgggatgccacaagaagtttaccagagcagacaacatgaagcagcatctggaaacccactacaaggacaagacccgaagctcatcgggcagccagcgcgccagcaaggcctctctggcctccaatgcgcgacgcagctcgactgcatctagccggagcatgtcctccaccacggggcccgactcctttgttttctgggacgagagtgagcttcgccagaagcagagcctctcgccgacgtctccgctttcgcagccgacccgcgactgggagtctggcatgggcgctatcgaacttccgttgctcaatcgcccctccggcgtcggtcgcatgcccagcggcctcgacactctggccatggccgtggagatggattgccgtgaaggtcgaggattctga

SEQ ID NO: 74 - protein, 273 aa, Trichoderma reesei
MHGSDASGRRVSLLNDDEVTRVIARPPAFTWPSAQSQHYGSDSSLPNTPALLRSDSFDSQLSEAAISPLTPPGDSYYGSVRPLTYAFGRDSAASCAPERPVKRYPCRYRDLHGCEKTFTTSGHASRHSKIHTAEKAVHCQYPGCHKKFTRADNMKQHLETHYKDKTRSSSGSQRASKASLASNARRSSTASSRSMSSTTGPDSFVFWDESELRQKQSLSPTSPLSQPTRDWESGMGAIELPLLNRPSGVGRMPSGLDTLAMAVEMDCREGRGF

SEQ ID NO: 75 - protein, 56 aa, Trichoderma reesei
CRYRDLHGCEKTFTTSGHASRHSKIHTAEKAVHCQYPGCHKKFTRADNMKQHLETH

SEQ ID NO: 76 - DNA, 1585 bp, Trichoderma reesei
  source 1..1585: mol_type = other DNA
tgagcaaacgggtggccgcagaggaggcgcctggagcgtcgcctcgcaagagactgaatagcggcagcattggggcctcacgtacgacttggagctcgctagccatcgaacccccctttgaaacacggctaatcgatatatctcaacagatctcgacatggacgatggggatgacgactacaccccgagttcaagccgggcctcgagcgcagaggtcgattccaccatcacagccgcgacgaccccccggacgaagttcccgtcagactacaagacgctggcatgctcatggccaggatgcaccaagagcttcaaccgtccggctcggttgagggaccacatgaactcccacaccaactcccgcccccataagtgcccctacgacggctgcacaaaggactacatcgaagacaagcacctgaagcaacacatcaaggccgtccacaccaacgaccggaaatatgtctgccagcgagaaggctgcggaaagagcttcgtgaccggcacgaggctcaagcgccaccagctcgtccacgagggagccgaccggttccgctgccaggattgcggccagagcttccggaaaaaggagacactcaacaagcacgtgcgcaaggagcaccaaggcttgccggcgcatcaatgcccggagcccacctgctcgtcggcgttcgactccaagggggcgctgaaccggcatcgcgagagggagcacgggccgctcaagtattggtgcatggaatgccctccgcagatgcgcgaggacggcacgacgtacagggtaggcttcacgacggacacgcagctgcagtcgcacatgcggcaggaacaccagaattgcatgttctgcgactacaagtccagctcgctatgggagctggagacgcacattgagattcaccattccggaaagacggtcgaggaccgcaagacggtggcctgcccccacgcgggctgcgacaagagattcaccaaaaaatccaacctcaacgtccacatccgcacggcgcacgaaggcttccgcttcatctgcggccaagtcgaattctccggccccgggctcgagagctggaccaacgaccaaggctgcggcgacaagttcagcacaaaggtccgcctggaagaccacatccgcttcatacacctgggccaagaacgcccaaagctctccaaggcggagaaacccccgacgacggacctcatcgacgagatatccggcgtggccaacatcaagaagcagaagctgcgctgcccccagtgcggcgagggcttcattcggtatttcgacctcaacgcccacattgcgtttgcgcatgagtcgaaccccgccgttgacgaccacgccgtcgattcagcagcagcatcctacactgacccgtccatctttgctcatgggcaggggaaccctctcgagcagcacccttggacaggggagtttgcccaggatgacattttctctgcgcaaatgtcgtttgggacggggcaggaggggtggatggaggacgatgcgacgctcttgatgttggcgcgtggggaggcaggcttagatggggcatctatcgatcccacactgggaggcttttag

SEQ ID NO: 77 - protein, 505 aa, Trichoderma reesei
MSKRVAAEEAPGASPRKRLNSGSIGASHLDMDDGDDDYTPSSSRASSAEVDSTITAATTPRTKFPSDYKTLACSWPGCTKSFNRPARLRDHMNSHTNSRPHKCPYDGCTKDYIEDKHLKQHIKAVHTNDRKYVCQREGCGKSFVTGTRLKRHQLVHEGADRFRCQDCGQSFRKKETLNKHVRKEHQGLPAHQCPEPTCSSAFDSKGALNRHREREHGPLKYWCMECPPQMREDGTTYRVGFTTDTQLQSHMRQEHQNCMFCDYKSSSLWELETHIEIHHSGKTVEDRKTVACPHAGCDKRFTKKSNLNVHIRTAHEGFRFICGQVEFSGPGLESWTNDQGCGDKFSTKVRLEDHIRFIHLGQERPKLSKAEKPPTTDLIDEISGVANIKKQKLRCPQCGEGFIRYFDLNAHIAFAHESNPAVDDHAVDSAAASYTDPSIFAHGQGNPLEQHPWTGEFAQDDIFSAQMSFGTGQEGWMEDDATLLMLARGEAGLDGASIDPTLGGF

SEQ ID NO: 78 - protein, 335 aa, Trichoderma reesei
GCTKSFNRPARLRDHMNSHTNSRPHKCPYDGCTKDYIEDKHLKQHIKAVHTNDRKYVCQREGCGKSFVTGTRLKRHQLVHEGADRFRCQDCGQSFRKKETLNKHVRKEHQGLPAHQCPEPTCSSAFDSKGALNRHREREHGPLKYWCMECPPQMREDGTTYRVGFTTDTQLQSHMRQEHQNCMFCDYKSSSLWELETHIEIHHSGKTVEDRKTVACPHAGCDKRFTKKSNLNVHIRTAHEGFRFICGQVEFSGPGLESWTNDQGCGDKFSTKVRLEDHIRFIHLGQERPKLSKAEKPPTTDLIDEISGVANIKKQKLRCPQCGEGFIRYFDLNAH

SEQ ID NO: 79 - DNA, 1763 bp, Trichoderma reesei
  source 1..1763: mol_type = other DNA
atgaaggccaagcaaggctgctggacatgtaaacgtgggttcactgttctccagctggcacgctctggcggcagcagctcggatgcctctgctgacaggtgccgtaaaagagcggaaaatagggtgcgacagggatctgcccgtgtgccacaactgcacccgcacgggtagacaatgccagggctacggacttcgtctgctgtggccggatcgccatgatggccggcgcaaggacagtggtttcgtggtctatgagcctccagagaacccgctcgaggcttccaagagctacggagtgcacttcctcaacgtcaatcacaatgatgtcgcctctgcgctggacccgacatcttacaatgccctcatcacgaaaactgttgccaagcctacacgggctctcaatctctatccgacaatgatcggccaagatgctctgttcatgtcctactgtaagggctcagttaaccccagtccctccatctctcttcccaagatccagtagttaacctgacctgaagacgagcgcgtcattgctccgatgatctcgaccactcaagtccagaacggctttcggacagatcttgtccagatggtgttatcacggggagacagcgcaacaaaagcgctatggaactcaatgatggctgtggctgcctttcatcacagtggctctgcagcagccctcacgtacaagacgaatgccgtgcgctacctttccgcttccttgactcccggacatgaaacgagctcggtcgacctcaaccaaacgcaattcgccgcgtccatgatgctatgcatctacagtgtaagtcacaaggagcgaagtgccacggggacgtatgcactcgtatgctcacggactccaggtctttgacgagaccgacggccactggaccacacacctcgacggtgccaagtatatgctggaagggttatacccggaatatcgaaggcagcttagagcggatttcctcttcacctggtttctgtatcacgaagtgttggcatgcttcagtcagcccctccgacaagcccacaactgcatcgatgttttattcctgctgggggcttccaattgcgatgctactgtggtgagtctgcgcaattgcatccccttgtctctgcctacctggtatatggcgtaatgctccgctaacacgagcggttgccagatacttggagcaccggggtgttctctggagacaattgcaatcatccatcgaataaacaaattccgcatagccatcatacagaaagaagtcgacccactgtcgcccaagctgatccagcagcgtttcgaactggagcacactttgcacaaccttgtccagcgattggatccagaaggcgaagctggtgaagttccagctaggcgcacgcgcattctagccaccgcagagctctatcgcatagggacaatactgtatctgctccacgtcatccccttgcccggagacgaagaggcaaagacaacttatctggaggagttctttgacatagtggaccgtctgggagttgccaccagcccatggcccattttcatggttgcatgcgaatgtcaaacagacgaacagcgcattaggattctacggatattggatctcatgaataacgtgagaaacattggcaacattcgggtcatgcgcaagcttattgagacatgctggaagcattatgaccttcaggctgattcgacgtacacgcaccgcacgaaatggtgggaatttattcactatgagacggcagttccttggttcatatga

SEQ ID NO: 80 - protein, 491 aa, Trichoderma reesei
MKAKQGCWTCKQRKIGCDRDLPVCHNCTRTGRQCQGYGLRLLWPDRHDGRRKDSGFVVYEPPENPLEASKSYGVHFLNVNHNDVASALDPTSYNALITKTVAKPTRALNLYPTMIGQDALFMSYYERVIAPMISTTQVQNGFRTDLVQMVLSRGDSATKALWNSMMAVAAFHHSGSAAALTYKTNAVRYLSASLTPGHETSSVDLNQTQFAASMMLCIYSVFDETDGHWTTHLDGAKYMLEGLYPEYRRQLRADFLFTWFLYHEVLACFSQPLRQAHNCIDVLFLLGASNCDATVILGAPGCSLETIAIIHRINKFRIAIIQKEVDPLSPKLIQQRFELEHTLHNLVQRLDPEGEAGEVPARRTRILATAELYRIGTILYLLHVIPLPGDEEAKTTYLEEFFDIVDRLGVATSPWPIFMVACECQTDEQRIRILRILDLMNNVRNIGNIRVMRKLIETCWKHYDLQADSTYTHRTKWWEFIHYETAVPWFI

SEQ ID NO: 81 - protein, 28 aa, Trichoderma reesei
CWTCKQRKIGCDRDLPVCHNCTRTGRQC

SEQ ID NO: 82 - protein, 292 aa, Trichoderma reesei
MTPHVPDQPVKKQTKWSSEEDALIIELRGKNMKWEDVSKRLPGRSAISCRLHYQNYLERRSEWDEERKNKLARLYERFKADMWAKVAEELAVPWRAAEAMHWQLGEADMARRAGVVPFTLANVNNEAGSNRSVAGRSQAPIQPQETSREMKPPSPPSSYSRSQLMPPIAPMTPAPRTPRRGHPPPPPPPAPAAALPHPLGDYRRSPGPGLAPIQPHPPSHMSGPLPSVAQLTTGMSPYRPPSGPPTPTSLPAMTTTPLPRYLPLEPAVSAGTKRRASPDMTRPDSAHRRRLL

SEQ ID NO: 83 - protein, 884 aa, Trichoderma reesei
MDRMGMFQVPPLVGPPPLTNQPPQIFGSFLPDGLPSVQQLPPDLAAHIFPDSSLLLDDSNDAKRRRIARACDQCRRKKIKCDGKLPACTHCTNYKTECIFTQIEKKRATPKGAKYIEALENRLARMEKLLRLAGILVNDDDDLAELEKRLTEKAQQTAGAQSSAPSSPSRLTFESDGTASPRSSVTSPGFQKDKDRRQDDKHKPIATPVSLQGEEEVEALSEMMCSLVTNNCGETRYIGSSSGFSIFSPKGIQWINNKTGDAAFQNMISQVSEDDHKWTNWKPEIFRDLFQRPVFQPLPSKAEAMSLLTDYFENFNCMFPLFHQPTFMHLVERQYSDDPYQGSGWWASLNCALAIAHRLRVMSNLVPQEEDDKAWGYLKNAMGVFAELTMRNTDLLSVQALLGMALFMQGTPNPQPTFLLIAAAIRLSHSIGLHKKGTGFNLNPIEIEQRKRVFWIAYMLDKDLCLRSGRPPAQDDDDMNVELPDADPEDNIGNIPLADGKGKMNLFRVMCEFAVIESNVYKRLYAAKAAKQSDGELLATIGELDQQLEEWKDRIPIDFRPEHEIRASHTPLILHIVMVHFTYYNCLTTIHRMSVHHGFWTSRLANYAISGQNVRPLNPRVYSSAMLSTAAARASISLLKYIPQGDFSCVWLILYFPVSALVTLFGNILQNPLDQRAKSDVRLMNLVVTFLSMLGQEAEQGGVHRMLGICSEFCRIAQRVIEKAEKEQSSRRKRKAADTPGSIDAHNHHTTATTTPQTNVHDTLTPEFAAARASIHNAANKATPLGSSPSVDSMGWTQDSQMPQDIEFESFSDLTGFGADVQSPPVPSGRGFPQPVLPQDLFSLSMSLDWNWVEMSGGAYPTVENGNFGGLENTGVLGPREQRQ

SEQ ID NO: 84 - protein, 422 aa, Trichoderma reesei
MDPEPDLGTAKSGSPKPASGTRVSLACLPCRTRHVRCDATKPRCNRCCEEDKECSYAKSRRGGLDRAALAARRSQAAAQAAAKKSSNSNSNSNSNAHSSSGSSSSNGQPSPAGSNDGTRLTSGLFPAHHPEPPPATAIDPSLCFHRYHPCAPPRRFLERYLQDQIRQDELRPFVYVMRFVGSIYLSSHQPSLRPRCQELEELAASVIARVPPTAINFFMVPCHAIYSACLYWRGNTASSRHHMNTAIQLALDFGMQRREFAVDHGDGDPVLEECWRRTWWQVYIIDAYYAAIKRDAGFATFHVDATTDLPCEEDEYERGEIPRPKTLEEFESREFSSEDTVFSSFAYLIGGVRGMASAMARVLDLPASNASDSGGPSPKVLESVDSITEGWLLLLPESKRDIFSADGQVDELIFQAMMALHA

SEQ ID NO: 85 - protein, 612 aa, Trichoderma reesei
MAFPPQYREAPPSLPSIIVPGSGANRYRDQGIPFSRSPAMAIPGMDMRDDVPPPLPPPRHLPFGEAPTQYRDEVKDKRDFSRGSSFASRSSFASGYGSMSPALLDERSSYRRRETGSINSDEGYASYASTERSRDSLSSEPFGLHHNKFQFQSAAELHGDSMKKMLTPIRTLDRSPPRSLLGGPVSSLSQGLQNEGRFAPSFNNIPIQLPIHSRAPFETQPRDSPTFSAVSPLSTPFHRSPIDHRSPYDSSEPDRSPRGRSRRNNSDDATSTQGSFADDMEIEETSSLKRLHIEDAYAGAGQKRRAASPASDDLGLHMMPGQGAARGSPTPRLATGPPPATSLLSGASARPSFFMSSITIPPNTTSSSYGHRSPGGISPGALSPTSCTSPYNTPASLNPSPKTAVANRASMHARTASGASPRKLPEMQKSGGKFQGFYMCECCPKKPKKFETAEELSAHEAEKQYECSFCGNRFKNKNEAERHQNSLHVRRHSWSCSALSGYSRAFHESTAHPGEADACGYCGEEFPRSGHGPKPGPVHGGIQTRHATEFDWEERIRHLQEVHKFRECNSSKKFYRADHFRQHLKHSHAGTSGKWTNMLENACLMEEDPSPR

SEQ ID NO: 86 - protein, 696 aa, Trichoderma reesei
MVKGNASGGIYSATYSGIPVYEFQFGGDLKEHVMRRRHDDWINATHILKAAGFDKPARTRILERDVQKDVHEKIQGGYGKYQGTWIPLEAGEALAQRHSVYERIRPIFQFVPGDESPPPAPRHASKPKAPKSRPPLPKWNSGRSKAAAGMAPSVTNPAAAAMPVVEEYDNSEVVMGGVEEDTPDNLTVASASYVAEDDRYDMGHMSTGHRKRKREEQLQDLTEQQHAVYGDELLDYFLLSRNEQPAVKPDPPPNFQPNWLIDAENHTALHWASAMGDVDVIKQLKRFNADVAVRNIRGETPFMRSVNFTNCYDKQTFPAVMKELFETVDVRDSQGCTVIHHAAIMKNGRVYSPSCSRYYLDNILNKLQEALEPSAFQRLIDVQDNDGNTALHLAAMRNARKCIRALLGRNASTDIANLEGLRAEDLIMELNAAKKDRAPQRSSSPFAPDSQRRATFRDAFSSSGGAGGDSKVRKPAPSFQSAAATTVHSRISPLIMEKFQQLAKSYDEEWHEKDVAEAEARRILTNTQAELNLVRQQITELEAQLEPDDVAAKVMSEANLVRHNVLSLITHQNRLHIQQAVENEMSKLNGNASGGDGGGGDDSYEERLALARQLSQMLMEQRQAETEYVDALSMVGVGDKIEKYRRLLKRCLDTKDGEVLDANLDSLIDMMEEEREVHDMDGSTTAGGEPMDFAVM

SEQ ID NO: 87 - protein, 729 aa, Trichoderma reesei
MSYPSPGAEALDAGPFYHTGTRDETHEQHHEPEEHQQQHDQHDPTQVHAQEQDHGQITTHEPPSPQQHHISRPPNLEELQLAAQLGQGLAGTSLLPSNDPGMNVEDPNMRSIMPHPEPDQHQPPSYVHEAPASEHMVTHALPVAVGPPMPSQYSMGEAIPPRKRSKVSRACDECRRKKIKCDAQSDTGDSPCSSCARSSMRCLFSRVPQKRGPSKGYIKELADRIHSIENKLESEGSLTQDEIDRLLTPGHHRPYNGLASSEEPVRKRPFSSISAGDFSTPAPPRQAPWGPEPRPILPAPAATDSFSGPTYTNSSLAPQPSAIKADSAPPKPPVASMDSQVVEAEEVPDFDEESLHDYLAHVQAIYPILPSSKARIQSLLSQCPSTVRTAFITALQAVGQSSGDTQVASSLLHEWESSEAPHSRVVDIVHAQTLLLLIIDADWRASSQLPFLLGRAVALANTMKLWKFTPIESASETDSDDQLCVRIWWSLVLMDRWHAAGTGKPSLIPDSSVVAPAGLENTLGEVCYYLIRLSKLLNRTAFVISTLQPGSSTAEPAMAAILADYIENYREDLPGHIEAASYPLVHLAYWHCKLLVTLLTPGATPTETLWPTSELATLLLANAHLRSPFTNHFVSLVTMSLSKLAKMDSSREGAVRLMKEILEKPSGGHWDSVREKLTEQLRPTSSVEATASQGLQHLADLATAHEGIAPGSDDITFGPTLASGYLDVA

SEQ ID NO: 88 - protein, 518 aa, Trichoderma reesei
MSNRAAQACDGCRVRKVKCNSNQPCSQCSHLGLGCIYSPSTKRKPSDDASPAGVGLAAGRGNINRTGAVAPGYPRPSLAAAPASEPFVFTAGFFIGMLPKYEEQVYPGSPIITSYEVRRAIDNMHVSLEDAAFVYATGAVTINLTTPTVHGEAGARMTELIRLSLSAHRRAESMSDVRNGRLTEVRTTLKRIMTCIFLEVSMMAFNRFDRSFATLREAMTMVQMIHVQQCSHNAMPLEPRDVPKFQRVYWEVFIHERFLTILSGFPCSMAPLPTGLPMRDTLIPAHIDVGFNRLIRLFQIMDGPFLLYWNAQQNPAQPMPGVSLQWIESKQAQLDQDEIGASEAEQALIESGRGSFTELQHVDLFVTRLWMRTLLWQFGLTHGFLRSAPSQNIHEGLSIHFPAHRLSVQLRALVSQLENVSTIVTHGMGLLQKIFEITSTVADVLALPLSYGQTQEDMRSRIEDFVFLVKFLFSFERAEQEQRDYLRHKLDVLQQQYTMVNFADMVGLSPTVHAPYRV

SEQ ID NO: 89 - protein, 855 aa, Trichoderma reesei
MPPHLQYGSPKSASSPDSAKAGAASSPDVSSGSQKDSRKRKISTDDLDDDDLLEEGGKPVKKTAHNMIEKRYRTNINDKIAALRDSVPSLRIMSKSARGEDTTEDREELHGLTPAHKLNKATVLSKATEYIRHLEKRNNRLIDENSAMHQRIAAFEKLFMAGAMNGSMNGSVPPMQQPTPTQYPQDGQTPQQSQQQQQQQLSQMAPSPMDNPQENNGMPTGLIEVPEDMKRILSAQMNNQPYPVPQQLFRPNPTVVGQQQIRPMQQQQQQQGQQGPQPGWSASPYFGKLMVGSLAGLMILEAVREDEPSSEEPQGRGLFALPLQLFRHIPSHLDVRFAGYDAISIKFMLLFGLVLWVFIPSLFSRMDRTPKKQQSAAVQSAPSLASPISVRRRAWETAIQTVWVPHHSFFLEAAALMLKTLKLSVRNVFGVHAYQLLTGLTPEQEVARVRAWATALDAQLTGGDSEICMSRLILTLLASGTVPDTPSGLMLKALHVRVLLWDLSQKWYQLGLVNYIATKIAERQWNAARDLNQRLTHHSPQQDEMWEKKRAEYMLPDHLAVLVEQECDKVLTPAVIQRAHNLAFNVETTHDAIPIDGMDSVVDDTSIGSPMDALAAWWSTAKVHDILTTTLYGDEAEPDNEMMELAVKVAPMGSRAHARAVMARSVLAHQSRGKNIAAAVQVLRVDAGGSSLIDPMSFACADANPFADRATAPPPQPNSPDPDLQLCLRCATASAHIKRLGGPVASSKQYVRDSIEKVIALATRTRMSLLSFTSVMEVLEQILAHKDTTNSFAPLVESLAAILRLWMGSPLASQCGVPPDLQDKMINKCLGITKMVVGMEVDTGYESMTDDEGFR

SEQ ID NO: 90 - protein, 893 aa, Trichoderma reesei
MTFFSPSCRRVEHVQRHERTHTKEKPFSCGWDRCGKTFGRRDLLVRHEKLVHLNEGHKDPSRPRKFSGASLHKPSLSDSHVDLDAMGIHRLRPPPPPPPTTTTLLPPAPSPPHQQQQQQQQPQPPPPPYHHGSMSSDGGSHLGPQNPQVAARTAACNLDLLSDAALASEVNTMQPLLNDMPQPPPTHSRVKSYPDTIEGYGDRARADPPILAAGFGPQPQQPAYEDYNLFLDDYTTPQHFLPPHFEPDQQMNFFARSPAVFQRGNSKPNSQLPSRFGSLAPDAQEPSSRLHEEPSRSPLRISVVDHGIIKNRLEEFSAVLPADFVFPSRYTLTRFLEGYISGFHEQLPFLHLPTLSPTELAPELLLAILAVGAQYRFESKRGYALWYAAKSVAMEQIRRRHSSEIHALLPNASSYSPHSTRPSPSTSYRHSFASAQSERPITQDTHREPYSPNTPQSRLETIQAVLLLFSVGLWGAKTILNEALSLQSTLAILIREEGLTEDISQASVTDWDAWIKREGATRTKLIAYSFFNLCSIAYDLPPLLVTSELKLSLPLPARLWRAETAWQWQELRQSTPMVEITVHEAIARLFGRTNQGLPNHLSSLGFYVLIHVLIQHVYFLKQTSLAAGLPFEAQRTLKPEDVEEVTQALRVWHTTVEQHHYLLATGSGPFASTDFSPGGLLAYNATALLRLAYIRLYSDTPPSKELQTRDSMLMASAMISTPLLVRSVRLHGAVFQAVHALSMLVKAGVNYVARTKSTEWSIQHSLCNFECALLLAKWLLTLGSLTPSDAPITLDERNLLETVRRMLDETEFAVPVDPSLTGANANPQLEAATTDPMKLRQLASAVVRLWGETFKGTHIFDMIKTMGASLDGYADLVDKPRDRVALAPNAAMA

SEQ ID NO: 91 - protein, 514 aa, Trichoderma reesei
MSTRRYRSAIACQTCRVRKVRCSPPLGTPDTRVSAASSSSNPSTADEEQLGVEMVEAALGHPRREGRVPYYIGKRGPTSTMHICSTDHVIPKHLLMPAAKQTILTDDEKAFLRSKGVYSFLSEASADSLIRAYFHHVHPIMPILEADKLLEYHRLKRLNEYNLILLWGLYTIASNYMPAEVYENEGFASRKAFRAEMFSRAVCAYHNTGEKNNLILLQGALLLGFWNSDIEDHMQPWQWSGKAINLCQILGLHRDIDSVGYNSSITEQQRSLFRRLWWTCFWRDRWLSVSLGRPLRINLDESDTSDPLVSDIAIDLEGVPESITSAYLPRDLPRLAEYWIQLIRMTKLLGKTLTTCYQLRRPKPTISQIDALEAELMRLNMPDHPDPMLALLVTFYRPCSVEAPSDLPTYRQREWQQEMQGKAMSAASKTCNIVYALAQGNFFSYAGPMTPTLLVPAMQVYLLNCKFGDALSRRLGLNMLNMCMMILEELQKTYSVASVCRGIFGKAIQQLFPD

SEQ ID NO: 92 - protein, 300 aa, Trichoderma reesei
MGKKKRGHPDVEEILARPWCYYCERDFEDLKLLISHQKAKHFKCDRCGRRLNTAGGLSVHLNQVHKETLSQVENALPNRQGLEVEIFGMEGIPQEVLEQHRNRIIQNFYQAQEDRRIATGNPPPGQSKNPRKKIKIETAEELLKRFAEYRAQRKAGVTTGGAMEGVVQTNAPFPQQAFNQPGFPSQQPAYGYPADNLAARPPSNLAGAPGLPQRPSQPGFWGGSAQAAATGDEIDQLIRMAEAGVKPQKRPDEGEEAGEGTKTKKEKDKKGRMVYDDAEFSPEEKMAALPRYAYIPPVGA

SEQ ID NO: 93 - protein, 306 aa, Trichoderma reesei
MMYVSGETAEPSVETTGIIEDIVRQQVIELLRNCTELAARRGSKSISTNDLIFQIRHDHAKVSRLRTFLSWKDVRKNVKDSDDKGADADIAAGDDAVGGVVPGGPVDEAAKKNKKAKVGLPWDPSSFYSVEVPERDDEEDEEEEEMNYITLQRLRKADERTKAMTREEYVTWSEYRQASFTWRKGKRFREWAGFGVVTDSKPSDDIVDILGFLTFEMVQTLTEVALKVKEQEDIWRAQTGASSGDSGTSKKRKHQQGLFDPPSEGRSPIEPRHVQEAFRRFQQRPKKTRAMLNGTRLAQHTSLNII

SEQ ID NO: 94 - protein, 1006 aa, Trichoderma reesei
MHASHAKRDSELSSAGSSLSTSSLFASGHGPFFSNASEPLGPSPLMHLPRPALSSKLPPLRIPTPSTERRDSLITYAYSSQRSAAPRSAVDADVEPLLPPLKRSRVGSLPRLQSIGELRLFHDQQPCLRCRATQKEVGTSSSFLFSSLLFFSIHWCLWAWTNSCFVSLPEIQCDRGQPCSACSNLPLISSEDHWAVIGTCIFPAHIGDHVMTCMDYRDAFWWSGQQSYRSFDGHHDAQQLTRDMADRPPPILCALAASWNCQETSSSPLELLRYTGYICPSREVEESQYPVLYHAKVLLRETAFYCLLHPKPVMQLDPSYGYRPPSDRLDFTGEHSRLVEQAMVVFLQAFEAIALRSSPLQSKEWLAVFYSLCIFSIVRTLLMDLASVSVLPYSAFRHRSPLRDSPQNTIDSVYKALVHLFITGGPMLTDGSPSDVPQEDIPVFELTRRIMRMDTWQARDIRSSADFLMSLGSGRTKDVYGFNGFIRPLQPIVSPGNDAQLSTPQSVSAREPRWSSVGSSAFPPSSSHREILLGPHPEALDDANISRGAVTSSLSQPVPEHVGRARRHTVAEAPTNINSSSSIFGNHLTPMRFKPAYQRPPLRRVFCRKCHEYPEGFRGEHELRRHTDAKHAALVKRWVCCEPEGQSPATPQPAIPLSSCKACVMQKHYGAYYNAAAHLRRAHFNPHRGGKASGDWPPMSLLKDWMREVRQSVADAAGDADNDMSSEGEEDEDIKQSTRTMVSRSPAGRLAPPPPPPPPLSLPPPSSSQPPIPHHLPRPMPTPPAPENRSRCPFPDCGRVFKDLAAHMLTHQEERPEKCPIESCEYHTKGFARKYDKNRHALTHYRGTMVCPFCPGVGSAYAKAFNRADVFKRHLAAAHQVEQAPLHSHTTNRGGRLITLSANSGASAICSICNNRFAAPQDFYEHLDDCVLSVIVPAGTRAAMDTNNDANDDERKTIRGGSAGAGAGGAGAAGERTAGMGNGNGNGTGNGNGTGTGTGSIDGTQA

SEQ ID NO: 95 - protein, 490 aa, Trichoderma reesei
MTKPPKKKPGANGQLPVPNIADRNANSNDDTPSRGAVALSIGLLSNVPYSVATASNVFGIGSEHPFANYWTCEGGLPEVISVLPEKLQADILLSRYFECVDPVYPMIHRQTFYADYEHFWQMSMAEKHQVDAAFIAQIFVMLALGTQFVTSTTAQERKQTAEFYASASNQALRMFSYLSTASIRSIQAMVLMTYFLINDNHASDGWAFAGILIRQAYAMGLHRDPNIVTPDASPFEKQQRRKVWQAVLLQDTFLTVLLSLPPSATHTDVSVDDLLDHGSSIASSDPTDNAYIRGSWTLANLVQETICSPRSLDVPICATARHKSKLIADFRAVYRSFPDVFRSWDTESLTALAGTNKRVVRQTLFLTSNYFHNLMLVHASESPDVPVNVRGTLEAAHDAIGSFFLLFNLLESEARVWWVFNHRAFLEALCIGNVLREAAKEPLGKELLAKDPLFVRAKADITRMIQIMQQMAEDSEVARTRVQVLSQFLI

SEQ ID NO: 96 - protein, 253 aa, Trichoderma reesei
MEGIQTHPANAAQAKAFTAPGSLSFPGASNELTPPSTNPDGSQRMAPNGQQSAAGNGVTPATPAATPAASQGPSGITPTLQNIVATVNLDCRLDLKTIALHARNAEYNPKRFAAVIMRIREPKTTALIFASGKMVVTGAKSEDDSKLASRKYARIIQKLGFNAKFTDFKIQNIVGSCDIKFPIRLEGLASRHHNFSSYEPELFPGLIYRMIKPKIVLLIFVSGKIVLTGAKVREEIYQAFEMIYPVLQDFRKV

SEQ ID NO: 97 - protein, 437 aa, Trichoderma reesei
MIAPATIASPTIEVRIPAFSEFTQQTNRRALIAHFTNVLSHLIVLREDEGNPFQRLVLPMSQQSPAVSNAIFALACAHLEHRGVHNSEKSVYFHNQAIHGLAALLAKGCGANRNEVLAAIILLIYYEVLVQSGRSNIVDGHLKGAMAVMCSSQALTDPTAVFLERAFRFYDVIAALSFDSTPLLPAPESGNLAMFPPVNCRGATVPDGNVDTLLGMATSLWPIIYRLSTLGGLKRKLALAELQGDEAETAMLRADFEMTASSVELALEEWTLPADESLPKDTENPILAGVSAQRQMQSILNSAMAYRHSGFVYLYRNIYGYDRRHAVVQHHAHISLTHCVGTVKNKGPMGALLWPLFVASCEANDEADRELSRQAFAAVCERQGMTNIERARCIIEEVWRRADCGEDENDLMMHLGEQRNDLWRRVSQDMGVTVVFG

SEQ ID NO: 98 - protein, 941 aa, Trichoderma reesei
MTICRDPRQSQPSRVPRQPEVKLPKSTAADLGTRIHEDISNGNYECAVCTDEVLRKSHVWSCNVCWTVVHLKCAKKWYHNQMKQDAARPAESQLENLWRCPACNSKLSDEPGSYHCWCGKDINPRPSSTSLPPHSCGQTCSKPRGTCPHPCGLQCHAGPCPPCTLVGPMQSCFCGKNTSQKLCRETDYENGWSCQEICGDLLPCGEHFCPNPCHPGLCGDCDLKVDAKCYCGRVEKKIPCYERQEVRPSYSVEDSSWFEGSFSCLNPCERKFDCGTHSCSATCHAQDEQPAHCPYSPDVVTHCPCGKTPLENLLETPRQSCQDPVPHCDKVCDKVLPCGHLCKAKCHTGDCGFCMEKVEIACRCGRTTSTSICHQGDVQQPWCMRTCQANLSCGRHKCGERCCPGEKKAAERQAASRRKNRQPGDSVVEAEHICIRTCGRPLKCGSHNCQQMCHRGPCASCPEAIFHEISCNCGRTVLQPPQPCGTHAPECRFNCPRRPACGHPPVEHNCHMDDISCPKCPFLVDKWCACGKEKLHSQPCHLQAGHCGRPCGKTLKCGYTKLFCDHACQNPCHGQTPCNESAPCAAKATIACPCGQRQQEVKCLASSSNPTPSRPELKCDDECLRLERNRRLAAALNIDPASHTNDHVPYSDITLRLFKENLAWAEAQEREFRVLAKSPEEQRLRFKPMPSHQRQFLHALAEDYGLESKSEDVEPYRYVVIWKGAKFVSAPSKTLAQCLKIREAQAAEAAAAAAAAASSSRAPTPPLAIAVVEPFNAFLLVSPRFGLTIEDVEAAIRPDLASQSGLSFTTSFLPSDEVLIRATAHYSSSFLTPTAVEQALQNMKPRLEGAVKRQDIAGNILLCHVDANEQVSRREDISRSDAASGWSAVAGRAATPAKKEEQAAASSGGGSGKRVLLGLRKKKVETPAKSWTAQLDGDVEC

SEQ ID NO: 99 - protein, 567 aa, Trichoderma reesei
MIDIYRGELPVPKPPTKSKRVRTGCLTCRGRHLKCDEAVPICRNCEKGGRKCIRGIRLNFSNTTVHDTSYLVPVSTRNFMFQDDSRRIASEYKGGSSRYADPPQSPPADCNIKAEMQSSSPDLGPTEHILQGHVWHVYISPEGSNSTRSIYSDSLILQATEWRKGGQVESGPMSLSEVEDRSNVSEPLEDDSVTCPTTTLSTPEREFLDTPEQVRYMQIYVEEVAAWMDSFNKGKYFGECLPYRALDSPLLLYSLLACGIRRLALRHPDMAEIAAAYYSTANMHLFRYHENPERDVEDCSIVAVILKLYNVMFREGPQSGGHSLAVLNSIISESRWNTDSDGIGSACFWLNAYLDIVGSLDVGYCLYVDSWSINMDFSSEGGEGMAYRGEELWVQRMLFILAKVISYRLCVLSFTEIDLRENRTLEDRLPEWKHLKLLCDQWNDACPRNMRPLGYMYPDQAENNSAFPKAWLPKREAVFCRLLYHTAQFILTQTHPLEQIMLSEEMIALQLHHAREVCGIVAHTEDRYLGPISVQSLLIASSTLTEDGEQMEVLDILQRMKAQIEYR

SEQ ID NO: 100 - protein, 798 aa, Trichoderma reesei
MSQQSQPGRTASFNRGYSGTVEGPQIYSASYSGVDVYEMEVNNIAVMRRRHDSWLNATQILKVAGVDKGKRTKILEKEIQIGEHEKVQGGYGKYQGTWIKFDRGVEVCRQYGVEELLRPLLTYDMGQDGGVAGRGDFNTPTKEQAMAAQRKRMYNAGADGRPNGLSGTFFKNISSTASHAVAAISKARFGSPGPRNRNGPTRAPSFSRQPSMQNGDDFPGNSQQSYASDYTNPASSQHQQPQLNGSVSMPDNEPPRKRARVTMTPADSFASYSQNIDPYGSNFAGSPTEPNESFVYTQNTLNDRDAVVDEGNGPSPPLPYEMSPDVEMKRSMLMGLFMDSATEELSHDMLQTFTPLELDMPIDPQSHTALHWAATLARMPLLRALIAAGASPRRVNASGETALMRACLVTNSMDQGSFPDLLEVLGSTIEFRDHKGRTVLHHIAVTSAVKGRNAASRYYLESLLEWVVRQGSVPNSQATQLNGHGLNGSQSIPKMGIARFMSEIVNAQDSSGDTALNIAARIGNRSIISQLLEVGADPEIANRVGLRPLDFGIGGENAEGKPNGDAAVEKNGAPGSNQRSRESSDEIIESITHLLNETGATFQQEMKAKQASLDNLHATLRTTSTQLGEARRSVEQITATLKKQQLARQKVANLTHAREDDQFRIMNEQSNAAQSNPSSSWETELAAMLEAADEASASGGGGGFASEGMLPSATILRARIRAIESRKEVTRKMVQALKGRSRDVEIKYRRVVALCTGVPETEVDAVVDGLLKAVESEKDELEIGRVRRFLGGVEGVVH

SEQ ID NO: 101 - protein, 894 aa, Trichoderma reesei
MEEPAVTTTNASLPEPQGSSSQQNDGGKDEANRKKTGTRKRTKTGCLTCRRRRIKCDEGRPICHNCIKSRRECEGYSQRVIFKEPLGSFSSPFNQAIYPGPPSIVDESLATHRQSSRGLFPAIAPRPPTFDQRHHGLPLQSSAAPYQQQYHHEPLVQGSSAYNMGPGQLLQAPYNAQFPNPLLATEVDGGHAHYTHAPPEAQFFSIQDGGPARDMLGDFRRLTSDHALNVHTGAAMTQPLNDRWGLDMLDDEASLPDSDDDLPDVRGNLLPIKSMVPERWTMSAVDANVFSAFAQNDDVLAHMETPYSSEFWASGLNTIFMHFINVTGPGISIFEGDISCASQRARSSTAAGTGQSLWSYNPPTAALRHYHRAIRRIAKNVKSPSRRTQPATLAATLLLGYFEVWSSDHTKWCNHLFGARILFREMSLGDMTRACFPAKRMRQRREAGGYQLGHVSSYFYHSHAHAAPAQSGQRSLDYGFLNAITGLAVSPEDYGDAEHQPSDERRSPVSDKEIERYDIICDLFWWYCKMDVYQSILGGTRLFMEYEHWTQIPPRAPFSTYSSAYGTYDHLILLLGRLSDFVSKDLSRKQKVIDAQCGGAGTGSPPMFPGMFPMFGKVQAPMGFSPPRDGTPHSDSQEDLDPEASYEAALQEWNAIYHSFEVFKSRLGPEFQPLGDELDGQTRTKTPFGDPIYFRTYSIAGIWMNYYMGFIHLYRSHPSMPPVAMRAAGMTAKKTAAFALKIGQIASGLAADCSEYTEINTYLGAALIESSFCVFVAGIQYNDTAQRHWVIQRMHDVARLTGWQSAMQIACGCESAWIKACQMGGPEYARPTIINDISSTIWKNPRRIDKKIDELDSSEERRLVLAKAERTHYAIGILAIETELARLELRDDLN

SEQ ID NO: 102 - protein, 587 aa, Trichoderma reesei
MPSNGKPCHNCRRRRLRCDRSWPTCHKCAVSGQECLGYGKVFVWQQTIDSRGILKPSSSRRTPLGGPLSSSAGSAFSSTTTSYFPPFLEIAPSRRPDTGPSSLTHDAAAGEGKSVAGGAQKAPVRRLSPPLNDHGGNGLASGASEPLQDPASSPVQEVLRPAALGSLTDPLFQDLDRNSRYYLAHFADRVCKDLVARDGPGANPFRDLIPLTNKHPLLLQILIATSAIHWSNIFRPITAIPTGLTDPGGYLAQLRSKDLVSRQALIDALTAKQKAMGHLQEVLDTLDPGGSEVVLAAMHFFIKFDLIDLEKNDTKGWRAHLEGASSILALLRPGSNGSEASRMLRDCVVADCFIYHILGSTLASGSLAANIARYAFELLPVMKRVEVNSYLSCPPEILHIILEASKLSYETPCTDWSLTAADEALALIDQALAFDIPAWADKLRQNPRVQDIESRIHIASAHRSAACLYILQALPLARAVRPVDTEFLVGDILENLGQISIEDPYYKATSWPTFIAGAETRDAEKRTWTMKRLLGIWETCPWGYLFTAIELLKAAWEMQDACQGADDEAGINWLQGLKSMGFDALIV

SEQ ID NO: 103 - protein, 514 aa, Trichoderma reesei
MAQHGFTQFGSGPNGGHIDPNDLAMSGGYSSPSFANTNFNTASNTNGFSSGSAVFADDELLDGLPSDGQSGLHGQGQDFHGMNMGYQNTFQSHRGSSLQIDPSHINGYSNTPDGDPIQSPYAYNTHYRALGNPLQSPLSYSQSPLAAADMADGNDPNFLNAKARARMSQQMQRKASNTRHPMTPKTNSMHSIPIGSAQDSPGFGPQSLRTDKSPSGHWLNTPSGSIPASFNSGFSSPMQPSMVQINEVMMKGGTSMPAKLGVGNAMSTQEMKRKRRRESHNLVERRRRDNINERIQDLSRLVPTHRLEDEKIRKLISNGTPLSPTLTGVSSATSGLAGPGARRAAGAGAGNITTGLPIEDKDKGPNKGDILNGAVSWTRDLMWMLHLKLQQQEELMNTIVELGGTLPFEPTDDERRMQSELMDAITRNDTGSFSYSRTSGSGLRVPHHTDYRGEPTNGLGGSLDSVGITPEGSGSGGMPGDIGDTAQFWHDPDDDANAPMHFKEEDEYDMDLTQ

SEQ ID NO: 104 - protein, 572 aa, Trichoderma reesei
MAQLQDPDQDQDQDSLTPAPESQPAAQPQQAQPQAQKGRIRAKKACIECRRRKRKCDGVFPCSMCTHYQYNCGFDGEIVTSGLQQKRPHDALLEPGEGSQNASETPEETGRPSPLVRGIIEPAKRRYMNQNSAVAFPNQLGVELESAHPPRLHSFAWNCGIRPEEPGSIHAPLTDYVTWEECRHYTDIYFATVGVPFHLFDKDEFLQQCDTYFNRRNQDLILGALIGAVVSLGSLFAFRDGHHLEPQIVKHVKDVLEDSTFSRRPSIDQVNAWILRTLYLRSTTRPHLTWLASCTVMHLVEAVGLHRDLESDLVTQSGETRAREDEGSKRTFWLAWCINTIIAYEYGRTKIHFDGEDSRPTPFNDTSETGLQIRMARILPSENANADAMAVSKSLEGAIQKLVDLGDVRGFPALTRGDLCFCLYRRLRLLKISISKDTLSHILQIGQHAVDAAYDLAQRGVPWWNVLGTTFQFFCVLLAIDNRDSLTQVSWVLSRLDGIVQILNTHMAFEAFETAKTLMRDLLVKKRQEVALLEMSEGSDIPFSNRELTPDWDMMLNPFYTTGGFNFKDFGV

SEQ ID NO: 105 - protein, 697 aa, Trichoderma reesei
MDEEDQSRASSPTSDRDNEPDEVMEDVEAEGDREEDGEREDEGDGDNDNDNDNDNEQAADRGGDNGDVDGENTTDVVAADEGQATDDPSADGDDRVRGKQPESPSTQSDALQLDQLPSLYRTWRPKIRPEVYTARLYDIVPTMAAPMSTSINAMAITPDLRYWITGGADGYIRKYDGPGTINGKLPLTVAQRHPFVDSVTKAGILMSYWPNEEPAVPGRSDQEHVLSSVYSLAVHSEALWLLSGLESGAINLQSVRHDEGKRICSLQKHTNAVSVLTLAPNERSVLSGAWDKNIFDWDLDTGDIKRSFDGSGGQISSIELRPASGAPVPADADEPLPSSTISTNNGAPMAGASFTGSATVKSPNGLDRSINVATRGETDAAAAASPAHESLFGGSDDAGSLFGETTGEQPFGHDDDEFGGGMNMAGGDEAGMDHSADKAMETMGRMEADKQLKQSESADDAMDVDLPIGNAGEVPAQQQTQSLDGEPSAEASSERLGVKRPRSPMIVSSQPPPTQYDPTQVSQTTFLSSAIDGTIRVWDRRVPSAVARISNRVGVPPWCMSACWSPDGNKIYAGRRNGTVEEFDIAKAKRGWQPERVLRFPAGSGAVSAIKPMVNGRHLVCASHDILRLYDLRESRAFKHSTVPFLIIPGPPRAGVISSLYIDPTSRFMLSAAGNRGWEGTNTEVLIGYEIHVVSGA

SEQ ID NO: 106 - protein, 554 aa, Trichoderma reesei
MNEIHNLETKSNVYPLLLKCKSLGRIIKANRTPAWPTIPTRELPEKEVCDKLIDCYLETTESVYRILHLPTFKRDYEELWEPGAYPDMAFIVLVKLVLAMGAMSYDDEFSLRPLAIRWVYEAQTWAAEPEFKARLGIQFIQTQILLLLAREHVGVSGDSVWVTAGELVRRAMHMGLHRDPHHLPKRSLFATEMHRRLWNTIIEMLLRSSLTSGGPPLISLDDFDTEPPGNFDDEQLMTEGAVQKPDNEYTSMSIAIALRRTISARLTVLRFLNDLNSCGTYERTLELDAELRSAYKTLCQTLTRFKSPPGQASVPSALALRIVDFVMQRYISALHTPYLIPALRQTKYAVSRKLAIEAALKTWSAVYPSSAIMAPHHRREDDASAFLNGDALSRLACCGPGFFRIASLQASLLISAELDNQLREEQGLVPLPFRADLMSVIHEAKEWCLRSIEVGETNVKGYLVSSVILAHLEGLMRGLDPHELPPLFLKAAEESVEKSLEILEEKAGLGHGQGRAVDAREEMDMVGIRPQSPDWDFLVSFKYGLYLVGVMIVM

SEQ ID NO: 107 - protein, 769 aa, Trichoderma reesei
MASVSSQTASPHSSSPYDFDSTGDTDESWQYIDYSSGASVTGSIGFLPSPASGSLNGFTIVGHVSTPSQGGLSPGSLVDMDPGVFLPASTTTTTTTYQPETEFVTADLFSSGVGTLSGGSAEASFTQGDAFMTPQQYLFMPSGATADLQTQQLNGGLNLGPFMSDFQVDDSFSMMNQTSRPVPQQNALQSLPSDPSMSPWNQTSPGLSGHESKFVFEEVISSPSQLSTTSSSPKSPAVKSEGSSSSSSSSSSSSSRPGKSIRKVIGGKVEKTKSAEPSSSKFVIVTPTTISARAGKPNPYECFEAMRTTQKGRKGPLANETKESALQVRRVGACFCCHSRKVKCDKERPCKHCKKLMLQVPQVVCWQFQDFLPVLFPNFIRGHFKKDAMAGFIRDNVEGFTVNGVEQLCNVELSSGPRFATTLTLQAKFFTAKTCDVLQHWHLSTGEDRVNLQSRGSAPIGIEINKGPLRDEMKKLTKGYVQALVNEPLLADQLTDSFKSTRLPAKILSIVQIFAQKSDSTMVKRALSIYTMHYVMTRHLCLTRQSILALQHTGLVPQNTPWVTPRVLARQIKSLIDELILRDMQQLFELFSKSLKPKLRREWAPCLASFLVLCLFMEAVETAADNFVVSQNEVDLRNTCDPKYKRSLALDTCKELENMPFKQFAYQFHHLYQTHTKDANTRSFNPLFDDSFVEQGELDSAAVEMVQSLRELFHGEDWQDMQFLADDELLLHREEHPYPVDTSFLYTGRLVAKFLLSFTNESVIFASKI

SEQ ID NO: 108 - protein, 496 aa, Trichoderma reesei
MLAITVRFSQKPPEGFSSQIQTSNYFAAHTRSLILPSIDEPSLQRIQVLLMLTGHSWGSGEGRRAWVYLGMAVRMAQIMGLFEDAPVTSPDTQDFIHGEERRRTAWTCFLMDSLLSGGKGRERLLSADSMQIQLPCDTSNFIFGESVLCPFIEGFRSDGTVKLPRGNLGIVAYSMRVADVWGAVAKWSCSSHPNNTPPWQAESELQRLLVRLNAWRDSLPPRLRYDLSLLHAHSVQNQGQAYCYMHCIYFMSVIFLYRSYLPELEMHEETEHENGDRKQWTEFSCRELAKVADQVCEMLQDIRGFGFFFLRGLVPWIGFTIYTAIGTLLYFWHFPHVSEGDPQIESWRQRIIDGCVFLKDMRQAWPMADTWRETIKAMHIFYSNVKAKGNQAMSPSARREMRNAIIDYGALQPSPVQPADSGSEEDAMMNSNELAASEISPNESELISHEFGVVPPADYDLFDTHFDIDSAMQASFADATQGFWEGFPGNVEVSGW

SEQ ID NO: 109 - protein, 117 aa, Trichoderma reesei
MTSTPAVPAADEEPKYGGYSRFEIELEFVQSLANPYYLNHLASQKLLSQPAFVAYLDYLQYWSKPPYLKYLTYPGPTLRHLELLQRERFRQDIMSPDLVQRLVEEEMKASVQWHREP

SEQ ID NO: 110 - protein, 347 aa, Trichoderma reesei
MSRRHHQQHIRQPAVAPAPASAPALERSRAVEELHPVLLDLILRMNIVPPGEIGSLSEAALANAVTLAISMLPEDEQTRIVSRIMTDDEIAEERLNHPRLQNLQSRVALSQNERRLLLRASPEPDDDGANEDEAGANGCIICFDRNAVAVPCGCRYCASCLRELVRVGLRSEADFPPRCCRPFDEATVRLAGRPALVHLFRQLSAEYAVPPAQRLYCHNPGCASFIPASAIQAAARDEANATAVGTCPSCSSATCRECGGRAHRGLPCRAEGDDEAMWDMMDANGLVGCPQCGIVITLMDGCNHMTCVCGAEFCFLCGEDWEFDCRCPLYNARELRVPIRRRPGRLP

SEQ ID NO: 111 - protein, 488 aa, Trichoderma reesei
MVYRGPSKDCLPCRRRKLKCDLRKDGCGQCLRAGISCFGYRDSNDLVFRDQTRSVERKMLAAKATTVSLQFGTLDHPWHYCARHDARHSFFEHYVFGLSSSFDILPLLYEKAKPDDHLSASVDAASLAYAANRYHDASQVLFRLAAQQYTIALRRINAALSEPELASADSTLQSILLLDLYEKIVGRDLKAATPWLAHLNGALALIKARGYNDMTRSRVSRTLANKLFTTLLISCLILGHRIPEGVVELGRRLEMFHAKDDLKWQISKMNQQTINFRVDIAEGKLSKRQIVAGANRLHDAYREIEEASSPRWQSRRVMASEVDLAHRSLIFGGEYLDLYKDHYTTQVRNTIRIMHLQLQSYIQENLEDDGSDEYARAREESLSRTEHFAEQVIASVPQFIIPGIHPNNSIPFSSVQTLKCGILLPALFTAAGASINPDLRLWTIRTLRYMAAWGNMKSARMTADMLEEHPELDFQSMYAKLGGYAFTS

SEQ ID NO: 112 - protein, 236 aa, Trichoderma reesei
MSRIQFGVLAYDYQVLDVAGPSDLLTCASKELMEGLRYLGPIDQATIDRAPQFDFHHIGVTRDPVQLTSSMSIVPTTTVDECPELDILLLGGPNPIGFELHPKFADLIRRHVAAGKLLFTNCTGASVAAVAGVLDGKNATVNNIEFEYAKKHYPNVKWTMDKKWVIDGNIWTGSGAVAGMDMFSYWLKENYGMDVLIQAALALDYEPRDINGLLNVIPKRYDANGQQISTHVFSYY

SEQ ID NO: 113 - protein, 717 aa, Trichoderma reesei
MAPELSNQKRSAESVVDASQDHPEAAASRKTRKVSRACDYCKFRKAKCTGDQPCAKCISRGRICLYEARYTRGMPPTPPPSSDTSHRQAVGGGRASLSESFTTSEATTVISSSDTTPSDLGMAEIQGQVFDPTSSLAFLHRASRRLSAQGDSQGIDDSRSVAENQLMFMAGDKPLASEGDNIRQQPTLPDPEDARTLLTLYFDVCIATYRILHRPSTESWLGIMESNLADGVAVWQTVGRAKAAIIYAILAIAKFHQEKSKGCPTKECQALRLGDQFFRFSMRLADQETGYPRLESAQVRIVQVLYLLTTSRFNQSWYVFGNALQLISALGLQRRADWRRRHKSAADYIEKQCSLRTFWTAYVLDNYLGVVFGRPRHFHDDNIDQDLPDRINDEDMSADGPVGDFDDRRGCHTDALVFHARIAKIIGAISQEMYTENHSSEVERIAAAKDLTQLIRDWHQSLPAHLGAVHPSILIPSYRRPALALGLAHSHAIMHANRLFLMRTPASAYVTQVGDCIRAAKDVLESVDTMARGSPIFHAFWWTHYVTFCALVVTYVWVMQQYRMGTTDDSGYRTKLMQLAESCHGHLARATASNSPSRRYAVILEGFKAAAMAESTSQRSRMHSTAPNEHQEAHSTGTPVMDTDNGAGFTFMFQSSLPTEMPTFLDWQLTDWLDLDSSVSIPIICMTISVVDFLQAFWPSFNLDEAVRSPDGFEIGA

SEQ ID NO: 114 - protein, 650 aa, Trichoderma reesei
MSTFTALNGAPSKLTESPAAPADQSSSPGERLAGSVIATSAPAAAEMPTTSSSQRESWTPQSAERTIHVATYPPEIEGSHKRKRSVSVEIRRELPAQERTPDTATAPPHNEARDHYGTPQRDYRSYGDEQRERDKEMWYSHSSRDERNPYETQQNSAISTQSNTEEQLGEALRRAAGQMDHSDYTNTSPDGDDRSVSLYGGSYTDHRGDSMLQHDPKKRKRNFSNRTKTGCLTCRRRKKKCDEQKPECGSNCIRGAFVCAGYPPQRGPGWQKPDNKAAAVPLESKDPSYVPPACETGVVEPLPRPASPNXPPSKPASDYSRXRSANGVDHPQCIRGESRQQALCAAVYARQRLPHARERKHSGTTLWGEDGKGVPARAPAAXFEPDRARNTPSGQPTAADQHSASDEDKXPDSSPTASCNLKRADGCTARAFSLAISSATDSKGGDAFGKALLPVXXRALPXAGAMRCRLLEIQQPHDAPDKRRLSRRASALVPRDPAAPRPCPRVSHRGVSGDQRWTGWSSCCRRNSLCLRLWLXHLHWQRRCHWPKLHHQRRLRGQDRGQLCPRAKCQHLYRLLTHXSXETSRGAGAAARQTYCHXAGLLDRWRCHHLARPDHWQGKHRRSRIYRYQGCAPLHDSGWKSSTCPSRHCV

SEQ ID NO: 115 - protein, 755 aa, Trichoderma reesei
MARRRASKACLHCRQRKVRCDVTLRGVPCLNCQLDHQSCAVQERKSKRSLSNAKHTFHKDGGSISEGISAERGHDNPSGIDSIDQWAKRLSALTPVSLRGSPNATFNALRNDAKSGHSPCINLDGFSPRTACTISRLHIGSEGTGKPSACAAFTDQPTPTLDGVIYSNFETLPFIRPPDLLHVSALDIRFMQLNGCFDLPSMPILNEFIRIYFLHVHPIIPLLDEGEFWDSYSCANEKRISLLVFQAIIFAACAILYDFEIECDPVSLGQAALLFTFWPGSLRPGPSKQNSQWLSKAIGHAKSLRAHHLTSNTGRDKLPQIPPQTRISLRRLWWCCYIRDRIIALGLRRTLRIPEKYPTLELGDFEDEIYRSRVYKAESKRRFFDIFMQISKLCIVVTDLLQLCSVSEHGLECETNISPTDRHEAIANCTIQLQEWYDSARQQFPDDAEQPGIRPHFVIIQTNLMFTYYFAAKLAIFHQEIFYAVRDSGNASGEGSYSALAGKSDQVRDAVDNVIKHLANPVRLGLAQYLPISVVAFVAMPLMMQIINAKIIARGVANTRAEVHRQQLHSLITLIKQCHPRLDGIDAVCVTIRRLTDLAQARFLSGSASQITECLDLIDHKPLEYLRLFLNLDLNLSNATMLDNIEFLEVREELLRINKESLGADTQQTPSSTIDSKSFSTQPAAPTGQNIVNENLPVAEVDTPVDFDDSLLDIDQLLGGDGPPEADSAFMALEPEFLMNQQMEWEMDAWLLGGT

SEQ ID NO: 116 - protein, 407 aa, Trichoderma reesei
MSIDNKPPTQNPGPNPHHRDESDDSTGSTPEREQAPPMTAGSGSNIINVSQDGQQPKRKGGRKPIYATSEERKQRNRQAQAAFRERRTEYIKQLEETIRVHESNLHNLQAAHRTAAEECLMLRYKNSLLERILLEKGIDVQAELQAKTGSPNLAPTHMPQNLVQPPPLQRTMMHRHHARKSTSSIAPKTEPGTGLPPPLQPHKTAPSPKNRPAPSSHSNSPNMASSAFSPAASDTMSMRGSISGLSRQQMTPISVTTPTSRQPMVQPGSRGAMNSGASFYPTPSFQNHIEQLGKEINRPRWMSVGALLCWMLTWCRTEQEYEADMVEDSEIETPSGPGPYPGGFNGEQQPMLLSPASTGPGHQVAPGSQYPSMTQLLDQQPDWDPFGLSASMAFPGQPFQFDQTHMR

SEQ ID NO: 117 - protein, 665 aa, Trichoderma reesei
MPRSSFSHTPLLRVSRPVSACSRCRAAKVKCDGKLPACTACEKAGRENACSAANDQFARGKERSYVAALELRIEKLEKRIAYAKSRKASVANHESDSLSTAHVDRRDSLASIRAAIHRKAARTRENSDVNSLISDFGFLSVNATTRDFEPSFINMTFARLVLAAASRDPIPGMPESPLPHKSYATEIFHHFMKNMYPLFPCFSETAALNVLNDIYSQQSERVITASDHWLLYMILAIGSTMQSQHVNDDYYNLGVSFVSRALDYADIALAPGYATQIQSLLLLTQYAILDPNHFDSWLLIGFTARAVIDLGFHQDPPMSTVVDRAALDMRRKIFYCTYALDRSISMVHARTFSFTDDAIHVAFPNPANHDRKGKEAESLPISGPQSVDPGLLLFQLRRAQSYWYQELYQSYATPLQDPSSFVWQMCLDMREWGESLPSSLAPGIRQMFEQELRYSYVYCIAPSARAPEITNYHRTLIFEYSLAYLLTMQDITQSGLNGAYYTYHDALKVYFMANQFLAVLRDSEDMLLSGAHIPKPMSRPGAPQAPPIPPPSFRNGRPGEDNIARSIRCLESVPQTLDVYGRRWEDSIMLKQSFEQLSADTMERLKERRKRQNMKVEHPSYPGSSPVAPSPRVPTATSRSPQQPREIRWVGVDVSQMMQGRGHPQ

SEQ ID NO: 118 - protein, 366 aa, Trichoderma reesei
MAPTYSPPDSPLSSAMDSSEPYEEDAHDHDHDDETALRPSKRQKVEAGSTTSSAVVPDAEHEPVPEPDPLEGMSDVSSDTSGDIPSSPVNARLEEEDFQDQVTVCDWDGCPAGDQGNMDKLVEHIHNSHIENRQKKYTCEWRSCNRKGLPHASGYALKAHMRSHTREKPFYCYLPECDRSFTRSDALAKHMRTVHETEALRPSDPVPKSMQSGPTGRTGKLKIIIKTPQSQSAQDDGDDANGDVPPSECFTVLPPDLFTEEELQIPLPKLWRKCHWEATWAEEVGEALRNECKQWEDLYYKEWLDKEVLLSQVIQSEVDWHERRQAIISGAADVQIPSSTEEREEEAANGGGGDKPIAAAAGGDED

SEQ ID NO: 119 - protein, 809 aa, Trichoderma reesei
MADRSETPESDTGSGSRHDPDQPPARKRQRVRLSCLECRRRKLSCDRGFPCERCIKSGTPDRCSYESRNGEVVNASSGIPPSFAQLDSRRFGIADAVAGFSSRDTELARQDHDRIRRLELEIAQLKNLLTRPGAASLDGSTVVGSTNSPSTHKDEGAEAEARPRAEEQEIIQKSGMAGENGELRFFRGKGFRTRYFGPHNASMAFVELTGLCPFMRETADEWLRPVILHDRKDRKRRQEERETIFEQSDPELEALLPSKEETDALISVYLDQFEQIHRIVHVPTFRKEYAEFWVPGTKQRYAAFTALVLSMMAVASCVHTHESLKFVGMMSTARHKAEKWIVACDDWAGRQSQKHRRLIHYQIACLLYLGKRVNTIKKKRFWTSSGQLIRDGIAVGLHREPSHMGGKITVYNQEMRRRIWATVQEFDMQASFDHGLPTLVSQLHFDTNPPRNLDDEDFDESTTQLPPSKPAKEYTFSSFQNLARRSLPLRLELSRLLTGPVSEIDYDQVIRYTNDLTHEIDALPSWDMNPESNSDSKKNPLIAYTLLHVQLRQYITPLHQPYLKLRKQNSKYQYSEIIYYNAARDMVLLHDKLYEQGVRALNFLREDALTTAINLCSVTMLQPRGSTNMIMINSHHTVKLIEKCIAMKEDRLLRCGNNEPWGYSIMCAALGLLEAHLGTKTTEAAKSTSAERFVSLHYKLLANQEPPLSSVPLPGTPLHPPPIGPIAGPGIGPVPGLEMPDGTKVSIKLFHSLLDTSVTPFSFPALSTVPVEPSWAVSDATQPFNLDPSLELLGLNLNEIWGESWELGL

SEQ ID NO: 120 - protein, 666 aa, Trichoderma reesei
MAVSQSGKAKTAPKASNGATRRGAQMHRRSRTGCYTCRLRRKKCDEGTPLCTACKHLGLACEYKRPMWWSNNDARKKHKEDIKGIIKRKKLSEKSAHTSMSTSVTSSPPGLSHSVPSSATYTDPMDRNRSASIDSHFSNGFNFNGPMHHADYSAYNSQIAPEFMIGSYSPYEIDVKTERQMFINDIPTLKESQVSTFSTYCPPPPPGTVLQSGALPGSWVEHVHEERRESLAEEALNVNFFDFAHGPSYDSRQVKIELDENDQRLFDHFIQFVLPTIFPILETNQHGSVASDLILPALQSNNTYLHCCLSIAAQHLKSLTTGPSEDIDHDIMRHRYATIWSLCESLKRDENYQQILEATLGLIFFQCVVGRSSSQELLDIPWHQHFQAAINLVTKLDLPGQVLDPATPMTQTPFNMTLTAWIDILGATMQGTSPTFAHTYREKHLSQLNPSMGLRELMGCEDRVMYLISEIACLESLKRDGMDDFTLCQHVSALGEQISLTEVNEHTPKMPFNGNGTLSPKQLSKNITAAFRLAARIYLCSLVPGFSPQQHSPKGLVDKLTNVLQFIPSGPEGYDRSLVWVYLLCGSISLPGSEFRVLFEDRVAQLGEQADCGSFGRMVMVLRGLWLHNDNLSRMGTPESVGSEPAQPYLHWRDLMRMQNMDYLLM

SEQ ID NO: 121 - protein, 519 aa, Trichoderma reesei
MASTAPAMFSFSHTEPSTDLLSGSSWGANAEGPQNLHDYPDNGSSHSGEAEEYLFTSGHNSPRGPRLDQAQPADVAWTAAPKAVMSIATGAQSMSRASSSRSHGSAVSSTQMSNMSVRGNAQAFRSGPHPAGSMAGMDSCLLLDSDAHGVASHMYWPDYTLEMNLGADGVAFPVPNVNPLHVVPSQMHLANDNVPDASSPSTWDCFSSSISRTSTPATIDDAWQPAPFSPETSPELCRSPRDRKIPMMPEDMHARAVSHMEDASALPQAYTARRQGSEGESARDHALYKNVTPQADGLFHCPWEGQPNCNHKPEKLKCNYDKFVDSHLKPYRCKAEACEGARFSSTACLLRHEREAHGLHGHGDKPFLCMYEGCERSILGSGFPRQWNLRDHMKRVHNDHGSAGGSLTTAAQQAAKGRKRKSDASETQVPTSRKASIKSMPAPEPKQPPVKPLIEQWLDQRKALEEIVHCMNKPDDAQSLHHIAEAQKRLDIMFKMATDVANATPKTEMQPGRRHYLIG

SEQ ID NO: 122 - protein, 597 aa, Trichoderma reesei
MDIADIYIRVGIEASPTESASFDEPFGLGTDEADAWTSTLTPPAAATQGSNPRRKHYAPRTRDGCLTCRARRKRCDAAKPNGRTCIRINAACQWPQKLHLSHYNRQLESAGGERIAVVRSSTNASPDTRSNDTDFGGSTNRAYETKTGTAPITCVPKSPAPEHRYGPELDPSQAALERHPLSYYISVIIPQMTIVSSTGNFFTSLYIPMAFQHTAVFDLTIACSAAYLSKSASNPASQLRHVAAQRQMLAHKSIQTPLYNRDSAELSSSLLEIITLFSIGLGEKGADKSLRWMQRVNCVRELLKQHETSVAAAWTTWETVCIRGHFRYYDWMCLVMEDALDRTTRGDLSEDLFGGTTRLRDLPAARGHVWEQEFLHLQQKGFRPQRMDDAVHFMLPQSLNGKYSVTPWSGSTYPGSSLSIISCRDCWVQPCCKGHLREKPPFTGGCFCRRVRYSYDAEPVLKAICHCGGSTYSPSFIVPNSDFVITAGNPKVFSKTADSRVSIDNYLCRTCGTTLWRDSDNYKDLTTVKAGTLDEAHGVISAKIPAAETPGSLSVRLPLRSCGSSQATTWGDSVASLASLNPPKRWQTVHVKWCLHP

SEQ ID NO: 123 - protein, 438 aa, Trichoderma reesei
MQPDPSKDATPDANPAHLRRRACIPCTRAKRRCDMNRPSCERCLDKEVACRYSATRPYARRAAKSPQTNSPGSNRCEESHATPLLDSSLLASQDAEDCNDTSCGRSYSKAADGITILPYSTVSWFLQAEHWVIYHGDVQSPQLPAIRGSVLQQYFPRVRKWLMQWIELDHCPMIHRSLFAETGMPPCVQDAYAAMALYAMKNENNEETVMQHIRDKVDSLVQRYSDAEQALLVGGFASVSPLSTIQHLSRVLSLLIYVFIGLYDGHIRQRAEAEKLIPLLRDWTAQLWNSVNVDVTLQNAFGGDYLVARDNTEAVVKLWRIWILMENVRRVWMVSTCTTCIYLVARDGTGHCDGTIDFTARRGLWDAASAPLWKNMLERKDPLFVVPFQTAWLFEKTTVEDIDPFGLTMMSLTLNSDKLDSWIAESTDTHLEALLMTV

SEQ ID NO: 124 - protein, 787 aa, Trichoderma reesei
MLYALALSHPTPPNLAPSTEWRIGPPRAIDSNPTSTVHFPTPSSGPFTPRLPASLWLLASGPDPGDGQKPSINVVSFPYTLLFPSTGHATLYATTSPLHKETKKKSTMMRAPLALWPSDILTRPVPADGLATRRCQTCLRLGLECVQFDIFCPINVVTPTRDTRSSRSASDADAEPDADESNHSSPASTSSGSSGGSTEDAESSSGRELVPMKARRLSRSSTQSQPELQFQTQFQLVHPGRRRRGNRENQDRQAVALRARGGSREIPSLSEFSYLSTSEYFYLQYYLERLSNVLVNANSRVNPLKSLILPRISSSSLLLDAICATAAVHRSSASEVDRSEYATMATRYYVRVLSAVRELIPRVSSSKSGSGSGSGRAMDLITDGNEASPVDDGSDIGEDDAASSSGKGKGKGKAKNNRSKAIEDKDTAEMAILASIFLCKYEIIKDGVENWRLHLRGIESLCQSLDADQAASMSETLTYVRSFMSYHKNIARITETSPQEADEFEHEPFEQDRLFPVDPYMGFSQSLIILLGRVNDLLAINVQHEPEKLEQGIRELLRLLALRSWDAHRFAIPEGMKPSTIENSSHIAEAYRCAVIACVHAALELLAEREPRERGGVVSSRPGSTPLDLSLPSESLSQLSLSPQALTQAQVQAQMQAQLEAQHRAQFLAQAQHKMPINWGYLHALLPMTKADALTECLAAVARVPLGCPEEAGLLPLLFIVACETNRPEQAREALQRVEALGSHIGLGNVRCASALLNQVWKRRSEKGNFSDWRGLLADANWDLIIT

SEQ ID NO: 125 - protein, 457 aa, Trichoderma reesei
MSGRNLRWESLGIFLAAASRAALDTSSFAPLYNSDEGRRGLIKALTYIGDCCLETCLALDCLNDLQLVLQYENLIVHSQVDGDQSYHSWRRMGDVASSLFALGYHEKIDEDTSDIPLFVAELRRSCFARIYAADKSLAIFLGRPPRIMKEFCYFQLPTKFTSLWENDGGNGKSNNAPLSSPMVTGVDGEMCNNEQVLNYTSDTFCSALFAVIKEEILQLFRKRHVSSETSIISRRCSELRLKIDRQWQDLPPHYRLTTSLKDCPLGPFARDFLAGARLEYLHTHFLLGFLSQRKVAEPDEALLRTASEMLSIVVETIILRDRMANSGTCLIWKVAQYGLPAAGIISLALLNSTAPDSRQFSRSKMIQALSVLVAEITIGSWIQPGEPNFALFSQATRTIQSLLDSLTALKVPSGPNDSQQLLGSEVADNWDPYINSQSWELEMDFWASLAEHPTLAN

SEQ ID NO: 126 - protein, 466 aa, Trichoderma reesei
MPPEASPEPKASRACRHCRLYKRECDQKLPGCSTCSSKWTICEYDDDYVPIETGEADTELLWSEPLAVERSLCGLELTPAGEEQLVWSVTAATGPFEPAPDSNFLMEIVKDIFSYGGTSVDQVIADYFTNVHEWMPIVDKPMVVSALQSIRSTGRTGTKDDAMALLLLCMDMMNHHCHHSEHGANSMLYRATRRLFSLVETLGRSGLLAKLHAGLLLTAYECGHGMAAEACLTLATAIALSCPNGTATLLIPTRASLPKDVVPYVAEVHHAAGNDIEIKFKARVSAALCIGEAIASNHVVPDARDCAILERLIHDFVLRHGPSSDNKDDEDDKDLYPVSEVIAMGLSAVVSLYKRMVQKLGSTPDAKLSVDFKFAYDIVFEMCRVEGALIQTRKETHGRRLCFSGLSCLYRAAIGLDEISPDGALPQDARQLRDNLKWFSQHWSIANQLLWQHTCNMRQRAPYHPF

SEQ ID NO: 127 - protein, 747 aa, Trichoderma reesei
MSDADSQQIPPIEIGSFIALRPDKSEFIGSASGVFFANTVFRAFATATSTSGGAAAKRPQGDVDGLGTTATPDPGSAHEYVMAEEKAQEQAVEDDDSLGVGAVGAEEASKSPGIRSYGIAAPGLGIPPPPAVAKRLLVEYFRRWHPFLPFLHGPTFFDKVNRFYDLEGKSAEPMSQRNKVYQAIIFQCVFNIADSNHNQKQERELPEESRIKSSVALTSLLGILSSGHDIPSLQALLAVELYLMTQMSLRAASTVHGAMTRILYHCGLHRCPFRYVQLPRDICDMRKRIFWSAYVIDRHLSQVLGHPLALNDEEIDVCLPGAPEVHNPARPFQNIPHTRSPVTEDDMHAYAPVDHPTFGSVSSAGDLTDGSTIGTHEDSINTDQLVYRPQSLGESILGFLVTYSRLLGQALDLFHKSIHSRSITWDKVLEMTSKIHAWWNTLPPSFQGEGLTSIFVEPQSHHGPPFAILYHQLILIMNRPFLSLPTNRTDFQFSLQTAVNASRAIVKKLKLRQGDSPLVAWPGMLSAAWMAGLVIAYAGLLKLYPMEKTILDLNCAIRLLGMMSRNWTSAHHCRVTLKMLLDRLTSKPFMDRINPALSADPSLSILTDWSQPVFSTYQNLLEGSRSKRRRVEQNNVNSNNTGNNAGNNSIGSNPGLMGNAMSGYPASHFIGPDSGFVGGDNLWDYPSFQPVLEYTGPDFGFDADQLSQQGEWERFLSENMNPESSGLLFNNVGWDSYVQNLGDRLNL

SEQ ID NO: 128 - protein, 787 aa, Trichoderma reesei
METEAPRNSAPMSGAGQGSVVSSWSCFNCRRRKARCNRQSPCAFCSKAGVECSYPFTGRMPTRQHNPAAASVVPVQARPSRKRSELQVQELLDRLRQLENVVDEMKAQEKNGTSRHIESGDAIAEEGESIPRAGADSAASGSNTTDSCSDLSHKLSKSFGSLHVCGGGTLYTSNGFCAALHDELRSVRHAFEASDVDDLNGLECNPSVHDDRPDSIPFPFAQNCSPDSILHPAPSLIPFLWKTYVENVDPFIKILHVPSMDNVIQQTKDNLDELSPGMRALLFSISLAAVVSMSDADVEKEFGNAKEDVLAHFVMGTETALSEAGILKSTDLCVAQASLIYLESAGHRYGMRTVWMMSGILVRAAMSVGLHRDGVAFPKMSCFQAEMRRRLWWHIYCFDARVSQCYAPEIMITNSMLDTKEPTNCNDADLDVNMEKDPVAREGFTDVSFTLMACELRRLYSHVLSLMSALLDTGERQQAAQHNALHRIEKARHWAKTTVLGDSERRRPIQPFMDFLFNMLLDQLRIIVRDTNIFKRWASTDDTNSREQSFFSALALLGDLRQWRNQYSTRHWGWILVNFQDWHPLGIALIHLQTQKWDPACEEAWTLAVQTLNAIPPAMMTQNPLRQSIVNLVTATRQHRERQIEEQRCQAEAPSSISKTSECSIPISSLADIWTSCVNEYPAPITEGLAAQRPATETAVTTIIHTATSNAIKDNTFEEVMNAEQLRSDACYPPWCFEMAGGMNSEEDHFQSLDSLPVDAKQGQSSREESVVARLDLSNGALWKAAS

SEQ ID NO: 129 - protein, 441 aa, Trichoderma reesei
MATVKPVLRRSHTKSKLGCQACKQRHVKCDEFRPSCHRCLSSRIDCKYPAQSESEDEDEKAASALRWPDDIEESCAQWKESGRPPFMFMASSPSWHNMPLKDLRYIYKTALVANVLELSKTADICLVWGDMRTYFQLATHYDFVAQTMAAAAAQRLAVTTKSYEASQDAYHYRKQALCGLYRAMSCFSKDNADAVLATSLGCSYIMPDYRSLMALAGDISTVVARMRPWLKRSAFHRIFTYEPTYPEAEEQASSSPPETGDMEYRFVLVKKLLSEGIGAVNGLSFCFQSDRDLSAILRQLRDVMRLVHERLGADISPEDQFRLVYPFTSWFVKNSAASYVALSAKNPFLLVFLLHFYSVVVALTVALPRIDVPLFAKYRMRAILEICSLLREEPGFLCQRCNGFHAYEEIMAFPLSSVSVYQQVGRQAGGFEGIVAERFSP

SEQ ID NO: 130 - protein, 578 aa, Trichoderma reesei
MSQFDLHAVTRKTYAIPRQKYWDRDYVQALEDQVRLLSASLQQQSKDSSSASASASPASQPSPGSWTAAAGKQPPQPQLPYGPKMLSALHDFGSVKWADITGRDGLPMLAGPGRFSFFSHTLLPTFEDGSSRPSPPPKATVPSTETFLLEIASDLGLKQHLKAHFLEHINPYYKFVDPVWLNFSDLFPHNDTALQLLYSALFAAAAYSSPVATRGVAEAFMAYAEGLVQQCYLEHLCLPVLQALIVIAWYKHMLLDAAKGHLYHYMAIGLSNHLKIRDIPPHERTVSDVATIRTFWSLFFLDRIATPKLGCPTGIPWEPDNITPYIDTVSPELVDVAALSFDHHCRLYHIQQRYIDVMYAPNFHHLPAPEKQAIYAKANNAMLALRKHIDRRLYISRNAKPHRVQVVFWITYHSILINLYRPVLDPQDPGMAHNIPSALRSATASAMAITRLLKGLQASDDVKHLPPFIIYHVFRAALVHGLNLIAAEEAGGQRVSSGNFWACFRVLGELSGVWKELSEGAMPFVLMATRGWGFQEEVMSVNKAADEDEEDGDDDDGFEAGIDDLVGQVEGGQYGSIS

SEQ ID NO: 131 - protein, 1012 aa, Trichoderma reesei
MAYLATMDPDDFIDFSDCIDSVTVPKESCESCIQQHIHCQVTPESNTSSGTCTSCMRVGRVCSLAATREGAIETQTRASSQDHQNMLQQLAGHTYSRSDSSFTINESQGEDATDVVERKSKAGARFSRDTLRVLRNWLTSNHRHPYPSNEDKETLARQTGLNKTQIGNWIANARRRGKVRAPRSTSSSPGRLPIGIDIPPRVAPLTREMSPMERWRNSPPDQEPASISAIATAVSSSDFSGGANYRNTSIDDGLAGSIDRLSSAGSSRTSHSSNGSLSTVNSHNSRESLVSLPSLKSGGRRRRRRQTSKAVNISSMLPTIHTYQCTFCTETFKTKHDWQRHEKSLHLSLERWICSPDGPIQFCAEWNCLACVFCGLKNPTPEHAEQHNDFVCAEKSEQEKTFYRKDHLRQHLNIVHDAKFQKWPMNDWKVATPPVRSRCGFCGLLLASWDARVGHLAQHFKNGKSMADWRGDWGFQEEILRIVENGMPPYLIHHERNTLDPFEASRGEIRCGKDLEDYVKLWLVDYVNDRTVSGMSITDGQLLLEAQRIVQKVDTEDTSPTGPAVSWFRDLIMLHGLSRDATNEGPYTTPKTAAWTAQIEKLHASKCANSDLGTLGCEKEKMLLQYVKSRRTFGQTPTDAELQFQACRILEDVEPKSNFKCQEAVQWFKFLINSSTHWLSDFKRRAGNLAAAGQFETSTAGAVVSLQALHWGVSNESINHTSPMTAKDSLPSSSIETPKSTNSQSQPLRYFLSDANCYGRLEKELTRFVTSCLSPNNPLQHIPTDEELQYQARWIIYDDDDPWNQTAADNAEWLARFKRDTGLVTSDGVNGLAATGPPRPWRIKDGGTGFMPPFVKPKAGATIEVTEDSPVYIDYRLYNVGKDTAERYIRALCDGEYQPPPSVFCSRELEYGLNAYIEECITKLYVPSDDDLRSKAREILGVESTAADDPRLLEAFKAMHVLWHTQINDMTDFMPEGHVDADNMGSMGSMGANGWEVDVEQMSAFDLLSSPR

SEQ ID NO: 132 - protein, 698 aa, Trichoderma reesei
MQSLAPSAAADARALRKACDQCRQRKIRCDKNEPCSNCVTAERRCTSTAWQRPKELRQRVLISSQYEKKIDRIEARLANIESLLKNISSATPIDPSKFIHTPQTDSAVPTTTSTADYDSSDEESVLGGDSGLTVHTTFASEFLERAVKKASLPAVNPKMETALANLSQLVELQKHRSISHGPRFPLQQPVPPGGVSKLPMPPMATVVALLKHVKAAPPTFFTILCTLTGIKDFADLCRTVYFPAEDFSDATFAVVNAMLYNLFVEQHSLAKDEALREEYNSYVTICKTNLETTLANFPLFLSPKIENVQALLLGCLYSIDVSRPSVAWHLITMAAWLCQAGGYHRTESLRNDPPQIAQQKKVIFWHVYTLDKGLGLRLGRASVIAECDIDIKREFEVNGFDHLTSTTFPTLWVEMSSLQSRIYEQLYSPAALASPQADLIQRARALAAECSRLEVETEETREKVYTLLKSIGTSDVVDVFIKGDEVQFYVTKTLIYRVIPAPENSVTRFCDECLDAARHAMKTHQECVRFMDISTYMRSMYIHWSLLLTPFAPFFVLFCYVIETTSLVDLRVLQDFVDSLEGGRGLSEPIDKLYRLCRVMCDIATFYVEAKGQQQQSDNSISIGDEFEMYLSQLGFIPNDDQAMTNTDTTDAGAPLQGSGQVAQLADWFVGSRNMIGLLEEDLSQIESHRWMQPDDEL

SEQ ID NO: 133 - protein, 699 aa, Trichoderma reesei
MSEPAADAFAWDRSDARSRQPPIVNTIRRIRQACTNCRCSGERPRCMNCRRVDRVCHYEPYSATNPPSSTSSAGNGAKGRSPPATGSTSTLFPRAGVVDPELLNRINTIESQLAQLHSQGLLGFLNGENRGASGAFPDLTAASRTYQDLSPDFTEPSPPQHQFPPSLPETTEPVSDISNAMRFNTTPPPPVMAHLIDTYFLHVHNQPYSYFHEQSFRERLNYGLVPKCLLFAILASALKFSDSEYFRGSRREATEAYAREAWLALLNDHLTVENNPSLPVAQASNILGVVDFTCGRTSSGWLKIGLAVRIAQDLQLMREPSSTLSPIEQEEHRRAFWSIYLLDKLVSVGQSRPPAISEEDIHVQLPTDEDTFRRGLWKKTPTLHQLINWKSETETVNSTFTQTILAATALGRCVRYVLHGREIDEMPPWDPRSDFTLLNTFLLLIDHHLQIETTTIPQIVEQHRKPDGTLDFQSLEHVVFARIVFHTTHCLLNHPFLLRMRLKKGNCKVPPKFLTRSFEVSCTHARSISCVVEDAIASGCHVQSSFYAYASILAGGILSLAIHIDQEKGQVTDPELLNHYQKSIQILERVGRRWDHASKMHLQLMLFDANYHSLAALLDSTTPDVDPKLESALWLMVDYVAMSAHNDSAITPPPLSTDTFMGPLDTQMGMENNDAFGGMAPPNANPDPNVGFLGNSVPL

SEQ ID NO: 134 - protein, 717 aa, Trichoderma reesei
MKASKTASPAPGDGDGKKSRAKHACRECNTRRVRCDVMEKQPCSNCRAAGASCQVLPSRRGRYPRRSRRLLQQVRSESVTQASSDTGQSTPLDSVSHATAVSSHQMASSDTQDQVPDHQPGHLPAASSPSSPTPTATPGTRFFGESSFLTLVPGEDEGTSTQSNAPGDSGRNSQKPRFTFPMPPTPQSTSQTAQGGTHGLSTGLMRYLADEGALTLPDLESCLPALQAYFAWFHPSYPILDRAQIARRLASATSFSSVDMSRMLLQAMLLIGATYCDAATIRAMGFQDRSQAKTAFYTRARLLFHADWEKDETVLIQSLFLMSFWRGAPADVRDVRYWLGVVITLAESYGLHRSSRSMSKDSYTACMRRRIWWSIYVRERQASASLGLPSRIRDEDCDIEPLTPMDLESEASPVDSSFGSCQPEHVTYAIKMVEIAKLLGRIIDLHFVPGHASLKSTSSDTVHDLNASLEAWMASLPEDMKYSPDHGTDSVWACLLHLAYNFLRLESTTPGSQVVIKAACRISRIAEDMSTQGTLRITSIFSALCIHTITIRRGSGLTRRLAEHRAETCLLCLKEVQKYWRININVLDFFLQYLDRSIAARLHGMQSDAPPPPSRGAGREAKANDAKSNTVVDAVPLDANSSSMSYPRVEYDAAAAPALDLSIPNLGPGHYLAQEEFREQYLGLVSQGDDVLGDWDLLLQGEDFSQTGSGFNVLERS

SEQ ID NO: 135 - protein, 155 aa, Trichoderma reesei
MSNLDWDSATKIGSRVRGPGASDRETVIRGKSALNAAQRAGAAIATEKKYSTANSAGGTEGQRLTKVDRSDDIIKPKTVGKEVGKAIEQGRQKFEPTMTQAELGKKIGETAATVASYERGTATPDQNILSKMERVLNVKLRGANIGAPRLGPKKK

SEQ ID NO: 136 - protein, 728 aa, Trichoderma reesei
MSTAKFQSPSYRLFEPGFVTNKPIIVDVDDEFGSPETLTLRYEEAVQAAQRGDLDRDSPPNEFSMAAYSKAQLPSMNGYDTTRYQDASYGPYSTQSFSAQQTEKFAQMNQQAYAGNNGVAQYMPSGLSVISCEPLAGIPGTKVHLKISSSEDLFSLSSTQPFMLFGTMKCPASLVRDPLDSSGFVYSCSVDAPQPVVTGSNHSVPLSFVIESLAGGDMSRTTFVGTFQYLEGPEDDITRTDKLPKDDTTTPAAELDQVSPSPKTEAASSAATNTYEYPQQQSQYGNAFPQANNSDMITTTYRASSSFTDPHYHQHHRRSHGGWGGSGYGSTLTRTSTAFDPIGIPGRSAGHQLPIPSSGNSGTPQLVRTSTITNASGNGSYHPMSLYTSKAVLKIHGKLSEMARDWTQEEWDNRRRIVMFKKAQNGAVLSVSFKPVAVNERPTNSICISCIWWAEKSECYVTSVDTIHLLEQLVAAPNRFSVEEKNRIRRNLEGFHPSTVSKAKPESEEFFKIIMAFPHPKPRNIEKDVKVFPWTALEPALKKIIGKYSASPSSILTPVSTSSFAPPSHHGLGASQHGLSSQHADAHGQYPMHSAYTSAHHEAIPSPRSQAAWGPASAYTTSAARGLSPTLRTHHSPQQPSSMRINTSTSQLPAVSAYDTRGVAASPYGTAGLHTGMGHHPATATPPRWDTAPSTYADSGYPGLTSQQPTGGPSVYGTSAYGDGPSRA

(19) United States
(12) Patent Application Publication
(10) Pub. No.: US 2025/0320512 A1
(43) Pub. Date: Oct. 16, 2025

(54) COMPOSITIONS AND METHODS FOR ENHANCED PROTEIN PRODUCTION IN FUNGAL CELLS

(71) Applicant: DANISCO US INC., Palo Alto, CA (US)

(72) Inventors: Barbara Urszula Kozak, Utrecht (NL); Igor Nikolaev, Noordwijk (NL); Jonathan M. Palmer, Palo Alto, CA (US); Sharief Barends, Voorschoten (NL)

(21) Appl. No.: 18/711,420

(22) PCT Filed: Nov. 11, 2022
(86) PCT No.: PCT/US2022/079686

§ 371 Date: May 17, 2024

Related U.S. Application Data
(60) Provisional application No. 63/284,875, filed on Dec. 1, 2021.

(51) Int. Cl. C12N 15/80; C07K 14/37; C12N 9/42

(52) U.S. Cl. CPC C12N 15/80; C07K 14/37; C12N 9/2437

(57) ABSTRACT

The present disclosure is generally related to recombinant fungal strains for use in the commercial scale production of proteins (polypeptides) of interest. Certain embodiments are related to recombinant fungal strains deficient in the production of one or more native (endogenous) regulatory proteins and/or overexpressing one or more regulatory proteins of the disclosure.

### CROSS REFERENCE TO RELATED APPLICATIONS

This application is a U.S. National Stage Application under 35 U.S.C. § 371 of International Application No. PCT/US2022/079686, filed Nov. 11, 2022 which claims the benefit of U.S. Provisional Application No. 63/284,875, filed Dec. 1, 2021, each of which is incorporated by reference in its entirety.

### FIELD

The present disclosure is generally related to the fields of molecular biology, biochemistry, regulatory proteins, industrial fermentation, protein production, filamentous fungi and the like. Certain embodiments of the disclosure are related to mutant fungal cells and methods thereof for use in the enhanced production of proteins of interest.

### SEQUENCE LISTING

The sequence listing text file submitted herewith contains the file “NB41575-WO-PCT_SequenceListing.xml” created on Feb. 20, 2025, which is 228,748 bytes in size. This sequence listing complies with 37 C.F.R. § 1.52(e) and is incorporated herein by reference in its entirety.

### BACKGROUND

Many of the biopolymer degrading hydrolytic enzymes, such as cellulases, hemi-cellulases, ligninases, pectinases and the like have received attention because of their potential applications in food, feed, textile, pulp and paper industries and the like. For example, industrial filamentous fungal production strains, in particular Aspergillus and Trichoderma strains, can produce high amounts of these extracellular enzymes. Likewise, the existence of hypersecreting strains and strong promoters, such as cellulase (gene) promoters, render filamentous fungal cells particularly suitable for heterologous protein production.

Thus, filamentous fungi are capable of expressing native and heterologous proteins to high levels, making them well-suited for the large-scale production of enzymes and other proteins for industrial, pharmaceutical, animal health, and food and beverage applications and the like. Despite current knowledge in the art related to filamentous fungal strains, there is a continued and ongoing need in the art for improved strains for use in the production of proteins of interest. As described hereinafter, the recombinant filamentous fungal strains of the disclosure are well-suited for use in industrial scale fermentation processes for the enhanced production of endogenous and/or heterologous proteins of interest.

### SUMMARY

As described herein, the instant disclosure provides, inter alia, compositions and methods for constructing, obtaining, screening, identifying and the like recombinant (genetically modified) filamentous fungal strains deficient in the production of certain native regulatory proteins, recombinant fungal strains overexpressing certain genes encoding native regulatory proteins, recombinant fungal strains deficient in the production of certain native regulatory proteins and overexpressing certain genes encoding native regulatory proteins, recombinant fungal strains producing proteins of interest and the like. Thus, certain embodiments are related to, inter alia, recombinant fungal cells (strains) deficient in the production of one or more regulatory proteins comprising at least 80% identity to a regulatory protein set forth in TABLE 1, recombinant fungal cells overexpressing one or more regulatory proteins comprising at least 80% identity to a regulatory protein set forth in TABLE 10, recombinant fungal cells deficient in the production of one or more regulatory proteins protein set forth in TABLE 1 and overexpressing one or more regulatory proteins set forth in TABLE 10, and the like.

In other aspects, recombinant fungal cells express proteins of interest. In certain embodiments, proteins of interest include, but are not limited to, enzymes, peptides, antibodies and/or functional antibody fragments thereof, receptor proteins, animal feed proteins, human food proteins, protein biologics, therapeutic proteins, immunogenic proteins and the like. Certain other aspects of the disclosure are therefore related to, inter alia, methods for constructing, obtaining, screening, identifying, etc. recombinant fungal strains comprising enhanced protein production characteristics/phenotypes, e.g., total protein production, enzymatic activities, cellulose (PASC) hydrolysis, protein production rates, and the like.

### BRIEF DESCRIPTION OF THE DRAWINGS

FIG. 1 shows the amino acid sequences of certain Trichoderma sp. regulatory proteins identified in the filamentous fungal regulatory protein deletion library described in the Examples section below. More particularly, as presented in FIG. 1A, the regulatory protein “Trire2_4933” comprises a predicted full-length amino acid sequence of SEQ ID NO: 2 and a predicted DNA binding domain (hereinafter, “DBD”) subsequence therein, as shown in SEQ ID NO: 3; the regulatory protein “Trire2_5675” comprises a predicted full-length amino acid sequence of SEQ ID NO: 5 and a predicted DBD subsequence therein, as shown in SEQ ID NO: 6; the regulatory protein “Trire2_48438” comprises a predicted full-length amino acid sequence of SEQ ID NO: 8 and a predicted DBD subsequence therein, as shown in SEQ ID NO: 9; and the regulatory protein “Trire2_49232” comprises a predicted full-length amino acid sequence of SEQ ID NO: 11 and a predicted DBD subsequence therein, as shown in SEQ ID NO: 12. As presented in FIG. 1B, the regulatory protein “Trire2_55105” comprises a predicted full-length amino acid sequence of SEQ ID NO: 14 and a predicted DBD subsequence therein, as shown in SEQ ID NO: 15; the regulatory protein “Trire2_60565” comprises a predicted full-length amino acid sequence of SEQ ID NO: 17 and a predicted DBD subsequence therein, as shown in SEQ ID NO: 18; and the regulatory protein “Trire2_60931” comprises a predicted full-length amino acid sequence of SEQ ID NO: 20 and a predicted DBD subsequence therein, as shown in SEQ ID NO: 21. As presented in FIG. 1C, the regulatory protein “Trire2_67209” comprises a predicted full-length amino acid sequence of SEQ ID NO: 23 and a predicted DBD subsequence therein, as shown in SEQ ID NO: 24; the regulatory protein “Trire2_68097” comprises a predicted full-length amino acid sequence of SEQ ID NO: 26, and a predicted DBD subsequence therein, as shown in SEQ ID NO: 27; and the regulatory protein “Trire2_68425” comprises a predicted full-length amino acid sequence of SEQ ID NO: 29 and a predicted DBD subsequence therein, as shown in SEQ ID NO: 30. As presented in FIG. 1D, the regulatory protein “Trire2_69695” comprises a predicted full-length amino acid sequence of SEQ ID NO: 32 and a predicted DBD subsequence therein, as shown in SEQ ID NO: 33; the regulatory protein “Trire2_71823” comprises a predicted full-length amino acid sequence of SEQ ID NO: 35 and a predicted DBD subsequence therein, as shown in SEQ ID NO: 36; and the regulatory protein “Trire2_72993” comprises a predicted full-length amino acid sequence of SEQ ID NO: 38 and a predicted DBD subsequence therein, as shown in SEQ ID NO: 39. As presented in FIG. 1E, the regulatory protein “Trire2_76705” comprises a predicted full-length amino acid sequence of SEQ ID NO: 41 and a predicted DBD subsequence therein, as shown in SEQ ID NO: 42; the regulatory protein “Trire2_76872” comprises a predicted full-length amino acid sequence of SEQ ID NO: 44 and a predicted DBD subsequence therein, as shown in SEQ ID NO: 45; and the regulatory protein “Trire2_77291” comprises a predicted full-length amino acid sequence of SEQ ID NO: 47 and a predicted DBD subsequence therein, as shown in SEQ ID NO: 48; and the regulatory protein “Trire2_78162” comprises a predicted full-length amino acid sequence of SEQ ID NO: 50 and a predicted DBD subsequence therein, as shown in SEQ ID NO: 51. As presented in FIG. 1F, the regulatory protein “Trire2_103122” comprises a predicted full-length amino acid sequence of SEQ ID NO: 53 and a predicted DBD subsequence therein, as shown in SEQ ID NO: 54; the regulatory protein “Trire2_105239” comprises a predicted full-length amino acid sequence of SEQ ID NO: 56, and a predicted DBD subsequence therein, as shown in SEQ ID NO: 57; and the regulatory protein “Trire2_105849” comprises a predicted full-length amino acid sequence of SEQ ID NO: 59 and a predicted DBD subsequence therein, as shown in SEQ ID NO: 60; and the regulatory protein “Trire2_108013” comprises a predicted full-length amino acid sequence of SEQ ID NO: 62 and a predicted DBD subsequence therein, as shown in SEQ ID NO: 63. As presented in FIG. 1G, the regulatory protein “Trire2_108775” comprises a predicted full-length amino acid sequence of SEQ ID NO: 65 and a predicted DBD subsequence therein, as shown in SEQ ID NO: 66; the regulatory protein “Trire2_119986” comprises a predicted full-length amino acid sequence of SEQ ID NO: 68 and a predicted DBD subsequences therein, as shown in SEQ ID NO: 69; and the regulatory protein “Trire2_120428” comprises a predicted full-length amino acid sequence of SEQ ID NO: 71 and a predicted DBD subsequence therein, as shown in SEQ ID NO: 72. As presented in FIG. 1H, the regulatory protein “Trire2_120597” comprises a predicted full-length amino acid sequence of SEQ ID NO: 74 and a predicted DBD subsequence therein, as shown in SEQ ID NO: 75; the regulatory protein “Trire2_121757” comprises a predicted full-length amino acid sequence of SEQ ID NO: 77 and a predicted DBD subsequence therein, as shown in SEQ ID NO: 78; and the regulatory protein “Trire2_122783” comprises a predicted full-length amino acid sequence of SEQ ID NO: 80 and a predicted DBD subsequences therein, as shown in SEQ ID NO: 81.

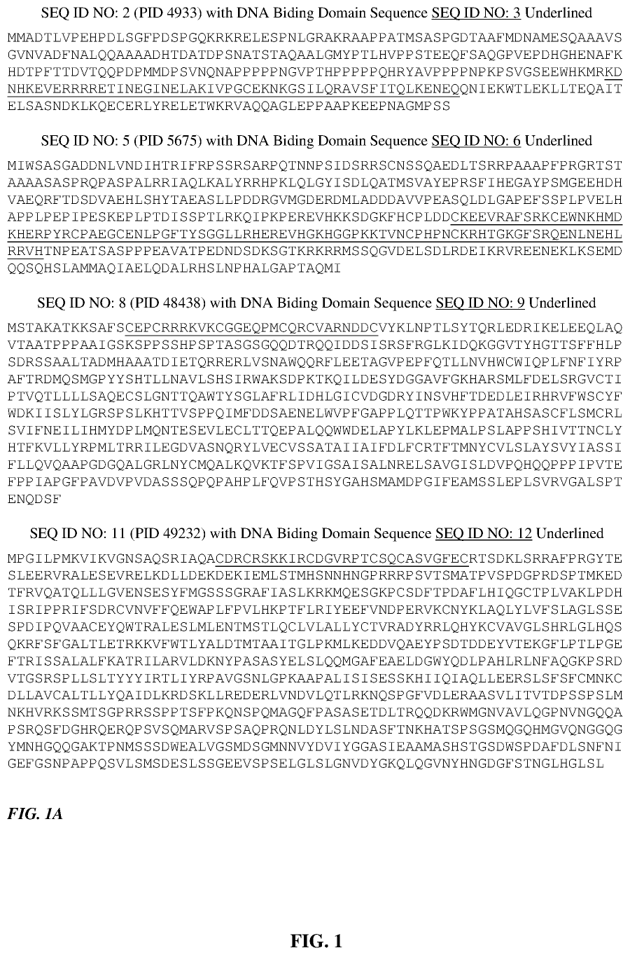

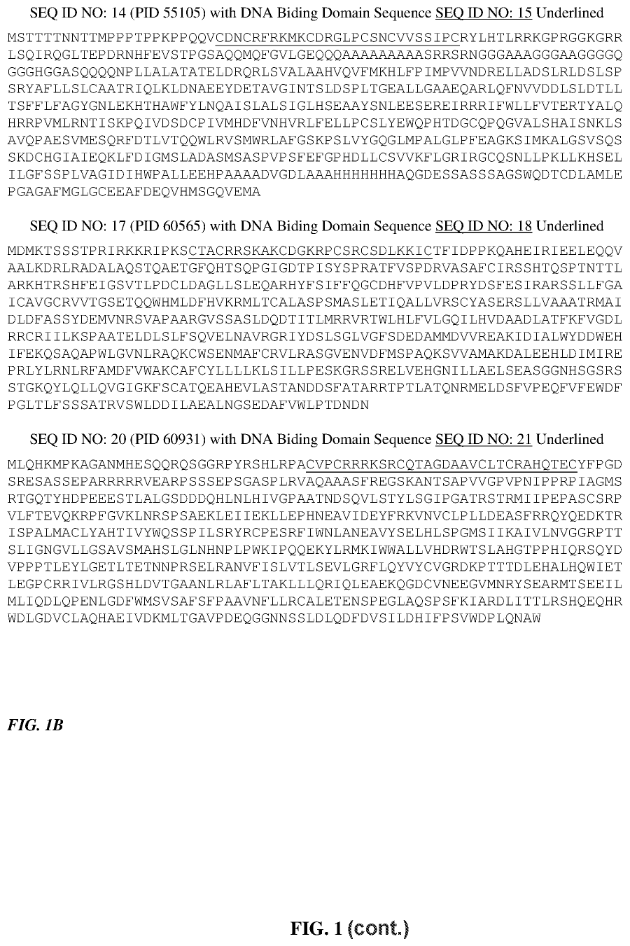

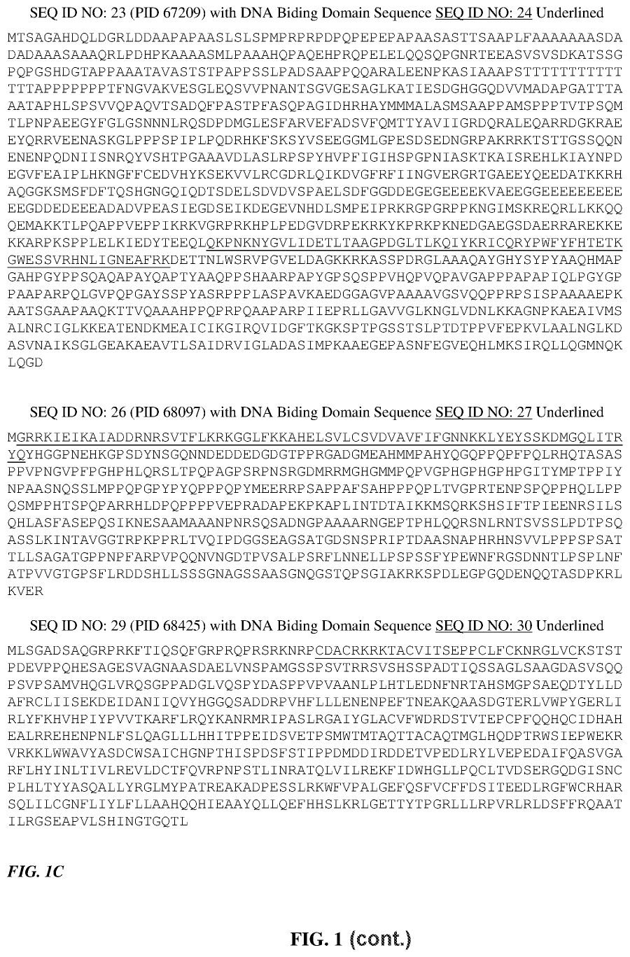

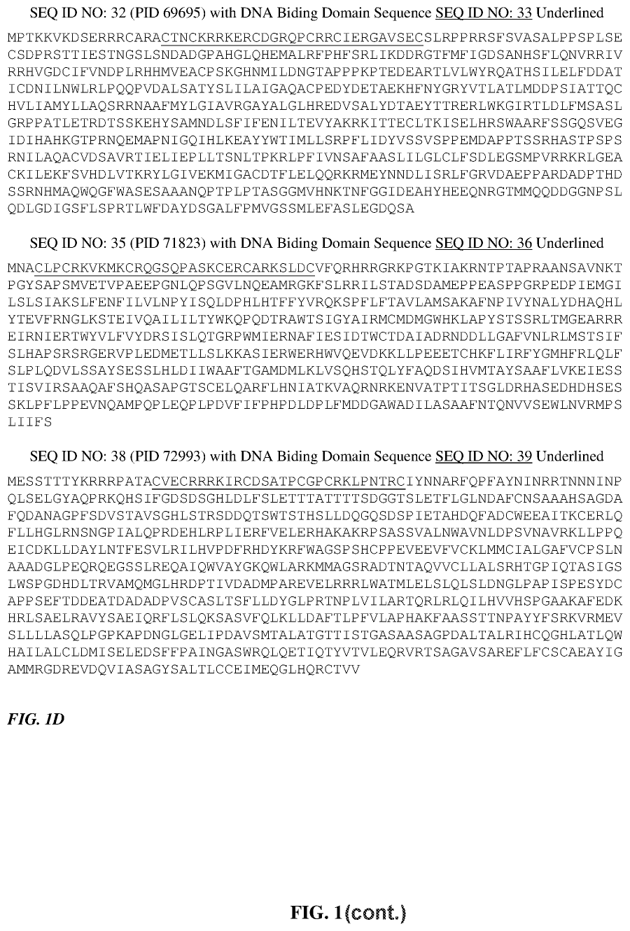

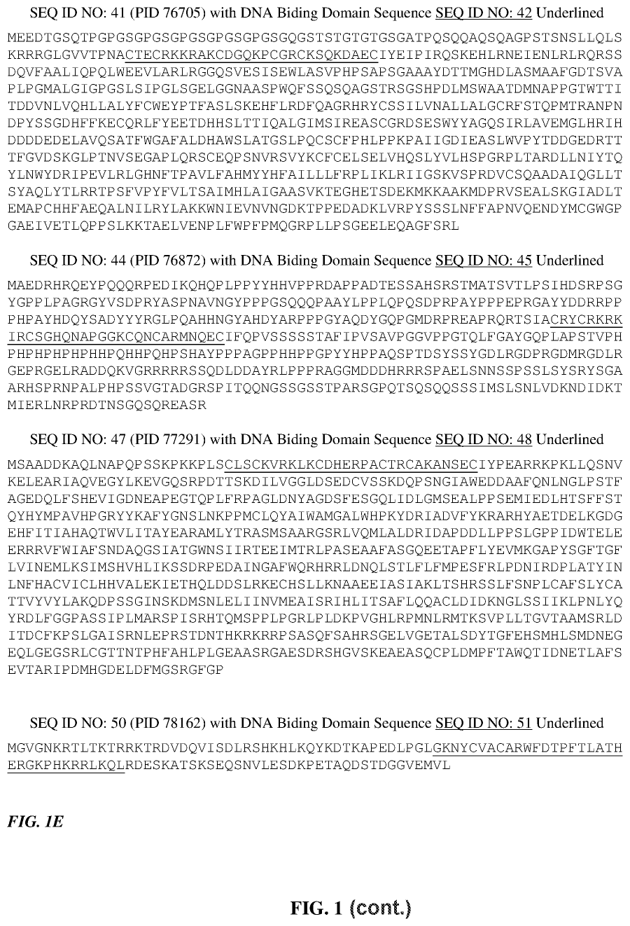

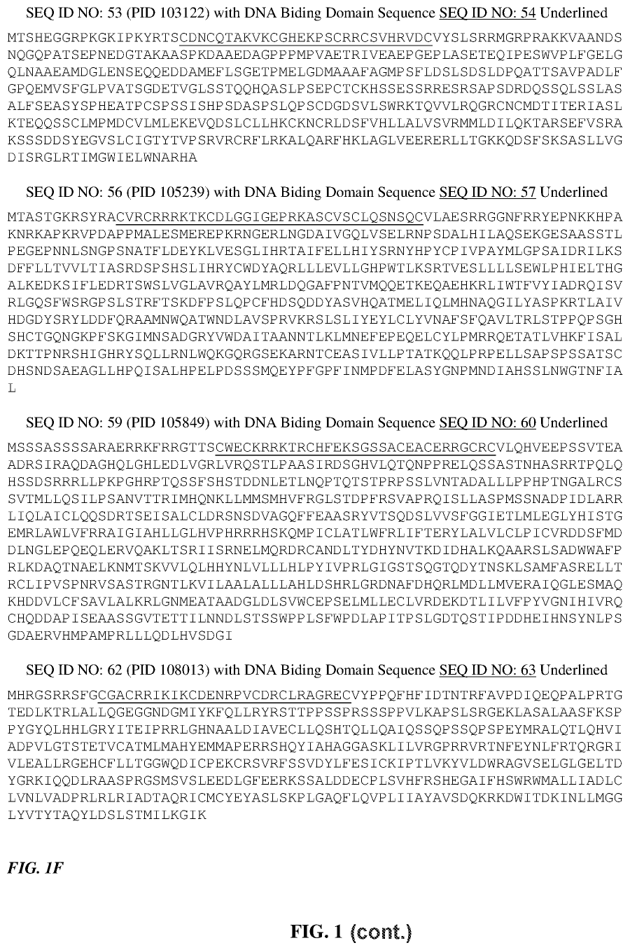

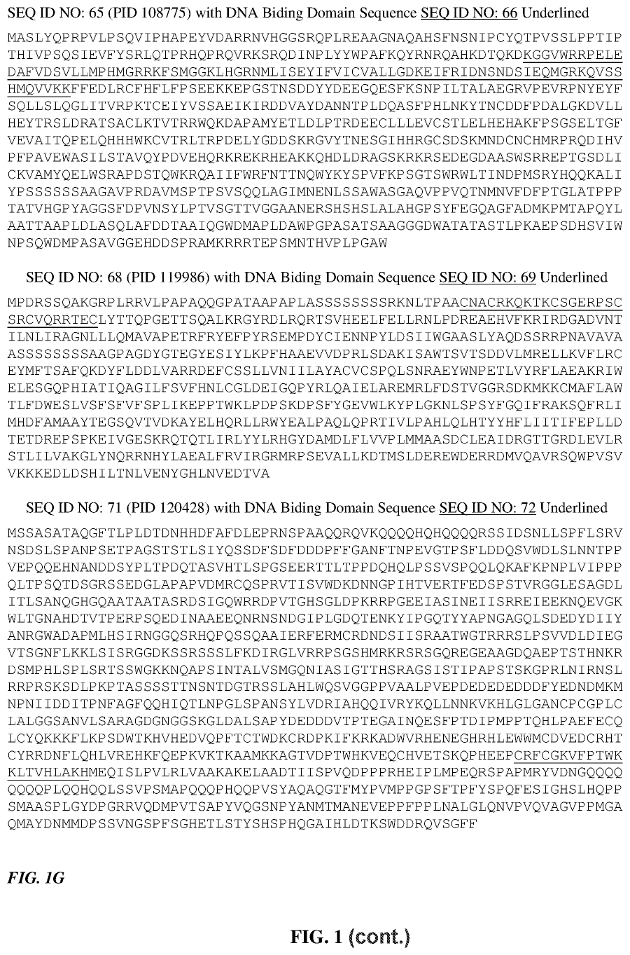

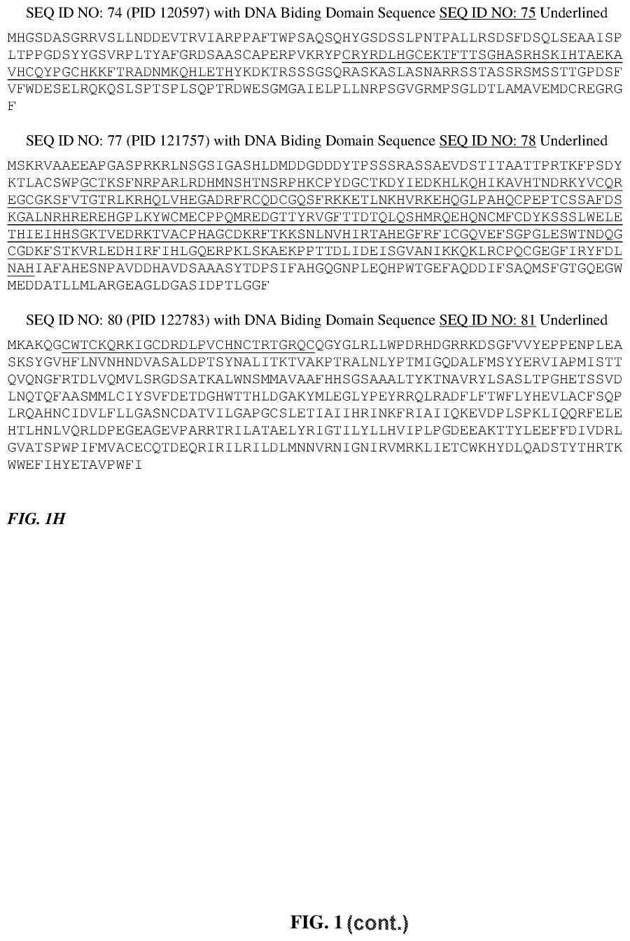

### BRIEF DESCRIPTION OF THE BIOLOGICAL SEQUENCES

SEQ ID NO: 1 is a Trichoderma reesei nucleic acid (DNA) sequence encoding a regulatory protein named “Trire2_4933 (PID 4933)”.

SEQ ID NO: 2 is the predicted amino acid sequence of the Trire2_4933 (PID 4933) regulatory protein encoded by the nucleic acid sequence of SEQ ID NO: 1.

SEQ ID NO: 3 is the amino acid sequence of a putative DNA binding domain (hereinafter, “DBD”) present in the Trire2_4933 (PID 4933) regulatory protein of SEQ ID NO: 2.

SEQ ID NO: 4 is a T. reesei nucleic acid sequence encoding a regulatory protein named “Trire2_5675 (PID 5675)”.

SEQ ID NO: 5 is the predicted amino acid sequence of the Trire2_5675 (PID 5675) regulatory protein encoded by the nucleic acid sequence of SEQ ID NO: 4.

SEQ ID NO: 6 is the amino acid sequence of a putative DBD present the Trire2_5675 (PID 5675) regulatory protein of SEQ ID NO: 5.

SEQ ID NO: 7 is a T. reesei nucleic acid sequence encoding a regulatory protein named “Trire2_48438 (PID 48438)”.

SEQ ID NO: 8 is the predicted amino acid sequence of the Trire2_48438 (PID 48438) regulatory protein encoded by the nucleic acid sequence of SEQ ID NO: 7.

SEQ ID NO: 9 is the amino acid sequence of a putative DBD present the Trire2_48438 (PID 48438) regulatory protein of SEQ ID NO: 8.

SEQ ID NO: 10 is a T. reesei nucleic acid sequence encoding a regulatory protein named “Trire2_49232 (PID 49232)”.

SEQ ID NO: 11 is the predicted amino acid sequence of the Trire2_49232 (PID 49232) regulatory protein encoded by the nucleic acid sequence of SEQ ID NO: 10.

SEQ ID NO: 12 is the amino acid sequence of a putative DBD present the Trire2_49232 (PID 49232) regulatory protein of SEQ ID NO: 11.

SEQ ID NO: 13 is a T. reesei nucleic acid sequence encoding a regulatory protein named “Trire2_55105 (PID 55105)”.

SEQ ID NO: 14 is the predicted amino acid sequence of the Trire2_55105 (PID 55105) regulatory protein encoded by the nucleic acid sequence of SEQ ID NO: 13.

SEQ ID NO: 15 is the amino acid sequence of a putative DBD present the Trire2_55105 (PID 55105) regulatory protein of SEQ ID NO: 14.

SEQ ID NO: 16 is a T. reesei nucleic acid sequence encoding a regulatory protein named “Trire2_60565 (PID 60565)”.

SEQ ID NO: 17 is the predicted amino acid sequence of the Trire2_60565 (PID 60565) regulatory protein encoded by the nucleic acid sequence of SEQ ID NO: 16.

SEQ ID NO: 18 is the amino acid sequence of a putative DBD present the Trire2_60565 (PID 60565) regulatory protein of SEQ ID NO: 17.

SEQ ID NO: 19 is a T. reesei nucleic acid sequence encoding a regulatory protein named “Trire2_60931 (PID 60931)”.

SEQ ID NO: 20 is the predicted amino acid sequence of the Trire2_60931 (PID 60931) regulatory protein encoded by the nucleic acid sequence of SEQ ID NO: 19.

SEQ ID NO: 21 is the amino acid sequence of a putative DBD present the Trire2_60931 (PID 60931) regulatory protein of SEQ ID NO: 20.

SEQ ID NO: 22 is a T. reesei nucleic acid sequence encoding a regulatory protein named “Trire2_67209 (PID 67209)”.

SEQ ID NO: 23 is the predicted amino acid sequence of the Trire2_67209 (PID 67209) regulatory protein encoded by the nucleic acid sequence of SEQ ID NO: 22.

SEQ ID NO: 24 is the amino acid sequence of a putative DBD present the Trire2_67209 (PID 67209) regulatory protein of SEQ ID NO: 23.

SEQ ID NO: 25 is a T. reesei nucleic acid sequence encoding a regulatory protein named “Trire2_68097 (PID 68097)”.

SEQ ID NO: 26 is the predicted amino acid sequence of the Trire2_68097 (PID 68097) regulatory protein encoded by the nucleic acid sequence of SEQ ID NO: 25.

SEQ ID NO: 27 is the amino acid sequence of a putative DBD present the Trire2_68097 (PID 68097) regulatory protein of SEQ ID NO: 26.

SEQ ID NO: 28 is a T. reesei nucleic acid sequence encoding a regulatory protein named “Trire2_68425 (PID 68425)”.

SEQ ID NO: 29 is the predicted amino acid sequence of the Trire2_68425 (PID 68425) regulatory protein encoded by the nucleic acid sequence of SEQ ID NO: 28.

SEQ ID NO: 30 is the amino acid sequence of a putative DBD present the Trire2_68425 (PID 68425) regulatory protein of SEQ ID NO: 29.

SEQ ID NO: 31 is a T. reesei nucleic acid sequence encoding a regulatory protein named “Trire2_69695 (PID 69695)”.

SEQ ID NO: 32 is the predicted amino acid sequence of the Trire2_69695 (PID 69695) regulatory protein encoded by the nucleic acid sequence of SEQ ID NO: 31.

SEQ ID NO: 33 is the amino acid sequence of a putative DBD present the Trire2_69695 (PID 69695) regulatory protein of SEQ ID NO: 32.

SEQ ID NO: 34 is a T. reesei nucleic acid sequence encoding a regulatory protein named “Trire2_71823 (PID 71823)”.

SEQ ID NO: 35 is the predicted amino acid sequence of the Trire2_71823 (PID 71823) regulatory protein encoded by the nucleic acid sequence of SEQ ID NO: 34.

SEQ ID NO: 36 is the amino acid sequence of a putative DBD present the Trire2_71823 (PID 71823) regulatory protein of SEQ ID NO: 35.

SEQ ID NO: 37 is a T. reesei nucleic acid sequence encoding a regulatory protein named “Trire2_72993 (PID 72993)”.

SEQ ID NO: 38 is the predicted amino acid sequence of the Trire2_72993 (PID 72993) regulatory protein encoded by the nucleic acid sequence of SEQ ID NO: 37.

SEQ ID NO: 39 is the amino acid sequence of a putative DBD present the Trire2_72993 (PID 72993) regulatory protein of SEQ ID NO: 38.

SEQ ID NO: 40 is a T. reesei nucleic acid sequence encoding a regulatory protein named “Trire2_76705 (PID 76705)”.

SEQ ID NO: 41 is the predicted amino acid sequence of the Trire2_76705 (PID 76705) regulatory protein encoded by the nucleic acid sequence of SEQ ID NO: 40.

SEQ ID NO: 42 is the amino acid sequence of a putative DBD present the Trire2_76705 (PID 76705) regulatory protein of SEQ ID NO: 41.

SEQ ID NO: 43 is a T. reesei nucleic acid sequence encoding a regulatory protein named “Trire2_76872 (PID 76872)”.

SEQ ID NO: 44 is the predicted amino acid sequence of the Trire2_76872 (PID 76872) regulatory protein encoded by the nucleic acid sequence of SEQ ID NO: 43.

SEQ ID NO: 45 is the amino acid sequence of a putative DBD present the Trire2_76872 (PID 76872) regulatory protein of SEQ ID NO: 44.

SEQ ID NO: 46 is a T. reesei nucleic acid sequence encoding a regulatory protein named “Trire2_77291 (PID 77291)”.

SEQ ID NO: 47 is the predicted amino acid sequence of the Trire2_77291 (PID 77291) regulatory protein encoded by the nucleic acid sequence of SEQ ID NO: 46.

SEQ ID NO: 48 is the amino acid sequence of a putative DBD present the Trire2_77291 (PID 77291) regulatory protein of SEQ ID NO: 47.

SEQ ID NO: 49 is a T. reesei nucleic acid sequence encoding a regulatory protein named “Trire2_78162 (PID 78162)”.

SEQ ID NO: 50 is the predicted amino acid sequence of the Trire2_78162 (PID 78162) regulatory protein encoded by the nucleic acid sequence of SEQ ID NO: 49.

SEQ ID NO: 51 is the amino acid sequence of a putative DBD present the Trire2_78162 (PID 78162) regulatory protein of SEQ ID NO: 50.

SEQ ID NO: 52 is a T. reesei nucleic acid sequence encoding a regulatory protein named “Trire2_103122 (PID 103122)”.

SEQ ID NO: 53 is the predicted amino acid sequence of the Trire2_103122 (PID 103122) regulatory protein encoded by the nucleic acid sequence of SEQ ID NO: 52.

SEQ ID NO: 54 is the amino acid sequence of a putative DBD present the Trire2_103122 (PID 103122) regulatory protein of SEQ ID NO: 53.

SEQ ID NO: 55 is a T. reesei nucleic acid sequence encoding a regulatory protein named “Trire2_105239 (PID 105239)”.

SEQ ID NO: 56 is the predicted amino acid sequence of the Trire2_105239 (PID 105239) regulatory protein encoded by the nucleic acid sequence of SEQ ID NO: 55.

SEQ ID NO: 57 is the amino acid sequence of a putative DBD present the Trire2_105239 (PID 105239) regulatory protein of SEQ ID NO: 56.

SEQ ID NO: 58 is a T. reesei nucleic acid sequence encoding a regulatory protein named “Trire2_105849 (PID 105849)”.

SEQ ID NO: 59 is the predicted amino acid sequence of the Trire2_105849 (PID 105849) regulatory protein encoded by the nucleic acid sequence of SEQ ID NO: 58.

SEQ ID NO: 60 is the amino acid sequence of a putative DBD present the Trire2_105849 (PID 105849) regulatory protein of SEQ ID NO: 59.

SEQ ID NO: 61 is a T. reesei nucleic acid sequence encoding a regulatory protein named “Trire2_108013 (PID 108013)”.

SEQ ID NO: 62 is the predicted amino acid sequence of the Trire2_108013 (PID 108013) regulatory protein encoded by the nucleic acid sequence of SEQ ID NO: 61.

SEQ ID NO: 63 is the amino acid sequence of a putative DBD present the Trire2_108013 (PID 108013) regulatory protein of SEQ ID NO: 62.

SEQ ID NO: 64 is a T. reesei nucleic acid sequence encoding a regulatory protein named “Trire2_108775 (PID 108775)”.

SEQ ID NO: 65 is the predicted amino acid sequence of the Trire2_108775 (PID 108775) regulatory protein encoded by the nucleic acid sequence of SEQ ID NO: 64.

SEQ ID NO: 66 is the amino acid sequence of a putative DBD present the Trire2_108775 (PID 108775) regulatory protein of SEQ ID NO: 65.

SEQ ID NO: 67 is a T. reesei nucleic acid sequence encoding a regulatory protein named “Trire2_119986 (PID 119986)”.

SEQ ID NO: 68 is the predicted amino acid sequence of the Trire2_119986 (PID 119986) regulatory protein encoded by the nucleic acid sequence of SEQ ID NO: 67.

SEQ ID NO: 69 is the amino acid sequence of a putative DBD present the Trire2_119986 (PID 119986) regulatory protein of SEQ ID NO: 68.

SEQ ID NO: 70 is a T. reesei nucleic acid sequence encoding a regulatory protein named “Trire2_120428 (PID 120428)”.

SEQ ID NO: 71 is the predicted amino acid sequence of the Trire2_120428 (PID 120428) regulatory protein encoded by the nucleic acid sequence of SEQ ID NO: 70.

SEQ ID NO: 72 is the amino acid sequence of a putative DBD present the Trire2_120428 (PID 120428) regulatory protein of SEQ ID NO: 71.

SEQ ID NO: 73 is a T. reesei nucleic acid sequence encoding a regulatory protein named “Trire2_120597 (PID 120597)”.

SEQ ID NO: 74 is the predicted amino acid sequence of the Trire2_120597 (PID 120597) regulatory protein encoded by the nucleic acid sequence of SEQ ID NO: 73.

SEQ ID NO: 75 is the amino acid sequence of a putative DBD present the Trire2_120597 (PID 120597) regulatory protein of SEQ ID NO: 74.

SEQ ID NO: 76 is a T. reesei nucleic acid sequence encoding a regulatory protein named “Trire2_121757 (PID 121757)”.

SEQ ID NO: 77 is the predicted amino acid sequence of the Trire2_121757 (PID 121757) regulatory protein encoded by the nucleic acid sequence of SEQ ID NO: 79.

SEQ ID NO: 78 is the amino acid sequence of a putative DBD present the Trire2_121757 (PID 121757) regulatory protein of SEQ ID NO: 80.

SEQ ID NO: 79 is a T. reesei nucleic acid sequence encoding a regulatory protein named “Trire2_122783 (PID 122783)”.

SEQ ID NO: 80 is the predicted amino acid sequence of the Trire2_122783 (PID 122783) regulatory protein encoded by the nucleic acid sequence of SEQ ID NO: 79.

SEQ ID NO: 81 is the amino acid sequence of a putative DBD present the Trire2_122783 (PID 122783) regulatory protein of SEQ ID NO: 80.

SEQ ID NO: 82 is the predicted amino acid sequence of a regulatory protein named “Trire2_1941 (PID 1941)”.

SEQ ID NO: 83 is the predicted amino acid sequence of a regulatory protein named “Trire2_2148 (PID 2148)”.

SEQ ID NO: 84 is the predicted amino acid sequence of a regulatory protein named “Trire2_3605 (PID 3605)”.

SEQ ID NO: 85 is the predicted amino acid sequence of a regulatory protein named “Trire2_4748 (PID 4748)”.

SEQ ID NO: 86 is the predicted amino acid sequence of a regulatory protein named “Trire2_5664 (PID 5664)”.

SEQ ID NO: 87 is the predicted amino acid sequence of a regulatory protein named “Trire2_5927 (PID 5927)”.

SEQ ID NO: 88 is the predicted amino acid sequence of a regulatory protein named “Trire2_21997 (PID 21997)”.

SEQ ID NO: 89 is the predicted amino acid sequence of a regulatory protein named “Trire2_22774 (PID 22774)”.

SEQ ID NO: 90 is the predicted amino acid sequence of a regulatory protein named “Trire2_22785 (PID 22785)”.

SEQ ID NO: 91 is the predicted amino acid sequence of a regulatory protein named “Trire2_36703 (PID 36703)”.

SEQ ID NO: 92 is the predicted amino acid sequence of a regulatory protein named “Trire2_44290 (PID 44290)”.

SEQ ID NO: 93 is the predicted amino acid sequence of a regulatory protein named “Trire2_44781 (PID 44781)”.

SEQ ID NO: 94 is the predicted amino acid sequence of a regulatory protein named “Trire2_45866 (PID 45866)”.

SEQ ID NO: 95 is the predicted amino acid sequence of a regulatory protein named “Trire2_48773 (PID 48773)”.

SEQ ID NO: 96 is the predicted amino acid sequence of a regulatory protein named “Trire2_49918 (PID 49918)”.

SEQ ID NO: 97 is the predicted amino acid sequence of a regulatory protein named “Trire2_52438 (PID 52438)”.

SEQ ID NO: 98 is the predicted amino acid sequence of a regulatory protein named “Trire2_52924 (PID 52924)”.

SEQ ID NO: 99 is the predicted amino acid sequence of a regulatory protein named “Trire2_53106 (PID 53106)”.

SEQ ID NO: 100 is the predicted amino acid sequence of a regulatory protein named “Trire2_53484 (PID 53484)”.

SEQ ID NO: 101 is the predicted amino acid sequence of a regulatory protein named “Trire2_55274 (PID 55274)”.

SEQ ID NO: 102 is the predicted amino acid sequence of a regulatory protein named “Trire2_56214 (PID 56214)”.

SEQ ID NO: 103 is the predicted amino acid sequence of a regulatory protein named “Trire2_58130 (PID 58130)”.

SEQ ID NO: 104 is the predicted amino acid sequence of a regulatory protein named “Trire2_59353 (PID 59353)”.

SEQ ID NO: 105 is the predicted amino acid sequence of a regulatory protein named “Trire2_59609 (PID 59609)”.

SEQ ID NO: 106 is the predicted amino acid sequence of a regulatory protein named “Trire2_60558 (PID 60558)”.

SEQ ID NO: 107 is the predicted amino acid sequence of a regulatory protein named “Trire2_61476 (PID 61476)”.

SEQ ID NO: 108 is the predicted amino acid sequence of a regulatory protein named “Trire2_65070 (PID 65070)”.

SEQ ID NO: 109 is the predicted amino acid sequence of a regulatory protein named “Trire2_65895 (PID 65895)”.

SEQ ID NO: 110 is the predicted amino acid sequence of a regulatory protein named “Trire2_68028 (PID 68028)”.

SEQ ID NO: 111 is the predicted amino acid sequence of a regulatory protein named “Trire2_70414 (PID 70414)”.

SEQ ID NO: 112 is the predicted amino acid sequence of a regulatory protein named “Trire2_70991 (PID 70991)”.

SEQ ID NO: 113 is the predicted amino acid sequence of a regulatory protein named “Trire2_71080 (PID 71080)”.

SEQ ID NO: 114 is the predicted amino acid sequence of a regulatory protein named “Trire2_71689 (PID 71689)”.

SEQ ID NO: 115 is the predicted amino acid sequence of a regulatory protein named “Trire2_72076 (PID 72076)”.

SEQ ID NO: 116 is the predicted amino acid sequence of a regulatory protein named “Trire2_73417 (PID 73417)”.

SEQ ID NO: 117 is the predicted amino acid sequence of a regulatory protein named “Trire2_73559 (PID 73559)”.

SEQ ID NO: 118 is the predicted amino acid sequence of a regulatory protein named “Trire2_73689 (PID 73689)”.

SEQ ID NO: 119 is the predicted amino acid sequence of a regulatory protein named “Trire2_76039 (PID 76039)”.

SEQ ID NO: 120 is the predicted amino acid sequence of a regulatory protein named “Trire2_76590 (PID 76590)”.

SEQ ID NO: 121 is the predicted amino acid sequence of a regulatory protein named “Trire2_77878 (PID 77878)”.

SEQ ID NO: 122 is the predicted amino acid sequence of a regulatory protein named “Trire2_1057844 (PID 105784)”.

SEQ ID NO: 123 is the predicted amino acid sequence of a regulatory protein named “Trire2_105880 (PID 105880)”.

SEQ ID NO: 124 is the predicted amino acid sequence of a regulatory protein named “Trire2_105917 (PID 105917)”.

SEQ ID NO: 125 is the predicted amino acid sequence of a regulatory protein named “Trire2_105980 (PID 105980)”.

SEQ ID NO: 126 is the predicted amino acid sequence of a regulatory protein named “Trire2_105989 (PID 105989)”.

SEQ ID NO: 127 is the predicted amino acid sequence of a regulatory protein named “Trire2_106009 (PID 106009)”.

SEQ ID NO: 128 is the predicted amino acid sequence of a regulatory protein named “Trire2_106720 (PID 106720)”.

SEQ ID NO: 129 is the predicted amino acid sequence of a regulatory protein named “Trire2_109277 (PID 109277)”.

SEQ ID NO: 130 is the predicted amino acid sequence of a regulatory protein named “Trire2_110901 (PID 110901)”.

SEQ ID NO: 131 is the predicted amino acid sequence of a regulatory protein named “Trire2_119826 (PID 119826)”.

SEQ ID NO: 132 is the predicted amino acid sequence of a regulatory protein named “Trire2_121164 (PID 121164)”.

SEQ ID NO: 133 is the predicted amino acid sequence of a regulatory protein named “Trire2_121310 (PID 121310)”.

SEQ ID NO: 134 is the predicted amino acid sequence of a regulatory protein named “Trire2_121602 (PID 121602)”.

SEQ ID NO: 135 is the predicted amino acid sequence of a regulatory protein named “Trire2_122457 (PID 122457)”.

SEQ ID NO: 136 is the predicted amino acid sequence of a regulatory protein named “Trire2_123713 (PID 123713)”.

### DETAILED DESCRIPTION

### I. Overview

As described herein, certain embodiments of the disclosure are related to mutant (recombinant) fungal cells (strains) for use in the commercial scale production of proteins of interest. Certain aspects are therefore related to compositions and methods for constructing and obtaining recombinant fungal strains expressing proteins of interest. In certain aspects, the disclosure provides, inter alia, compositions and methods for constructing, obtaining, screening, identifying and the like recombinant (genetically modified) filamentous fungal strains deficient in the production of certain native regulatory proteins, recombinant fungal strains overexpressing certain genes encoding native regulatory proteins, recombinant fungal strains deficient in the production of certain native regulatory proteins and overexpressing certain genes encoding native regulatory proteins, recombinant fungal strains producing proteins of interest and the like.

### II. Definitions

Prior to describing the present strains, compositions and methods in further detail, the following terms and phrases are defined. Terms not defined should be accorded their ordinary meaning as used and known to one skilled in the art.

All publications and patents cited in this specification are herein incorporated by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the present compositions and methods. The upper and lower limits of these smaller ranges may independently be included in the smaller ranges and are also encompassed within the present compositions and methods, subject to any specifically excluded limit in the stated range. Where the stated range includes one or both of the limits, ranges excluding either or both of those included limits are also included in the present compositions and methods.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. For example, in connection with a numerical value, the term “about” refers to a range of ⁻10% to ⁺10% of the numerical value, unless the term is otherwise specifically defined in context. In another example, the phrase a “pH value of about 6” refers to pH values of from 5.4 to 6.6, unless the pH value is specifically defined otherwise.

In accordance with this Detailed Description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

It is further noted that the claims may be drafted to exclude any optional element. As such, this statement is intended to serve as antecedent basis for use of such exclusive terminology as “solely”, “only”, “excluding”, “not including” and the like in connection with the recitation of claim elements, or use of a “negative” limitation or “proviso”. For example, in certain embodiments, the proviso “wherein the medium does not comprise an inducing substrate” may be used to exclude inducing substrates such as cellulose, lactose, gentibiose, sophorose and the like.

It is further noted that the term “comprising”, as used herein, means “including, but not limited to”, the component(s) after the term “comprising”. The component(s) after the term “comprising” are required or mandatory, but the composition comprising the component(s) may further include other non-mandatory or optional component(s).

It is also noted that the term “consisting of,” as used herein, means “including and limited to”, the component(s) after the term “consisting of”. The component(s) after the term “consisting of” are therefore required or mandatory, and no other component(s) are present in the composition.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, the term “Ascomycete fungal cell” refers to any organism in the Division Ascomycota in the Kingdom Fungi. Examples of Ascomycetes fungal cells include, but are not limited to, filamentous fungi in the subphylum Pezizomycotina, such as Trichoderma sp., Aspergillus sp., Myceliophthora sp., Penicillium sp., and the like.

As used herein, the term “filamentous fungus” refers to all filamentous forms of the subdivision Eumycota and Oomycota. For example, filamentous fungi include, without limitation, Acremonium, Aspergillus, Emericella, Fusarium, Humicola, Mucor, Myceliophthora, Neurospora, Penicillium, Scytalidium, Thielavia, Tolypocladium, and Trichoderma species.

In certain embodiments, a filamentous fungus is a Trichoderma sp. cell (strain) including, but not limited to, Trichoderma harzianum, Trichoderma koningii, Trichoderma longibrachiatum, Trichoderma reesei and Trichoderma viride. As known to one skilled in the art, Trichoderma reesei was previously classified as “Hypocrea jecorina”.

In other embodiments, a filamentous fungus is an Aspergillus sp. cell (strain) such as Aspergillus aculeatus, Aspergillus awamori, Aspergillus clavatus, Aspergillus flavus, Aspergillus foetidus, Aspergillus fumigatus, Aspergillus japonicus, Aspergillus nidulans, Aspergillus niger, Aspergillus oryzae and Aspergillus terreus.

As used herein, the terms “wild-type” and “native” are used interchangeably and refer to genes, proteins, fungal cells or strains as found in nature.

As used herein, the terms “recombinant” or “non-natural” refer to an organism, microorganism, cell, nucleic acid molecule, or vector that has at least one engineered genetic alteration, or has been modified by the introduction of a heterologous nucleic acid molecule, or refer to a cell (e.g., a microbial cell) that has been altered such that the expression of a heterologous or endogenous nucleic acid molecule or gene can be controlled. Recombinant also refers to a cell that is derived from a non-natural cell or is progeny of a non-natural cell having one or more such modifications. Genetic alterations include, for example, modifications introducing expressible nucleic acid molecules encoding proteins, or other nucleic acid molecule additions, deletions, substitutions or other functional alteration of a cell's genetic material. For example, recombinant cells may express genes or other nucleic acid molecules that are not found in identical or homologous form within a native (wild-type) cell, or may provide an altered expression pattern of endogenous genes, such as being over-expressed, under-expressed, minimally expressed, or not expressed at all.

“Recombination”, “recombining” or generating a “recombined” nucleic acid is generally the assembly of two or more nucleic acid fragments wherein the assembly gives rise to a chimeric gene or polynucleotide.

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype.

As used herein, the term “gene” means the segment of DNA involved in producing a polypeptide (protein) chain, that may or may not include regions preceding and following the coding region (e.g., 5′ untranslated (5′ UTR) or “leader” sequences, 3′ UTR or “trailer” sequences, promoter sequences, terminator sequences and the like) as well as intervening sequences (introns) between individual coding segments (exons). For example, a gene (DNA) sequence of interest (GOI) may encode a regulatory protein, a structural protein, commercially important industrial proteins or peptides, such as enzymes (e.g., proteases, mannanases, xylanases, amylases, glucoamylases, cellulases, oxidases, phytases, lipases) and the like. The gene of interest may be a naturally occurring gene, a mutated (modified) gene or a synthetic gene.

As used herein, the term “promoter” refers to a nucleic acid sequence that functions to direct transcription of a downstream gene coding sequence (CDS; or open reading frame (ORF)). The promoter will generally be appropriate to the host cell (e.g., a fungal cell) in which the target gene is being expressed. The promoter together with other transcriptional and translational regulatory nucleic acid sequences (also termed “control sequences”) is necessary to express a given gene. In general, the transcriptional and translational regulatory sequences include, but are not limited to, promoter and terminator sequences including a core promoter and enhancer or activator or repressor sequences, transcriptional and translational start and stop sequences. In certain embodiments, the promoter is an inducible promoter, a constitutive promoter, a tunable promoter, a synthetic promoter, a tandem promoter and combinations thereof. In certain embodiments, the inducible promoter is an inducible cellulase gene promoter.

As used herein, the term “promoter activity” is the ability of a nucleic acid to direct transcription of a downstream (3′) polynucleotide in a host cell. To test promoter activity, the (promoter) nucleic acid may be operably linked to a downstream polynucleotide to produce a recombinant nucleic acid. The recombinant nucleic acid may be introduced into a cell, and transcription of the polynucleotide may be evaluated. In certain cases, the polynucleotide may encode a protein, and transcription of the polynucleotide can be evaluated by assessing production of the protein in the cell.

As used herein, the term “operably linked” refers to a functional linkage between two or more nucleic acid sequences. Thus, a nucleic acid sequence is operably linked when it is placed into a functional relationship with another nucleic acid sequence. For example, a promoter sequence or a terminator sequence is operably linked to a gene coding sequence (CDS) if it affects the transcription of the CDS; a ribosome binding site is operably linked to a coding sequence if it is positioned so as to facilitate translation; a nucleic acid sequence encoding a secretory leader (i.e., a signal peptide) is operably linked to a nucleic acid sequence (e.g., an ORF) encoding a polypeptide if it is expressed as a pre-protein that participates in the secretion of the polypeptide. Generally, “operably linked” means that the DNA (nucleic acid) sequences being linked are contiguous, and, in the case of a secretory leader, contiguous and in reading phase. However, enhancers do not have to be contiguous. Linking two or more nucleic acid sequences (i.e., operably linking) is accomplished using any of the methods to one of skill in the art.

As used herein, exemplary parental Trichoderma reesei strains include, but are not limited to, T. reesei strain QM6a (ATCC® 13631), T. reesei strain RL-P37 (NRRL Deposit No. 15709) and T. reesei strain RUT-C30 (ATCC® 56765); exemplary parental Aspergillus niger strains include, but are not limited to, A. niger strain ATCC® 1015; exemplary parental Aspergillus oryzae strains include, but are not limited to A. oryzae strain RIB40 (ATCC® 42149); and exemplary parental Myceliophthora thermophila strains include, but are not limited to, M. thermophila strain ATCC® 42464.

For example, Trichoderma strains Rut-C30 and RL-P37 are mutagenized derivatives of Trichoderma natural isolate QM6a (Le Crom et al., 2009; Sheir-Neiss and Montenecourt, 1984), with strain NG14 being the last common ancestor. In certain aspects of the disclosure, an exemplary filamentous fungal strain is derived/obtained from T. reesei strain RL-P37 and comprises a deletion of the T. reesei pyr2 gene (abbreviated hereinafter, “Δpyr2”), as generally described by Sheir-Neiss and Montenecourt (1984) and PCT Publication No. WO2011/153449.

As used herein, the phrases “lignocellulosic degrading enzymes”, “cellulase enzymes”, and “cellulases” are used interchangeably, and include glycoside hydrolase (GH) enzymes such as cellobiohydrolases, xylanases, endoglucanases, and β-glucosidases, that hydrolyze glycosidic bonds of cellulose (hemi-cellulose) to produce sugars (e.g., glucose, xylose, arabinose, etc.).

As used herein, “endoglucanase” proteins may be abbreviated as “EG”, “cellobiohydrolase” proteins may be abbreviated “CBH”, “β-glucosidase” proteins may be abbreviated “BG” and “xylanase” proteins may be abbreviated “XYL”. Thus, as used herein, a gene (or ORF) encoding a EG protein may be abbreviated “eg”, a gene (or ORF) encoding a CBH protein may be abbreviated “cbh”, a gene (or ORF) encoding a BG protein may be abbreviated “bg”, and a gene (or ORF) encoding a XYL protein may be abbreviated “xyl”. In certain embodiments, cellobiohydrolases include enzymes classified under Enzyme Commission No. (EC 3.2.1.91), endoglucanases include enzymes classified under EC 3.2.1.4, endo-β-1,4-xylanases include enzymes classified under EC 3.2.1.8, β-xylosidases include enzymes classified under EC 3.2.1.37, and β-glucosidases include enzymes classified under EC 3.2.1.21.

As used herein, a “cellulase gene promoter” includes, but is not limited to, a cellobiohydrolase (cbh) gene promoter sequence, an endoglucanase (eg) gene promoter sequence, a β-glucosidase (bg) gene promoter sequence, a xylanase (xyl) gene promoter sequence, and the like.

As used herein, the terms “modification” and “genetic modification” are used interchangeably and include: (a) the introduction, substitution, or removal of one or more nucleotides in a gene, or the introduction, substitution, or removal of one or more nucleotides in a regulatory element required for the transcription or translation of the gene, (b) a gene disruption, (c) a gene conversion, (d) a gene deletion, (e) the down-regulation and/or up-regulation of a gene, (f) specific mutagenesis and/or (g) random mutagenesis of any one or more the genes/DNA sequences disclosed herein.

As used herein, the phrases “modified filamentous fungal cell(s)”, “mutant filamentous fungal cell(s)”, “recombinant fungal cell(s)”, “modified filamentous fungal strain(s)”, and the like may be used interchangeably and refer to filamentous fungal cells that are derived (i.e., obtained) from a parental filamentous fungal cell belonging to the Pezizomycotina subphylum. For example, a “modified” filamentous fungal cell may be derived (obtained) from a parental filamentous fungal cell, wherein the modified cell comprises at least one genetic modification which is not found in the parental cell.

As used herein, a “functional gene” is a gene capable of being used by cellular components to produce an active gene product, typically a protein. In contrast, a “non-functional gene” cannot be used by cellular components to produce an active gene product (i.e., a functional protein), or has a reduced ability to be used by cellular components to produce an active gene product (i.e., a functional protein).

As used herein, a “functional protein” is a protein that possesses a function or activity, such as an enzymatic function/activity, a binding function/activity (e.g., DNA binding), a surface-active property, and the like, and which has not been mutagenized, truncated, or otherwise modified to abolish or reduce that function/activity.

As used herein, a “regulatory gene” is a gene whose function has an effect on production of proteins by the fungal host. In certain aspects of the disclosure, the overexpression of a regulatory gene (encoding a regulatory protein) has an effect on protein production by the filamentous fungal (host) cells. A “regulatory gene” encodes a “regulatory protein”.

As used herein, a “regulatory protein” includes, but is not limited to, transcription factor proteins, protein kinases, phosphatases, proteins involved in histone modification or chromatin remodeling, and similar functions related to regulation of gene or protein activity.

More particularly, as used herein, the terms “regulatory gene(s)” “regulatory protein(s)” and/or other “gene transcription regulatory proteins” are not meant to be limiting, but rather used herein to help classify, characterize and/or identify the exemplary genes and/or proteins of the instant disclosure. Thus, as further specified below in Section III and in the Examples that follow, the DNA and/or protein (PRT) sequences of the disclosure are particularly referred to herein as regulatory genes and/or regulatory proteins, regardless of overall protein function. For example, a regulatory protein set forth in TABLE 1, TABLE 10 and/or TABLE 16 may include proteins (e.g., enzymes) involved in one or more metabolic pathway activities (e.g., such as to alleviate a pathway bottleneck) and the like, wherein such proteins (enzymes) may be referred to herein as regulatory proteins, regardless of overall protein function.

As further detailed and described below, certain aspects of the disclosure are related to genetic modifications which render mutant fungal cells deficient in the production of one or more regulatory proteins of the disclosure. More particularly, as set forth below in Examples 2-4 (see, TABLE 1), mutant fungal cells deficient in the production of one or more regulatory proteins of TABLE 1 comprise enhanced protein production characteristics/phenotypes (e.g., total protein production, enzymatic activities, cellulose (PASC) hydrolysis, protein production rates, etc.).

As further detailed and described below, certain aspects of the disclosure are related to the overexpression (over-expression) of certain regulatory genes encoding regulatory proteins. As used herein, “overexpression” (abbreviated, “OE”) of a gene encoding a regulatory protein can be carried out, for example, by introducing into a fungal host an additional copy (or copies) of a specific gene encoding a regulatory protein, or by expressing the gene encoding a regulatory protein under the control of a heterologous promoter resulting in increased expression of the gene, or otherwise genetically modifying the fungal host so that either the gene is more abundantly expressed and/or the activity of the gene product is increased. The effect of overexpression (OE) of a gene encoding a regulatory protein can be studied by culturing the modified host under conditions suitable for protein production. The effect on the production of an endogenous protein or heterologous protein can be studied by determining for example a specific enzyme activity, determining the amount of total protein, or determining the amount of specific endogenous or heterologous protein produced.

More specifically, as set forth in Examples 5 and 6 (see, TABLE 10) mutant fungal cells deficient in the production of one or more regulatory proteins (presented in TABLE 10) comprise reduced protein production characteristics/phenotypes (e.g., total protein production, enzymatic activities, cellulose (PASC) hydrolysis, protein production rates, etc.). Thus, as described and contemplated herein, in certain aspects, the disclosure provides, inter alia, mutant fungal cells overexpressing one or more regulatory proteins of TABLE 10.

As used herein, “disruption of a gene”, “gene disruption”, “inactivation of a gene” and “gene inactivation” are used interchangeably and refer broadly to any genetic modification that substantially prevents a host cell from producing a functional gene product (e.g., a functional protein). Exemplary methods of gene disruptions include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and any combinations and variations thereof which disrupt/inactivate the target gene(s) and substantially reduce or prevent the production of the functional gene product (i.e., the functional protein).

As used herein, “deletion of a gene,” refers to its removal from the genome of a host cell. Where a gene includes control elements (e.g., enhancer elements) that are not located immediately adjacent to the coding sequence of a gene, deletion of a gene refers to the deletion of the coding sequence, and optionally adjacent enhancer elements, including but not limited to, for example, promoter and/or terminator sequences.

As used herein, a “heterologous gene” refers to polynucleotide (DNA) sequences having at least a portion of the sequence which is not native or existing in a native form to the cell in which it is introduced and/or expressed.

As used herein, a “heterologous nucleic acid construct” or “heterologous DNA sequence” has a portion of the sequence which is not native or existing in a native form to the cell in which it is expressed.

As used herein, a “heterologous protein” is encoded by a heterologous gene, a heterologous nucleic acid (polynucleotide) sequence, a heterologous DNA sequence, and the like.

Thus, in certain embodiments, a heterologous gene, a heterologous nucleic acid construct, a heterologous DNA sequence, etc. encoding a protein of interest (POI) is introduced (e.g., transformed) into a filamentous fungal cell (strain). For example, a heterologous gene construct encoding a POI may be introduced into the filamentous fungal cell (strain) before, during, or after performing other genetic modification described herein.

Heterologous, with respect to a control sequence refers to a control sequence (e.g., promoters, enhancers, terminators) that does not function in nature to regulate the same gene the expression of which it is currently regulating. Generally, heterologous nucleic acid sequences are not endogenous to the cell or part of the genome in which they are present, and have been added to the cell, by infection, transfection, transformation, microinjection, electroporation, or the like. A “heterologous” nucleic acid construct may contain a control sequence/DNA coding sequence combination that is the same as, or different from a control sequence/DNA coding sequence combination found in the native cell.

As used herein, the term “coding sequence” (abbreviated, “CDS”) refers to a polynucleotide sequence, which directly specifies the amino acid sequence of its (encoded) protein product. The boundaries of the CDS are generally determined by an open reading frame (ORF), which usually begins with a start codon (ATG). The coding sequence typically includes DNA, cDNA, and recombinant nucleotide sequences. For example, an ORF generally refers to polynucleotide sequence (whether naturally occurring, non-naturally occurring, or synthetic) comprising an uninterrupted reading frame consisting of (i) an initiation codon, (ii) a series of codons representing amino acids of the encoded protein product, and (iii) a termination codon, the ORF being read (or translated) in the 5′ to 3′ direction.

As used herein, the term “DNA construct” or “expression construct” refers to a nucleic acid sequence, which comprises at least two DNA polynucleotide fragments. A DNA or expression construct can be used to introduce nucleic acid sequences into a fungal host cell. The DNA may be generated in vitro (e.g., by PCR) or any other suitable techniques. In some embodiments, the DNA construct comprises a sequence of interest (e.g., encoding a protein of interest). In certain embodiments, a polynucleotide sequence of interest is operably linked to a promoter and/or a terminator. In some embodiments, the DNA construct further comprises at least one selectable marker. In further embodiments, the DNA construct comprises sequences homologous to the host cell chromosome. In other embodiments, the DNA construct comprises non-homologous sequences to the host cell chromosome.

As used herein, a “flanking sequence” refers to any sequence that is either upstream or downstream of the sequence being discussed (e.g., for genes A-B-C, gene B is flanked by the A and C gene sequences). In certain embodiments, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), but in preferred embodiments, it is on each side of the sequence being flanked. The sequence of each homology box is homologous to a sequence in the filamentous fungal chromosome. These sequences direct where in the filamentous fungal chromosome the new construct gets integrated and what part, if any, of the chromosome will be replaced by the incoming sequence.

As used herein, the term “down-regulation” of gene expression includes any methods that result in lower (down-regulated) expression of a functional gene product.

The term “vector” is defined herein as a polynucleotide designed to carry nucleic acid sequences to be introduced into one or more cell types. Vectors include cloning vectors, expression vectors, shuttle vectors, plasmids, phage or virus particles, DNA constructs, cassettes and the like. Expression vectors may include regulatory sequences such as promoters, signal sequences, a coding sequences and transcription terminators.

An “expression vector” as used herein means a DNA construct comprising a coding sequence that is operably linked to suitable control sequences capable of effecting expression of a protein in a suitable host. Such control sequences may include a promoter to effect transcription, an optional operator sequence to control transcription, a sequence encoding suitable ribosome binding sites on the mRNA, enhancers and sequences which control termination of transcription and translation.

As used herein, the term “secretory signal sequence” denotes a DNA sequence that encodes a polypeptide (i.e., a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

As used herein, the term “isolated” or “purified” refers to a filamentous fungal cell, a nucleic acid or a polypeptide that is removed from at least one component with which it is naturally associated.

As used herein, the term “protein of interest” (POI) refers to a polypeptide that is desired to be expressed in a filamentous fungal cell. Such a protein can be an enzyme, a substrate-binding protein, a surface-active protein, a structural protein, and the like, and can be expressed at high levels, and can be for the purpose of commercialization. For example, as generally set forth below, a POI includes, but is not limited to, cellulases, hemicellulases, xylanases, peroxidases, proteases, lipases, phospholipases, esterases, cutinases, polyesterases, pectinases, keratinases, reductases, oxidases, phenol oxidases, lipoxygenases, ligninases, pullulanases, tannases, pentosanases, mannanases, α-glucanases, β-glucanases, hyaluronidases, chondroitinases, laccases, amylases, glucoamylases, acetyl esterases, aminopeptidase, arabinases, arabinosidases, arabinofuranosidases, carboxypeptidases, catalases, nucleases, deoxyribonucleases, ribonucleases, epimerases, α-galactosidases, β-galactosidases, glucan lysases, endo-β-glucanases, glucose oxidases, glucuronidases, invertases, isomerases, and the like.

A protein of interest (POI) can be encoded by an “endogenous” gene. For example, in certain embodiments, a POI is encoded by a gene endogenous to the filamentous fungal cell (strain), such as the aforementioned wild-type genes encoding the native suite of cellulases (e.g., cellobiohydrolases, xylanases, endoglucanases and β-glucosidases).

As used herein, the term “increased productivity” and variations thereof mean an increase of at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% (e.g., greater than 20%) in the production of a protein of interest by a modified (mutant) filamentous fungal cell overexpressing a regulatory gene encoding a regulatory protein of the disclosure, when cultivated under the same conditions of medium composition, temperature, pH, cell density, dissolved oxygen, and time as the parent (control) filamentous fungal cell which does not overexpress the regulatory protein.

As used herein, the term “increased amount” when used in phrases such as a recombinant cell “produces an ‘increased amount’ of a protein of interest”, and variations thereof mean an increase of at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% (e.g., greater than 20%) in the amount of a protein of interest produced by a modified (mutant) filamentous fungal cell overexpressing a regulatory gene encoding a regulatory protein of the disclosure, when cultivated under the same conditions of medium composition, temperature, pH, cell density, dissolved oxygen, and time as the parent (control) filamentous fungal cell which does not overexpress the regulatory protein.

As used herein, the terms “polypeptide” and “protein” (and/or their respective plural forms) are used interchangeably to refer to polymers of any length comprising amino acid residues linked by peptide bonds. The conventional one-letter or three-letter codes for amino acid residues are used herein. The polymer can be linear or branched, it can comprise modified amino acids, and it can be interrupted by non-amino acids. The terms also encompass an amino acid polymer that has been modified naturally or by intervention (e.g., disulfide bond formation, glycosylation, lipidation, acetylation, phosphorylation, or any other manipulation or modification, such as conjugation with a labeling component). Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins.” Such proteins can be derived from organisms of different genera and/or species, or even different classes of organisms (e.g., bacteria and fungi). Related proteins also encompass homologs determined by primary sequence analysis, determined by secondary or tertiary structure analysis, or determined by immunological cross-reactivity.

As used herein, the phrase “substantially free of an activity,” or similar phrases, means that a specified activity is either undetectable in an admixture or present in an amount that would not interfere with the intended purpose of the admixture.

As used herein, the term “derivative polypeptide” refers to a protein which is derived or derivable from a protein by addition of one or more amino acids to either or both the N- and C-terminal end(s), substitution of one or more amino acids at one or a number of different sites in the amino acid sequence, deletion of one or more amino acids at either or both ends of the protein or at one or more sites in the amino acid sequence, and/or insertion of one or more amino acids at one or more sites in the amino acid sequence. The preparation of a protein derivative can be achieved by modifying a DNA sequence which encodes for the native protein, transformation of that DNA sequence into a suitable host, and expression of the modified DNA sequence to form the derivative protein.

Related (and derivative) proteins include “variant proteins.” Variant proteins differ from a reference/parental protein (e.g., a wild-type protein) by substitutions, deletions, and/or insertions at a small number of amino acid residues. The number of differing amino acid residues between the variant and parental protein can be one or more, for example, 1, 2, 3, 4, 5, 6, 7, 8, 9, 10, 15, 20, 30, 40, 50, or more amino acid residues. Variant proteins can share at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99%, or more, amino acid sequence identity with a reference protein. A variant protein can also differ from a reference protein in selected motifs, domains, epitopes, conserved regions, and the like.

As used herein, the term “homologous” protein refers to a protein that has similar activity, function and/or structure to a reference protein. It is not intended that homologs necessarily be evolutionarily related. Thus, it is intended that the term encompass the same, similar, or corresponding protein(s) (i.e., in terms of structure and function) obtained from different organisms. In some embodiments, it is desirable to identify a homolog that has a quaternary, tertiary and/or primary structure similar to the reference protein. For example, regulatory protein homologues from Aspergillus niger, Aspergillus oryzae and/or Thermothelomyces thermophilus (i.e., comprising substantial amino acid sequence identity to a full-length T. reesei regulatory protein of the disclosure) are described in Example 10 (see, TABLE 6).

The degree of homology between sequences can be determined using any suitable method known in the art (see, e.g., Smith and Waterman, 1981; Needleman and Wunsch, 1970; Pearson and Lipman, 1988; programs such as GAP, BESTFIT, FASTA, and TFASTA in the Wisconsin Genetics Software Package (Genetics Computer Group, Madison, WI); and Devereux et al., 1984). For purposes of the present invention, the degree of identity between two amino acid sequences is determined using the Needleman-Wunsch algorithm (Needleman and Wunsch, 1970) as implemented in the Needle program of the EMBOSS package (Rice et al., 2000), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

As used herein, the phrases “substantially similar” and “substantially identical”, in the context of at least two nucleic acids or polypeptides, typically means that a polynucleotide or polypeptide comprises a sequence that has at least about 40% identity, at least about 50% identity, at least about 60% identity, at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Sequence identity can be determined using known programs such as BLAST, ALIGN, and CLUSTAL using standard parameters.

As used herein, the terms “inducer”, “inducers”, or “inducing substrates” are used interchangeably and refer to any compounds that cause filamentous fungal cells to produce “increased amounts” of total protein. Examples of inducing substrates include, but are not limited to, sophorose, lactose, gentibiose and cellulose.

As used herein, the term “induction” refers to the increased transcription of a gene resulting in the synthesis of a protein of interest in a filamentous fungal cell at a markedly increased rate in response to the presence of the “inducer” (i.e., inducing substrate).

To measure the “induction” of a gene of interest (GOI), encoding a protein of interest (POI), modified filamentous fungal cells are supplemented with a candidate inducing substrate (inducer) and are compared vis-à-vis to parental filamentous fungal (control) cells supplemented with same inducing substrate (inducer). Thus, the parental (control) cells are assigned a relative protein activity value of 100%, wherein induction of the GOI encoding the POI in the modified host cells is achieved when the activity value (i.e., relative to the control cells) is greater than 100% (e.g., 100.1% to 100.9%), greater than 101%, greater than 105%, greater than 110%, greater than 150%, greater than 200-500% (i.e., relative to the control), or higher.

As used herein, “aerobic fermentation” refers to growth in the presence of oxygen.

As used herein, the term “cell broth” refers collectively to medium and cells in a liquid/submerged culture.

As used herein, the term “cell mass” refers to the cell component (including intact and lysed cells) present in a liquid/submerged culture. Cell mass can be expressed in dry or wet weight.

As used herein, the phrase “elevated fermentation (cultivation) temperature” is a fermentation temperature greater than the standard fermentation conditions described in the Examples

It will be understood that the methods of the present disclosure are not limited to a particular order for obtaining the modified (mutant) filamentous fungal cell (strain). The modification of a gene may be introduced into the parent strain at any step in the construction of the strain for the production of an endogenous protein of interest (POI) and/or the production of a heterologous POI.

### III. Regulatory Proteins

As generally understood by one skilled in the art, regulatory proteins are involved in the regulation of cell homeostasis at different levels, including, but not limited to, the process of transcribing DNA into RNA. For example, many regulatory proteins called transcription factors, are typically sequence-specific DNA-binding proteins that control, regulate, mediate, and the like, the rate of gene transcription by binding to specific (target) sites in the promoter regions of the (regulated) genes (Latchman, 1993). More particularly, one distinct feature of transcription factors is that they have DNA-binding domains (DBDs) that give them the ability to bind to specific sequences of DNA called enhancer or promoter sequences. Thus, some regulatory proteins bind to a DNA promoter sequence near the transcription start site and help to form the transcription initiation complex. Other regulatory proteins bind to regulatory sequences, such as enhancer sequences, and can either stimulate or repress transcription of the related gene, wherein these regulatory sequences can be thousands of base pairs (bp) upstream (5′) or downstream (3′) from the gene being transcribed.

Protein kinases are regulatory proteins that can selectively modify other proteins by (covalently) adding phosphates to them (i.e., phosphorylation). Histone acetylases/deacetylases are regulatory proteins that can modify histone proteins via covalent addition/removal of acetyl groups (COCH₃), respectively. For example, the regulation of transcription is the most common form of gene control, wherein the action of regulatory proteins allows for unique spatiotemporal regulation of gene expression patterns.

The production of cellulases, hemi-cellulases, ligninases, pectinases and the like are believed to be mainly regulated at the transcriptional level in filamentous fungi (Aro et al., 2005). For example, Stricker et al. (2008) described the similarities and differences in the transcriptional regulation of expression of cellulases and hemi-cellulases in Aspergillus niger and Trichoderma reesei, including the action of XlnR and Xyr1. As described by Kubicek et al. (2009), a number of regulatory components are involved in cellulase regulation in T. reesei in either a positive (Xyr1, Ace2, Hap2/3/5) or negative (Ace1, Cre1) way. Although the action of some regulatory genes on the production of proteins have been described, there is still a need for improved filamentous fungal strains capable of enhanced production of endogenous and heterologous proteins of interest.

As generally known in the art, filamentous fungal strains are typically grown as mycelial submerged cultures in bioreactors, which are adapted to introduce and distribute oxygen and nutrients into the culture medium (i.e., fermentation broth) and maintain optimal pH and temperature, among other things. In particular, the power required to mix, aerate, cool, etc. the fermentation broth can significantly increase the cost of production, and incur higher capital expenditures in terms of motors, power supplies, cooling equipment and the like. For example, the evolution of heat during fermentation processes is closely related to the utilization of carbon and energy source (Wang et al., 1979). As generally described by Wang et al. (1979), the amount of heat is related to the stoichiometry for growth and product formation, while the rate of heat evolution is proportional to kinetics of the process, wherein interest in heat evolution stems from the need to remove it during the fermentation process (e.g., to maintain optimal product formation). Thus, running the process at a higher temperature may be beneficial as it can reduce fermentation time and releases fermentation capacities.

In general, most filamentous fungi will grow and efficiently produce proteins or other metabolites only within a temperature range of about 20° C. to 40° C. For example, the fermentation temperature can vary somewhat, but for filamentous fungi such as Trichoderma reesei, the temperature generally will be within the range of about 20° C. to 40° C., generally preferably in the range of about 25° C. to 34° C. Thus, the ability to ferment filamentous fungal strains at elevated temperatures for the production of proteins is of particular interest in reducing the time and costs of protein production. As described hereinafter, the mutant fungal cells (strains) of the disclosure are well-suited for use in industrial scale fermentation processes for the enhanced production of proteins of interest.

More particularly, as described herein and set forth below in the Examples section, Applicant initially identified more than four hundred (400) regulatory proteins which were predicted via bioinformatics tools using a wild-type Trichoderma reesei QM6a strain (ATCC No. 13631) from the JGI database (Nordberg et al., 2014). As set forth in Example 1, Applicant generated a library of regulatory protein deletion constructs (via fusion PCR), wherein a total of three-hundred forty-three (343) regulatory protein deletant (deficient) strains were obtained and screened for improved characteristics. Applicant further screened the three-hundred forty-three (343) regulatory protein deficient strains for altered protein production characteristics/phenotypes (e.g., total protein production, enzymatic activities, cellulose (PASC) hydrolysis, protein production rates, etc.). More specifically, as described in the Examples section (see, Examples 2-4), T. reesei regulatory protein deletant strains with improved protein production phenotypes under the specified conditions are presented below in TABLES 2-9.

As further described in the Examples section, mutant fungal strains deficient in the production of one or more regulatory proteins of TABLE 10, comprise reduced protein production characteristics/phenotypes under the conditions specified in Examples 5 and 6 (TABLES 11-15). Thus, in certain aspects, the disclosure is related to mutant fungal strains overexpressing one or more regulatory proteins of TABLES 10, wherein the OE mutant strains comprise improved protein production phenotypes under the specified conditions.

In certain aspects, a parental fungal strain comprises a native gene encoding a regulatory protein comprising at least about 50% or greater sequence identity, at least about 55% or greater sequence identity, at least about 60% or greater sequence identity, at least about 65% or greater sequence identity, or at least about 70% or greater sequence identity to a regulatory protein set forth in TABLE 1, TABLE 10 and/or TABLE 16, or a DNA binding domain (DBD) subsequence thereof. In certain embodiments, a parental fungal cell comprises a functional regulatory protein comprising about 70% to 99% amino acid sequence identity to a regulatory protein set forth in TABLE 1, TABLE 10 and/or TABLE 16, or a DBD subsequence thereof.

As generally described in Example 7, fungal regulatory protein homologues from Aspergillus niger, Aspergillus oryzae and Myceliophthora thermophila are presented in TABLE 16. In certain aspects, a regulatory protein of the disclosure comprises at least about 50% or greater sequence identity (e.g., about 51-99% identity or 100% identity) to a regulatory protein homologue presented in TABLE 16.

Thus, in certain aspects, a parental fungal strain comprises a native gene encoding a regulatory protein comprising at least about 50% or greater sequence identity, at least about 55% or greater sequence identity, at least about 60% or greater sequence identity, at least about 65% or greater sequence identity, or at least about 70% or greater sequence identity to a regulatory protein set forth in any one of TABLES 1-16, or a DNA binding domain (DBD) subsequence thereof.

In certain embodiments, a parental fungal cell comprises a functional regulatory protein comprising about 70% to 99% amino acid sequence identity to a regulatory protein set forth in any one of TABLES 1-16, or a DBD subsequence thereof.

In certain embodiments, a polynucleotide sequence of the disclosure (or a subsequence thereof), and/or protein sequence of the disclosure (or a subsequence thereof) and/or a DNA binding domain (DBD) subsequence thereof, or a fragment thereof, can be used to design nucleic acid probes to identify and clone DNA (polynucleotides) encoding regulatory proteins from strains of different genera or species according to methods well known in the art. In particular, such probes can be used for hybridization with the genomic DNA or cDNA of a fungal cell of interest, following standard Southern blotting procedures, in order to identify and isolate the corresponding gene therein.

Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with 32P, 3H, 35S, biotin, or avidin). Thus, a genomic DNA or cDNA library prepared from such other strains can be screened for DNA that hybridizes with the probes described above and encodes a regulatory protein. Genomic or other DNA from such other strains can be separated by agarose or polyacrylamide gel electrophoresis, or other separation techniques. DNA from the libraries or the separated DNA can be transferred to and immobilized on nitrocellulose or other suitable carrier material. In order to identify a clone or DNA that hybridizes with SEQ ID NO: 1, 4, 7, 10, 13, 16, 19, 22, 25, 28, 31, 34, 37, 40, 43, 46, 49, 52, 55, 58, 61, 64, 67, 70, 73, 76, or 79, or a subsequence thereof, the carrier material is used in a Southern blot.

Thus, certain embodiments are related to mutant fungal strains derived from parental fungal strains comprising native genes encoding one or more functional regulatory proteins comprising amino acid sequences comprising at least about 50% identity to one or more fungal regulatory proteins disclosed herein, wherein the genes encoding the regulatory proteins are genetically modified as described herein, thereby rendering the mutant fungal cells deficient in the production of the one or more functional regulatory proteins (i.e., relative to the parental cell). In related aspects, mutant fungal strains of the disclosure (deficient in the production of one or more functional regulatory proteins) produce increased amounts of proteins of interest relative to the parental fungal strains when cultivated under the same conditions.

As generally shown in TABLE 1 and TABLE 10 below, the DNA binding domains (DBDs) are amino acid (sub sequences) present in the full-length regulatory protein's primary (1°) amino acid sequence. However, the specific DBD amino acid sub-sequences presented herein are not meant to be limiting, but rather provide exemplary amino acid sub-sequences (i.e., DBD domains/motifs) suitable for genetic modifications described herein. For example, as contemplated and described herein, regulatory proteins are typically sequence-specific DNA-binding proteins that control, regulate, mediate, etc. the rate of gene transcription by binding to specific (target) sites (i.e., DBDs) in the promoter regions of the (regulated) genes. Thus, in certain embodiments, a mutant strain is derived/obtained from a parental fungal strain, wherein the mutant strain comprises a genetic modification which disrupts, deletes, or otherwise mutagenizes the DBD of one or more regulatory proteins (see, TABLE 1), thereby rendering the mutant strain deficient in the production of one or more regulatory proteins (relative to the parental strain). In certain other embodiments, a mutant cell is derived/obtained from a parental fungal strain, wherein mutant strain comprises an introduced nucleic acid (e.g., an expression cassette) overexpressing one or more regulatory proteins set forth in TABLE 10. For example, in certain embodiments, a mutant cell comprises an introduced expression cassette encoding one or more copies of one or more functional regulatory proteins (i.e., relative to the parental strain). In related aspects, a mutant fungal cell of the disclosure, among other things, is deficient in the production of at least one regulatory protein of TABLE 1 and overexpresses at least one regulatory protein of TABLE 10.

### IV. Recombinant Nucleic Acids and Molecular Biology

As described in Section III above, certain aspects are related to mutant fungal strains derived from parental strains comprising native genes encoding one or more regulatory proteins of the disclosure, wherein the mutant strains comprise genetic modifications rendering the mutant strains deficient in the production of one or more regulatory proteins encoded by the native genes. Certain other aspects are related to mutant fungal strains comprising one or more introduced nucleic acids (e.g., expression cassettes) overexpressing one or more regulatory proteins of the disclosure. In related aspects, mutant fungal strains comprise genetic modifications rendering the mutant strains deficient in the production of one or more regulatory proteins and comprise one or more introduced nucleic acids overexpressing one or more regulatory proteins of the disclosure. In any of these embodiments, the mutant and/or parental strains may comprise additional genetic modifications describe herein.

In particular aspects, Applicant screened over 300 regulatory protein deficient (mutant) strains for enhanced protein production characteristics/phenotypes (see, Examples 1-4; TABLES 2-9). In certain other aspects, Applicant has identified mutant fungal strains deficient in the production of one or more regulatory proteins (TABLE 10), wherein the mutant strains comprise reduced protein production characteristics/phenotypes under the conditions specified (see, Examples 5-6; TABLES 11-15). In certain embodiments, the disclosure provides mutant fungal strains overexpressing one or more regulatory proteins of TABLES 10, wherein the OE mutant strains comprise enhanced protein production protein production characteristics/phenotypes (under the specified conditions). Thus, certain embodiments are related to recombinant microbial strains, recombinant polynucleotides, plasmids, vectors, expression cassettes and the like. In certain aspects, mutant (recombinant) filamentous fungal strains described herein express one or more (heterologous or endogenous) proteins of interest.

As briefly described herein and as set forth below in the Examples, the instant disclosure generally relies on routine techniques in the field of recombinant genetics, wherein the recombinant polynucleotides, filamentous fungal strains and the like described may be constructed using routine methods known in the art (e.g., Sambrook et al., 1989; 2011; 2012; Kriegler 1990 and Ausubel et al., 1994).

Thus, in certain aspects, one or more genetic elements, e.g., a promoter sequence, a gene CDS, a 5′-UTR sequence, a vector, a polynucleotide, and the like, may be genetically modified, as generally understood by one skilled in the art. Is certain embodiments, genetic modifications include, but are not limited to, (a) the introduction, substitution, or removal of one or more nucleotides in a gene, or the introduction, substitution, or removal of one or more nucleotides in a regulatory element required for the transcription or translation of the gene, (b) a gene disruption, (c) a gene conversion, (d) a gene deletion, (e) the down-regulation of a gene, (f) the overexpression (OE) of a gene, (g) specific mutagenesis and/or (h) random mutagenesis of any one or more the genes disclosed herein.

In certain embodiments, a modified (mutant) filamentous fungal cell may be constructed via CRISPR-Cas9 editing. For example, a gene of interest can be modified, disrupted, deleted, or down-regulated by means of nucleic acid guided endonucleases, that find their target DNA by binding either a guide RNA (e.g., Cas9 and Cpf1) or a guide DNA (e.g., NgAgo), which recruits the endonuclease to the target sequence on the DNA, wherein the endonuclease can generate a single or double stranded break in the DNA. This targeted DNA break becomes a substrate for DNA repair, and can recombine with a provided editing template to disrupt or delete or modify the gene. For example, the gene encoding the nucleic acid guided endonuclease (for this purpose Cas9 from S. pyogenes) or a codon optimized gene encoding the Cas9 nuclease is operably linked to a promoter active in the fungal cell and a terminator active in a fungal cell, thereby creating a fungal Cas9 expression cassette. Likewise, one or more target sites unique to the gene of interest are readily identified by a person skilled in the art. For example, to build a DNA construct encoding a gRNA-directed to a target site within the gene of interest, the variable targeting domain (VT) will comprise nucleotides of the target site which are 5′ of the (PAM) protospacer adjacent motif (NGG), which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a fungal cell expression cassette for the gRNA is created by operably linking the DNA encoding the gRNA to a promoter active in fungal cell and a terminator active in fungal cell.

In certain embodiments, the DNA break induced by the endonuclease is repaired/replaced with an incoming sequence. For example, to precisely repair the DNA break generated by the Cas9 expression cassette and the gRNA expression cassette described above, a nucleotide editing template is provided, such that the DNA repair machinery of the cell can utilize the editing template. For example, about 500 bp 5′ of the targeted gene can be fused to about 500 bp 3′ of the targeted gene to generate an editing template, which template is used by the fungal host's machinery to repair the DNA break generated by the RNA-guided endonuclease (RGEN). Even shorter stretches of nucleotides in a form of double or single stranded DNA can be used as an editing template.

The Cas9 expression cassette, the gRNA expression cassette and the editing template can be co-delivered to filamentous fungal cells using many different methods (e.g., PEG mediated protoplast transformation, protoplast fusion, electroporation, biolistics). The transformed cells are screened by PCR amplifying the target gene with a forward and reverse primer. These primers can amplify the wild-type locus or the modified locus that has been edited by the RGEN.

Those of skill in the art are well aware of suitable methods for introducing polynucleotides into filamentous fungal cells (e.g., Aspergillus sp., Trichoderma sp., etc.), wherein standard techniques for transformation of filamentous fungi and culturing the fungi (which are well known to one skilled in the art) are used to transform a fungal host cell of the disclosure. Thus, the introduction of a DNA construct or vector into a fungal host cell includes techniques such as transformation, electroporation, nuclear microinjection, transduction, transfection (e.g., lipofection mediated and DEAE-Dextrin mediated transfection), incubation with calcium phosphate DNA precipitate, high velocity bombardment with DNA-coated microprojectiles, gene gun or biolistic transformation, protoplast fusion and the like. General transformation techniques are known in the art (see, e.g., Ausubel et al., 1987, Sambrook et al., 2001 and 2012, and Campbell et al., 1989). The expression of heterologous proteins in Trichoderma has been described, for example, in U.S. Pat. Nos. 6,022,725; 6,268,328; Harkki et al., 1991 and Harkki et al., 1989. Reference is also made to Cao et al. (2000), for transformation of Aspergillus strains.

In certain other embodiments, the recombinant nucleic acid (or polynucleotide expression cassette thereof or expression vector thereof) further comprises one or more selectable markers. Selectable markers for use in filamentous fungi include, but are not limited to, alsl, amdS, hphB, pyr2, pyr4, pyrG, sucA trpC, argB, a bleomycin resistance marker, a blasticidin resistance marker, a pyrithiamine resistance marker, a neomycin resistance marker, an adenine pathway gene, a thymidine kinase marker and the like. In a particular embodiment, the selectable marker is pyr2, which compositions and methods of use are generally set forth in PCT Publication No. WO2011/153449.

Generally, transformation of Trichoderma sp. cells uses protoplasts or cells that have been subjected to a permeability treatment, typically at a density of 10⁵ to 10⁷/mL, particularly 2×10⁶/mL. A volume of 100 μL of these protoplasts or cells in an appropriate solution (e.g., 1.2 M sorbitol and 50 mM CaCl₂)) is mixed with the desired DNA. Generally, a high concentration of polyethylene glycol (PEG) is added to the uptake solution. Additives, such as dimethyl sulfoxide, heparin, spermidine, potassium chloride and the like, may also be added to the uptake solution to facilitate transformation. Similar procedures are available for other fungal host cells. See, e.g., U.S. Pat. Nos. 6,022,725 and 6,268,328, both of which are incorporated by reference.

In certain aspects, mutant strains comprise a genetic modification which replaces (substitutes) a native promoter sequence of an endogenous gene encoding a native regulatory protein of the disclosure with a heterologous promoter sequence. For example, in certain embodiments, a mutant strain comprises a knocked-in heterologous promoter sequence which drives the expression of the endogenous gene encoding the native regulatory protein. In other aspects, a mutant strain comprises a knocked-out (or mutated) native promoter sequence of an endogenous gene encoding the functional regulatory protein, thereby rendering the mutant strain deficient in the production of the native regulatory protein.

In other aspects, mutant (or modified) strains comprise one or more introduced nucleic acids overexpressing one or more regulatory proteins of the disclosure. For example, in certain aspects, a mutant strain comprises an introduced polynucleotide (expression cassette) comprising a heterologous promoter (pro) sequence upstream (5′) and operably linked a downstream (3′) nucleic acid encoding a regulatory protein of the disclosure. Heterologous promoter (pro) sequences suitable for driving the expression or overexpression of a regulatory protein include any promoter sequences known to one skilled in the art, wherein particularly preferred promoters include any promoter sequences capable of increasing the expression of the regulatory protein in the desired fungal cell. In related embodiments, the cassette may further comprise a downstream (3′) transcriptional terminator sequence operably linked to the gene CDS.

For example, a regulatory gene expression cassette comprising an upstream promoter operably linked to the regulatory gene CDS is schematically presented below (Scheme A), wherein the promoter [pro] and regulatory gene CDS [reg_gene] sequences are shown in operable “-” combination in the 5′ to 3′ direction:
Scheme A: 5′-[pro]-[reg_gene]-3′

Likewise, a regulatory gene expression cassette comprising an upstream promoter operably linked to a regulatory gene CDS operably linked to a downstream terminator sequence is schematically presented below in Scheme B, wherein the promoter [pro], regulatory gene CS [reg_gene] and terminator [term] sequences are shown in operable “-” combination in the 5′ to 3′ direction.
Scheme B: 5′-[pro]-[reg_gene]-[term]-3′

As presented in Schemes A or B, the promoter and/or terminator sequences are not meant to be limiting, but are rather selected so as to be functional in the desired fungal cell/strain. For example, a promoter sequence can be any nucleotide sequence that shows transcriptional activity in the filamentous fungal cell, including mutant/variant promoters, truncated promoters, tandem promoters, hybrid promoters, synthetic promoters, inducible promoters, tuned promoters, conditional expression systems and combinations thereof. Often, suitable promoters can be obtained from genes encoding extracellular or intracellular polypeptides either native or heterologous (foreign) to the filamentous fungal cell. Examples of promoters suitable for driving the expression of one or more regulatory genes of the disclosure include, but are not limited, to a Trichoderma reesei cDNA1 promoter, an eno1 promoter, a pdc1 promoter, a pki1 promoter, a tef1 promoter, a rp2 promoter, and other T. reesei promoters described in Fitz et al. 2018 (incorporated herein by referenced in its entirety), the Aspergillus oryzae thiA promoter, the A. nidulans gpdA promoter, and the like.

In certain embodiments, the instant disclosure is directed to the expression/production of one or more proteins of interest which are endogenous to the filamentous fungal host cell. In other embodiments, the disclosure is directed to expressing/producing one or more proteins of interest which are heterologous to the filamentous fungal host cell.

In certain embodiments, a heterologous gene is cloned into an intermediate vector, before being transformed into a filamentous fungal (host) cells for expression. These intermediate vectors can be prokaryotic vectors, such as, e.g., plasmids, or shuttle vectors. The expression vector/construct typically contains a transcription unit or expression cassette that contains all the additional elements required for the expression of the heterologous sequence. For example, a typical expression cassette contains a 5′ promoter operably linked to the heterologous nucleic acid sequence encoding the POI and may further comprise sequence signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette may also contain a transcription termination region downstream of the structural gene to provide for efficient termination. The termination region may be obtained from the same gene as the promoter sequence, or may be obtained from different genes. Although any fungal terminator is likely to be functional in the present invention, preferred terminators include: the terminator from Trichoderma cbhI gene, the terminator from Aspergillus nidulans trpC gene (Yelton et al., 1984; Mullaney et al., 1985), the Aspergillus awamori or Aspergillus niger glucoamylase genes (Nunberg et al., 1984; Boel et al., 1984) and/or the Mucor miehei carboxyl protease gene (EPO Publication No. 0215594).

The particular expression vector used to transport the genetic information into the cell is not particularly critical. Any of the conventional vectors used for expression in eukaryotic or prokaryotic cells may be used. Standard bacterial expression vectors include bacteriophages λ and M13, as well as plasmids such as pBR322 based plasmids, pSKF, pET23D, and fusion expression systems such as MBP, GST, and LacZ, as well as yeast 2μ plasmids and centromeric yeast plasmids. Epitope tags can also be added to recombinant proteins to provide convenient methods of isolation, e.g., c-myc.

The elements that can be included in expression vectors may also be a replicon, a gene encoding antibiotic resistance to permit selection of bacteria that harbor recombinant plasmids, or unique restriction sites in nonessential regions of the plasmid to allow insertion of heterologous sequences. The particular antibiotic resistance gene chosen is not dispositive either, as any of the many resistance genes known in the art may be suitable. The prokaryotic sequences are preferably chosen such that they do not interfere with the replication or integration of the DNA in the fungal host.

The methods of transformation of the present disclosure may result in the stable integration of all or part of the transformation vector into the genome of the filamentous fungus. However, transformation resulting in the maintenance of a self-replicating extra-chromosomal transformation vector is also contemplated. Any of the known procedures for introducing foreign (heterologous) nucleotide sequences into host cells may be used. These include the use of calcium phosphate transfection, polybrene, protoplast fusion, electroporation, biolistics, liposomes, microinjection, and any of the other known methods for introducing cloned genomic DNA, cDNA, synthetic DNA or other foreign genetic material into a host cell (see, e.g., Sambrook et al., supra). Also of use is the Agrobacterium-mediated transfection method such as the one described in U.S. Pat. No. 6,255,115.

After the expression vector(s) is/are introduced into the cells, the transfected cells are cultured under conditions favoring expression of genes under control of cellulase gene promoter sequences. Large batches of transformed cells can be cultured as described herein. Finally, product is recovered from the culture using standard techniques.

### V. Proteins of Interest

As stated above, certain embodiments are related to genetically mutant and/or modified (recombinant) fungal cells comprising genetic modifications which express a gene encoding a protein of interest (POI). More particularly, certain embodiments are related to compositions and methods for the expression/production of such proteins of interest in the modified (mutant) fungal cells of the disclosure. Thus, in certain embodiments, recombinant fungal cells produced enhanced amounts of proteins of interest, including, but not limited to, enzymes, antibodies, receptor proteins, animal feed proteins, human food proteins protein biologics and the like.

In certain aspects, proteins of interest include are enzymes. In certain embodiments, proteins of interest are enzymes selected from the group consisting of amylases, cellulases, hemicellulases, xylanases, peroxidases, proteases, lipases, phospholipases, esterases, cutinases, polyesterases, phytase, pectinases, keratinases, reductases, oxidases, phenol oxidases, lipoxygenases, ligninases, pullulanases, tannases, pentosanases, mannanases, α-glucanases, β-glucanases, hyaluronidases, chondroitinases, laccases, amylases, glucoamylases, acetyl esterases, aminopeptidase, arabinases, arabinosidases, arabinofuranosidases, carboxypeptidases, catalases, nucleases, deoxyribonucleases, ribonucleases, epimerases, α-galactosidases, β-galactosidases, glucan lysases, endo-β-glucanases, glucose oxidases, glucuronidases, invertases, and isomerases.

A protein of interest (POI) may be an endogenous POI or a heterologous POI.

In certain embodiments, a POI is selected from an Enzyme Commission (EC) Number selected from the group consisting of EC 1, EC 2, EC 3, EC 4, EC 5 or EC 6.

For example, in certain embodiments a POI is an oxidoreductase enzyme, including, but not limited to, an EC1 (oxidoreductase) enzyme selected from EC 1.10.3.2 (e.g., a laccase), EC 1.10.3.3 (e.g., L-ascorbate oxidase), EC 1.1.1.1 (e.g., alcohol dehydrogenase), EC 1.11.1.10 (e.g., chloride peroxidase), EC 1.11.1.17 (e.g., peroxidase), EC 1.1.1.27 (e.g., L-lactate dehydrogenase), EC 1.1.1.47 (e.g., glucose 1-dehydrogenase), EC 1.1.3.X (e.g., glucose oxidase), EC 1.1.3.10 (e.g., pyranose oxidase), EC 1.13.11.X (e.g., dioxygenase), EC 1.13.11.12 (e.g., lineolate 13S-lipozygenase), EC 1.1.3.13 (e.g., alcohol oxidase), EC 1.14.14.1 (e.g., monooxygenase), EC 1.14.18.1 (e.g., monophenol monooxigenase), EC 1.15.1.1 (e.g., superoxide dismutase), EC 1.1.5.9 (formerly EC 1.1.99.10, e.g., glucose dehydrogenase), EC 1.1.99.18 (e.g., cellobiose dehydrogenase), EC 1.1.99.29 (e.g., pyranose dehydrogenase), EC 1.2.1.X (e.g., fatty acid reductase), EC 1.2.1.10 (e.g., acetaldehyde dehydrogenase), EC 1.5.3.X (e.g., fructosyl amine reductase), EC 1.8.1.X (e.g., disulfide reductase) and EC 1.8.3.2 (e.g., thiol oxidase).

In certain embodiments a POI is a transferase enzyme, including, but not limited to, an EC 2 (transferase) enzyme selected from EC 2.3.2.13 (e.g., transglutaminase), EC 2.4.1.X (e.g., hexosyltransferase), EC 2.4.1.40 (e.g., altemasucrase), EC 2.4.1.18 (e.g., 1,4 alpha-glucan branching enzyme), EC 2.4.1.19 (e.g., cyclomaltodextrin glucanotransferase), EC 2.4.1.2 (e.g., dextrin dextranase), EC 2.4.1.20 (e.g., cellobiose phosphorylase), EC 2.4.1.25 (e.g., 4-alpha-glucanotransferase), EC 2.4.1.333 (e.g., 1,2-beta-oligoglucan phosphor transferase), EC 2.4.1.4 (e.g., amylosucrase), EC 2.4.1.5 (e.g., dextransucrase), EC 2.4.1.69 (e.g., galactoside 2-alpha-L-fucosyl transferase), EC 2.4.1.9 (e.g., inulosucrase), EC 2.7.1.17 (e.g., xylulokinase), EC 2.7.7.89 (formerly EC 3.1.4.15, e.g., [glutamine synthetase]-adenylyl-L-tyrosine phosphorylase), EC 2.7.9.4 (e.g., alpha glucan kinase) and EC 2.7.9.5 (e.g., phosphoglucan kinase).

In other embodiments a POI is a hydrolase enzyme, including, but not limited to, an EC 3 (hydrolase) enzyme selected from EC 3.1.X.X (e.g., an esterase), EC 3.1.1.1 (e.g., pectinase), EC 3.1.1.14 (e.g., chlorophyllase), EC 3.1.1.20 (e.g., tannase), EC 3.1.1.23 (e.g., glycerol-ester acylhydrolase), EC 3.1.1.26 (e.g., galactolipase), EC 3.1.1.32 (e.g., phospholipase A1), EC 3.1.1.4 (e.g., phospholipase A2), EC 3.1.1.6 (e.g., acetylesterase), EC 3.1.1.72 (e.g., acetylxylan esterase), EC 3.1.1.73 (e.g., feruloyl esterase), EC 3.1.1.74 (e.g., cutinase), EC 3.1.1.86 (e.g., rhamnogalacturonan acetylesterase), EC 3.1.1.87 (e.g., fumosin B1 esterase), EC 3.1.26.5 (e.g., ribonuclease P), EC 3.1.3.X (e.g., phosphoric monoester hydrolase), EC 3.1.30.1 (e.g., Aspergillus nuclease SI), EC 3.1.30.2 (e.g., Serratia marcescens nuclease), EC 3.1.3.1 (e.g., alkaline phosphatase), EC 3.1.3.2 (e.g., acid phosphatase), EC 3.1.3.8 (e.g., 3-phytase), EC 3.1.4.1 (e.g., phosphodiesterase I), EC 3.1.4.11 (e.g., phosphoinositide phospholipase C), EC 3.1.4.3 (e.g., phospholipase C), EC 3.1.4.4 (e.g., phospholipase D), EC 3.1.6.1 (e.g., arylsufatase), EC 3.1.8.2 (e.g., diisopropyl-fluorophosphatase), EC 3.2.1.10 (e.g., oligo-1,6-glucosidase), EC 3.2.1.101 (e.g., mannanendo-1,6-alpha-mannosidase), EC 3.2.1.11 (e.g., alpha-1,6-glucan-6-glucanohydrolase), EC 3.2.1.131 (e.g., xylan alpha-1,2-glucuronidase), EC 3.2.1.132 (e.g., chitosan N-acetylglucosaminohydrolase), EC 3.2.1.139 (e.g., alpha-glucuronidase), EC 3.2.1.14 (e.g., chitinase), EC 3.2.1.151 (e.g., xyloglucan-specific endo-beta-1,4-glucanase), EC 3.2.1.155 (e.g., xyloglucan-specific exo-beta-1,4-glucanase), EC 3.2.1.164 (e.g., galactan endo-1,6-beta-galactosidase), EC 3.2.1.17 (e.g., lysozyme), EC 3.2.1.171 (e.g., rhamnogalacturonan hydrolase), EC 3.2.1.174 (e.g., rhamnogalacturonan rhamnohydrolase), EC 3.2.1.2 (e.g., beta-amylase), EC 3.2.1.20 (e.g., alpha-glucosidase), EC 3.2.1.22 (e.g., alpha-galactosidase), EC 3.2.1.25 (e.g., beta-mannosidase), EC 3.2.1.26 (e.g., beta-fructofuranosidase), EC 3.2.1.37 (e.g., xylan 1,4-beta-xylosidase), EC 3.2.1.39 (e.g., glucan endo-1,3-beta-D-glucosidase), EC 3.2.1.40 (e.g., alpha-L-rhamnosidase), EC 3.2.1.51 (e.g., alpha-L-fucosidase), EC 3.2.1.52 (e.g., beta-N-Acetylhexosaminidase), EC 3.2.1.55 (e.g., alpha-N-arabinofuranosidase), EC 3.2.1.58 (e.g., glucan 1,3-beta-glucosidase), EC 3.2.1.59 (e.g., glucan endo-1,3-alpha-glucosidase), EC 3.2.1.67 (e.g., galacturan 1,4-alpha-galacturonidase), EC 3.2.1.68 (e.g., isoamylase), EC 3.2.1.7 (e.g., 1-beta-D-fructan fructanohydrolase), EC 3.2.1.74 (e.g., glucan 1,4-glucosidase), EC 3.2.1.75 (e.g., glucan endo-1,6-beta-glucosidase), EC 3.2.1.77 (e.g., mannan 1,2-(1,3)-alpha-mannosidase), EC 3.2.1.80 (e.g., fructan beta-fructosidase), EC 3.2.1.82 (e.g., exo-poly-alpha-galacturonosidase), EC 3.2.1.83 (e.g., kappa-carrageenase), EC 3.2.1.89 (e.g., arabinogalactan endo-1,4-beta-galactosidase), EC 3.2.1.91 (e.g., cellulose 1,4-beta-cellobiosidase), EC 3.2.1.96 (e.g., mannosyl-glycoprotein endo-beta-N-acetylglucosaminidase), EC 3.2.1.99 (e.g., arabinan endo-1,5-alpha-L-arabinanase), EC 3.4.X.X (e.g., peptidase), EC 3.4.1 1.X (e.g., aminopeptidase), EC 3.4.11.1 (e.g., leucyl aminopeptidase), EC 3.4.11.18 (e.g., methionyl aminopeptidase), EC 3.4.13.9 (e.g., Xaa-Pro dipeptidase), EC 3.4.14.5 (e.g., dipeptidyl-peptidase IV), EC 3.4.16. X (e.g., serine-type carboxypeptidase), EC 3.4.16.5 (e.g., carboxypeptidase C), EC 3.4.19.3 (e.g., pyroglutamyl-peptidase I), EC 3.4.21. X (e.g., serine endopeptidase), EC 3.4.21.1 (e.g., chymotrypsin), EC 3.4.21.19 (e.g., glutamyl endopeptidase), EC 3.4.21.26 (e.g., prolyl oligopeptidase), EC 3.4.21.4 (e.g., trypsin), EC 3.4.21.5 (e.g., thrombin), EC 3.4.21.63 (e.g., oryzin), EC 3.4.21.65 (e.g., thermomycolin), EC 3.4.21.80 (e.g., streptogrisin A), EC 3.4.22. X (e.g., cysteine endopeptidase), EC 3.4.22.14 (e.g., actinidain), EC 3.4.22.2 (e.g., papain), EC 3.4.22.3 (e.g., ficain), EC 3.4.22.32 (e.g., stem bromelain), EC 3.4.22.33 (e.g., fruit bromelain), EC 3.4.22.6 (e.g., chymopapain), EC 3.4.23.1 (e.g., pepsin A), EC 3.4.23.2 (e.g., pepsin B), EC 3.4.23.22 (e.g., endothiapepsin), EC 3.4.23.23 (e.g., mucorpepsin), EC 3.4.23.3 (e.g., gastricsin), EC 3.4.24.X (e.g., metalloendopeptidase), EC 3.4.24.39 (e.g., deuterolysin), EC 3.4.24.40 (e.g., serralysin), EC 3.5.1.1 (e.g., asparaginase), EC 3.5.1.11 (e.g., penicillin amidase), EC 3.5.1.14 (e.g., N-acyl-aliphatic-L-amino acid amidohydrolase), EC 3.5.1.2 (e.g., L-glutamine amidohydrolase), EC 3.5.1.28 (e.g., N-acetylmuramoyl-L-alanine amidase), EC 3.5.1.4 (e.g., amidase), EC 3.5.1.44 (e.g., protein-L-glutamine amidohydrolase), EC 3.5.1.5 (e.g., urease), EC 3.5.1.52 (e.g., peptide-N(4)-(N-acetyl-beta-glucosaminyl) asparagine amidase), EC 3.5.1.81 (e.g., N-Acyl-D-amino-acid deacylase), EC 3.5.4.6 (e.g., AMP deaminase) and EC 3.5.5.1 (e.g., nitrilase).

In other embodiments a POI is a lyase enzyme, including, but not limited to, an EC 4 (lyase) enzyme selected from EC 4.1.2.10 (e.g., mandelonitrile lyase), EC 4.1.3.3 (e.g., N-acetylneuraminate lyase), EC 4.2.1.1 (e.g., carbonate dehydratase), EC 4.2.2.- (e.g., rhamnogalacturonan lyase), EC 4.2.2.10 (e.g., pectin lyase), EC 4.2.2.22 (e.g., pectate trisaccharide-lyase), EC 4.2.2.23 (e.g., rhamnogalacturonan endolyase) and EC 4.2.2.3 (e.g., mannuronate-specific alginate lyase).

In certain other embodiments a POI is an isomerase enzyme, including, but not limited to, an EC 5 (isomerase) enzyme selected from EC 5.1.3.3 (e.g., aldose l-epimerase), EC 5.1.3.30 (e.g., D-psicose 3-epimerase), EC 5.4.99.11 (e.g., isomaltulose synthase) and EC 5.4.99.15 (e.g., (l 4)-a-D-glucan 1-a-D-glucosylmutase).

In yet other embodiments, a POI is a ligase enzyme, including, but not limited to, an EC 6 (ligase) enzyme selected from EC 6.2.1.12 (e.g., 4-coumarate: coenzyme A ligase) and EC 6.3.2.28 (e.g., L-amino-acid alpha-ligase).

Optimal conditions for the production of the proteins will vary with the choice of the host cell, and with the choice of the protein(s) to be expressed. Such conditions may be readily ascertained by one skilled in the art through routine experimentation and/or optimization.

The protein of interest can be purified or isolated after expression. The protein of interest may be isolated or purified in a variety of ways known to those skilled in the art depending on what other components are present in the sample. Standard purification methods include, but are not limited to, electrophoretic, molecular, immunological and chromatographic techniques, including ion exchange, hydrophobic, affinity, and reverse-phase HPLC chromatography, and chromatofocusing. For example, the protein of interest may be purified using a standard anti-protein of interest antibody column. Ultrafiltration and diafiltration techniques, in conjunction with protein concentration, are also useful. The degree of purification necessary will vary depending on the intended use of the protein of interest. In certain instances, no purification of the protein will be necessary.

In certain other embodiments, to confirm that a genetically modified fungal cell of the disclosure produces an increased level of a protein of interest, various methods of screening may be performed. The expression vector may encode a polypeptide fusion to the target protein which serves as a detectable label or the target protein itself may serve as the selectable or screenable marker. The labeled protein may be detected via western blotting, dot blotting (methods available at the Cold Spring Harbor Protocols website), ELISA, or, if the label is GFP, whole cell fluorescence and/or FACS. For example, a 6-histidine tag would be included as a fusion to the target protein, and this tag would be detected by western blotting. If the target protein expresses at sufficiently high levels, SDS-PAGE combined with Coomassie/silver staining, may be performed to detect increases in variant host cell expression over parental (control) cell, in which case no label is necessary. In addition, other methods may be used to confirm the improved level of a protein of interest, such as, the detection of the increase of protein amount per cell or protein amount per milliliter of fermentation medium using HPLC methods of protein separation or standard total protein measurements based on Coomassie Blue or BCA Reagents. The detection of specific productivity is another method to evaluate the protein production. Specific productivity (Qp) can be determined by the following equation:

$$Qp = gP / g DCW * hr$$

wherein “gP” is grams of protein produced in the tank, “gDCW” is grams of dry cell weight (DCW) in the tank, “hr” is fermentation time in hours from the time of inoculation, which include the time of production as well as growth time. Ultimately, if a protein of interest has enzymatic activity, its level of expression can be calculated from enzymatic assay.

### VI. Fermentation

Certain embodiments are related to compositions and methods for producing a protein of interest comprising growing, cultivating or fermenting a modified (mutant) filamentous fungal cell of the disclosure. In general, fermentation methods well known in the art are used to ferment the fungal cells. In some embodiments, the fungal cells are grown under batch, fed-batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation occurs without the addition of any components to the system. Typically, a batch fermentation qualifies as a “batch” with respect to the addition of the nutrients, while factors such as pH and oxygen concentration are controlled. The broth and culture compositions of the batch system change constantly up to the time the fermentation is stopped. Within batch cultures, cells progress through a static lag phase to a high growth log phase and finally to a stationary phase, where growth rate is diminished or halted. If untreated, cells proceed to apoptosis and eventually die. In general, in the batch phase, the bulk of the production of product occurs during the log phase

A suitable variation on the standard batch system is the “fed-batch fermentation” system. In this variation of a typical batch system, after the log phase is finished, the substrate is added in increments as the fermentation progresses. Fed-batch systems are often used to avoid catabolite repression. Continuous feeding of the substrate allows the process to keep its concentration below critical level that could lead to inhibition of cellular metabolism and protein production. Batch and fed-batch fermentations are common and well known in the art.

Continuous fermentation is a system where a defined fermentation medium is added continuously to a bioreactor, and an equal amount of conditioned medium is removed simultaneously for processing. Continuous fermentation generally maintains the cultures at a constant (high) density, where cells are primarily kept in log phase growth. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

Certain embodiments of the instant disclosure are related to fermentation procedures for culturing fungi. Fermentation procedures for production of cellulase enzymes are known in the art. For example, cellulase enzymes can be produced either by solid or submerged culture, including batch, fed-batch and continuous-flow processes. Culturing is generally accomplished in a growth medium comprising an aqueous mineral salts medium, organic growth factors, a carbon and energy source material, molecular oxygen, and, of course, a starting inoculum of the filamentous fungal host to be employed.

In addition to the carbon and energy source, oxygen, assimilable nitrogen, and an inoculum of the microorganism, it is necessary to supply suitable amounts in proper proportions of mineral nutrients to assure proper microorganism growth, maximize the assimilation of the carbon and energy source by the cells in the microbial conversion process, and achieve maximum cellular yields with maximum cell density in the fermentation media.

The composition of the aqueous mineral medium can vary over a wide range, depending in part on the microorganism and substrate employed, as is known in the art. The mineral media should include, in addition to nitrogen, suitable amounts of phosphorus, magnesium, calcium, potassium, sulfur, and sodium, in suitable soluble assimilable ionic and combined forms, and certain trace elements such as copper, manganese, molybdenum, zinc, iron, boron, and iodine, and others, again in suitable soluble assimilable form, all as known in the art.

The fermentation process can be an aerobic process in which the molecular oxygen needed is supplied by a molecular oxygen-containing gas such as air, oxygen-enriched air, or even substantially pure molecular oxygen, provided to maintain the contents of the fermentation vessel with a suitable oxygen partial pressure effective in assisting the microorganism species to grow in a thriving fashion.

The fermentation temperature can vary somewhat, but for filamentous fungi such as Trichoderma reesei, the temperature generally will be within the range of about 20° C. to 40° C., generally preferably in the range of about 25° C. to 34° C.

The microorganisms also require a source of assimilable nitrogen. The source of assimilable nitrogen can be any nitrogen-containing compound or compounds capable of releasing nitrogen in a form suitable for metabolic utilization by the microorganism. While a variety of organic nitrogen source compounds, such as protein hydrolysates, can be employed, usually cheap nitrogen-containing compounds such as ammonia, ammonium hydroxide, urea, and various ammonium salts such as ammonium phosphate, ammonium sulfate, ammonium pyrophosphate, ammonium chloride, or various other ammonium compounds can be utilized. Ammonia gas itself is convenient for large scale operations, and can be employed by bubbling through the aqueous ferment (fermentation medium) in suitable amounts. At the same time, such ammonia can also be employed to assist in pH control.

The pH range in the aqueous microbial ferment should be in the exemplary range of about 2.0 to 10.0. With filamentous fungi, the pH normally is within the range of about 2.5 to 8.0; with Trichoderma reesei, the pH normally is within the range of about 3.0 to 7.0. Preferences for pH range of microorganisms are dependent on the media employed to some extent, as well as the particular microorganism, and thus can be somewhat adjusted as can be readily determined by those skilled in the art.

Preferably, the fermentation is conducted in such a manner that the carbon-containing substrate can be controlled as a limiting factor, thereby providing good conversion of the carbon-containing substrate to products and avoiding contamination of the cells with a substantial amount of unconverted substrate. The latter is not a problem with water-soluble substrates, since any remaining traces are readily washed off. It may be a problem, however, in the case of non-water-soluble substrates, and require added product-treatment steps such as suitable washing steps.

As described above, the time to reach this level is not critical and may vary with the particular microorganism and fermentation process being conducted. However, it is well known in the art how to determine the carbon source concentration in the fermentation medium and whether or not the desired level of carbon source has been achieved.

The fermentation can be conducted as a batch or continuous operation, fed batch operation is much to be preferred for ease of control, production of uniform quantities of products, and most economical uses of all equipment.

If desired, part or all of the carbon and energy source material and/or part of the assimilable nitrogen source such as ammonia can be added to the aqueous mineral medium prior to feeding the aqueous mineral medium to the fermenter.

Each of the streams introduced into the reactor preferably is controlled at a predetermined rate, or in response to a need determinable by monitoring such as concentration of the carbon and energy substrate, pH, dissolved oxygen, oxygen or carbon dioxide in the off-gases from the fermenter, cell density measurable by dry cell weights, light transmittancy, or the like. The feed rates of the various materials can be varied so as to obtain maximal production rates and/or maximum yields.

In either a batch, or the preferred fed batch operation, all equipment, reactor, or fermentation means, vessel or container, piping, attendant circulating or cooling devices, and the like, are initially sterilized, usually by employing steam such as at about 121° C. for at least about 15 minutes. The sterilized reactor then is inoculated with a culture of the selected microorganism in the presence of all the required nutrients, including oxygen, and the carbon-containing substrate. The type of fermenter employed is not critical.

The collection and purification of (e.g., cellulase) enzymes from the fermentation broth can also be done by procedures known to one skilled in the art. The fermentation broth will generally contain cellular debris, including cells, various suspended solids and other biomass contaminants, as well as the desired cellulase enzyme product, which are preferably removed from the fermentation broth by means known in the art.

Suitable processes for such removal include conventional solid-liquid separation techniques such as, e.g., centrifugation, filtration, dialysis, microfiltration, rotary vacuum filtration, or other known processes, to produce a cell-free filtrate. It may be preferable to further concentrate the fermentation broth or the cell-free filtrate prior to crystallization using techniques such as ultrafiltration, evaporation or precipitation.

Precipitating the proteinaceous components of the supernatant or filtrate may be accomplished by means of a salt, e.g., ammonium sulfate or an organic solvent, like acetone, followed by purification by a variety of chromatographic procedures, e.g., ion exchange chromatography, affinity chromatography or similar art recognized procedures.

### VII. Exemplary Embodiments

Non-limiting embodiments of compositions and methods disclosed herein are as follows:

1. A recombinant fungal cell deficient in the production of one or more native regulatory proteins comprising at least 80% identity to a Trichoderma reesei regulatory protein set forth in TABLE 1.

2. A recombinant fungal cell deficient in the production of one or more native regulatory proteins comprising at least 80% identity to a regulatory protein homologue set forth in TABLE 16.

3. A recombinant fungal cell overexpressing one or more regulatory proteins comprising at least 80% identity to a Trichoderma reesei regulatory protein set forth in TABLE 10.

4. A recombinant fungal cell deficient in the production of one or more native regulatory proteins comprising at least 80% identity to a Trichoderma reesei regulatory protein set forth in TABLE 1 and overexpressing one or more regulatory proteins comprising at least 80% identity to a Trichoderma reesei regulatory protein set forth in TABLE 10.

5. A recombinant fungal cell deficient in the production of one or more native regulatory proteins comprising at least 80% identity to a regulatory protein homologue set forth in TABLE 16 and overexpressing one or more regulatory proteins comprising at least 80% identity to a Trichoderma reesei regulatory protein set forth in TABLE 10.

6. The recombinant fungal cell of any one of embodiments 1, 2, 4 or 5 wherein the cell is deficient in the production of one or more regulatory proteins comprising at least 80% identity to a DNA binding domain (DBD) shown in TABLE 1.

7. The recombinant fungal cell of any one of embodiments 1-6, expressing a protein of interest.

8. A polynucleotide (e.g., an expression cassette) comprising an upstream (5′) promoter (pro) operably linked to a downstream (3′) nucleic acid (reg_protein) encoding a regulatory protein comprising at least 80% sequence identity to a protein set forth in TABLE 10 (e.g., 5′-[pro]-[reg_protein]-3′).

9. The polynucleotide of embodiment 8, wherein the upstream promoter is a constitutive promoter or an inducible promoter.

10. The polynucleotide of embodiment 8, further comprising a terminator (term) sequence downstream (3′) and operably linked to nucleic acid (reg_protein) encoding the regulatory protein.

11. A recombinant fungal cell comprising at least one introduced polynucleotide of embodiment 8.

12. A recombinant fungal cell comprising at least one introduced polynucleotide of embodiment 8 and expressing an endogenous protein of interest.

13. A recombinant fungal cell comprising at least one introduced polynucleotide of embodiment 8 and expressing at least one introduced cassette encoding a heterologous protein of interest (POI).

14. The recombinant fungal of embodiment 13, wherein the introduced cassette comprises an upstream (5′) cellulase gene promoter (proCel) sequence operably linked to a downstream (3′) nucleic acid (POI) encoding the heterologous POI (e.g., 5′-[proCel]-[POI]-3′).

15. The recombinant fungal cell of any one of embodiments 11-14, further comprising a genetic modification rendering the recombinant cell deficient in the production of one or more native regulatory proteins comprising at least 80% identity to a Trichoderma reesei regulatory protein set forth in TABLE 1.

16. The recombinant fungal cell of any one of embodiments 11-14, further comprising a genetic modification rendering the recombinant cell deficient in the production of one or more native regulatory proteins comprising at least 80% identity to a regulatory protein homologue set forth in TABLE 16.

17. The recombinant fungal cell of embodiment 12, wherein the endogenous POI is a cellulase.

18. The recombinant fungal cell of embodiment 17, wherein the cellulase is selected from the group consisting of cellobiohydrolases, xylanases, endoglucanases and β-glucosidases.

19. The recombinant fungal cell of embodiment 13, wherein the heterologous POI is selected from the group consisting of enzymes, peptides, antibodies (and functional antibody fragments thereof), receptor proteins, animal feed proteins, human food proteins, protein biologics, therapeutic proteins, immunogenic proteins and the like.

20. A method for the enhanced production of a cellulase comprising obtaining a parental fungal cell expressing a cellulase, genetically modifying the parental cell to obtain a recombinant fungal cell deficient in the production of one or more native regulatory proteins comprising at least 80% identity to a regulatory protein set forth in TABLE 1, and cultivating the recombinant cell, wherein the recombinant cell produces an increased amount of a cellulase relative to the parental cell cultivated under the same conditions.

21. The method of embodiment 20, wherein the recombinant cell further comprises one or more introduced polynucleotides encoding one or more regulatory proteins comprising at least 80% identity to a regulatory protein set forth in TABLE 10.

22. A method for the enhanced production of a cellulase comprising obtaining a parental fungal cell expressing a cellulase, genetically modifying the parental cell to obtain a recombinant fungal cell overexpressing one or more regulatory proteins comprising at least 80% identity to a regulatory protein set forth in TABLE 10, and cultivating the recombinant cell, wherein the recombinant cell produces an increased amount of a cellulase relative to the parental cell cultivated under the same conditions.

23. The method of embodiment 22, wherein the recombinant cell further comprises genetic modifications rendering the cell deficient in the production of one or more native regulatory proteins comprising at least 80% identity to a regulatory protein of TABLE 1.

24. The method of any one of embodiments 20-23, wherein the cellulase is selected from the group consisting of cellobiohydrolases, hemi cellulases, endoglucanases and β-glucosidases.

25. A method for the enhanced production of a protein of interest (POI) comprising introducing into a parental fungal cell at least one expression cassette encoding a POI, genetically modifying the parental cell to obtain a recombinant fungal cell deficient in the production of one or more native regulatory proteins comprising at least 80% identity to a regulatory protein set forth in TABLE 1, and cultivating the recombinant cell, wherein the recombinant cell produces an increased amount of the POI relative to the parental cell cultivated under the same conditions.

26. The method of embodiment 25, wherein the cassette encoding the POI comprises an upstream (5′) cellulase gene promoter operably linked to a downstream (3′) nucleic acid encoding the POI.

27. The method of embodiment 25, wherein the cassette encoding the POI is integrated into the genome of the fungal cell.

28. The method of embodiment 25, wherein the POI is selected from the group consisting of enzymes, peptides, antibodies (and functional antibody fragments thereof), receptor proteins, animal feed proteins, human food proteins, protein biologics, therapeutic proteins, immunogenic proteins and the like.

### EXAMPLES

It should be understood that the following Examples, while indicating embodiments of the disclosure, are given by way of illustration only. From the above discussion and these Examples, one of skill in the art can make various changes and modifications of the disclosure to adapt it to various usages and conditions. Such modifications are also intended to fall within the scope of the claimed invention. Standard recombinant DNA and molecular cloning techniques used herein are well known in the art (Ausubel et al., 1987; Sambrook et al., 1989).

### Example 1

### Construction and Screening of a Regulatory Protein Deletion Library in Fungal Cells

### Overview

As generally set forth above in the Detailed Description, regulatory proteins are key regulators of cellular gene expression, wherein such regulatory proteins typically regulate gene expression by binding to specific (target) sites in the promoter regions of the (regulated) genes. For example, the binding of a regulatory protein to its targeted promoter sites can either up-regulate or down-regulate the transcription of genes involved in various cellular functions, processes and the like.

In the instant example, Applicant identified more than four hundred (400) regulatory proteins which were predicted via bioinformatics tools using Trichoderma reesei QM6a strain from the Joint Genome Institute (JGI) database (Nordberg et al., 2014). More particularly, in the Examples and data presented below (see, TABLES 1-15), a wild-type T. reesei strain (QM6a; ATCC®13631) was used throughout the study for molecular biology manipulations. In addition, to improve efficiency of gene deletions, a ku80 disrupted (Δku80) mutant impaired in non-homologous end-joining (NHEJ) machinery, was constructed via replacing the ku80 gene with the pyr4 marker (e.g., see PCT Publication No. WO2016/100272, which describes the construction of such Δku80 fungal mutants impaired in NHEJ). Thus, in certain aspects, a library of regulatory protein deletion constructs was generated by a fusion PCR approach between approximately two (2) kb long gene specific upstream (5′) and downstream (3′) flanking regions separated by a hygromycin selection marker (hphB), which was used for fungal transformation and selection of gene knockouts. Transformed colonies were selected on agar plates containing 2% malt extract, 0.2% peptone, 2% glucose, 1 M sorbitol, 5 mM Tris (pH 5.0) supplemented with 50 μg/ml hygromycin followed by a colony PCR analysis for correct integration of disruption cassettes in the specific loci. In total, three-hundred forty-three (343) regulatory protein deletant (mutant) strains were obtained and screened for improved characteristics as described in the Examples below.

### B. Preparation of Inoculum

Ten (10) μL of spore suspension were plated in twenty-four (24) well micro-titer plates (MTPs) with 2% malt extract agar media (Oxoid) and incubated at 30° C. in the presence of light. When sporulation was observed, spores were harvested and normalized to same OD₆₀₀ nm value. Fifty (50) μL of normalized spores' solution was used for inoculation of production plates.

### C. Protein Production and Preparation of Samples

T. reesei liquid cultures were grown in twenty-four (24) well polystyrene plates (Corning® Costart® CLS3527) with medium containing a glucose/sophorose mixture, or plates configured such as to release lactose or glucose from a solid, porous matrix. More particularly, each well contained 1.2 mL of a production medium (9 g/L casamino acids, 10 g/L (NH₄)₂SO₄, 4.5 g/L KH₂PO₄, 1 g/L MgSO₄·7H₂O, 1 g/L CaCl₂·2H₂O, 33 g/L PIPPS buffer (pH 5.5) and 0.25% T. reesei trace elements [175 g/L C₆H₈O₇, 200 g/L FeSO₄·7H₂O, 16 g/L ZnSO₄·7H₂O, 3.2 g/L CuSO₄·5H₂O, 1.4 g/L MnSO₄·H₂O and 0.8 g/L H₃BO₃]). In the case of polystyrene plates, a glucose/sophorose mixture at a final concentration of 2% (w/w) were used as a carbon source. In the case of glucose and lactose releasing plates, 1.6% (w/w) glucose was added to the production media. The plates were incubated at 28° C. or 34° C., 200 rpm (50 mm throw) with 80% humidity.

Sampling occurred after seventy-two (72) hours and one-hundred twenty (120) hours of incubation. Two hundred (200) μL of culture was collected and filtered with 0.2 μM Corning FILTREX™ CLS3505. Obtained supernatant was used for total protein measurements and enzymatic activity measurements.

D. Protein Determination with Bradford and HPLC

The concentration of total protein in the culture supernatants was determined by BioRad Bradford assay using bovine serum albumin (BSA) as a standard. The cellobiohydrolase 1 (CBH1) levels were measured by HPLC, using an Agilent 1200 HPLC equipped with an Acquity UPLC BEH200 SEC 1.7 μm (4.6×150 mm) column (Waters #186005225). Twenty-five (25) μL of sample was mixed with seventy-five (75) μL of de-mineralized water. Ten (10) μL of the 4× diluted sample was injected onto the column. To elute the sample, twenty-five (25) mM NaH₂PO₄ (pH 6.7) and one hundred (100) mM NaCl was run isocratically for five (5) minutes. To calculate performance index (PI), the ratio of the (average) total protein produced by a deletion mutant of the QM6a (Δku80) strain and (average) total protein produced by the wild-type QM6A (Δku80) strain under identical conditions were compared.

### E. Phosphoric Acid Swollen Cellulose (PASC) Hydrolysis Assay

Phosphoric acid swollen cellulose (PASC) was prepared from Avicel according to the published method of Wood (1971), and described in PCT Publication No. WO2014/093275. This material was diluted with buffer and water to achieve a 0.5% (w/v) mixture, such that the final concentration of sodium acetate was fifty (50) mM (pH 5.0). CBH1 activity was determined by adding five (5) μL, ten (10) μL, twenty (20) μL and forty (40) μL of supernatant (see C) to one-hundred forty (140) μL reaction mix (0.36% PASO; 29.4 mM NaOAc (pH 5.0); 143 mM NaCl) in a ninety-six (96)-well microtiter plate (Costar Flat Bottom PS 3641). The micro-titer plate was sealed and incubated at 50° C. with continuous shaking at 900 rpm for two (2) hours, followed by five (5) minutes cooling on ice. The hydrolysis reaction was stopped by the addition of one hundred (100) μL quench buffer (100 mM glycine buffer; pH 10). The hydrolysis reaction products were analyzed with a p-hydroxybenzoic acid hydrazide (PAHBAH) assay according to Lever (1972), with the following modifications.

PAHBAH Assay: Aliquots of one hundred-fifty (150) μL of PAHBAH reducing sugar reagent (for 100 mL reagent: 1.5 g p-hydroxybenzoic acid hydrazide (Sigma #H9882), 5 g potassium sodium tartrate tetrahydrate dissolved in 2% NaOH), were added to all wells of an empty microtiter plate. Ten (10) μL of the hydrolysis reaction supernatants were added to the PAHBAH reaction plate. All plates were sealed and incubated at 69° C. under continuous shaking of 900 rpm. After one (1) hour the plates were placed on ice for five (5) minutes and centrifuged at 720×g at room temperature for five (5) minutes. Absorbance of plates (endpoint) was measured at 410 nm in a spectrophotometer. A cellobiose standard was included as control and appropriate blank samples. To calculate performance index (PI), the (average) total sugar produced by wild-type QM6a (Δku80) strain was divided by the (average) total sugar produced by the regulatory protein deletion mutant of the QM6a (Δku80) strain.

### Example 2

### Improved Protein Production at 28° C.

A subset of one-hundred sixty-six (166) regulatory protein deletion mutants were screened for improved protein production under various conditions. For example, presented below in TABLE 1 are T. reesei gene (DNA) sequences (SEQ ID), predicted protein sequences (SEQ ID), predicted DNA binding domain (DBD) sequences (SEQ ID) of certain T. reesei regulatory protein deletion mutants described herein.

TABLE 1
T. REESEI REGULATORY PROTEINS IDENTIFIED
IN DELETION LIBRARY FOR IMPROVED PROTEIN
PRODUCTION UNDER SPECIFIED CONDITIONS

| | | | | | Type/ |
|---|---|---|---|---|---|
| Name | PID | DNA | PRT\hat{ } | DBD\hat{ }\hat{ } | Family |
| Trire2 4933 | 4933 | 1 | 2 | 3 | Helix-loop-helix |
| Trire2 5675 | 5675 | 4 | 5 | 6 | Zn finger; C₂H₂ type |
| Trire2 48438 | 48438 | 7 | 8 | 9 | Zn(2)-C6 fungal-type superfamily |
| Trire2 49232 | 49232 | 10 | 11 | 12 | Zn(2)-C6 fungal-type superfamily |
| Trire2 55105 | 55105 | 13 | 14 | 15 | Zn(2)-C6 fungal-type superfamily |
| Trire2 60565 | 60565 | 16 | 17 | 18 | Zn(2)-C6 fungal-type superfamily |
| Trire2 60931 | 60931 | 19 | 20 | 21 | Zn(2)-C6 fungal-type superfamily |
| Trire2 67209 | 67209 | 22 | 23 | 24 | Fork-head |
| Trire2 68097 | 68097 | 25 | 26 | 27 | MADS superfamily |
| Trire2 68425 | 68425 | 28 | 29 | 30 | Zn(2)-C6 fungal-type superfamily |
| Trire2 69695 | 69695 | 31 | 32 | 33 | Zn(2)-C6 fungal-type superfamily |
| Trire2 71823 | 71823 | 34 | 35 | 36 | Zn(2)-C6 fungal-type superfamily |
| Trire2 72993 | 72993 | 37 | 38 | 39 | Zn(2)-C6 fungal-type superfamily |
| Trire2 76705 | 76705 | 40 | 41 | 42 | Zn(2)-C6 fungal-type superfamily |
| Trire2 76872 | 76872 | 43 | 44 | 45 | Zn(2)-C6 fungal-type superfamily |
| Trire2 77291 | 77291 | 46 | 47 | 48 | Zn(2)-C6 fungal-type superfamily |
| Trire2 78162 | 78162 | 49 | 50 | 51 | U1-like Zn finger |
| Trire2 103122 | 103122 | 52 | 53 | 54 | Zn(2)-C6 fungal-type superfamily |
| Trire2 105239 | 105239 | 55 | 56 | 57 | Zn(2)-C6 fungal-type superfamily |
| Trire2 105849 | 105849 | 58 | 59 | 60 | Zn(2)-C6 fungal-type superfamily |
| Trire2 108013 | 108013 | 61 | 62 | 63 | Zn(2)-C6 fungal-type superfamily |
| Trire2 108775 | 108775 | 64 | 65 | 66 | TEAD family; TEA/ATTS domain |
| Trire2 119986 | 119986 | 67 | 68 | 69 | Zn(2)-C6 fungal-type superfamily |
| Trire2 120428 | 120428 | 70 | 71 | 72 | Zn finger, C2H2 type |
| Trire2 120597 | 120597 | 73 | 74 | 75 | Zn finger, C2H2 type |
| Trire2 121757 | 121757 | 76 | 77 | 78 | Zn finger, C2H2 type |
| Trire2 122783 | 122783 | 79 | 80 | 81 | Zn(2)-C6 fungal-type superfamily |

The column heading “PID” is an abbreviation for “Protein Identification Number” of the JGI database Trichoderma reesei v2.0 (Nordberg et al., 2014), the column heading “PRT\hat{ }” is an abbreviation referring to the “full-length” amino acid “sequence identification number (SEQ ID NO)” of the regulatory protein and the column heading “DBD\hat{ }\hat{ }” is an abbreviation referring to the predicted amino acid sequence of the “DNA binding domain” (DBD) present in the full-length amino acid sequence of the regulatory protein.

More particularly, protein productivity was measured by total protein determination and enzymatic activity assay (Example 1), wherein regulatory proteins whose deletion resulted in increased total protein production at 28° C. are set forth below in TABLES 2-4.

For example, regulatory proteins whose deletion resulted in increased protein production at 28° C. in lactose releasing plates after one-hundred twenty (120) hours of incubation are set forth in TABLE 2, wherein the numerical values represent performance indexes (PIs) calculated for PASC enzymatic assay, HPLC measurements of CBH1, and total protein measured by Bradford, relative to the control (wild-type) strain QM6a Δku80.

TABLE 2
Regulatory Protein Deletions Resulting in Increased
Protein Production at 28° C. in Lactose
Releasing Plates After 120 Hours of Incubation

| Regulatory Protein | PASC | CBH1 | Total Protein |
|---|---|---|---|
| Trire2_76872 (SEQ ID NO: 44) | 1.73 | 2.51 | 1.27 |

As shown in TABLE 2, the mutant cell comprising a deletion of regulatory protein Trire2_76872 (SEQ ID NO: 44) comprises an enhanced protein productivity phenotype (i.e., relative to parental cell) when fermented at 28° C. under lactose releasing (inducing) conditions, wherein the mutant cell deficient in the production of regulatory protein Trire2_76872 (SEQ ID NO: 44) produces an increased amount of total protein and an increased amount of CBH1, which results in higher saccharification activity (as exemplified by PASC assay in comparison to parental strain).

Likewise, regulatory proteins whose deletion resulted in increased protein production at 28° C. in glucose releasing plates after one-hundred twenty (120) hours of incubation are set forth in TABLE 3, wherein the numerical values represent performance indexes (PIs) calculated for PASC enzymatic assay, HPLC measurements of CBH1 or total protein measured by Bradford relative to the control (wild-type) strain QM6a Δku80.

TABLE 3
Regulatory Protein Deletions Resulting in Increased
Protein Production at 28° C. in Glucose
Releasing Plates After 120 Hours of Incubation

| Regulatory Protein | PASC | CBH1 | Total Protein |
|---|---|---|---|
| Trire2 76872 (SEQ ID NO: 44) | 3.76 | 7.00 | 1.69 |
| Trire2 120428 (SEQ ID NO: 71) | 2.41 | — | 1.10 |
| Trire2 76705 (SEQ ID NO: 41) | 1.75 | 1.65 | 1.08 |
| Trire2 78162 (SEQ ID NO: 50) | 1.59 | 1.41 | 1.04 |
| Trire2 105239 (SEQ ID NO: 56) | 1.56 | 1.37 | 1.01 |

For example, as shown in TABLE 3, the mutant cells comprising a deletion of regulatory protein Trire2_76872 (SEQ ID NO: 44), Trire2_120428 (SEQ ID NO: 71), Trire2_76705 (SEQ ID NO: 41), Trire2_78162 (SEQ ID NO: 50) or Trire2_105239 (SEQ ID NO: 56), comprise enhanced protein productivity phenotypes (relative to parental cells) when fermented at 28° C. under glucose releasing conditions. More particularly, such mutant cells deficient in the production of the aforementioned regulatory proteins (TABLE 3) produced increased amounts of total protein and/or increased amounts of CBH1 under such glucose releasing conditions (relative to parental cell).

In addition, set forth below in TABLE 4 are regulatory proteins whose deletion resulted in increased protein production at 28° C. in polystyrene plates with 2% (w/w) glucose/sophorose as a carbon source after one-hundred twenty (120) hours of incubation, wherein numerical values represent performance indexes (PIs) calculated for PASC enzymatic assay, HPLC measurements of CBH1 or total protein measured by Bradford relative to the control (wild-type) strain QM6a Δku80.

TABLE 4
Regulatory Protein Deletions Resulting in Increased Protein
Production at 28° C. with 2% (w/w) Glucose/Sophorose
as Carbon Source After 120 Hours of Incubation

| Regulatory Protein | PASC | CBH1 | Total protein |
|---|---|---|---|
| Trire2 69695 (SEQ ID NO: 32) | 1.01 | 1.52 | 1.07 |
| Trire2 76705 (SEQ ID NO: 41) | 0.94 | 1.30 | 1.12 |
| Trire2 78162 (SEQ ID NO: 50) | 0.74 | 1.57 | 1.10 |
| Trire2 105849 (SEQ ID NO: 59) | 3.26 | 1.44 | 1.34 |
| Trire2 71823 (SEQ ID NO: 35) | 1.35 | 1.41 | 1.10 |
| Trire2 60931 (SEQ ID NO: 20) | 1.26 | 1.36 | 1.03 |
| Trire2 48438 (SEQ ID NO: 8) | 1.24 | 1.60 | 1.10 |
| Trire2 108013 (SEQ ID NO: 62) | 1.20 | 1.30 | 0.98 |
| Trire2 68097 (SEQ ID NO: 26) | 1.19 | 1.36 | 1.15 |
| Trire2 4933 (SEQ ID NO: 2) | 1.19 | 1.28 | 0.90 |
| Trire2 72993 (SEQ ID NO: 38) | 1.17 | 1.41 | 1.09 |
| Trire2 119986 (SEQ ID NO: 68) | 1.13 | 1.35 | 1.11 |
| Trire2 55105 (SEQ ID NO: 14) | 0.94 | 1.20 | 1.11 |
| Trire2 68425 (SEQ ID NO: 29) | 0.92 | 1.17 | 1.08 |
| Trire2 103122 (SEQ ID NO: 53) | 0.92 | 1.08 | 1.13 |
| Trire2 5675 (SEQ ID NO: 5) | 0.91 | 1.45 | 1.10 |
| Trire2 121757 (SEQ ID NO: 77) | 0.90 | 1.46 | 1.13 |
| Trire2 77291 (SEQ ID NO: 47) | 0.81 | 1.32 | 1.09 |

As presented in TABLE 4 above, the mutant cells comprising a deletion of regulatory protein Trire2_69695 (SEQ ID NO: 32), Trire2_76705 (SEQ ID NO: 41), Trire2_78162 (SEQ ID NO: 50), Trire2_105849 (SEQ ID NO: 59), Trire2_71823 (SEQ ID NO: 35), Trire2_60931 (SEQ ID NO: 20), Trire2_48438 (SEQ ID NO: 8), Trire2_108013 (SEQ ID NO: 62), Trire2_68097 (SEQ ID NO: 26), Trire2_4933 (SEQ ID NO: 2), Trire2_72993 (SEQ ID NO: 38), Trire2_119986 (SEQ ID NO: 68), Trire2_55105 (SEQ ID NO: 14), Trire2_68425 (SEQ ID NO: 29), Trire2_103122 (SEQ ID NO: 53), Trire2_5675 (SEQ ID NO: 5), Trire2_121757 (SEQ ID NO: 77) or Trire2_77291 (SEQ ID NO: 47), comprise enhanced protein productivity phenotypes (relative to parental cells) when fermented at 28° C. with 2% (w/w) glucose/sophorose as a carbon source. For example, the mutant cells deficient in the production of the aforementioned regulatory proteins (TABLE 4) produce increased amounts of total protein and/or increased amounts of CBH1 under such glucose/sophorose (inducing) conditions (relative to parental cell).

### Example 3

### Improved Protein Production at 34° C.

In the instant example, Applicant screened the one-hundred sixty-six (166) regulatory protein deletion library mutants (Example 2) to determine if any of the regulatory protein deletions further result in improved protein production at a higher cultivation temperature of 34° C. (e.g., see TABLES 5 and 6). The protein productivity was measured by total protein determination and enzymatic activity assay (Example 1). For example, regulatory proteins whose deletion resulted in increased protein production at 34° C. in lactose releasing plates after one-hundred twenty (120) hours of incubation are set forth in TABLE 5, wherein the numerical values represent performance indexes (PIs) calculated for PASC enzymatic assay, HPLC measurements of CBH1 or total protein measured by Bradford relative to the control (wild-type) strain QM6a Δku80.

TABLE 5
Regulatory Protein Deletions Resulting in Increased
Protein Production at 34° C. in Lactose
Releasing Plates After 120 Hours of Incubation

| Regulatory Protein | PASC | CBH1 | Total Protein |
|---|---|---|---|
| Trire2 76872 (SEQ ID NO: 44) | 2.19 | 2.37 | 1.66 |
| Trire2 67209 (SEQ ID NO: 23) | 1.42 | 1.08 | 1.08 |
| Trire2 122783 (SEQ ID NO: 80) | 1.17 | 1.03 | 1.03 |

As presented above in TABLE 5, the mutant cells comprising a deletion of regulatory protein Trire2_76872 (SEQ ID NO: 44), Trire2_67209 (SEQ ID NO: 23) or Trire2_122783 (SEQ ID NO: 80), comprise enhanced protein productivity phenotypes (relative to parental cells) when fermented at 34° C. under lactose releasing conditions. For example, the mutant cells deficient in the production of the aforementioned regulatory proteins (TABLE 5) produce increased amounts of total protein and/or increased amounts of CBH1 under such lactose (inducing) conditions (relative to parental cell).

Likewise, regulatory proteins whose deletion resulted in increased protein production at 34° C. in glucose releasing plates after one-hundred twenty (120) hours of incubation are set forth in TABLE 6, wherein the numerical values represent performance indexes (PIs) calculated for PASC enzymatic assay, HPLC measurements of CBH1 or total protein measured by Bradford relative to the control (wild-type) strain QM6a Δku80.

TABLE 6
Regulatory Protein Deletions Resulting in Increased
Protein Production at 34° C. in Glucose
Releasing Plates After 120 Hours of Incubation

| Regulatory Protein | PASC | CBH1 | Total Protein |
|---|---|---|---|
| Trire2 76705 (SEQ ID NO: 41) | 0.76 | 1.30 | 1.15 |
| Trire2 120597 (SEQ ID NO: 74) | 1.84 | 2.51 | 1.15 |
| Trire2 49232 (SEQ ID NO: 11) | 1.44 | 1.86 | 1.05 |
| Trire2 108775 (SEQ ID NO: 65) | 1.35 | 2.68 | 1.05 |
| Trire2 60565 (SEQ ID NO: 17) | 1.25 | 2.48 | 1.03 |

Thus, as presented above in TABLE 6, the mutant cells comprising a deletion of regulatory protein Trire2_76705 (SEQ ID NO: 41), Trire2_120597 (SEQ ID NO: 74), Trire2_49232 (SEQ ID NO: 11), Trire2_108775 (SEQ ID NO: 65) or Trire2_60565 (SEQ ID NO: 17), comprise enhanced protein productivity phenotypes (relative to parental cells) when fermented at 34° C. under glucose releasing conditions. For example, the mutant cells deficient in the production of the aforementioned regulatory proteins (TABLE 6) produce increased amounts of total protein and/or increased amounts of CBH1 at 34° C. in the absence of inducing substrates (relative to parental cell).

### Example 4

### Improved Protein Production Rates

In the present example, regulatory proteins whose deletion resulted in higher protein production rates were determined by comparison of the protein production at an earlier timepoint of seventy-two (72) hours, followed by final protein production measurements at one-hundred twenty (120) hours. More particularly, only those regulatory proteins whose deletion resulted in higher production at seventy-two (72) hours, and comparable or improved protein production at one-hundred twenty (120) hours in comparison to the control (wild-type) strain QM6a Δku80 were selected, as presented below in TABLES 7-9.

For example, regulatory proteins whose deletion resulted in increased protein production rates in lactose releasing plates after seventy-two (72) hours of incubation at 28° C. or 34° C. are set forth below in TABLE 7, wherein the numerical values represent performance indexes (PIs) calculated for PASC enzymatic assay, HPLC measurements of CBH1 or total protein measured by Bradford relative to the control (wild-type) strain QM6a Δku80.

TABLE 7
Regulatory Protein Deletions Resulting in Increased
Protein Production Rate in Lactose Releasing Plates
After 72 Hours of Incubation at 28° C. or 34° C.

| Regulatory Protein | 28° C. | | 34° C. | |
|---|---|---|---|---|
| Time | 72 h | 120 h | 72 h | 120 h |
| Trire2 76872 (SEQ ID NO: 44) | 1.32 | 1.27 | 1.23 | 1.66 |

As presented above in TABLE 7, mutant cells comprising a deletion of regulatory protein Trire2_76872 (SEQ ID NO: 44) comprise enhanced protein production rates (relative to parental cell) after seventy-two (72) hours of cultivation at 28° C. in a lactose releasing plates. In addition, as shown in TABLE 7, mutant cells comprising a deletion of regulatory protein Trire2_76872 (SEQ ID NO: 44) comprise enhanced protein production rates (relative to parental cell) after seventy-two (72) hours of cultivation at 34° C. in a lactose releasing plates. Thus, as exemplified above, mutant cells deficient in the production of the aforementioned regulatory protein (TABLE 7) demonstrate increased protein production rates (relative to parental cell).

Likewise, regulatory proteins whose deletion resulted in an increased protein production rate in glucose releasing plates after seventy-two (72) hours of incubation at 28° C. or 34° C. are set forth below in TABLE 8, wherein the numerical values represent performance indexes (PIs) calculated for PASC enzymatic assay, HPLC measurements of CBH1 or total protein measured by Bradford relative to the control (wild-type) strain QM6a Δku80.

TABLE 8
Regulatory Protein Deletions Resulting in Increased
Protein Production Rate in Glucose Releasing Plates
After 72 Hours of Incubation at 28° C. or 34° C.

| Regulatory Protein | 28° C. | | 34° C. | |
|---|---|---|---|---|
| Time | 72 h | 120 h | 72 h | 120 h |
| Trire2 76872 (SEQ ID NO: 44) | 1.62 | 1.65 | 1.53 | 1.85 |
| Trire2 78162 (SEQ ID NO: 50) | | | 1.05 | 1.09 |

As presented above in TABLE 8, the mutant cells comprising a deletion of regulatory protein Trire2_76872 (SEQ ID NO: 44) comprise enhanced protein production rates (i.e., relative to parental cells) after seventy-two (72) hours of cultivation at 28° C. in a glucose releasing plates. Likewise, as presented above in TABLE 8, the mutant cells comprising a deletion of regulatory proteins Trire2_76872 (SEQ ID NO: 44) or Trire2_78162 (SEQ ID NO: 50) comprise enhanced protein production rates after seventy-two (72) hours of cultivation at 34° C. in a glucose releasing plates. Thus, as exemplified above, mutant cells deficient in the production of the aforementioned regulatory proteins (TABLE 8) demonstrate increased protein production rates (relative to parental cell).

Additionally, regulatory proteins whose deletion resulted in an increased protein production rate in polystyrene plates with 2% glucose/sophorose as a carbon source after seventy-two (72) hours of incubation at 28° C. are set forth below in TABLE 9, wherein the numerical values represent performance indexes (PIs) calculated for PASC enzymatic assay, HPLC measurements of CBH1 or total protein measured by Bradford relative to the control (wild-type) strain QM6a Δku80.

TABLE 9
Regulatory Protein Deletions Resulting in Increased Protein
Production Rate in Polystyrene Plates with 2% Glucose/Sophorose
as a Carbon Source After 72 Hours of Incubation at 28° C.

| | |
|---|---|
| Regulatory Protein | 28° C. |

| Time | 72 h | 120 h |
|---|---|---|
| Trire2 78162 (SEQ ID NO: 50) | 1.02 | 1.10 |
| Trire2 105849 (SEQ ID NO: 59) | 1.11 | 1.34 |

As presented above in TABLE 9, the mutant cells comprising a deletion of regulatory proteins Trire2_78162 (SEQ ID NO: 50) or Trire2_105849 (SEQ ID NO: 59) comprise enhanced protein production rates after seventy-two (72) hours of cultivation at 28° C. in a medium with 2% (w/w) glucose/sophorose as a carbon source. Thus, as exemplified above, mutant cells deficient in the production of the aforementioned regulatory proteins (TABLE 9) demonstrate increased protein production rates (relative to parental cell).

### Example 5

### Reduced Protein Production at 28° C.

A subset of one-hundred sixty-six (166) regulatory protein deletion mutants were screened for reduced protein production under various conditions. The protein productivity was measured by total protein determination and enzymatic activity assay (Example 1). For example, presented below in TABLE 10 are predicted protein sequences (SEQ ID) of the T. reesei regulatory protein deletion mutants whose regulatory protein deletion resulted in decreased total protein production.

TABLE 10
T. REESEI REGULATORY PROTEINS IDENTIFIED IN DELETION LIBRARY
WITH REDUCED PROTEIN PRODUCTION UNDER SPECIFIED CONDITIONS

| Name | PID | PRT\hat{ } | Pfam /Family |
|---|---|---|---|
| Trire2 1941 | 1941 | 82 | Homeobox-like domain superfamily; SANT/Myb domain |
| Trire2 2148 | 2148 | 83 | Zn(2)-C6 fungal-type DNA-binding domain superfamily |
| Trire2 3605 | 3605 | 84 | Zn(2)-C6 fungal-type DNA-binding domain superfamily |
| Trire2 4748 | 4748 | 85 | Zinc finger C2H2 superfamily |
| Trire2 5664 | 5664 | 86 | HTH APSES-type DNA-binding domain superfamily; MBF |
| | | | transcription factor complex subunit Mbp1/Res1/Res2; KilA, |
| | | | N-terminal/APSES-type HTH; Ankyrin repeat-containing |
| | | | domain |
| Trire2 5927 | 5927 | 87 | Zn(2)-C6 fungal-type DNA-binding domain superfamily |
| Trire2 21997 | 21997 | 88 | Zn(2)-C6 fungal-type DNA-binding domain superfamily |
| Trire2 22774 | 22774 | 89 | Helix-loop-helix DNA-binding domain superfamily |
| Trire2 22785 | 22785 | 90 | Zinc finger C2H2 superfamily |
| Trire2 36703 | 36703 | 91 | Zn(2)-C6 fungal-type DNA-binding domain superfamily |
| Trire2 44290 | 44290 | 92 | Zinc finger C2H2 superfamily |
| Trire2 44781 | 44781 | 93 | Transcription initiation factor IID, subunit 13; TAF13 |
| Trire2 45866 | 45866 | 94 | Zinc finger C2H2 superfamily |
| Trire2 48773 | 48773 | 95 | Zn(2)-C6 fungal-type DNA-binding domain superfamily |
| Trire2 49918 | 49918 | 96 | TBP domain superfamily; TATA-box binding protein |
| Trire2 52438 | 52438 | 97 | Fungal transcription factor domain MHR |
| Trire2 52924 | 52924 | 98 | R3H domain superfamily; Transcription factor NFX1 family |
| Trire2 53106 | 53106 | 99 | Zn(2)-C6 fungal-type DNA-binding domain superfamily |
| Trire2 53484 | 53484 | 100 | HTH APSES-type DNA-binding domain superfamily; MBF |
| | | | transcription factor complex subunit Mbp1/Res1/Res2; KilA, |
| | | | N-terminal/APSES-type HTH; Ankyrin repeat-containing |
| | | | domain |
| Trire2 55274 | 55274 | 101 | Zn(2)-C6 fungal-type DNA-binding domain superfamily |
| Trire2 56214 | 56214 | 102 | Zn(2)-C6 fungal-type DNA-binding domain superfamily |
| Trire2 58130 | 58130 | 103 | Helix-loop-helix (bHLH) DNA-binding domain superfamily; |
| | | | Myc-type, |
| Trire2 59353 | 59353 | 104 | Zn(2)-C6 fungal-type DNA-binding domain superfamily |
| Trire2 59609 | 59609 | 105 | WD40/YVTN repeat-like-containing domain superfamily |
| Trire2 60558 | 60558 | 106 | Zn(2)-C6 fungal-type DNA-binding domain superfamily |
| Trire2 61476 | 61476 | 107 | Zn(2)-C6 fungal-type DNA-binding domain superfamily |
| Trire2 65070 | 65070 | 108 | Zn(2)-C6 fungal-type DNA-binding domain superfamily |
| Trire2 65895 | 65895 | 109 | Mediator complex, subunit Med31 domain superfamily |
| Trire2 68028 | 68028 | 110 | E3 ubiquitin ligase RBR family; IBR domain |
| Trire2 70414 | 70414 | 111 | Zn(2)-C6 fungal-type DNA-binding domain superfamily |
| Trire2 70991 | 70991 | 112 | Class I glutamine amidotransferase-like; DJ-1/PfpI |
| Trire2 71080 | 71080 | 113 | Zn(2)-C6 fungal-type DNA-binding domain superfamily |
| Trire2 71689 | 71689 | 114 | Zn(2)-C6 fungal-type DNA-binding domain superfamily |
| Trire2 72076 | 72076 | 115 | Zn(2)-C6 fungal-type DNA-binding domain superfamily |
| Trire2 73417 | 73417 | 116 | Basic-leucine zipper domain |
| Trire2 73559 | 73559 | 117 | Zn(2)-C6 fungal-type DNA-binding domain superfamily |
| Trire2 73689 | 73689 | 118 | Zinc finger C2H2 superfamily |
| Trire2 76039 | 76039 | 119 | Zn(2)-C6 fungal-type DNA-binding domain superfamily |
| Trire2 76590 | 76590 | 120 | Zn(2)-C6 fungal-type DNA-binding domain superfamily |
| Trire2 77878 | 77878 | 121 | Zinc finger C2H2 superfamily |
| Trire2 105784 | 105784 | 122 | Zn(2)-C6 fungal-type DNA-binding domain superfamily |
| Trire2 105880 | 105880 | 123 | Zn(2)-C6 fungal-type DNA-binding domain superfamily |
| Trire2 105917 | 105917 | 124 | Zn(2)-C6 fungal-type DNA-binding domain superfamily |
| Trire2 105980 | 105980 | 125 | Zn(2)-C6 fungal-type DNA-binding domain superfamily |
| Trire2 105989 | 105989 | 126 | Zn(2)-C6 fungal-type DNA-binding domain superfamily |
| Trire2 106009 | 106009 | 127 | Fungal transcription factor domain MHR |
| Trire2 106720 | 106720 | 128 | Zn(2)-C6 fungal-type DNA-binding domain superfamily |
| Trire2 109277 | 109277 | 129 | Zn(2)-C6 fungal-type DNA-binding domain superfamily |
| Trire2 110901 | 110901 | 130 | Zn(2)-C6 fungal-type DNA-binding domain superfamily |
| Trire2 119826 | 119826 | 131 | Homeobox-like domain superfamily; HOX_1 |
| Trire2 121164 | 121164 | 132 | Zn(2)-C6 fungal-type DNA-binding domain superfamily |
| Trire2 121310 | 121310 | 133 | Zn(2)-C6 fungal-type DNA-binding domain superfamily |
| Trire2 121602 | 121602 | 134 | Zn(2)-C6 fungal-type DNA-binding domain superfamily |
| Trire2 122457 | 122457 | 135 | Cro/C1-Lambda repressor-like DNA-binding domain |
| | | | superfamily; of a helix-turn-helix type; Multiprotein bridging |
| | | | factor 1 |
| Trire2 123713 | 123713 | 136 | Transcriptional regulator MedA |

The column heading “PID” is an abbreviation for “Protein Identification Number” of the JGI database Trichoderma reesei v2.0 (Nordberg et al., 2014) and the column heading “PRT\hat{ }” is an abbreviation referring to the amino acid “sequence identification number (SEQ ID NO)” of the regulatory protein.

For example, regulatory proteins whose deletion resulted in decreased protein production at 28° C. in lactose releasing plates after one-hundred twenty (120) hours of incubation are set forth below in TABLE 11, wherein the numerical values represent performance indexes (PIs) calculated for PASC enzymatic assay, HPLC measurements of CBH1, and total protein measured by Bradford, relative to the control (wild-type) strain QM6a Δku80.

TABLE 11
Regulatory Protein Deletions Resulting in Decreased
Protein Production at 28° C. in Lactose
Releasing Plates After 120 Hours of Incubation

| Regulatory Protein | PASC | CBH1 | Total Protein |
|---|---|---|---|
| Trire2 3605 (SEQ ID NO: 84) | 0.73 | 0.77 | 0.90 |
| Trire2 4748 (SEQ ID NO: 85) | 0.15 | 0.64 | 0.79 |
| Trire2 44781 (SEQ ID NO: 93) | 0.21 | 0.71 | 0.89 |
| Trire2 48773 (SEQ ID NO: 95) | 0.78 | 0.79 | 0.89 |
| Trire2 53484 (SEQ ID NO: 100) | 0.20 | 0.78 | 0.81 |
| Trire2 58130 (SEQ ID NO: 103) | 0.42 | 0.61 | 0.84 |
| Trire2 59609 (SEQ ID NO: 105) | — | 0.52 | 0.77 |
| Trire2 65070 (SEQ ID NO: 108) | 0.58 | 0.72 | 0.89 |
| Trire2 65895 (SEQ ID NO: 109) | 0.90 | 0.76 | 0.88 |
| Trire2 71080 (SEQ ID NO: 113) | 0.63 | 0.79 | 0.88 |
| Trire2 71689 (SEQ ID NO: 114) | 0.28 | 0.65 | 0.89 |
| Trire2 72076 (SEQ ID NO: 115) | 0.15 | 0.52 | 0.82 |
| Trire2 73559 (SEQ ID NO: 117) | 0.36 | 0.63 | 0.81 |
| Trire2 73689 (SEQ ID NO: 118) | 0.52 | 0.78 | 0.90 |
| Trire2 76039 (SEQ ID NO: 119) | 0.42 | 0.76 | 0.90 |
| Trire2 119826 (SEQ ID NO: 131) | 0.45 | 0.76 | 0.88 |
| Trire2 121164 (SEQ ID NO: 132) | 0.22 | 0.63 | 0.82 |
| Trire2 121602 (SEQ ID NO: 134) | 0.36 | 0.77 | 0.83 |
| Trire2 122457 (SEQ ID NO: 135) | 0.46 | 0.78 | 0.90 |

As shown in TABLE 11, the mutant cells comprising a deletion of regulatory protein Trire2_3605 (SEQ ID NO: 84), Trire2_4748 (SEQ ID NO: 85), Trire2_44781 (SEQ ID NO: 93), Trire2_48773 (SEQ ID NO: 95), Trire2_53484 (SEQ ID NO: 100), Trire2_58130 (SEQ ID NO: 103), Trire2_59609 (SEQ ID NO: 105), Trire2_65070 (SEQ ID NO: 108), Trire2_65895 (SEQ ID NO: 109), Trire2_71080 (SEQ ID NO: 113), Trire2_71689 (SEQ ID NO: 114), Trire2_72076 (SEQ ID NO: 115), Trire2_73559 (SEQ ID NO: 117), Trire2_73689 (SEQ ID NO: 118), Trire2_76039 (SEQ ID NO: 119), Trire2_119826 (SEQ ID NO: 131), Trire2_121164 (SEQ ID NO: 132), Trire2_121602 (SEQ ID NO: 134) or Trire2_122457 (SEQ ID NO: 135), comprise a diminished (reduced) protein productivity phenotype (i.e., relative to parental cells) when fermented at 28° C. under lactose (inducing) conditions. For example, mutant cells deficient in the production of the aforementioned regulatory proteins (TABLE 11) produce a decreased amount of total protein, and a decreased amount of CBH1, which results in lower saccharification activity (as exemplified by PASC assay in comparison to the parental strain).

Likewise, regulatory proteins whose deletion resulted in decreased protein production at 28° C. in glucose releasing plates after one-hundred twenty (120) hours of incubation are set forth in TABLE 12, wherein the numerical values represent performance indexes (PIs) calculated for PASC enzymatic assay, HPLC measurements of CBH1 or total protein measured by Bradford relative to the control (wild-type) strain QM6a Δku80.

TABLE 12
Regulatory Protein Deletions Resulting in Decreased
Protein Production at 28° C. in Glucose
Releasing Plates After 120 Hours of Incubation

| Regulatory Protein | PASC | CBH1 | Total Protein |
|---|---|---|---|
| Trire2 1941 (SEQ ID NO: 82) | — | 0.62 | 0.80 |
| Trire2 2148 (SEQ ID NO: 83) | 0.58 | 0.76 | 0.87 |
| Trire2 5664 (SEQ ID NO: 86) | — | 0.75 | 0.87 |
| Trire2 5927 (SEQ ID NO: 87) | 0.53 | 0.76 | 0.88 |
| Trire2 21997 (SEQ ID NO: 88) | 0.37 | 0.73 | 0.86 |
| Trire2 22785 (SEQ ID NO: 90) | 0.50 | 0.70 | 0.85 |
| Trire2 36703 (SEQ ID NO: 91) | 0.33 | 0.78 | 0.89 |
| Trire2 48773 (SEQ ID NO: 95) | 0.60 | 0.74 | 0.87 |
| Trire2 49918 (SEQ ID NO: 96) | 0.68 | 0.81 | 0.90 |
| Trire2 52438 (SEQ ID NO: 97) | 0.59 | 0.74 | 0.86 |
| Trire2 52924 (SEQ ID NO: 98) | 0.53 | 0.77 | 0.88 |
| Trire2 53106 (SEQ ID NO: 99) | 0.27 | 0.70 | 0.84 |
| Trire2 55274 (SEQ ID NO: 101) | 0.59 | 0.76 | 0.87 |
| Trire2 56214 (SEQ ID NO: 102) | 0.19 | 0.70 | 0.84 |
| Trire2 59353 (SEQ ID NO: 104) | 0.23 | 0.64 | 0.82 |
| Trire2 59609 (SEQ ID NO: 105) | — | 0.67 | 0.83 |
| Trire2 60558 (SEQ ID NO: 106) | — | 0.70 | 0.85 |
| Trire2 61476 (SEQ ID NO: 107) | 0.11 | 0.69 | 0.84 |
| Trire2 65895 (SEQ ID NO: 109) | 0.30 | 0.76 | 0.88 |
| Trire2 70414 (SEQ ID NO: 111) | 0.14 | 0.79 | 0.89 |
| Trire2 70991 (SEQ ID NO: 112) | 0.81 | 0.81 | 0.90 |
| Trire2 71689 (SEQ ID NO: 114) | 0.27 | 0.67 | 0.83 |
| Trire2 73417 (SEQ ID NO: 116) | 0.19 | 0.68 | 0.84 |
| Trire2 76590 (SEQ ID NO: 120) | 0.70 | 0.81 | 0.90 |
| Trire2 77878 (SEQ ID NO: 121) | — | 0.80 | 0.90 |
| Trire2 105784 (SEQ ID NO: 122) | 0.51 | 0.73 | 0.86 |
| Trire2 105880 (SEQ ID NO: 123) | 0.62 | 0.75 | 0.87 |
| Trire2 105917 (SEQ ID NO: 124) | 0.35 | 0.74 | 0.87 |
| Trire2 105980 (SEQ ID NO: 125) | 0.65 | 0.77 | 0.88 |
| Trire2 105989 (SEQ ID NO: 126) | 0.18 | 0.73 | 0.86 |
| Trire2 106009 (SEQ ID NO: 127) | 0.41 | 0.64 | 0.82 |
| Trire2 106720 (SEQ ID NO: 128) | 0.52 | 0.80 | 0.89 |
| Trire2 109277 (SEQ ID NO: 129) | 0.26 | 0.70 | 0.85 |
| Trire2 110901 (SEQ ID NO: 130) | 0.30 | 0.64 | 0.82 |
| Trire2 121310 (SEQ ID NO: 133) | 0.44 | 0.76 | 0.88 |

For example, as shown in TABLE 12, the mutant cells comprising a deletion of regulatory protein Trire2_1941 (SEQ ID NO: 82), Trire2_2148 (SEQ ID NO: 83), Trire2_5664 (SEQ ID NO: 86), Trire2_5927 (SEQ ID NO: 87), Trire2_21997 (SEQ ID NO: 88), Trire2_22785 (SEQ ID NO: 90), Trire2_36703 (SEQ ID NO: 91), Trire2_48773 (SEQ ID NO: 95), Trire2_49918 (SEQ ID NO: 96), Trire2_52438 (SEQ ID NO: 97), Trire2_52924 (SEQ ID NO: 98), Trire2_53106 (SEQ ID NO: 99), Trire2_55274 (SEQ ID NO: 101), Trire2_56214 (SEQ ID NO: 102), Trire2_59353 (SEQ ID NO: 104), Trire2_59609 (SEQ ID NO: 105), Trire2_60558 (SEQ ID NO: 106), Trire2_61476 (SEQ ID NO: 107), Trire2_65895 (SEQ ID NO: 109), Trire2_70414 (SEQ ID NO: 111), Trire2_70991 (SEQ ID NO: 112), Trire2_71689 (SEQ ID NO: 114), Trire2_73417 (SEQ ID NO: 116), Trire2_76590 (SEQ ID NO: 120), Trire2_77878 (SEQ ID NO: 121), Trire2_105784 (SEQ ID NO: 122), Trire2_105880 (SEQ ID NO: 123), Trire2_105917 (SEQ ID NO: 124), Trire2_105980 (SEQ ID NO: 125), Trire2_105989 (SEQ ID NO: 126), Trire2_106009 (SEQ ID NO: 127), Trire2_106720 (SEQ ID NO: 128), Trire2_109277 (SEQ ID NO: 129), Trire2_110901 (SEQ ID NO: 130) or Trire2_121310 (SEQ ID NO: 133), comprise diminished (reduced) protein productivity phenotypes (relative to parental cells) when fermented at 28° C. under glucose releasing conditions. For example, the mutant cells deficient in the production of the aforementioned regulatory proteins (TABLE 12) produce decreased amounts of total protein or decreased amounts of CBH1 under such glucose releasing conditions (relative to parental cell).

In addition, set forth below in TABLE 13 are regulatory proteins whose deletion resulted in decreased protein production at 28° C. in polystyrene plates with 2% (w/w) glucose/sophorose as a carbon source after one-hundred twenty (120) hours of incubation, wherein numerical values represent performance indexes (PIs) calculated for PASC enzymatic assay, HPLC measurements of CBH1 or total protein measured by Bradford relative to the control (wild-type) strain QM6a Δku80.

TABLE 13
Regulatory Protein Deletions Resulting in Decreased Protein
Production at 28° C. with 2% (w/w) Glucose/Sophorose
as Carbon Source After 120 Hours of Incubation

| Regulatory Protein | PASC | CBH1 | Total Protein |
|---|---|---|---|
| Trire2 1941 (SEQ ID NO: 82) | 0.90 | 0.80 | 0.90 |
| Trire2 22785 (SEQ ID NO: 90) | 0.15 | 0.63 | 0.82 |
| Trire2 44290 (SEQ ID NO: 92) | — | 0.60 | 0.80 |
| Trire2 44781 (SEQ ID NO: 93) | — | 0.64 | 0.83 |
| Trire2 45866 (SEQ ID NO: 94) | 0.34 | 0.73 | 0.87 |
| Trire2 48773 (SEQ ID NO: 95) | 0.66 | 0.78 | 0.89 |
| Trire2 72076 (SEQ ID NO: 115) | 0.17 | 0.45 | 0.73 |
| Trire2 76590 (SEQ ID NO: 120) | 0.26 | 0.64 | 0.83 |

As presented in TABLE 13, the mutant cells comprising a deletion of regulatory protein Trire2_1941 (SEQ ID NO: 82), Trire2_22785 (SEQ ID NO: 90), Trire2_44290 (SEQ ID NO: 92), Trire2_44781 (SEQ ID NO: 93), Trire2_45866 (SEQ ID NO: 94), Trire2_48773 (SEQ ID NO: 95), Trire2_72076 (SEQ ID NO: 115) or Trire2_76590 (SEQ ID NO: 120), comprise reduced protein productivity phenotypes (relative to parental cells) when fermented at 28° C. with 2% (w/w) glucose/sophorose as a carbon source. For example, the mutant cells deficient in the production of the aforementioned regulatory proteins (TABLE 13) produce decreased amounts of total protein or decreased amounts of CBH1 under such glucose/sophorose conditions (relative to parental cell).

### Example 6

### Reduced Protein Production at 34° C.

In the instant example, Applicant screened the one-hundred sixty-six (166) regulatory protein deletion library mutants (Example 2) to determine if any of the regulatory protein deletions further result in reduced protein production at a higher temperature of 34° C. (e.g., see TABLE 14 and TABLE 15). The protein productivity was measured by total protein determination and enzymatic activity assay (Example 1). For example, regulatory proteins whose deletion resulted in decreased protein production at 34° C. in lactose releasing plates after one-hundred twenty (120) hours of incubation are set forth in TABLE 14, wherein the numerical values represent performance indexes (PIs) calculated for PASC enzymatic assay, HPLC measurements of CBH1 or total protein measured by Bradford relative to the control (wild-type) strain QM6a Δku80.

TABLE 14
Regulatory Protein Deletions Resulting in Decreased
Protein Production at 34° C. in Lactose
Releasing Plates After 120 Hours of Incubation

| Regulatory Protein | PASC | CBH1 | Total Protein |
|---|---|---|---|
| Trire2 4748 (SEQ ID NO: 85) | 0.13 | 0.76 | 0.87 |
| Trire2 21997 (SEQ ID NO: 88) | 0.57 | 0.78 | 0.88 |
| Trire2 22774 (SEQ ID NO: 89) | 0.60 | 0.74 | 0.87 |
| Trire2 22785 (SEQ ID NO: 90) | 0.70 | 0.79 | 0.89 |
| Trire2 36703 (SEQ ID NO: 91) | 0.54 | 0.81 | 0.90 |
| Trire2 44290 (SEQ ID NO: 92) | — | 0.38 | 0.68 |
| Trire2 44781 (SEQ ID NO: 93) | 0.13 | 0.55 | 0.76 |
| Trire2 59609 (SEQ ID NO: 105) | — | 0.48 | 0.73 |
| Trire2 68028 (SEQ ID NO: 110) | 0.62 | 0.81 | 0.90 |
| Trire2 71689 (SEQ ID NO: 114) | 0.03 | 0.66 | 0.82 |
| Trire2 72076 (SEQ ID NO: 115) | 0.15 | 0.56 | 0.77 |
| Trire2 77878 (SEQ ID NO: 121) | 0.34 | 0.70 | 0.85 |

Thus, as presented above in TABLE 14, the mutant cells comprising a deletion of regulatory protein Trire2_4748 (SEQ ID NO: 85), Trire2_21997 (SEQ ID NO: 88), Trire2_22774 (SEQ ID NO: 89), Trire2_22785 (SEQ ID NO: 90), Trire2_36703 (SEQ ID NO: 91), Trire2_44290 (SEQ ID NO: 92), Trire2_44781 (SEQ ID NO: 93), Trire2_59609 (SEQ ID NO: 105), Trire2_68028 (SEQ ID NO: 110), Trire2_71689 (SEQ ID NO: 114), Trire2_72076 (SEQ ID NO: 115) or Trire2_77878 (SEQ ID NO: 121), comprise diminished (reduced) protein productivity phenotypes (relative to parental cells) when fermented at 34° C. under lactose releasing conditions. For example, the mutant cells deficient in the production of the aforementioned regulatory proteins (TABLE 14) produce decreased amounts of total protein and decreased amounts of CBH1 under such lactose inducing conditions (relative to parental cell).

Likewise, regulatory proteins whose deletion resulted in decreased protein production at 34° C. in glucose releasing plates after one-hundred twenty (120) hours of incubation are set forth in TABLE 15, wherein the numerical values represent performance indexes (PIs) calculated for PASC enzymatic assay, HPLC measurements of CBH1 or total protein measured by Bradford relative to the control (wild-type) strain QM6a Δku80.

TABLE 15
Regulatory Protein Deletions Resulting in Decreased
Protein Production at 34° C. in Glucose
Releasing Plates After 120 Hours of Incubation

| Regulatory Protein | PASC | CBH1 | Total Protein |
|---|---|---|---|
| Trire2 22774 (SEQ ID NO: 89) | 0.47 | 0.73 | 0.87 |
| Trire2 22785 (SEQ ID NO: 90) | 0.27 | 0.73 | 0.87 |
| Trire2 44290 (SEQ ID NO: 92) | — | 0.54 | 0.77 |
| Trire2 44781 (SEQ ID NO: 93) | — | 0.74 | 0.87 |
| Trire2 48773 (SEQ ID NO: 95) | 0.40 | 0.79 | 0.90 |
| Trire2 72076 (SEQ ID NO: 115) | — | 0.63 | 0.82 |
| Trire2 106009 (SEQ ID NO: 127) | 0.49 | 0.78 | 0.89 |
| Trire2 123713 (SEQ ID NO: 136) | 0.13 | 0.76 | 0.88 |

Thus, as presented above in TABLE 15, the mutant cells comprising a deletion of regulatory protein Trire2_22774 (SEQ ID NO: 89), Trire2_22785 (SEQ ID NO: 90), Trire2_44290 (SEQ ID NO: 92), Trire2_44781 (SEQ ID NO: 93), Trire2_48773 (SEQ ID NO: 95), Trire2_72076 (SEQ ID NO: 115), Trire2_106009 (SEQ ID NO: 127) or Trire2_123713 (SEQ ID NO: 136), comprise reduced protein productivity phenotypes (relative to parental cells) when fermented at 34° C. under glucose releasing conditions. For example, the mutant cells deficient in the production of the aforementioned regulatory proteins (TABLE 15) produce decreased amounts of total protein and decreased amounts of CBH1, under such glucose releasing conditions (relative to parental cell).

### Example 7

### Fungal Regulatory Protein Orthologues

In the instant example Applicant has performed a homology based search of 36 regulatory factors against fungal species Aspergillus niger ATCC 1015 v4.0, Myceliophthora thermophila (Sporotrichum thermophila) v2.0, and Aspergillus oryzae RIB40. As shown below, TABLE 16 lists orthologs identified for these selected group of fungal species.

TABLE 16
FILAMENTOUS FUNGAL REGULATORY PROTEIN HOMOLOGUES SHOWING
PERCENT IDENTITY TO TRICHODERMA REGULATORY PROTEINS

| T. reesei (SEQ\hat{ }) | A. niger (PID) | M. thermophili (PID) | A. oryzae (PID) |
|---|---|---|---|
| Trire2 4933 FL* (2) | — | 70.9% (2304897) | 53.1% (7375) |
| Trire2 4933 BD** (3) | 69.2% (1186540) | 98.1% (2304897) | 67.3% (7375) |
| Trire2 4933 BD** (2) | — | 56.8% (2088766) | — |
| Trire2 5675 FL* (5) | 55.6% (1179951) | 61.6% (2300604) | 53.1% (6190) |
| Trire2 5675 FL* (5) | 47.6% (1200945) | 51.7% (2304963) | — |
| Trire2 5675 BD** (6) | 65.0% (1179951) | 84.3% (2300604) | 65.5% (6190) |
| Trire2 5675 BD** (6) | 46.0% (1200945) | 51.3% (2304963) | — |
| Trire2 48438 FL* (8) | 55.2% (1206310) | — | 57.7% (1090) |
| Trire2 48438 BD** (9) | 65.9% (1206310) | — | 64.8% (1090) |
| Trire2 48438 BD** (9) | 52.4% (1128327) | — | 51.2% (7738) |
| Trire2 49232 FL* (11) | 54.3% (1188901) | 69.5% (2300537) | 52.7% (6142) |
| Trire2 49232 BD** (12) | 93.7% (1188901) | 95.2% (2300537) | 90.5% (6142) |
| Trire2 55105 FL* (14) | 55.2% (1116788) | 51.7% (111741) | 54.8% (7325) |
| Trire2 55105 FL* (14) | 54.8% (1148756) | — | 50.0% (9792) |
| Trire2 55105 FL* (14) | 55.2% (1156125) | — | — |
| Trire2 55105 FL* (14) | 51.7% (1117887) | — | — |
| Trire2 55105 FL* (14) | 50.0% (1093745) | — | — |
| Trire2 55105 BD** (15) | 61.5% (1125716) | 65.2% (117478) | 59.0% (4157) |
| Trire2 55105 BD** (15) | 59.3% (1156125) | 55.6% (111741) | 59.1% (3066) |
| Trire2 55105 BD** (15) | 59.3% (1148756) | 55.6% (2307957) | 55.6% (7325) |
| Trire2 55105 BD** (15) | 55.6% (1157619) | 53.8% (2311613) | 55.6% (3749) |
| Trire2 55105 BD** (15) | 55.6% (1204072) | 51.9% (2306976) | 51.9% (9792) |
| Trire2 55105 BD** (15) | 51.9% (1117887) | 51.9% (301920) | 51.9% (3816) |
| Trire2 55105 BD** (15) | 51.9% (1167351) | — | 51.9% (3368) |
| Trire2 55105 BD** (15) | 51.9% (1116788) | — | 51.9% (2288) |
| Trire2 55105 BD** (15) | 51.9% (1158467) | — | — |
| Trire2 60565 FL* (17) | — | — | 53.3% (4307) |
| Trire2 60565 BD** (18) | 64.5% (1128851) | — | — |
| Trire2 60931 FL* (20) | — | — | — |
| Trire2 60931 BD** (21) | 58.1% (1161181) | — | — |
| Trire2 67209 FL* (23) | — | 63.8% (2300332) | — |
| Trire2 67209 BD** (24) | — | 83.6% (2300332) | — |
| Trire2 68097 FL* (26) | 67.2% (1088940) | 60.5% (2295245) | 67.4% (802) |
| Trire2 68097 BD** (27) | 85.5% (1088940) | 76.5% (2295245) | 84.1% (802) |
| Trire2 68425 FL* (29) | 56.3% (1183850) | — | — |
| Trire2 68425 BD** (30) | 58.2% (1183850) | — | — |
| Trire2 69695 FL* (32) | — | — | 50.0% (4348) |
| Trire2 69695 BD** (33) | 62.5% (1160043) | 56.8% (2307148) | 58.8% (25) |
| Trire2 69695 BD** (33) | 52.6% (1123787) | 56.0% (2071661) | 56.7% (4348) |
| Trire2 69695 BD** (33) | 60.0% (1124751 | 52.4% (2089183) | 54.2% (8652) |
| Trire2 69695 BD** (33) | 58.3% (1124535) | — | 61.9% (3880) |
| Trire2 69695 BD** (33) | 50.0% (1220789) | — | 54.2% (2999) |
| Trire2 69695 BD** (33) | — | — | 57.1% (3368) |
| Trire2 69695 BD** (33) | — | — | 57.1% (968) |
| Trire2 69695 BD** (33) | — | — | 56.0% (9186) |
| Trire2 69695 BD** (33) | — | — | 52.0% (5668) |
| Trire2 72993 FL* (38) | 57.9% (1159485) | 59.6% (117478) | 58.5% (89) |
| Trire2 72993 BD** (39) | 53.8% (1159485) | 58.0% (117478) | 58.5% (89) |
| Trire2 72993 BD** (39) | 55.2% (1200706) | 54.1% (2305522) | 54.2% (2292) |
| Trire2 72993 BD** (39) | — | 52.4% (44521) | — |
| Trire2 76705 FL* (41) | — | 78.7% (2089183) | — |
| Trire2 76705 BD** (42) | — | 88.5% (2089183) | — |
| Trire2 76872 FL* (44) | 68.9% (1012915) | 60.7% (2310857) | — |
| Trire2 76872 BD** (45) | 66.7% (1012915) | 66.7% (2310857) | — |
| Trire2 76872 BD** (45) | 51.3% (1209182) | 54.6% (2296022) | — |
| Trire2 76872 BD** (45) | 58.6% (1127416) | — | — |
| Trire2 77291 FL* (47) | — | — | — |
| Trire2 77291 BD** (48) | 50.6% (1083714) | 53.9% (2296636) | 55.6% (3562) |
| Trire2 77291 BD** (48) | — | 50.0% (2305186) | — |
| Trire2 78162 FL* (50) | — | 59.1% (2135338) | — |
| Trire2 78162 BD** (51) | 50.0% (1228664) | 61.8% (2135338) | — |
| Trire2 103122 FL* (53) | 55.7% (1169563) | 55.8% (2309486) | — |
| Trire2 103122 BD** (54) | 57.6% (1169563) | 51.5% (2309486) | 57.7% (4087) |
| Trire2 103122 BD** (54) | 51.4% (1119789) | 51.5% (2299922) | — |
| Trire2 103122 BD** (54) | 51.4% (1165208) | — | — |
| Trire2 103122 BD** (54) | 60.0% (1119145) | — | — |
| Trire2 105239 FL* (56) | 55.0% (1095405) | — | |
| Trire2 105239 BD** (57) | 71.6% (1095405) | — | 57.9% (9404) |
| Trire2 105239 BD** (57) | 63.2% (1156261) | — | 54.2% (10329) |
| Trire2 105239 BD** (57) | 51.4% (1083117) | — | |
| Trire2 108775 FL* (65) | 58.2% (1181919) | — | 55.9% (3447) |
| Trire2 108775 FL* (65) | — | — | 52.7% (7195) |
| Trire2 108775 BD** (66) | 62% (1181919) | — | 59.3% (3447) |
| Trire2 108775 BD** (66) | — | — | 56.0% (7195) |
| Trire2 119986 FL* (68) | — | 54.8% (2312830) | 54.8% (10798) |
| Trire2 119986 FL* (68) | — | 51.6% (2132970) | 53.1% (5165) |
| Trire2 119986 FL* (68) | — | 51.5% (2303722) | 51.5% (305) |
| Trire2 119986 FL* (68) | — | 50.0% (2301134) | — |
| Trire2 119986 BD** (69) | 62.1% (1096755) | 65.5% (2296201) | 58.6% (2613) |
| Trire2 119986 BD** (69) | 55.2% (1150273) | 51.7% (2303722) | 58.6% (10224) |
| Trire2 119986 BD** (69) | 51.7% (1184246) | 51.7% (2301134) | 55.2% (5165) |
| Trire2 119986 BD** (69) | 58.6% (1206310) | 51.7% (2312830) | 55.2% (10224) |
| Trire2 119986 BD** (69) | 55.2% (1222568) | 51.7% (2132970) | 55.2% (8745) |
| Trire2 119986 BD** (69) | 51.7% (1118447) | 51.7% (107295) | 51.7% (305) |
| Trire2 119986 BD** (69) | 55.2% (1141737) | 51.7% (2296636) | 51.7% (3183) |
| Trire2 119986 BD** (69) | 55.2% (1127964) | — | 51.7% (4550) |
| Trire2 119986 BD** (69) | 51.7% (1185996) | — | 51.7% (11736) |
| Trire2 119986 BD** (69) | 51.7% (1112969) | — | — |
| Trire2 120428 FL* (71) | 58.2% (1092066) | 65.4% (105777) | 56.6% (105777) |
| Trire2 120428 BD** (72) | 84.6% (1092066) | 90.9% (105777) | 81.8% (105777) |
| Trire2 120597 FL* (74) | 66.7% (1040620) | 74.5% (2313746) | 64.9% (9497) |
| Trire2 120597 BD** (75) | 66.7% (1040620) | 81.4% (2313746) | 64.9% (9497) |
| Trire2 121757 FL* (77) | — | — | — |
| Trire2 121757 BD** (78) | — | — | 50.0% (1626) |
| Trire2 122783 FL* (80) | 51.1% (1150324) | 55.6% (2300906) | 51.5% (7102) |
| Trire2 122783 FL* (80) | — | 50.0% (2118798) | 51.5% (5746) |
| Trire2 122783 BD** (81) | 58.6% (1158341) | 54.5% (2300906) | 53.3% (7102) |
| Trire2 122783 BD** (81) | 51.5% (1160672) | — | 51.7% (6717) |
| Trire2 122783 BD** (81) | 51.5% (1180825) | — | 51.5% (4087) |

The first (1^{st}) column abbreviation “FL*” refers to the “full-length amino acid sequence” of the T. reesei regulatory protein, followed by its associated sequence identification number in parenthesis (SEQ ID NO); the first (1^{st}) column abbreviation “BD**” refers to the to “predicted amino acid sequences” of DNA binding domain (DBD) present in the full-length amino acid sequence of the regulatory protein, followed by its associated sequence identification number in parenthesis (SEQ ID NO); the 1^{st} column heading “SEQ\hat{ }” is an abbreviation for “Sequence Identification (ID) Number (SEQ ID NO)”; and “PID” is an abbreviation for Protein Identification Number of the JGI database Aspergillus niger ATCC 1015 v4.0, Myceliophthora thermophila (Sporotrichum thermophile) v2.0 and Aspergillus oryzae RIB40 (Nordberg et al., 2014).

### REFERENCES

EPO Publication No. 0215594
PCT Publication No. WO 2021/102238
PCT Publication No. WO2008/097619
PCT Publication No. WO2005/056772
PCT Publication No. WO2008/080017
PCT Publication No. WO2011/151512
PCT Publication No. WO2011/151513
PCT Publication No. WO2011/151515
PCT Publication No. WO2011/153449
PCT Publication No. WO2013/102674
PCT Publication No. WO2014/093275
PCT Publication No. WO2016/100272
U.S. Pat. No. 6,022,725
U.S. Pat. No. 6,255,115
U.S. Pat. No. 6,268,328
Aro et al., “Transcriptional regulation of plant cell wall degradation by filamentous fungi” FEMS Microbiology Reviews 29: 719-739. 2005.
Ausubel et al., “Current Protocols in Molecular Biology, published by Greene Publishing Assoc. and Wiley-Interscience (1987, 1989 and 2003).
Boel et al., “Two different types of intervening sequences in the glucoamylase gene from Aspergillus niger”, EMBO J. 3:1581-1585, 1984.
Campbell et al., Curr. Genet., 16:53-56, 1989.
Cao et al., “Penicillopepsin-JT2, a recombinant enzyme from Penicillium janthinellum and the contribution of a hydrogen bond in subsite S3 to kcat”, Protein Science (9):991-1001, 2000.
Devereux et al., Nucleic Acids Res. 12:387-395, 1984.
Druzhinina and Kubicek, “Genetic engineering of T. reesei cellulases and their production”, Microb. Biotechnol. 10, 1485-1499, 2017.
Fitz et al. “The Promoter Toolbox for Recombinant Gene Expression in Trichoderma reesei”, Frontiers in Bioengineering and Biotechnology, Volume 6, Article 135, 2018.
Harkki et al., “A Novel Fungal Expression System: Secretion of Active Calf Chymosin from the Filamentous Fungus Trichoderma reesei”, BioTechnol., 7: 596-603, 1989.
Harkki et al., “Genetic engineering of Trichoderma to produce strains with novel cellulase profiles”, Enzyme Microb. Technol., 13: 227-233, 1991.
Ilmen et al., “Regulation of cellulase gene expression in the filamentous fungus Trichoderma reesei”, Applied and Environmental Microbiology, 63(4)-1298-1306, 1997.
Kriegler, Gene Transfer and Expression: A Laboratory Manual, 1990.
Kubicek et al., “Metabolic engineering strategies for the improvement of cellulase production by Hypocrea jecorina”, Biotechnol. Biofuels 2:19, 2009.
Latchman, “Transcription factors: an overview”, Int. J. Exp. Path., 74, 417-422, 1993.
Lever, “A new reaction for colorimetric determination of carbohydrates”, Anal. Biochem., 47:273-279, 1972.
Li, Wan-Chen et al. “Trichoderma reesei complete genome sequence, repeat-induced point mutation, and partitioning of CAZyme gene clusters.” Biotechnology for biofuels Vol. 10 (170), 2017.
Mullaney et al., “Primary structure of the trpC gene from Aspergillus nidulans”, Molecular and General Genetics 199:37-45, 1985.
Needleman and Wunsch, “A general method applicable to the search for similarities in the amino acid sequence of two proteins”, J. Mol. Biol. 48: 443-453, 1970.
Nordberg et al., “The genome portal of the Department of Energy Joint Genome Institute: 2014 updates”, Nucleic Acids Res., 42(1):D26-31, 2014.
Nunberg et al., “Molecular cloning and characterization of the glucoamylase gene of Aspergillus awamori”, Mol. Cell Biol. 4:2306, 1984.
Pearson and Lipman, Proc. Natl. Acad. Sci. USA 85:2444, 1988.
Portnoy et al., “Differential Regulation of the Cellulase Transcription Factors XYR1, ACE2, and ACE1 in Trichoderma reesei Strains Producing High and Low Levels of Cellulase”, Eukaryotic Cell, 10(2): 262-271, 2011.
Rice et al., “EMBOSS: The European Molecular Biology Open Software Suite”, Trends in Genetics 16: 276-277, 2000.
Sambrook et al., Molecular Cloning, A Laboratory Manual, 2^{nd} Edition, Cold Spring Harbor Laboratory Press, Cold Spring, New York, 1989.
Sambrook et al., Molecular Cloning, A Laboratory Manual, 4^{th} Edition, Cold Spring Harbor Laboratory Press, Cold Spring, New York, 2012.
Sheir-Neiss and Montenecourt, “Characterization of the secreted cellulases of Trichoderma reesei wild type and mutants during controlled fermentations”, Applied Microbiology and Biotechnology, 20(1):46-53, 1984.
Smith and Waterman, Adv. Appl. Math. 2:482, 1981.
Stricker et al., “Regulation of transcription of cellulases- and hemicellulases-encoding genes in Aspergillus niger and Hypocrea jecorina (Trichoderma reesei), Appl. Microbiol. Biotechnol. (Review), 78(2):211-220, 2008.
Wang et al., “Fermentation & Enzyme Technology”, John Wiley & Sons, 1979.
Wood, “The cellulase of Fusarium solani. Purification and specificity of the β-(1→4)-glucanase and the β-d-glucosidase components”, Biochem. J., 121:353-362, 1971.
Wu et al., “The regulatory and transcriptional landscape associated with carbon utilization in a filamentous fungus”, PNAS, 117(11): 6003-6013, 2020.
Yelton et al., “Transformation of Aspergillus nidulans by using a trpC plasmid”, PNAS USA 81:1470-1474, 1984.

1. A recombinant fungal cell deficient in the production of a regulatory protein comprising at least 95% identity to the regulatory protein of SEQ ID NO: 44.

2-4. (canceled)

5. The recombinant fungal cell of claim 1, comprising an introduced expression cassette encoding a heterologous protein of interest (POI).

6-7. (canceled)

8. The recombinant fungal cell of claim 1 expressing an endogenous protein of interest (POI).

9. (canceled)

10. The recombinant fungal cell of claim 5, wherein the introduced cassette comprises an upstream (5′) cellulase gene promoter operably linked to a downstream (3′) nucleic acid encoding the heterologous POI.

11. (canceled)

12. The recombinant fungal cell of claim 8, wherein the endogenous POI is a cellulase.

13. The recombinant fungal cell of claim 9, wherein the heterologous POI is selected from the group consisting of enzymes, peptides, antibodies, receptor proteins, animal feed proteins, human food proteins, protein biologics, therapeutic proteins and immunogenic proteins.

14. A method for the enhanced production of a cellulase comprising:
(a) obtaining a parental fungal cell expressing a cellulase, genetically modifying the parental cell to obtain a recombinant fungal cell deficient in the production of a native regulatory protein comprising at least 95% identity to the regulatory protein of SEQ ID NO: 44, and
(b) cultivating the recombinant cell,
wherein the recombinant cell produces an increased amount of a cellulase relative to the parental cell cultivated under the same conditions.

15. The method of claim 14, wherein the recombinant cell further comprises an introduced polynucleotide encoding a regulatory protein comprising at least 95% identity to the regulatory protein of SEQ ID NO: 85.

16-17. (canceled)

18. The method of claim 14, wherein the cellulase is selected from the group consisting of cellobiohydrolases, hemicellulases, endoglucanases and β-glucosidases.

19. A method for the enhanced production of a heterologous protein of interest (POI) comprising:
(a) introducing into a parental fungal cell an expression cassette encoding the heterologous POI, genetically modifying the parental cell to obtain a recombinant fungal cell deficient in the production of a native regulatory protein comprising at least 95% identity to the regulatory protein of SEQ ID NO: 44, and
(b) cultivating the recombinant cell,
wherein the recombinant cell produces an increased amount of the POI relative to the parental cell cultivated under the same conditions.

20. The method of claim 19, wherein the cassette encoding the POI comprises an upstream (5′) cellulase gene promoter operably linked to a downstream (3′) nucleic acid encoding the POI.

21. The method of claim 19, wherein the POI is selected from the group consisting of enzymes, peptides, antibodies, receptor proteins, animal feed proteins, human food proteins, protein biologics, therapeutic proteins and immunogenic proteins.
